(19) United States
(12) Patent Application Publication
(10) Pub. No.: US 2026/0354016 A1
(43) Pub. Date: Nov. 5, 2026

(54) COMPOSITION AND ORGANIC ELECTROLUMINESCENT DEVICE COMPRISING SAME

(71) Applicant: FUYANG SINEVA NEW MATERIAL TECHNOLOGY CO., LTD., Anhui (CN)

(72) Inventors: Zhanqi WANG, Beijing (CN); Zhiqiang LI, Anhui (CN); Jinbo LU, Anhui (CN); Changgang HUANG, Beijing (CN)

(21) Appl. No.: 19/114,880

(22) PCT Filed: Sep. 25, 2023
(86) PCT No.: PCT/CN2023/121162

§ 371 Date: Mar. 25, 2025

(30) Foreign Application Priority Data
Sep. 30, 2022 (CN) 202211215054.0

(51) Int. Cl. H10K 85/60; C07B 59/00; C07D 403/10; C09K 11/02; H10K 50/18

(52) U.S. Cl. CPC H10K 85/6572; C07B 59/002; C07D 403/10; C09K 11/02; H10K 85/622; H10K 85/626; H10K 85/654; H10K 50/181

(57) ABSTRACT

The present application provides a composition and an organic electroluminescent device comprising same. The composition comprises two compounds represented by formula I and a compound represented by formula II; or the composition comprises a compound represented by formula I and two compounds represented by formula II. The compound represented by formula II is obtained by fusing a group represented by formula I-C with any two adjacent carbon atoms on ring E in a group represented by formula I-D. The composition provided in the present application can be used as a light-emitting layer material of the organic electroluminescent device, such that the organic electroluminescent device with excellent performance is prepared.

The present application claims priority to Chinese Patent Application No. CN 202211215054.0 filed on Sep. 30, 2022, and the disclosure of which is incorporated herein by reference in its entirety.

### TECHNICAL FIELD

The present application belongs to the technical field of organic electroluminescent material, and specifically relates to a composition and an organic electroluminescent device comprising the same.

### BACKGROUND

Organic electroluminescence phenomenon has been found as early as 1963, but it initially failed to attract significant attention. It wasn't until 1987 when the Tang research group of Kodak Company in the United States reported a DC low-voltage-driven, high-brightness, high-efficiency thin-film organic electroluminescent device (OLED) using organic fluorescent materials and hole transport materials that this technology regained prominence and ushered in a new research field.

OLED technology has outstanding advantages over other display technologies, such as low power consumption, fast response speed, flexibility, wide viewing angles, capability for large-area displays, full-color emission, etc., and compatibility with varieties of existing standards and technologies for cost-effective light-emitting devices. These features have revealed broad application prospects in achieving full-color flat-panel displays. Over the past few decades, OLED as a novel display technology has achieved substantial progress, finding widespread applications in flat-panel displays, flexible displays, solid-state lighting, and automotive displays.

Currently, organic electroluminescence (OLED) has emerged as a mainstream display technology, accompanied by the development of various novel OLED materials. However, critical performance aspects such as efficiency, operational lifetime, and driving voltage still require improvement. Consequently, the research focus in this field lies in developing more diverse and optimized blue-emitting host materials to meet the demands of high-performance OLED devices.

### SUMMARY

The following is a summary of the subject described in detail herein. This summary is not intended to limit the protection scope of the claims.

In view of the shortcomings of the prior art, an object of the present application is to provide a composition and an organic electroluminescent device comprising the same. In the present application, a composition with excellent performance is obtained by designing the specific components of the composition, and an organic electroluminescent device with excellent performance can be fabricated by using this composition as the material of the emissive layer of the organic electroluminescent device.

To achieve the object, the present application adopts the following technical solutions.

In a first aspect, the present application provides a composition, and the composition includes two compounds represented by Formula I and one compound represented by Formula II;
or the composition includes one compound represented by Formula I and two compounds represented by Formula II;
a structural formula of the compound represented by Formula I is as follows:

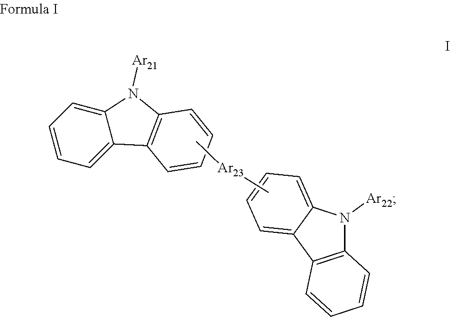

wherein Ar₂₁ and Ar₂₂ are each independently selected from C6 to C30 aryl or C6 to C20 heteroaryl;
Ar₂₃ is selected from any one of a single bond, phenylene, naphthylene, or biphenylene;
the hydrogen atom in the compound represented by Formula I can be substituted with at least one of —F, —CN, C6 to C20 aryl, C1 to C6 alkyl, or C1 to C6 alkoxy;
the compound represented by Formula I meets at least one of the following conditions:
(1) the compound represented by Formula I contains no deuterium atom;
(2) hydrogen atoms attached to two carbazolyl in the compound represented by Formula I are all substituted with deuterium atoms;
(3) at least one hydrogen atom in Ar₂₁ of the compound represented by Formula I is substituted with a deuterium atom;
(4) at least one hydrogen atom in Ar₂₂ of the compound represented by Formula I is substituted with a deuterium atom;
(5) Ar₂₃ in the compound represented by Formula I is phenylene and/or biphenylene, and at least one hydrogen atom in the Ar₂₃ is substituted with a deuterium atom;
(6) a hydrogen atom in the compound represented by Formula I is substituted with C6 to C20 aryl, and at least one hydrogen atom in the C6 to C20 aryl is substituted with a deuterium atom; and
(7) a hydrogen atom in the compound represented by Formula I is substituted with C1 to C6 alkyl and/or C1 to C6 alkoxy, and all hydrogen atoms in C1 to C6 alkyl and/or C1 to C6 alkoxy are substituted with deuterium atoms;
the compound represented by Formula II is obtained by fusing the group represented by Formula I-C with any two adjacent carbon atoms on ring E in the group represented by Formula I-D;

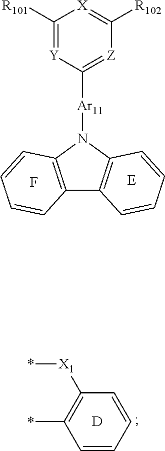

wherein “*” represents a fusion site;
Ar₁₁ is selected from any one of a single bond, phenylene, naphthylene, or biphenylene;
R₁₀₁ and R₁₀₂ are each independently selected from H, C6 to C30 aryl, or C6 to C20 heteroaryl;
X, Y, and Z are each independently selected from N or CR₃₀₄, wherein R₃₀₄ is selected from any one of H, phenyl, biphenyl, naphthyl, 9,9-dimethylfluorenyl, dibenzofuranyl, or dibenzothienyl, and at least one of X, Y, and Z is N;
X₁ is selected from O, S,

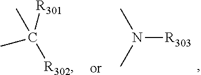

wherein R₃₀₁ and R₃₀₂ are each independently selected from C1 to C5 alkyl or phenyl, and R₃₀₃ is selected from phenyl or biphenyl, and the dashed line represents a linkage site;
the hydrogen atom in the compound represented by Formula II may be substituted with at least one of —F, —CN, C6 to C20 aryl, C1 to C6 alkyl, or C1 to C6 alkoxy;
the compound represented by Formula II meets at least one of the following conditions:
(a) the compound represented by Formula II contains no deuterium atom;
(b) hydrogen atoms on at least one of the ring F, ring D, and ring E in the compound represented by Formula II are all substituted with deuterium atoms;
(c) at least one hydrogen atom in R₁₀₁ of the compound represented by Formula II is substituted with a deuterium atom;
(d) at least one hydrogen atom in R₁₀₂ of the compound represented by Formula II is substituted with a deuterium atom;
(e) at least one hydrogen atom in Ar₁₁ of the compound represented by Formula II is substituted with a deuterium atom;
(f) hydrogen atoms in R₃₀₁ and R₃₀₂ of the compound represented by Formula II are all substituted with deuterium atoms;
(g) at least one hydrogen atom in R₃₀₃ of the compound represented by Formula II is substituted with a deuterium atom;
(h) R₃₀₄ in the compound represented by Formula II is a deuterium atom;
(i) in the compound represented by Formula II, when R₃₀₄ is selected from any one of phenyl, biphenyl, naphthyl, 9,9-dimethylfluorenyl, dibenzofuranyl, or dibenzothienyl, at least one hydrogen atom in the phenyl, biphenyl, naphthyl, 9,9-dimethylfluorenyl, dibenzofuranyl, or dibenzothienyl is substituted with a deuterium atom;
(j) a hydrogen atom in the compound represented by Formula II is substituted with C6 to C20 aryl, and at least one hydrogen atom in the C6 to C20 aryl is substituted with a deuterium atom; and
(k) a hydrogen atom in the compound represented by Formula II is substituted with C1 to C6 alkyl and/or C1 to C6 alkoxy, and all hydrogen atoms in C1 to C6 alkyl and/or C1 to C6 alkoxy are substituted with deuterium atoms.

In the present application, a composition with specific component is obtained by designing the specific components of the composition and further using the combination of at least two specific compounds, and an organic electroluminescent device with excellent performance can be fabricated by using this composition as the material of the emissive layer of the organic electroluminescent device.

In the present application, the C6 to C30 is selected from C6, C10, C12, C18, C24, or C30, etc.

The C6 to C20 is selected from C6, C10, C12, C18, or C20, etc.

The C1 to C6 is selected from C1, C2, C3, C4, C5, or C6.

The C1 to C5 is selected from C1, C2, C3, C4, or C5.

The following are optional technical solutions of the present application, but not as a limitation of the technical solutions provided in the present application. The object and beneficial effects of the present application can be better achieved and realized by the following optional technical solutions.

As an optional technical solution of the present application, the composition includes two compounds represented by Formula I and two compounds represented by Formula II.

It should be noted that the composition includes two compounds represented by Formula I (denoted as Compound 1-1 and Compound 1-2) and one compound represented by Formula II (denoted as Compound 2-1), and a volume ratio of Compound 1-1 to Compound 1-2 to Compound 2-1 is 1:(1-2):(1-6), which may be, for example, 1:1:1, 1:1:2, 1:1:3, 1:1:4, 1:2:1, 1:2:2, 1:2:3, 1:2:4, 1:2:5, or 1:2:6, etc.; or
the composition includes one compound represented by Formula I (denoted as Compound 1-1) and two compounds represented by Formula II (denoted as Compound 2-1 and Compound 2-2), and a volume ratio of Compound 1-1 to Compound 2-1 to Compound 2-2 is (1-6):(1-2):1, which may be, for example, 1:1:1, 2:1:1, 3:1:1, 4:1:1, 1:2:1, 2:2:1, 3:2:1, 4:2:1, 5:2:1, or 6:2:1, etc.; or
the composition includes two compounds represented by Formula I (denoted as Compound 1-1 and Compound 1-2) and two compounds represented by Formula II (denoted as Compound 2-1 and Compound 2-2), and a volume ratio of Compound 1-1 to Compound 1-2 is (2:8)-(8:2), which may be, for example, 2:8, 3:7, 4:6, 5:5, 6:4, 7:3, or 8:2, etc.; a volume ratio of Compound 2-1 to Compound 2-2 is (2:8)-(8:2), which may be, for example, 2:8, 3:7, 4:6, 5:5, 6:4, 7:3, or 8:2, etc., and a volume sum of Compound 1-1 and Compound 1-2 and a volume sum of Compound 2-1 and Compound 2-2 has a ratio of (2:8)-(8:2), which may be, for example, 2:8, 3:7, 4:6, 5:5, 6:4, 7:3, or 8:2, etc. In one embodiment, the compound represented by Formula I has a structure as represented by Formula I-1, or Formula I-2:

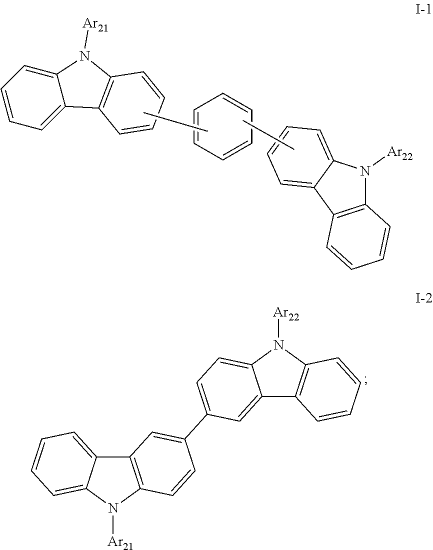

wherein Ar₂₁ and Ar₂₂ have the same protection scope as described above.

As an optional technical solution of the present application, the compound represented by Formula I meets condition (3) and/or condition (4).

In one embodiment, the compound represented by Formula I has a structure as represented by Formula I-1-D or Formula I-2-D:

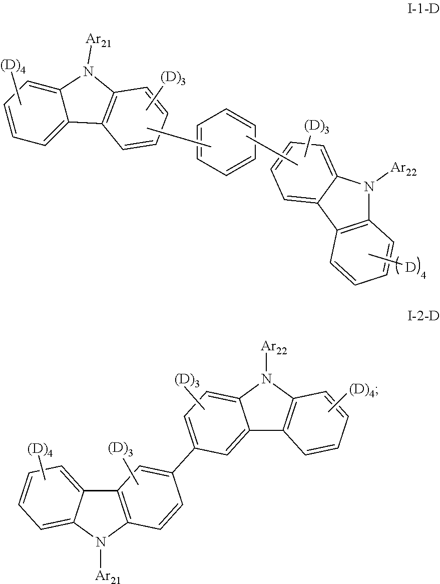

wherein Ar₂₁ and Ar₂₂ have the same protection scope as described above, and Ar₂₁ and Ar₂₂ contain no deuterium atom.

It should be noted that in the compound represented by Formula I-1-D and the compound represented by Formula I-2-D, (D)₄ means that four hydrogen atoms on the corresponding benzene ring are all substituted with deuterium atoms, and similarly, (D)₃ means that three hydrogen atoms on the corresponding benzene ring are all substituted with deuterium atoms, and neither Ar₂₁ nor Ar₂₂ contains deuterium atoms.

As an optional technical solution of the present application, Ar₂₁ and Ar₂₂ are each independently selected from any one or a combination of at least two of phenyl, biphenyl, naphthyl, fluorenyl, triphenylenyl, fluorenyl, dibenzofuranyl, dibenzothienyl, terphenyl, or quaterphenyl.

As an optional technical solution of the present application, the R₁₀₁ and the R₁₀₂ are each independently selected from any one of H, phenyl, naphthyl, triphenylenyl, fluoranthenyl, fluorenyl, anthryl, phenanthryl, biphenyl, naphthyl, dibenzofuranyl, or dibenzothienyl.

As an optional technical solution of the present application, the compound represented by Formula I is selected from any one of the following substituted or unsubstituted compounds:

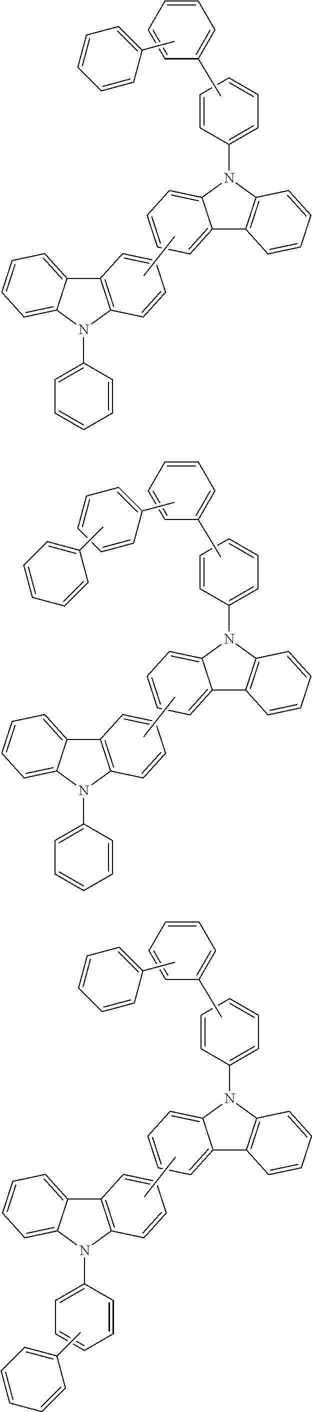

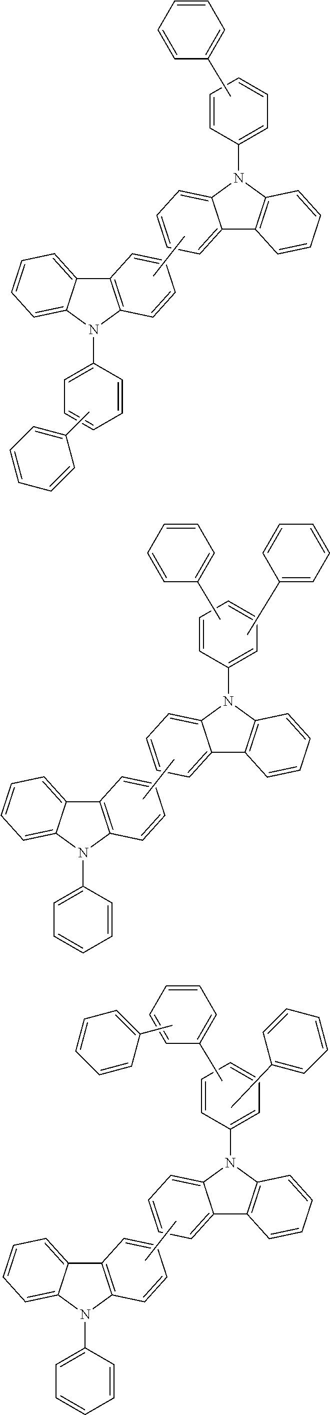

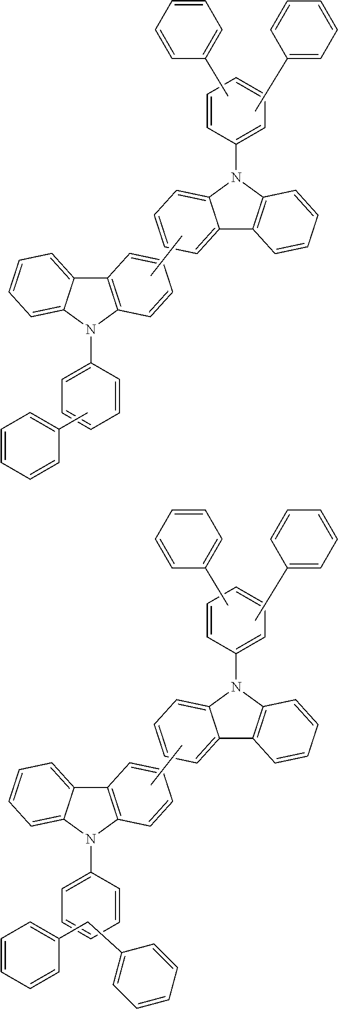

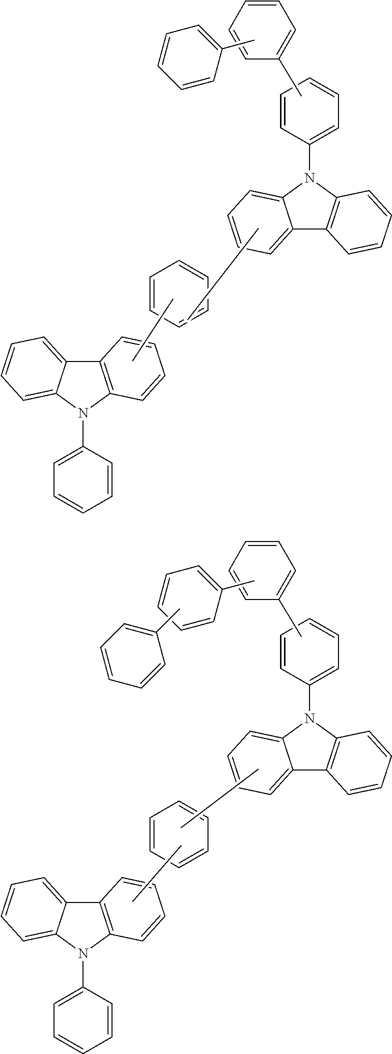

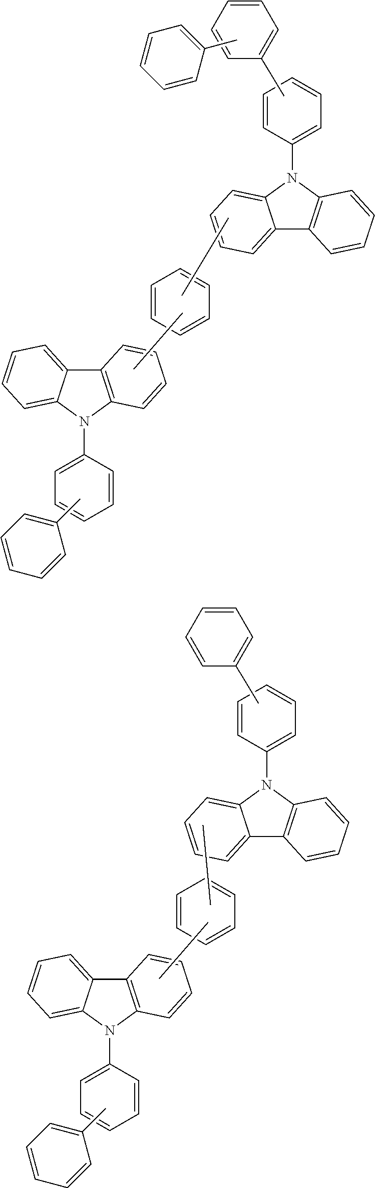

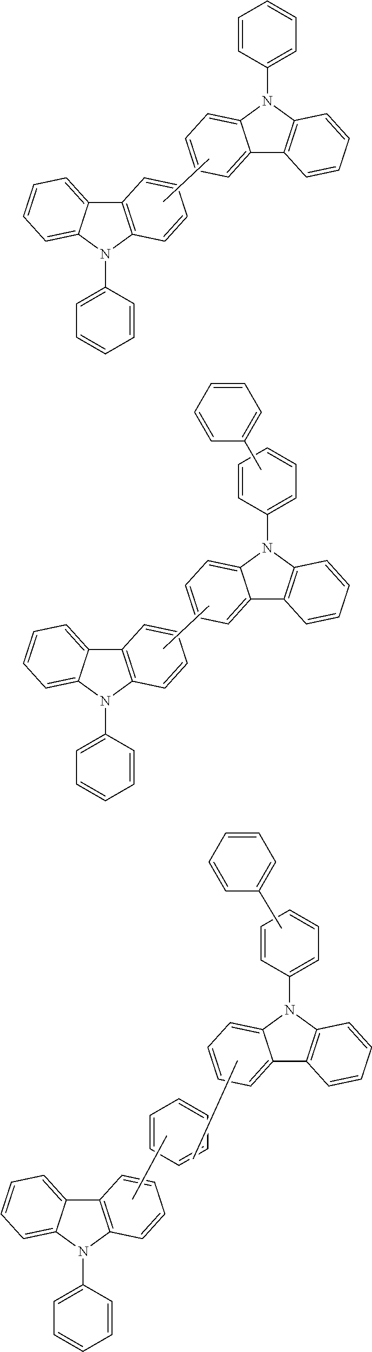

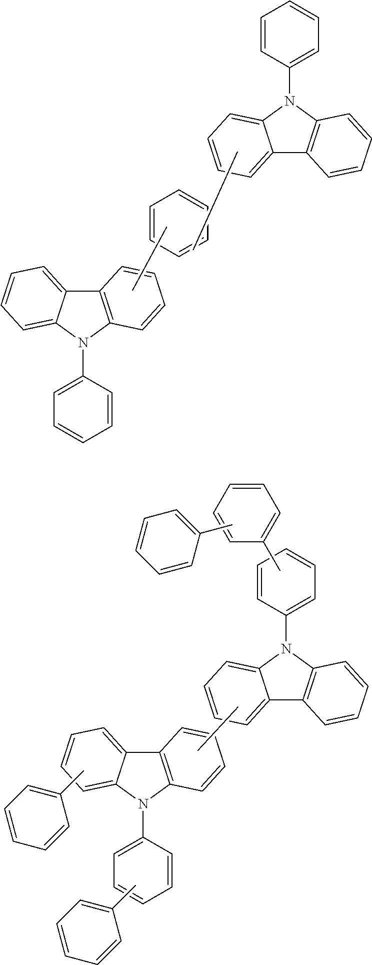

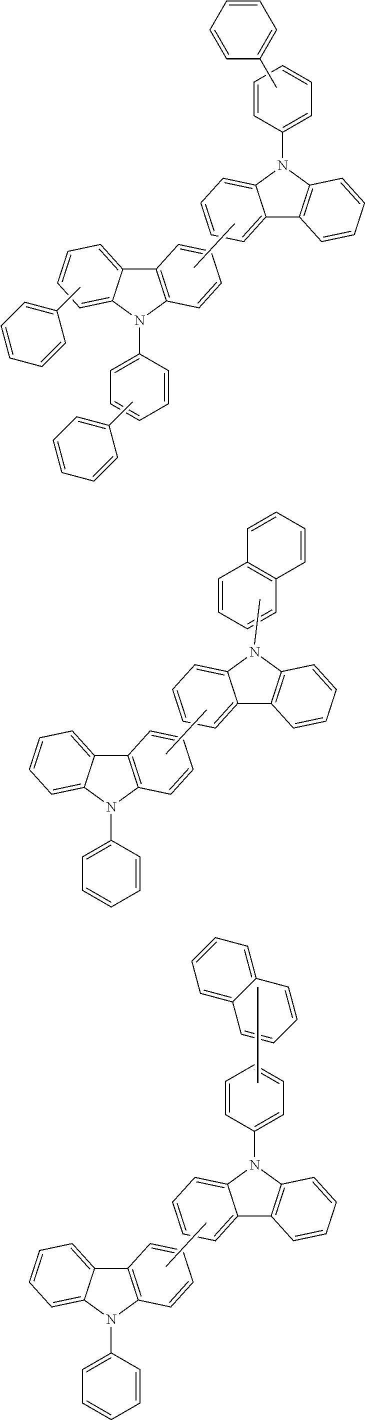

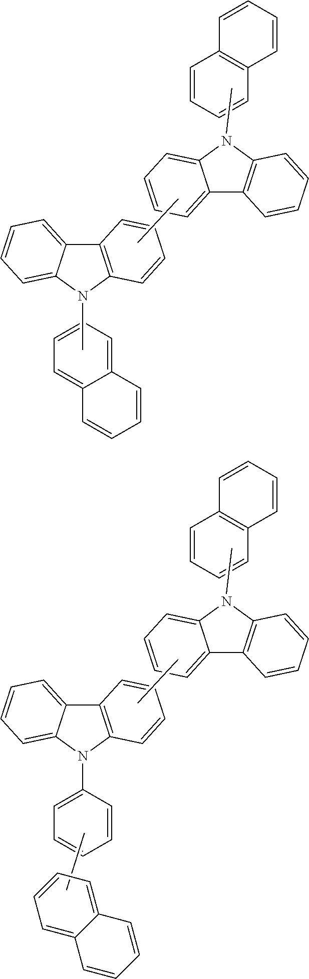

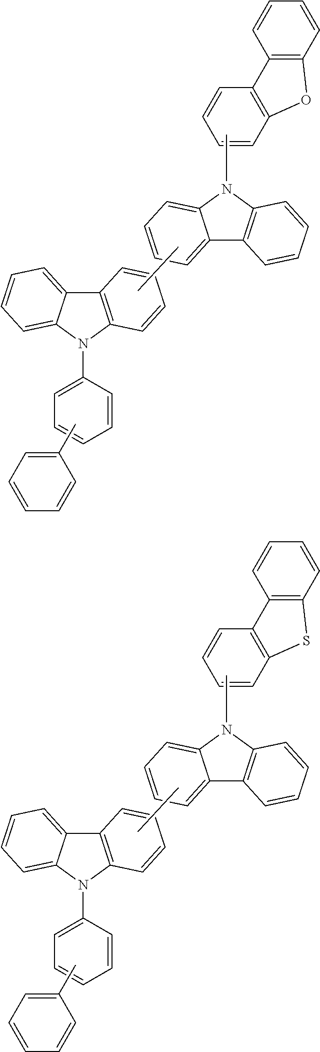

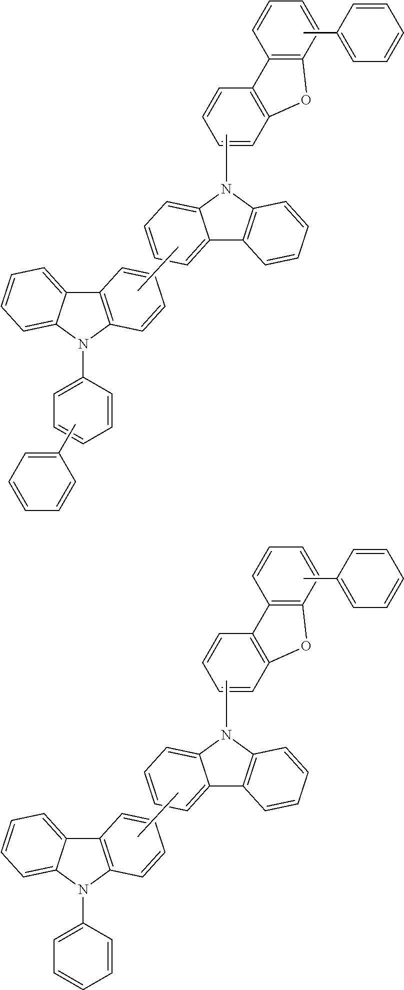

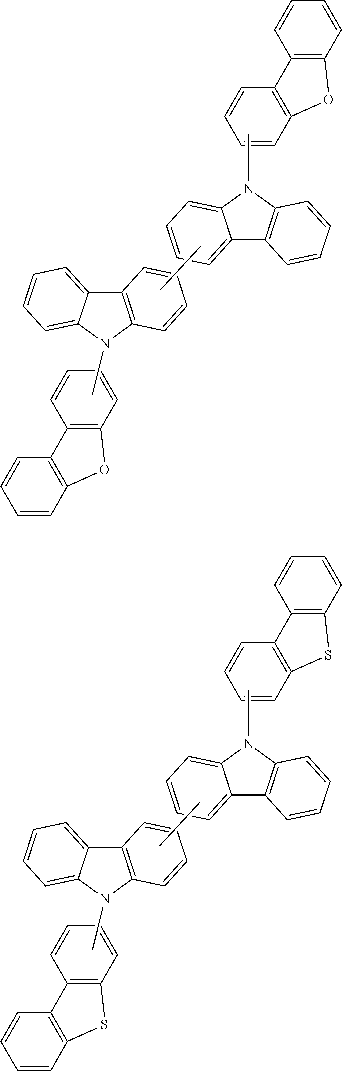

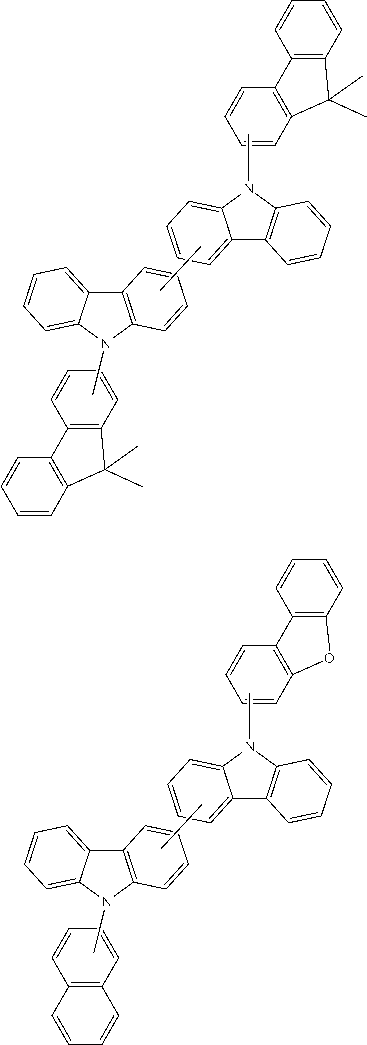

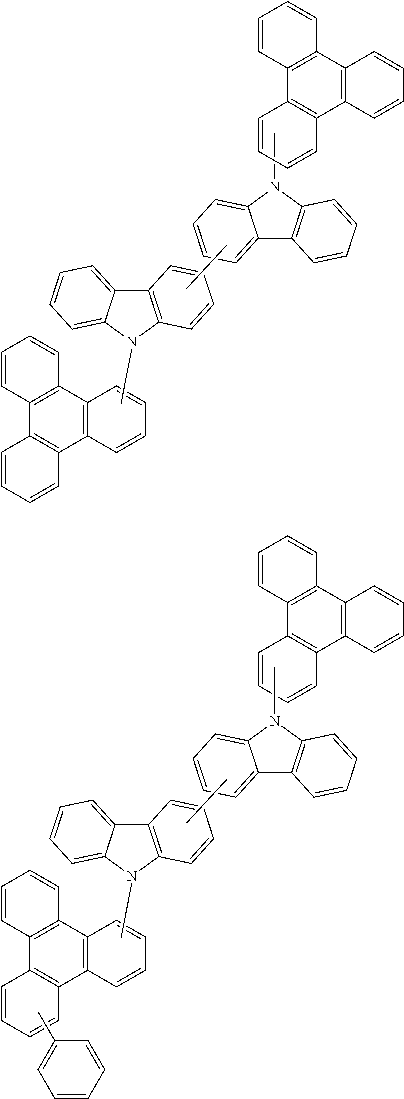

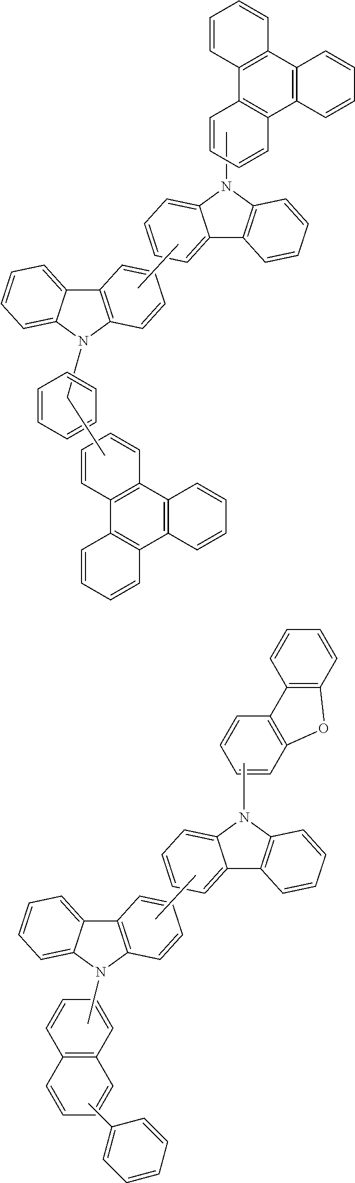

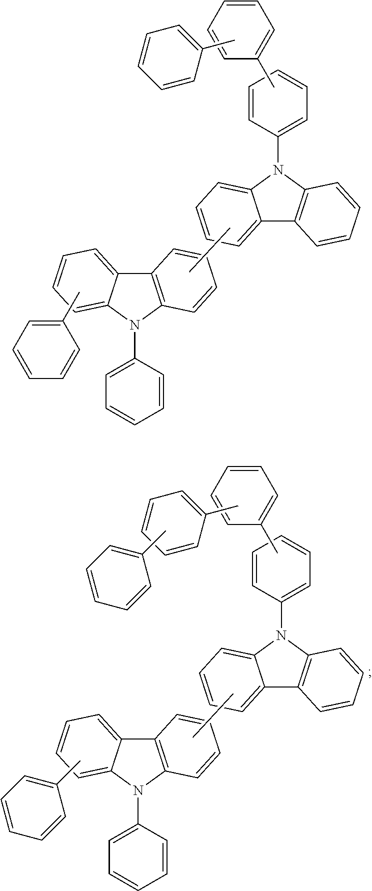

the “substituted” means that hydrogen atoms in the above compounds are each independently substituted with deuterium atoms.

In one embodiment, the compound represented by Formula I is selected from any one of the following substituted or unsubstituted compounds H-1 to H-40, H-17o, or H-17m:

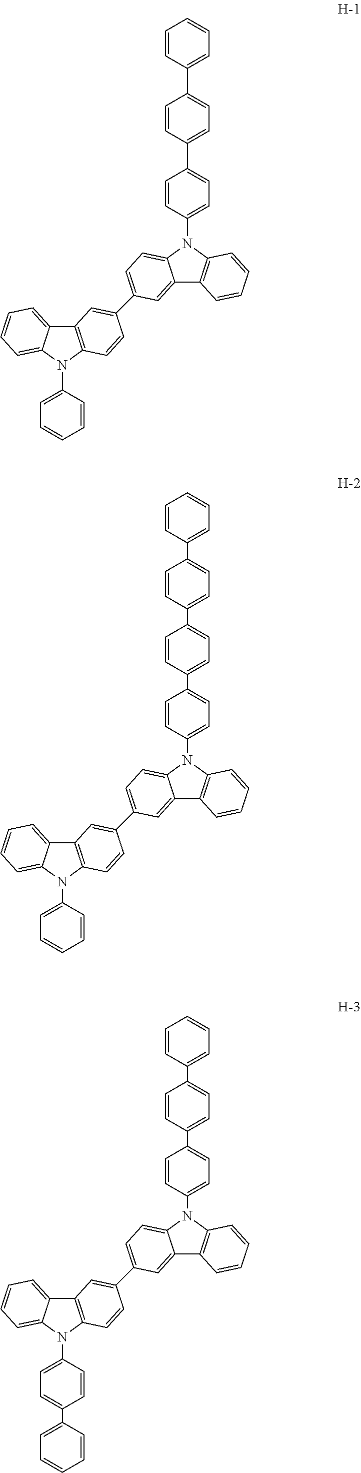 [SMILES: c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-c2ccc(-c3ccc(-c4ccc(-n5c6ccccc6c6cc(-c7ccc8c(c7)c7ccccc7n8-c7ccccc7)ccc65)cc4)cc3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1]

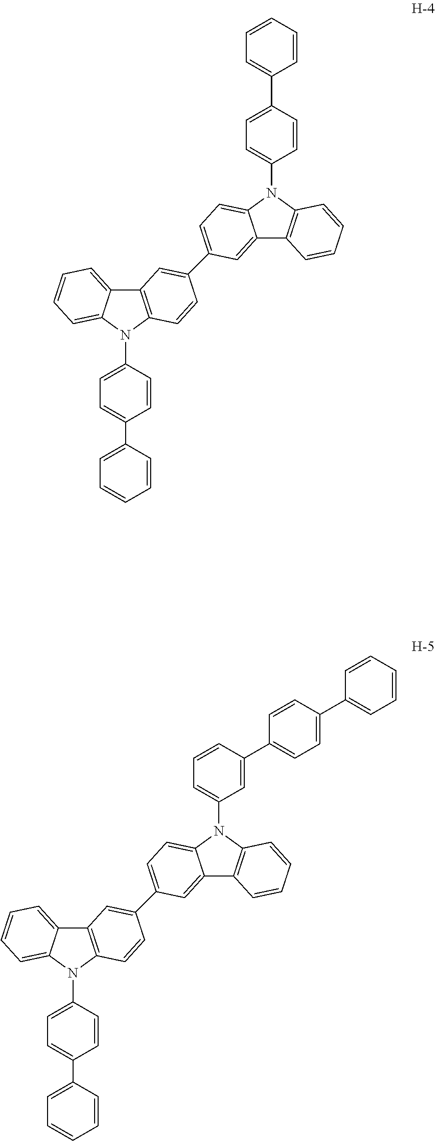 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1]

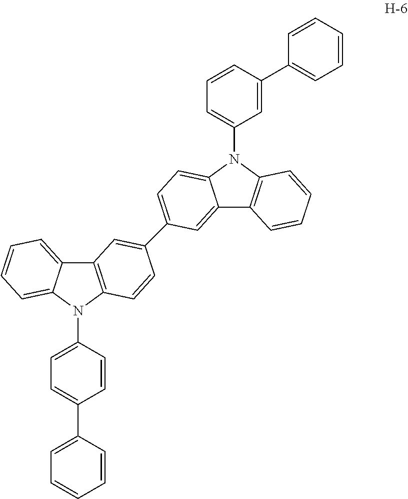 [SMILES: c1ccc(-c2ccc(-n3c4ccccc4c4cc(-c5ccc6c(c5)c5ccccc5n6-c5cccc(-c6ccccc6)c5)ccc43)cc2)cc1]

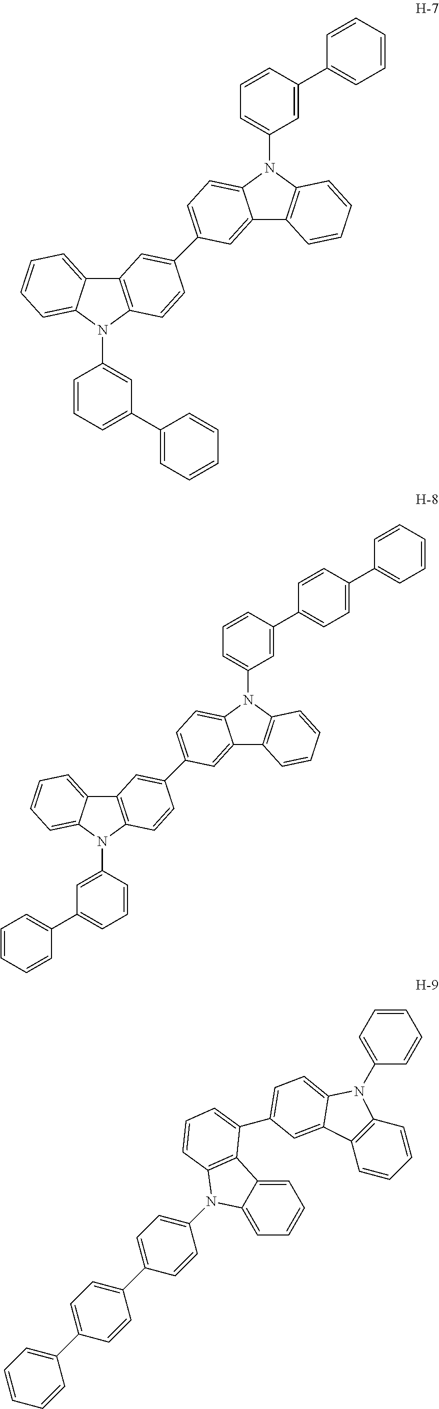 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2ccc(-c3ccc(-n4c5ccccc5c5c(-c6ccc7c(c6)c6ccccc6n7-c6ccccc6)cccc54)cc3)cc2)cc1]

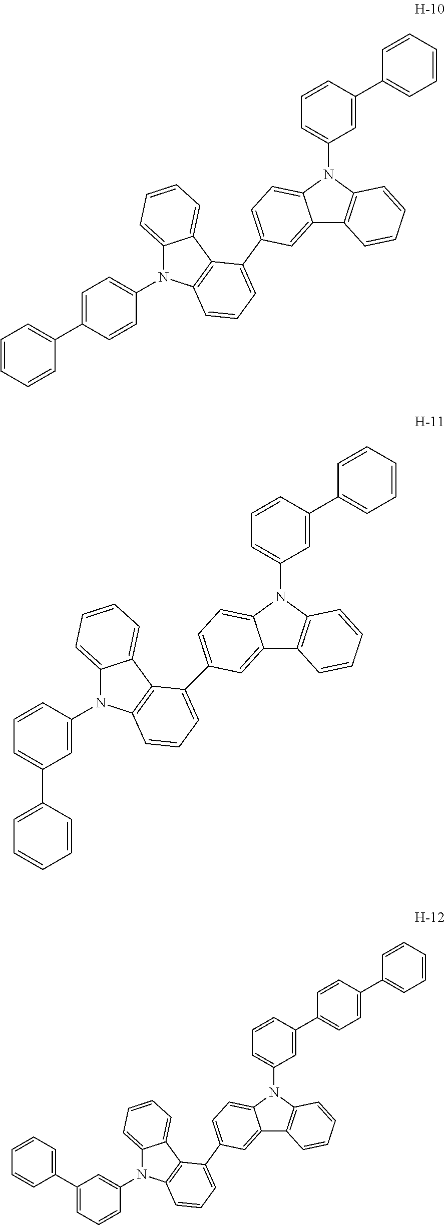 [SMILES: c1ccc(-c2ccc(-n3c4ccccc4c4c(-c5ccc6c(c5)c5ccccc5n6-c5cccc(-c6ccccc6)c5)cccc43)cc2)cc1 ; c1ccc(-c2cccc(-n3c4ccccc4c4cc(-c5cccc6c5c5ccccc5n6-c5cccc(-c6ccccc6)c5)ccc43)c2)cc1 ; c1ccc(-c2ccc(-c3cccc(-n4c5ccccc5c5cc(-c6cccc7c6c6ccccc6n7-c6cccc(-c7ccccc7)c6)ccc54)c3)cc2)cc1]

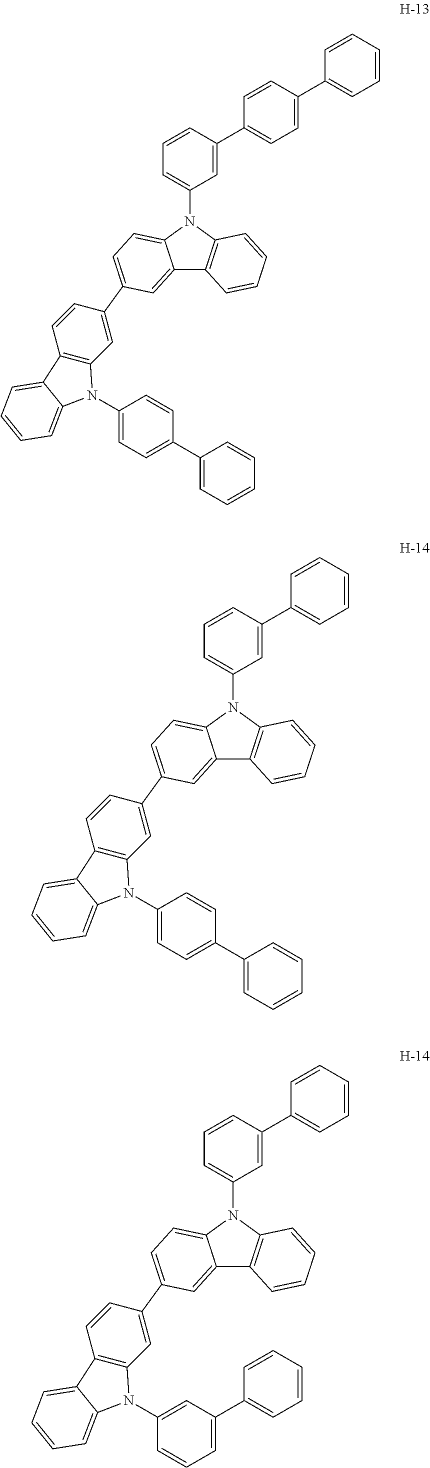 [SMILES: c1ccc(-c2ccc(-c3cccc(-n4c5ccccc5c5cc(-c6ccc7c8ccccc8n(-c8ccc(-c9ccccc9)cc8)c7c6)ccc54)c3)cc2)cc1 ; c1ccc(-c2ccc(-n3c4ccccc4c4ccc(-c5ccc6c(c5)c5ccccc5n6-c5cccc(-c6ccccc6)c5)cc43)cc2)cc1 ; c1ccc(-c2cccc(-n3c4ccccc4c4cc(-c5ccc6c7ccccc7n(-c7cccc(-c8ccccc8)c7)c6c5)ccc43)c2)cc1]

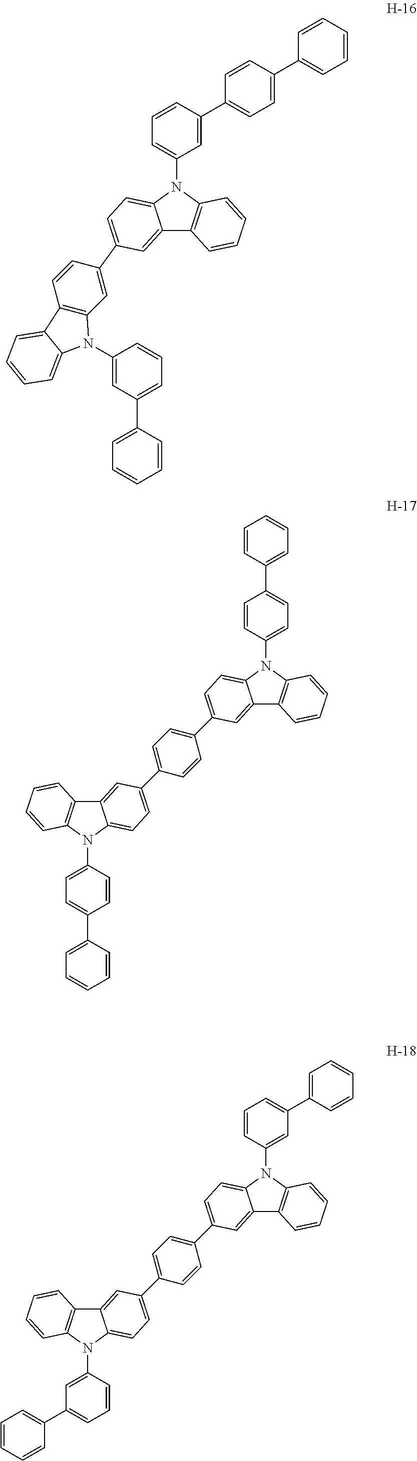 [SMILES: c1ccc(-c2ccc(-c3cccc(-n4c5ccccc5c5cc(-c6ccc7c8ccccc8n(-c8cccc(-c9ccccc9)c8)c7c6)ccc54)c3)cc2)cc1 ; c1ccc(-c2ccc(-n3c4ccccc4c4cc(-c5ccc(-c6ccc7c(c6)c6ccccc6n7-c6ccc(-c7ccccc7)cc6)cc5)ccc43)cc2)cc1 ; c1ccc(-c2cccc(-n3c4ccccc4c4cc(-c5ccc(-c6ccc7c(c6)c6ccccc6n7-c6cccc(-c7ccccc7)c6)cc5)ccc43)c2)cc1]

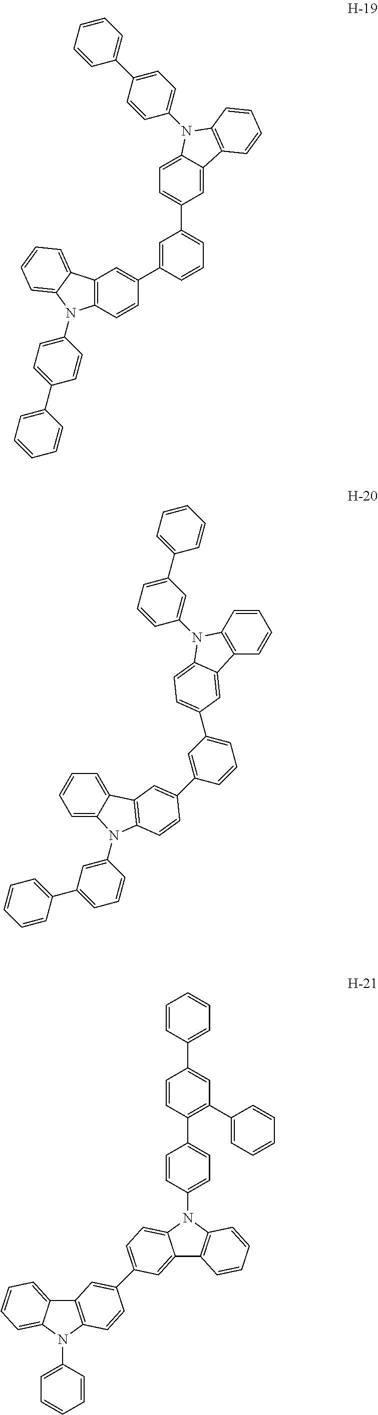 [SMILES: c1ccc(-c2ccc(-n3c4ccccc4c4cc(-c5cccc(-c6ccc7c(c6)c6ccccc6n7-c6ccc(-c7ccccc7)cc6)c5)ccc43)cc2)cc1 ; c1ccc(-c2cccc(-n3c4ccccc4c4cc(-c5cccc(-c6ccc7c(c6)c6ccccc6n7-c6cccc(-c7ccccc7)c6)c5)ccc43)c2)cc1 ; c1ccc(-c2ccc(-c3ccc(-n4c5ccccc5c5cc(-c6ccc7c(c6)c6ccccc6n7-c6ccccc6)ccc54)cc3)c(-c3ccccc3)c2)cc1]

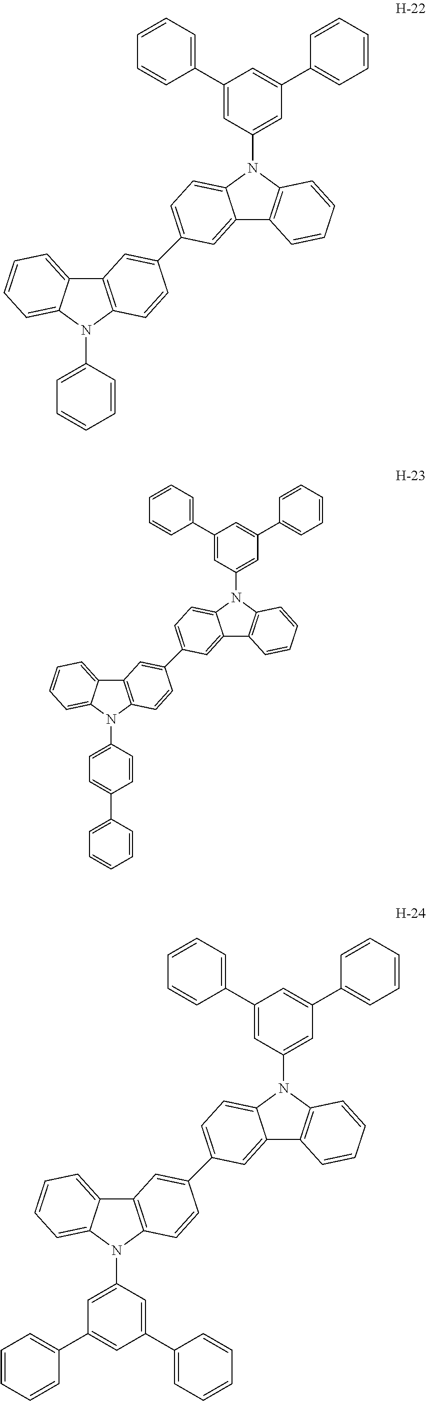 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1]

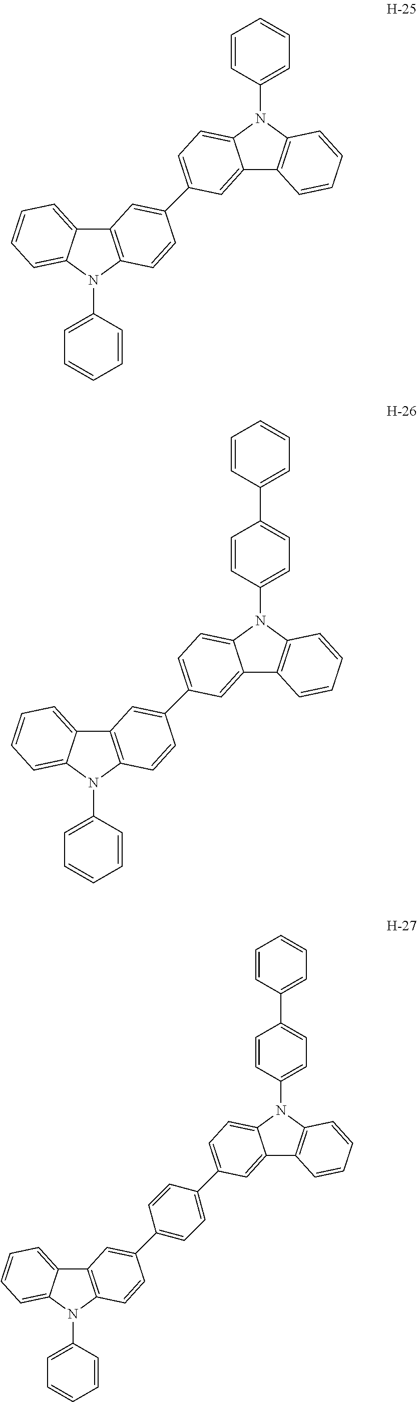 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-n3c4ccccc4c4cc(-c5ccc(-c6ccc7c(c6)c6ccccc6n7-c6ccccc6)cc5)ccc43)cc2)cc1]

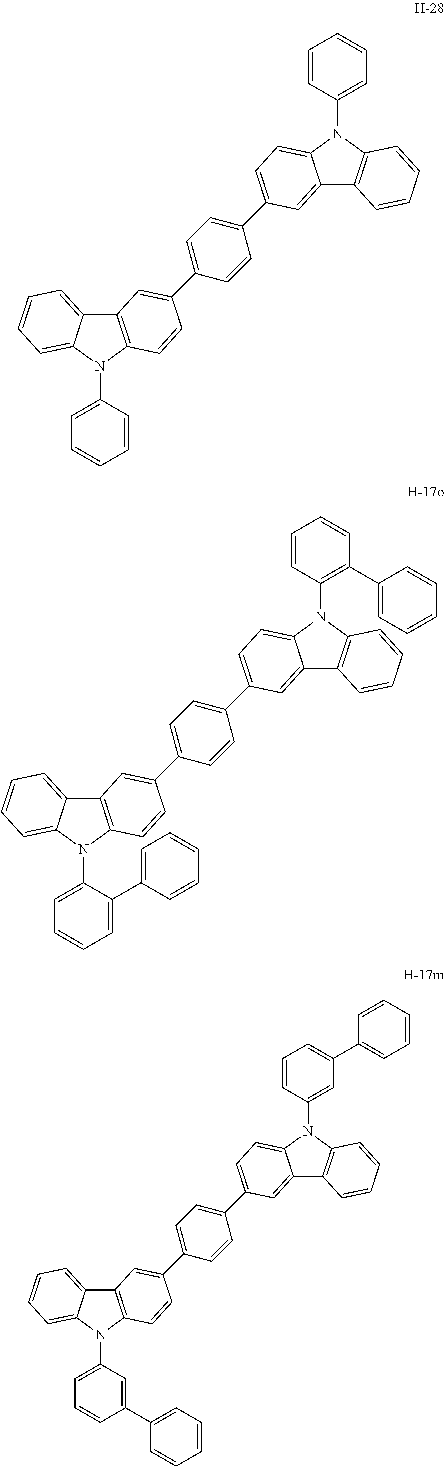 [SMILES: c1ccc(-n2c3ccccc3c3cc(-c4ccc(-c5ccc6c(c5)c5ccccc5n6-c5ccccc5)cc4)ccc32)cc1 ; c1ccc(-c2ccccc2-n2c3ccccc3c3cc(-c4ccc(-c5ccc6c(c5)c5ccccc5n6-c5ccccc5-c5ccccc5)cc4)ccc32)cc1 ; c1ccc(-c2cccc(-n3c4ccccc4c4cc(-c5ccc(-c6ccc7c(c6)c6ccccc6n7-c6cccc(-c7ccccc7)c6)cc5)ccc43)c2)cc1]

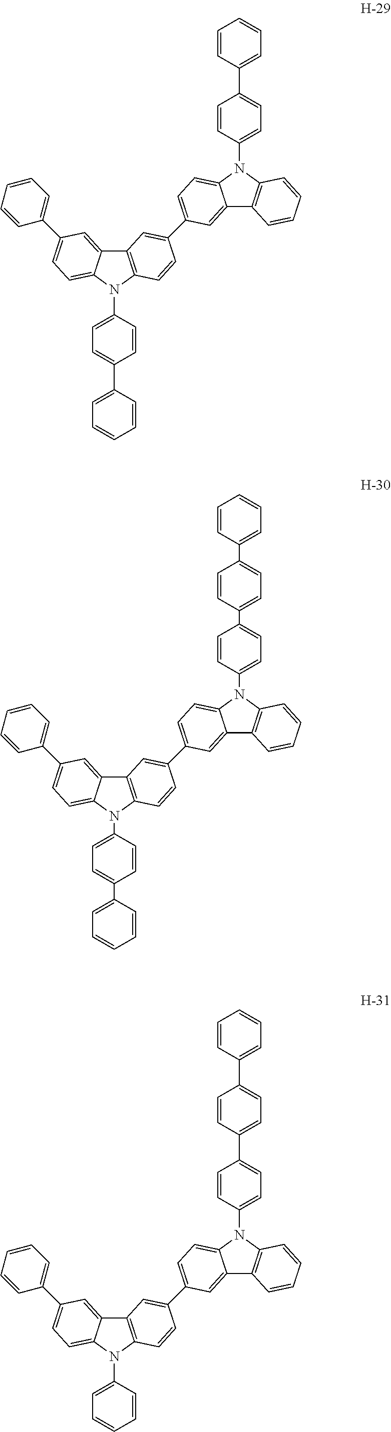 [SMILES: c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1 ; c1ccc(-c2ccc(-c3ccc(-n4c5ccccc5c5cc(-c6ccc7c(c6)c6cc(-c8ccccc8)ccc6n7-c6ccc(-c7ccccc7)cc6)ccc54)cc3)cc2)cc1 ; c1ccc(-c2ccc(-c3ccc(-n4c5ccccc5c5cc(-c6ccc7c(c6)c6cc(-c8ccccc8)ccc6n7-c6ccccc6)ccc54)cc3)cc2)cc1]

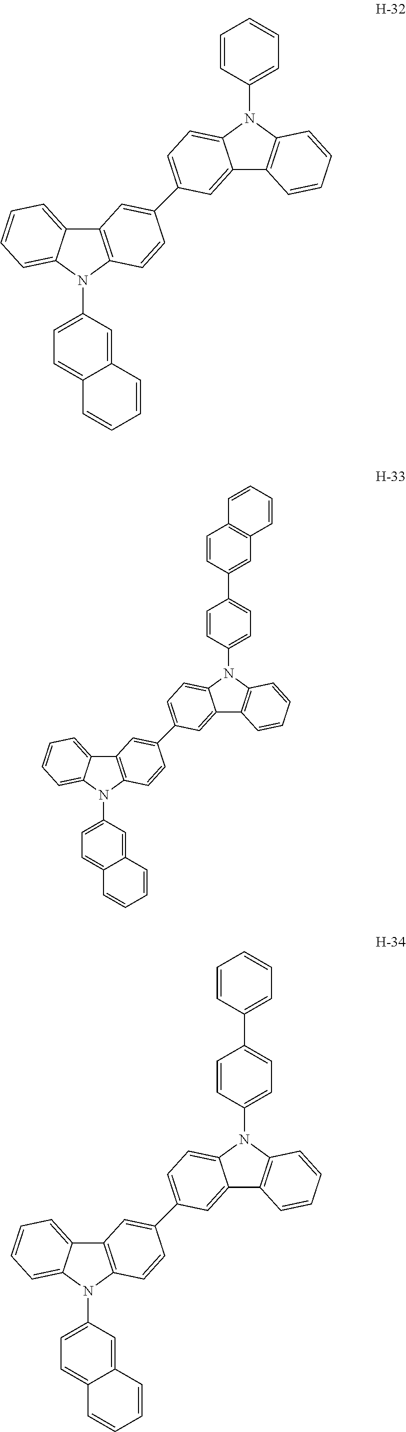 [SMILES: c1ccc(-n2c3ccccc3c3cc(-c4ccc5c(c4)c4ccccc4n5-c4ccc5ccccc5c4)ccc32)cc1 ; c1ccc2cc(-c3ccc(-n4c5ccccc5c5cc(-c6ccc7c(c6)c6ccccc6n7-c6ccc7ccccc7c6)ccc54)cc3)ccc2c1 ; c1ccc(-c2ccc(-n3c4ccccc4c4cc(-c5ccc6c(c5)c5ccccc5n6-c5ccc6ccccc6c5)ccc43)cc2)cc1]

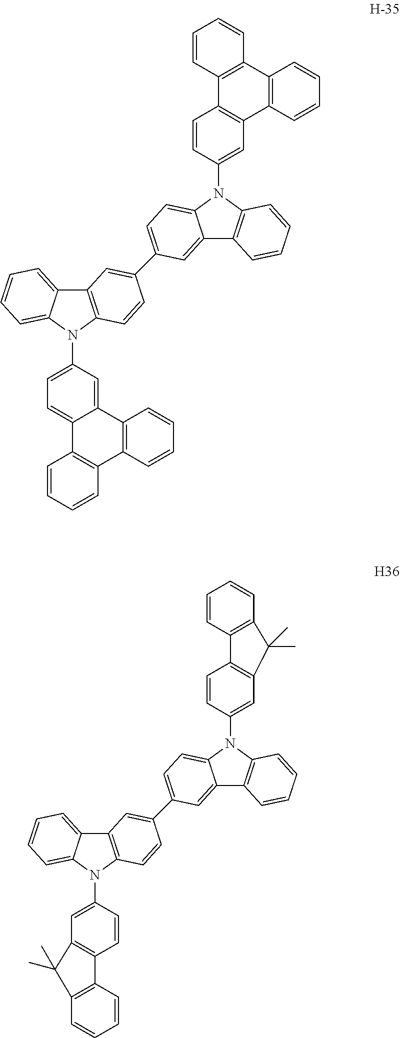 [SMILES: c1ccc2c(c1)c1ccccc1c1cc(-n3c4ccccc4c4cc(-c5ccc6c(c5)c5ccccc5n6-c5ccc6c7ccccc7c7ccccc7c6c5)ccc43)ccc21 ; CC1(C)c2ccccc2-c2ccc(-n3c4ccccc4c4cc(-c5ccc6c(c5)c5ccccc5n6-c5ccc6c(c5)C(C)(C)c5ccccc5-6)ccc43)cc21]

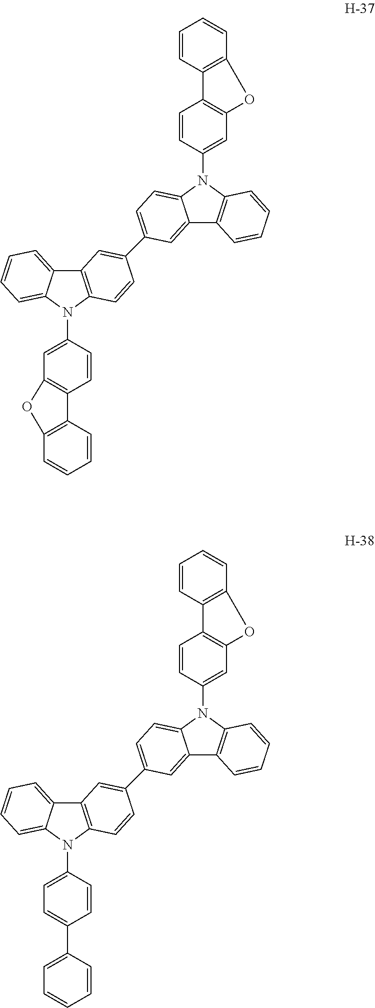 [SMILES: c1ccc2c(c1)oc1cc(-n3c4ccccc4c4cc(-c5ccc6c(c5)c5ccccc5n6-c5ccc6c(c5)oc5ccccc56)ccc43)ccc12 ; c1ccc(-c2ccc(-n3c4ccccc4c4cc(-c5ccc6c(c5)c5ccccc5n6-c5ccc6c(c5)oc5ccccc56)ccc43)cc2)cc1]

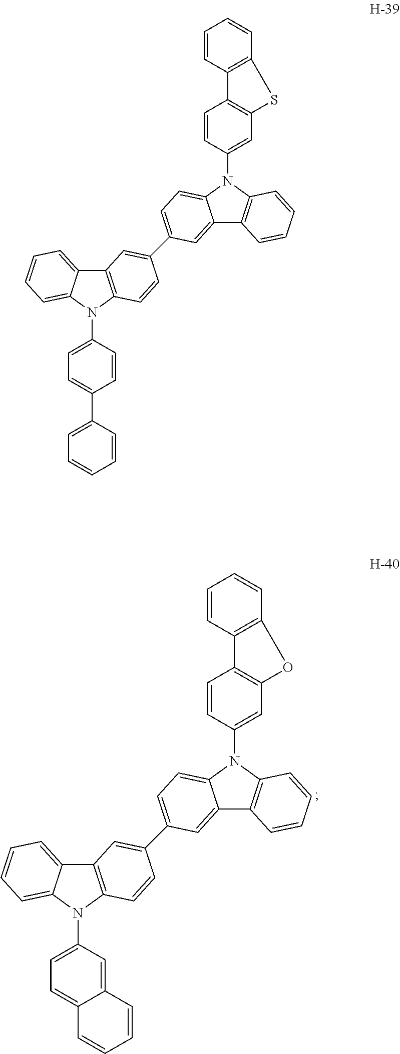 [SMILES: c1ccc(-c2ccc(-n3c4ccccc4c4cc(-c5ccc6c(c5)c5ccccc5n6-c5ccc6c(c5)sc5ccccc56)ccc43)cc2)cc1 ; c1ccc2cc(-n3c4ccccc4c4cc(-c5ccc6c(c5)c5ccccc5n6-c5ccc6c(c5)oc5ccccc56)ccc43)ccc2c1]

the “substituted” means that hydrogen atoms in the above compounds are each independently substituted with deuterium atoms.

As an optional technical solution of the present application, the compound represented by Formula II is selected from any one of the following substituted or unsubstituted compounds:

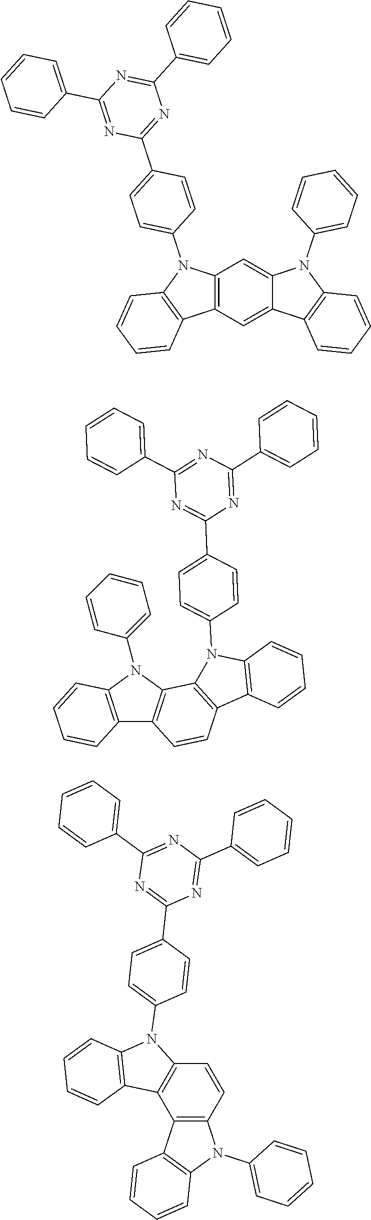

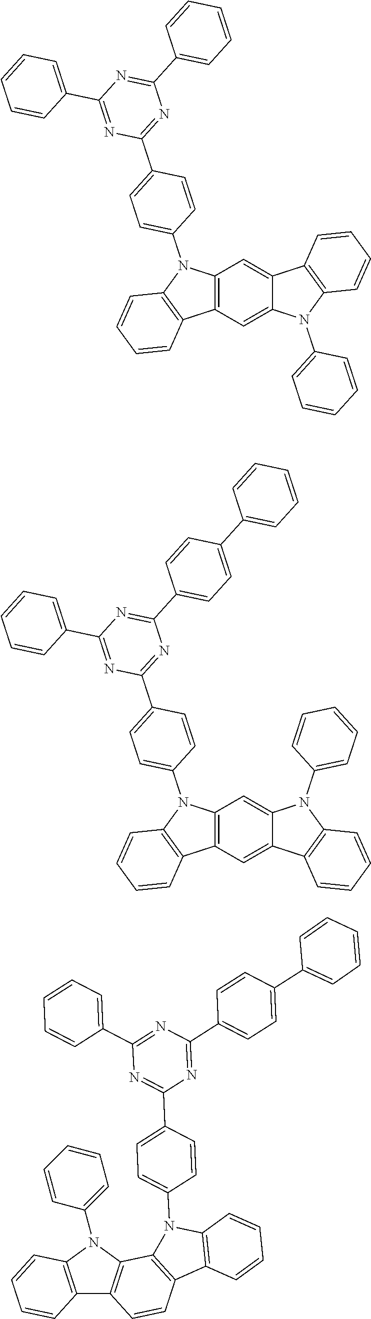

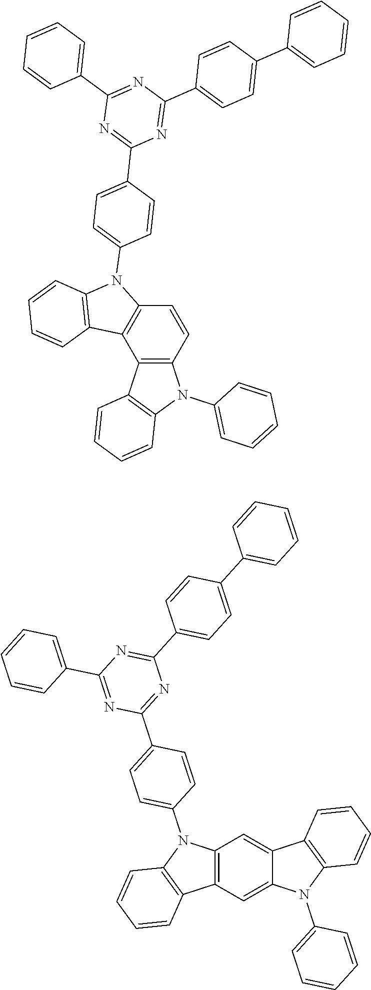 [SMILES: c1ccc(-c2ccc(-c3nc(-c4ccccc4)nc(-c4ccc(-n5c6ccccc6c6c7c8ccccc8n(-c8ccccc8)c7ccc65)cc4)n3)cc2)cc1 ; c1ccc(-c2ccc(-c3nc(-c4ccccc4)nc(-c4ccc(-n5c6ccccc6c6cc7c(cc65)c5ccccc5n7-c5ccccc5)cc4)n3)cc2)cc1]

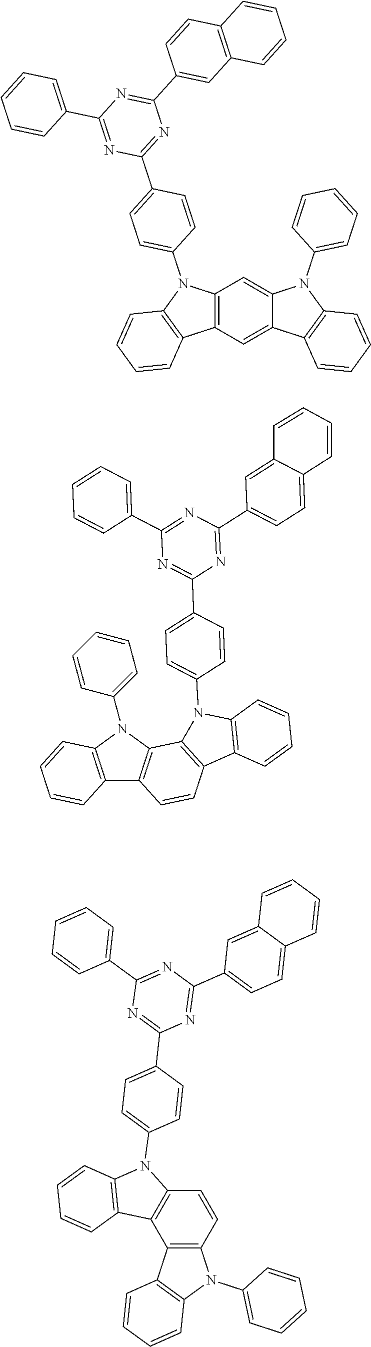

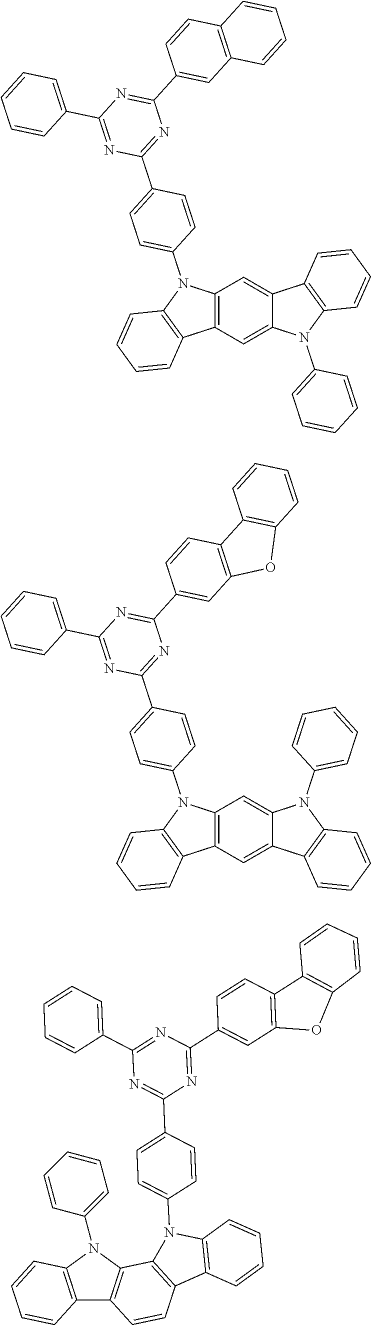

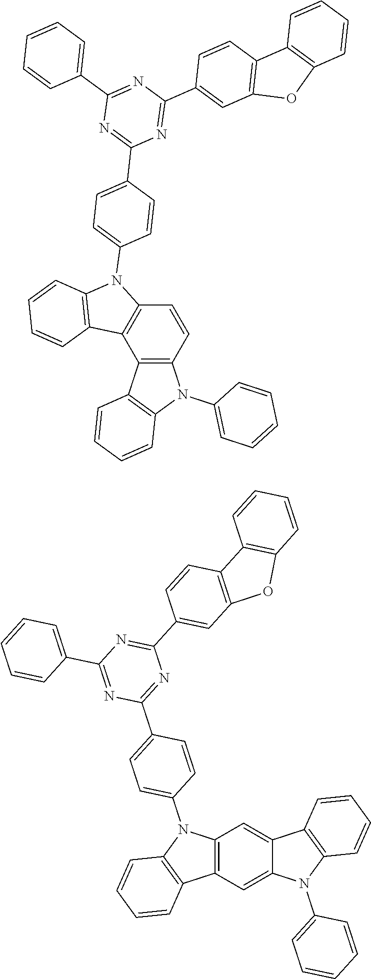

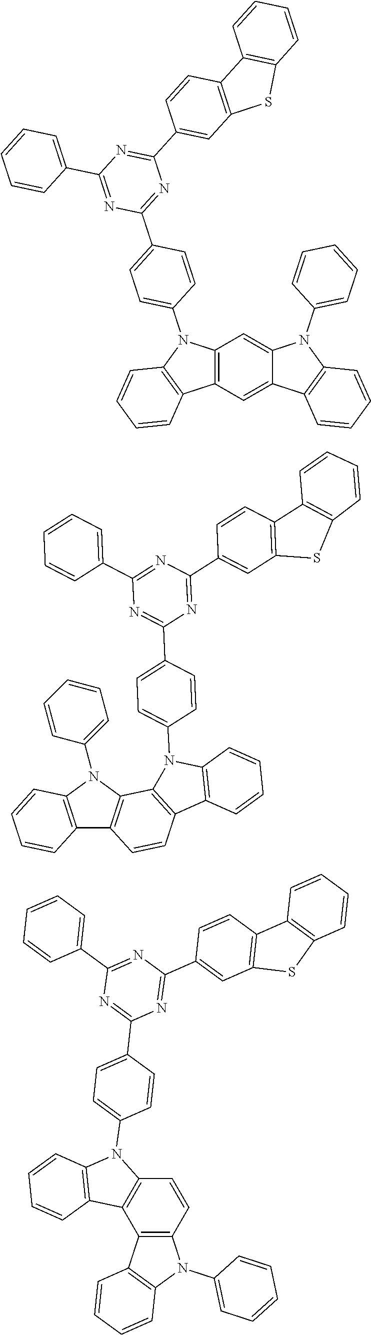

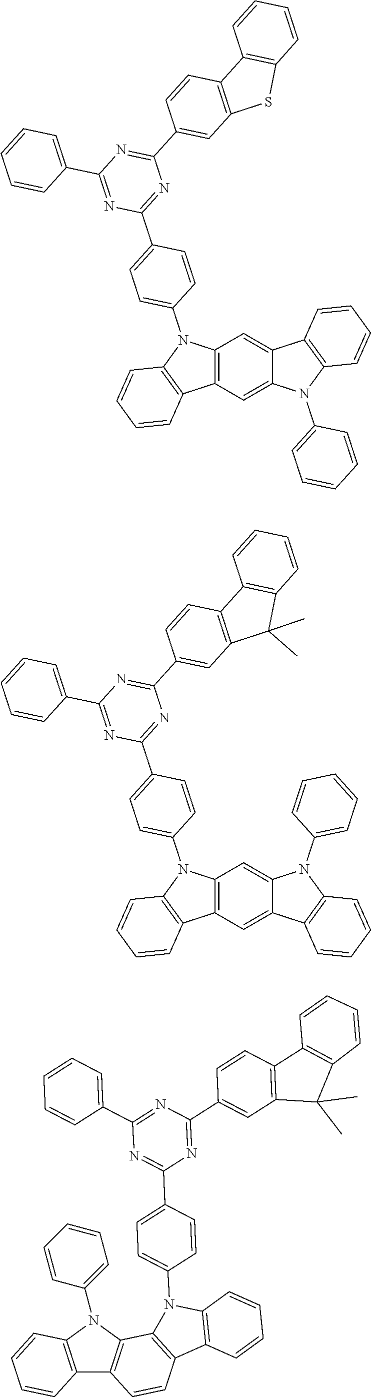

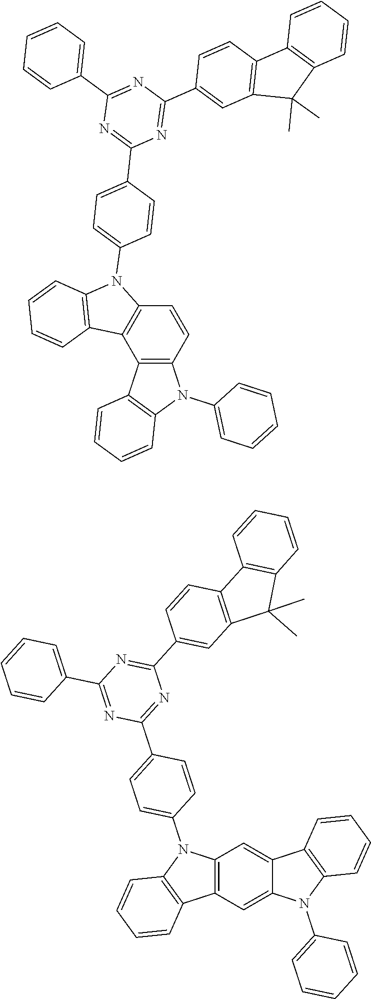 [SMILES: CC1(C)c2ccccc2-c2ccc(-c3nc(-c4ccccc4)nc(-c4ccc(-n5c6ccccc6c6c7c8ccccc8n(-c8ccccc8)c7ccc65)cc4)n3)cc21 ; CC1(C)c2ccccc2-c2ccc(-c3nc(-c4ccccc4)nc(-c4ccc(-n5c6ccccc6c6cc7c(cc65)c5ccccc5n7-c5ccccc5)cc4)n3)cc21]

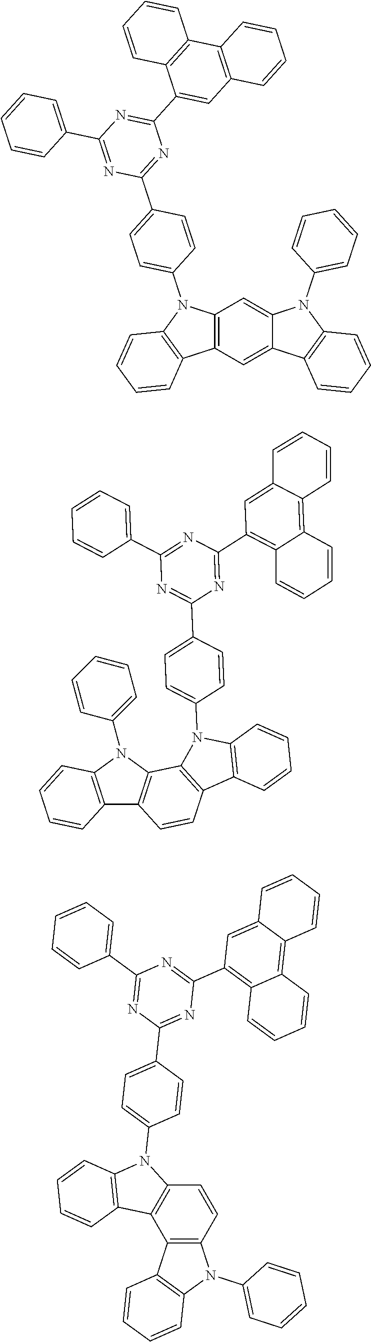

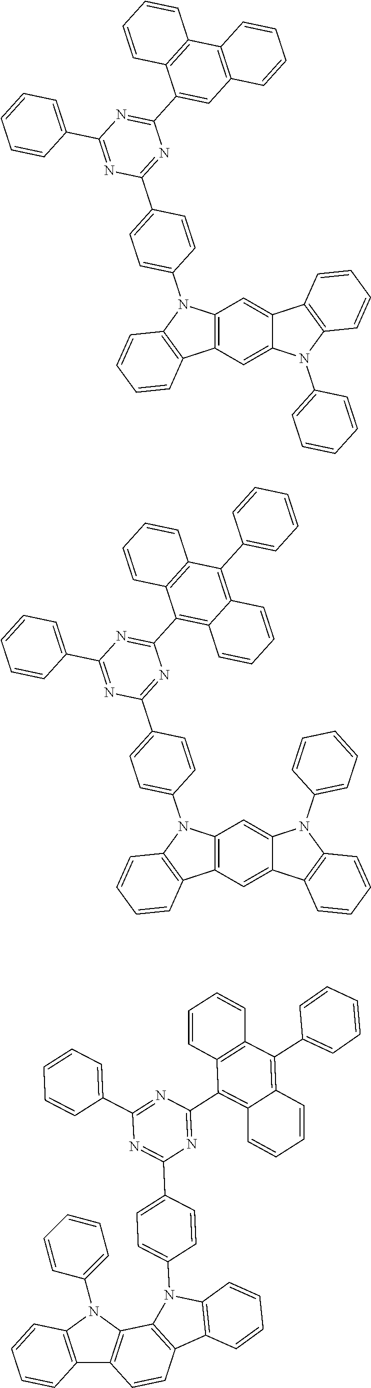 [SMILES: c1ccc(-c2nc(-c3ccc(-n4c5ccccc5c5cc6c(cc54)c4ccccc4n6-c4ccccc4)cc3)nc(-c3cc4ccccc4c4ccccc34)n2)cc1 ; c1ccc(-c2nc(-c3ccc(-n4c5ccccc5c5cc6c7ccccc7n(-c7ccccc7)c6cc54)cc3)nc(-c3c4ccccc4c(-c4ccccc4)c4ccccc34)n2)cc1 ; c1ccc(-c2nc(-c3ccc(-n4c5ccccc5c5ccc6c7ccccc7n(-c7ccccc7)c6c54)cc3)nc(-c3c4ccccc4c(-c4ccccc4)c4ccccc34)n2)cc1]

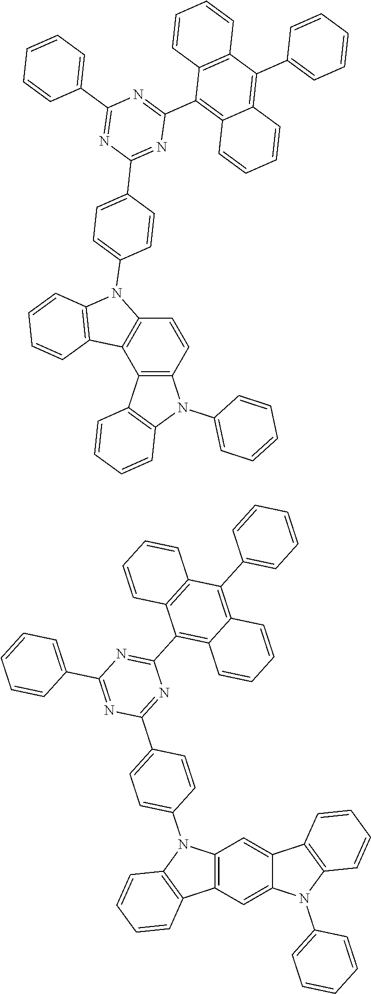

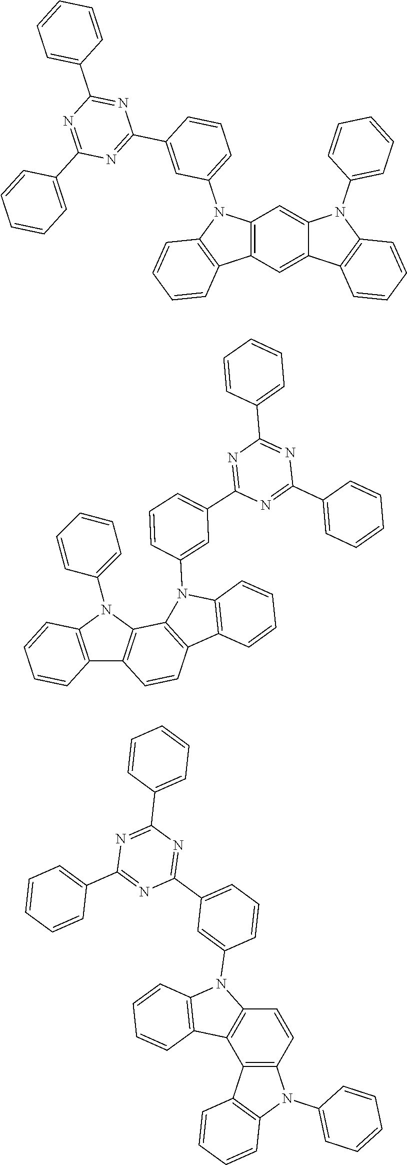

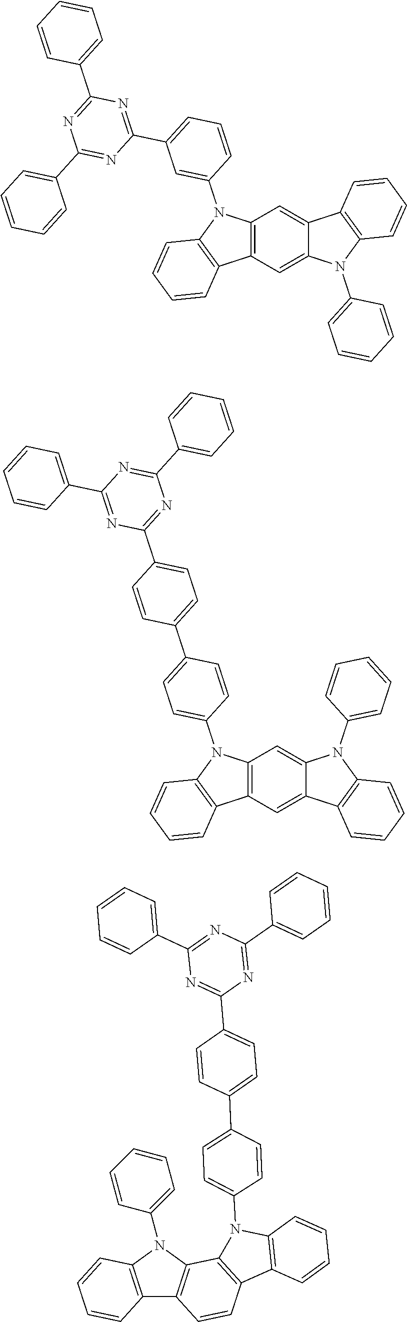 [SMILES: c1ccc(-c2nc(-c3ccccc3)nc(-c3cccc(-n4c5ccccc5c5cc6c(cc54)c4ccccc4n6-c4ccccc4)c3)n2)cc1 ; c1ccc(-c2nc(-c3ccccc3)nc(-c3ccc(-c4ccc(-n5c6ccccc6c6cc7c8ccccc8n(-c8ccccc8)c7cc65)cc4)cc3)n2)cc1 ; c1ccc(-c2nc(-c3ccccc3)nc(-c3ccc(-c4ccc(-n5c6ccccc6c6ccc7c8ccccc8n(-c8ccccc8)c7c65)cc4)cc3)n2)cc1]

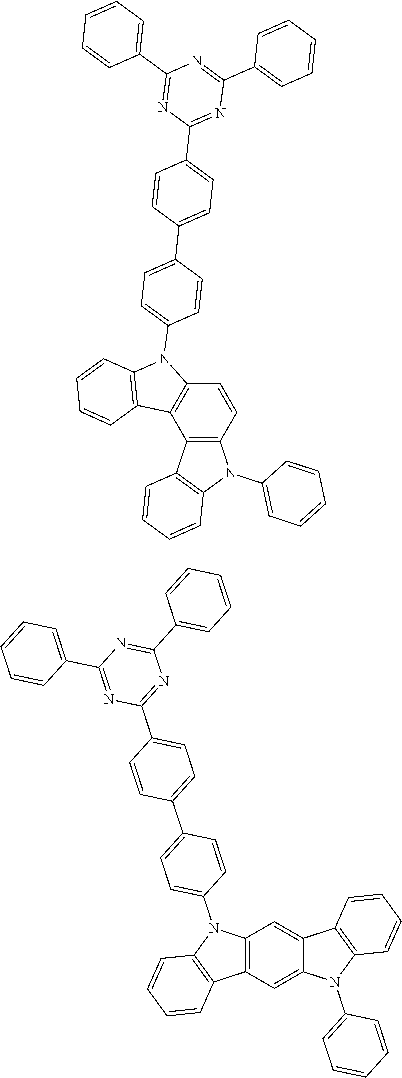

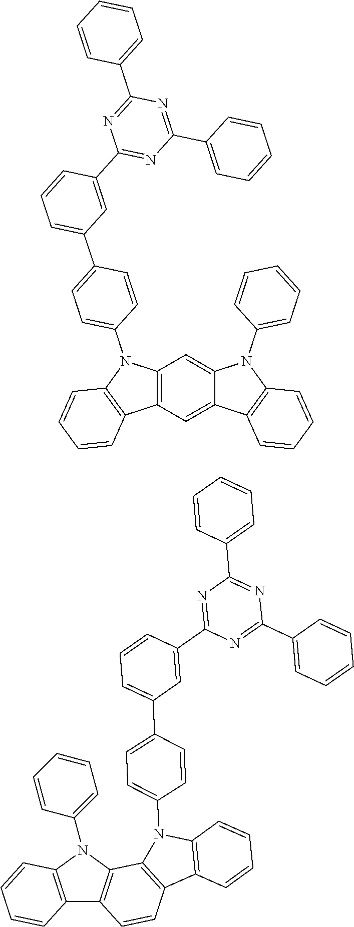 [SMILES: c1ccc(-c2nc(-c3ccccc3)nc(-c3cccc(-c4ccc(-n5c6ccccc6c6cc7c8ccccc8n(-c8ccccc8)c7cc65)cc4)c3)n2)cc1 ; c1ccc(-c2nc(-c3ccccc3)nc(-c3cccc(-c4ccc(-n5c6ccccc6c6ccc7c8ccccc8n(-c8ccccc8)c7c65)cc4)c3)n2)cc1]

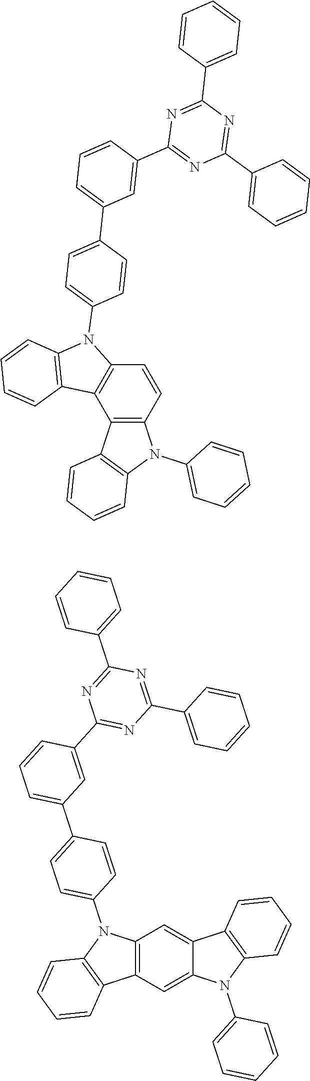 [SMILES: c1ccc(-c2nc(-c3ccccc3)nc(-c3cccc(-c4ccc(-n5c6ccccc6c6c7c8ccccc8n(-c8ccccc8)c7ccc65)cc4)c3)n2)cc1 ; c1ccc(-c2nc(-c3ccccc3)nc(-c3cccc(-c4ccc(-n5c6ccccc6c6cc7c(cc65)c5ccccc5n7-c5ccccc5)cc4)c3)n2)cc1]

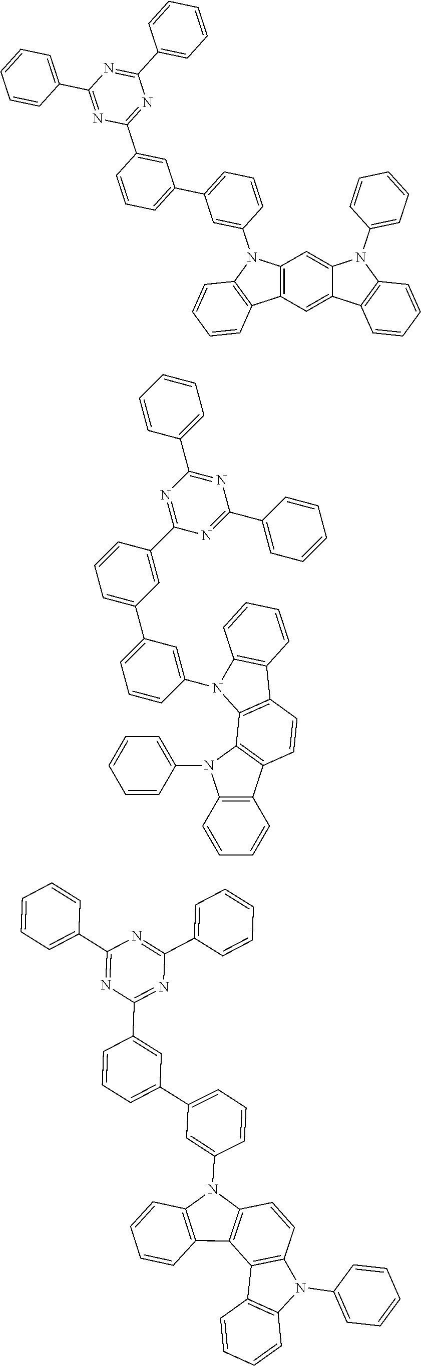

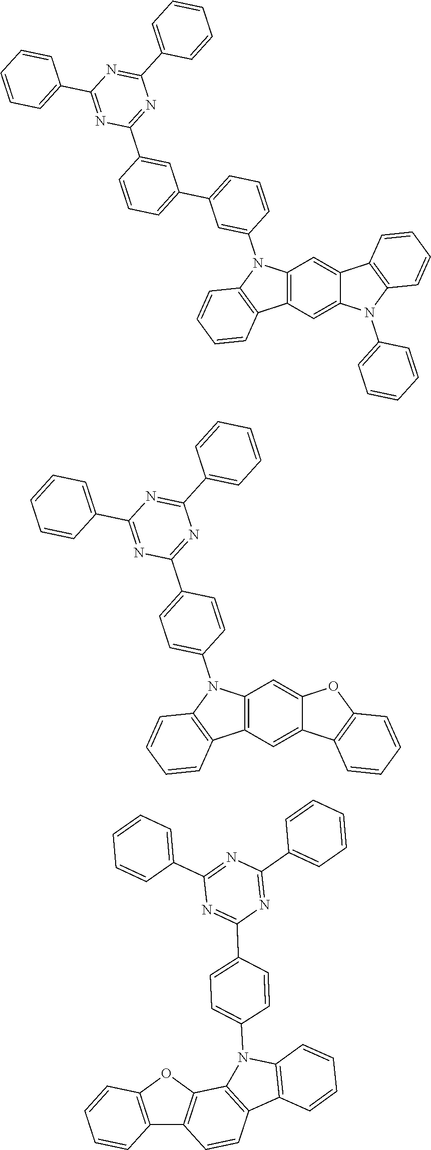

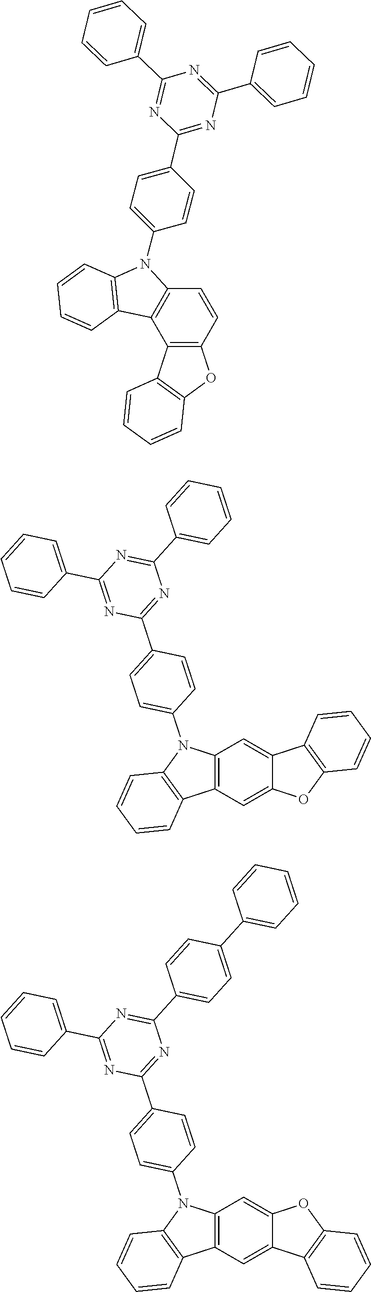 [SMILES: c1ccc(-c2nc(-c3ccccc3)nc(-c3ccc(-n4c5ccccc5c5c6c(ccc54)oc4ccccc46)cc3)n2)cc1 ; c1ccc(-c2nc(-c3ccccc3)nc(-c3ccc(-n4c5ccccc5c5cc6oc7ccccc7c6cc54)cc3)n2)cc1 ; c1ccc(-c2ccc(-c3nc(-c4ccccc4)nc(-c4ccc(-n5c6ccccc6c6cc7c(cc65)oc5ccccc57)cc4)n3)cc2)cc1]

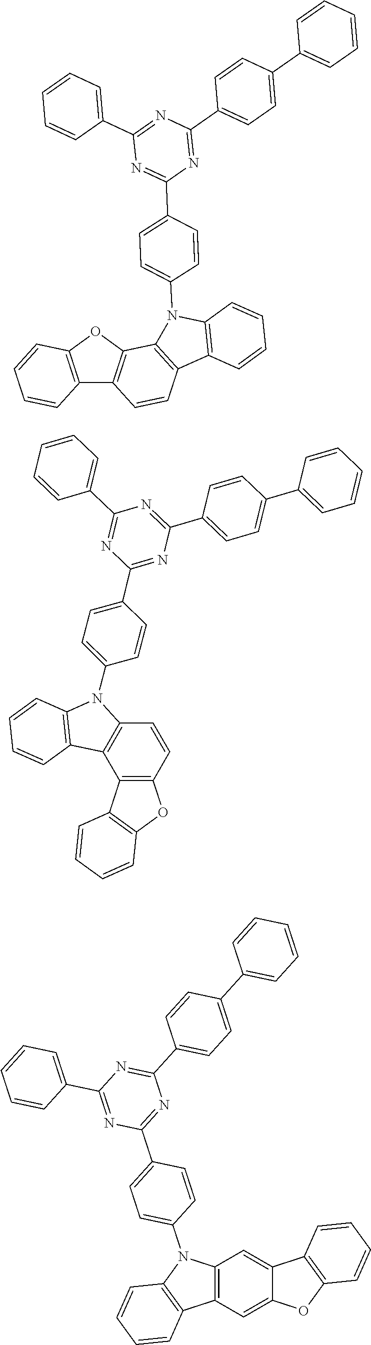

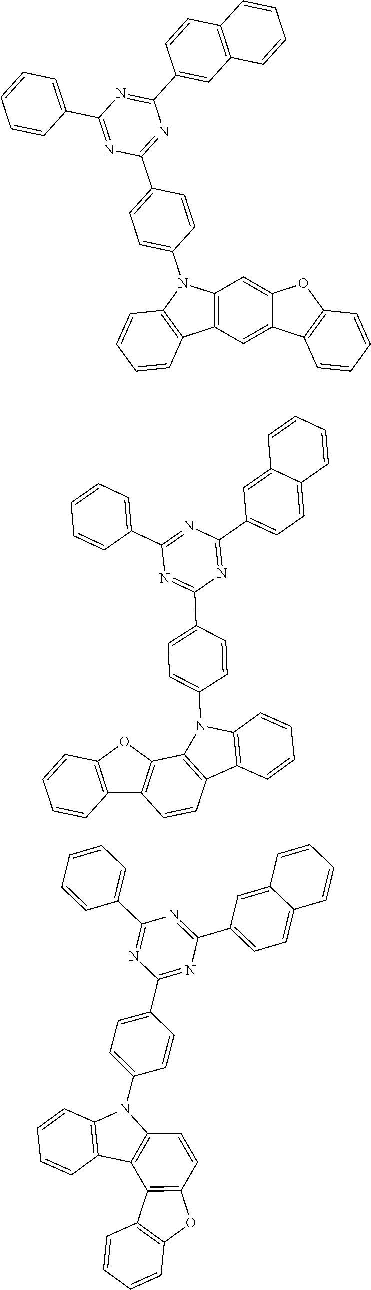

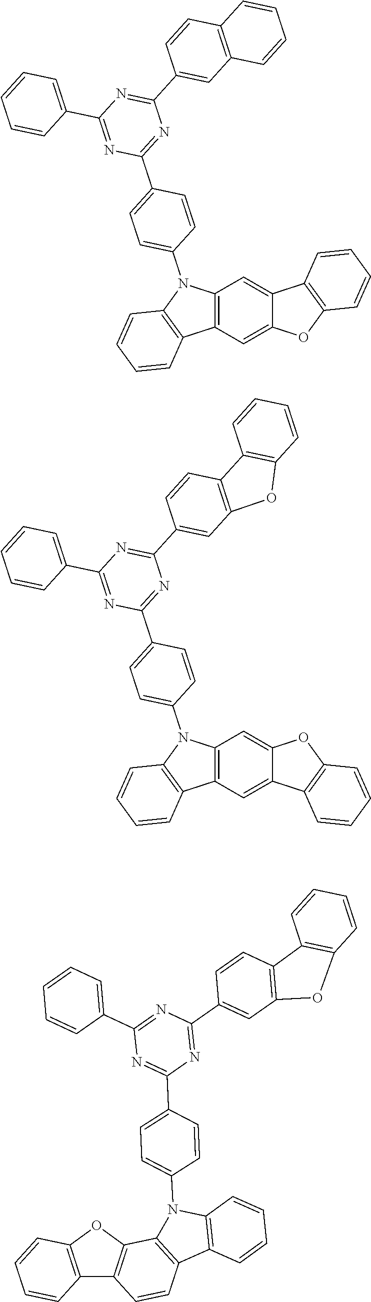 [SMILES: c1ccc(-c2nc(-c3ccc(-n4c5ccccc5c5cc6oc7ccccc7c6cc54)cc3)nc(-c3ccc4ccccc4c3)n2)cc1 ; c1ccc(-c2nc(-c3ccc(-n4c5ccccc5c5cc6c(cc54)oc4ccccc46)cc3)nc(-c3ccc4c(c3)oc3ccccc34)n2)cc1 ; c1ccc(-c2nc(-c3ccc(-n4c5ccccc5c5ccc6c7ccccc7oc6c54)cc3)nc(-c3ccc4c(c3)oc3ccccc34)n2)cc1]

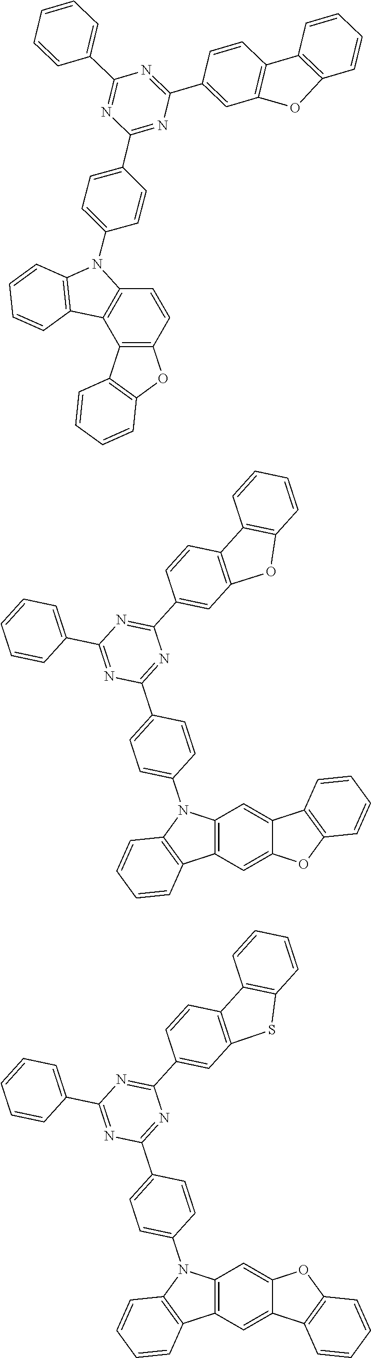 [SMILES: c1ccc(-c2nc(-c3ccc(-n4c5ccccc5c5c6c(ccc54)oc4ccccc46)cc3)nc(-c3ccc4c(c3)oc3ccccc34)n2)cc1 ; c1ccc(-c2nc(-c3ccc(-n4c5ccccc5c5cc6oc7ccccc7c6cc54)cc3)nc(-c3ccc4c(c3)oc3ccccc34)n2)cc1 ; c1ccc(-c2nc(-c3ccc(-n4c5ccccc5c5cc6c(cc54)oc4ccccc46)cc3)nc(-c3ccc4c(c3)sc3ccccc34)n2)cc1]

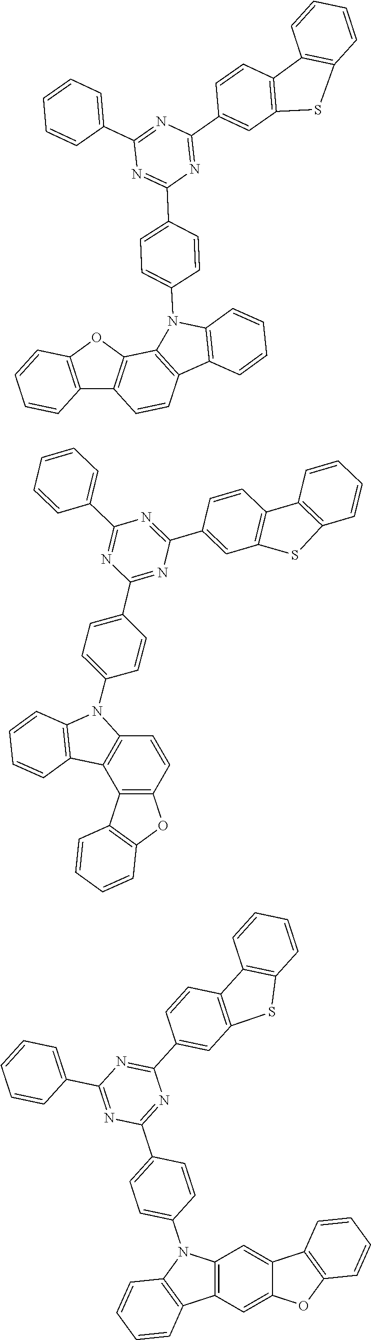

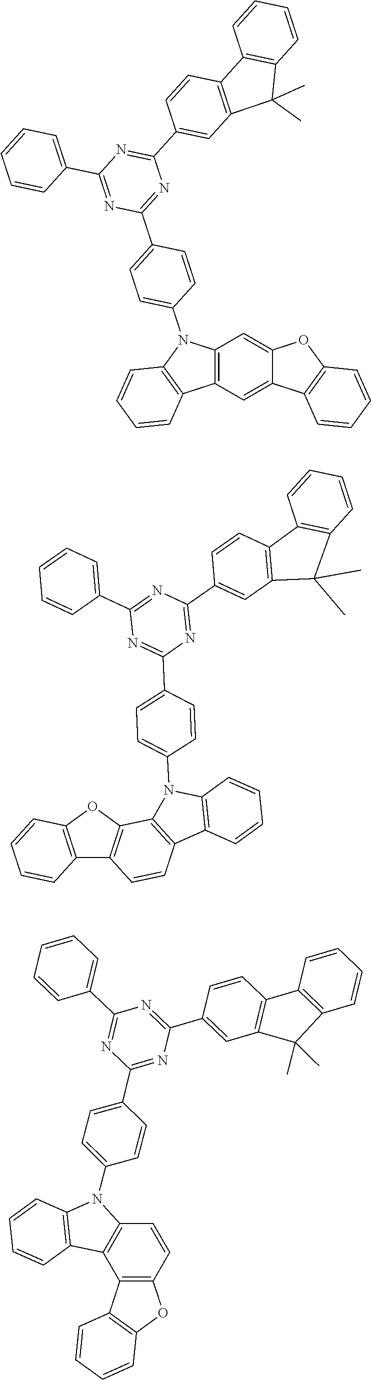 [SMILES: CC1(C)c2ccccc2-c2ccc(-c3nc(-c4ccccc4)nc(-c4ccc(-n5c6ccccc6c6cc7c(cc65)oc5ccccc57)cc4)n3)cc21 ; CC1(C)c2ccccc2-c2ccc(-c3nc(-c4ccccc4)nc(-c4ccc(-n5c6ccccc6c6ccc7c8ccccc8oc7c65)cc4)n3)cc21 ; CC1(C)c2ccccc2-c2ccc(-c3nc(-c4ccccc4)nc(-c4ccc(-n5c6ccccc6c6c7c(ccc65)oc5ccccc57)cc4)n3)cc21]

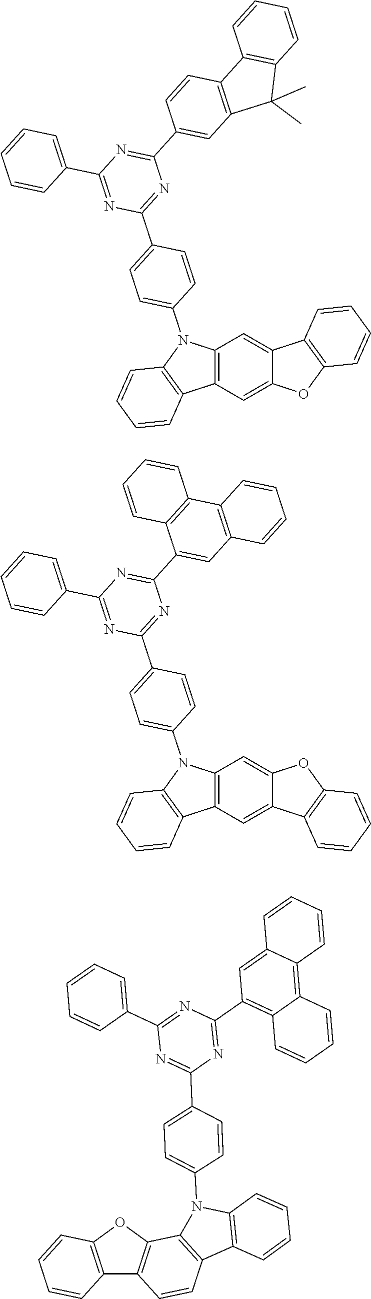

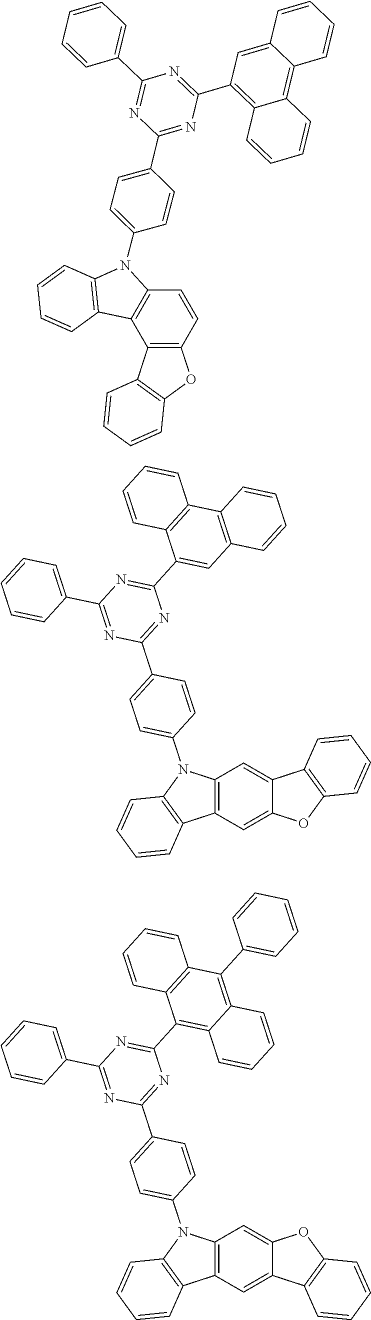

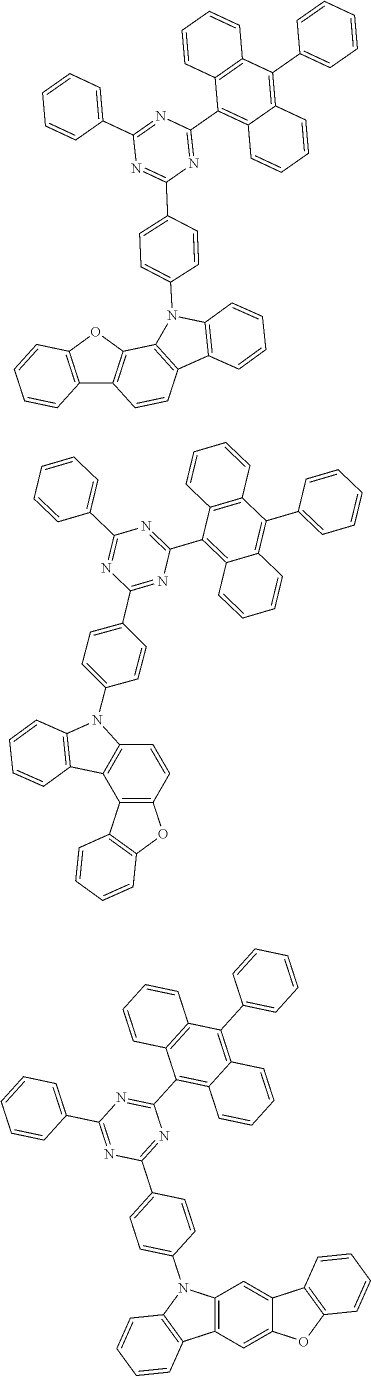 [SMILES: c1ccc(-c2nc(-c3ccc(-n4c5ccccc5c5ccc6c7ccccc7oc6c54)cc3)nc(-c3c4ccccc4c(-c4ccccc4)c4ccccc34)n2)cc1 ; c1ccc(-c2nc(-c3ccc(-n4c5ccccc5c5c6c(ccc54)oc4ccccc46)cc3)nc(-c3c4ccccc4c(-c4ccccc4)c4ccccc34)n2)cc1 ; c1ccc(-c2nc(-c3ccc(-n4c5ccccc5c5cc6oc7ccccc7c6cc54)cc3)nc(-c3c4ccccc4c(-c4ccccc4)c4ccccc34)n2)cc1]

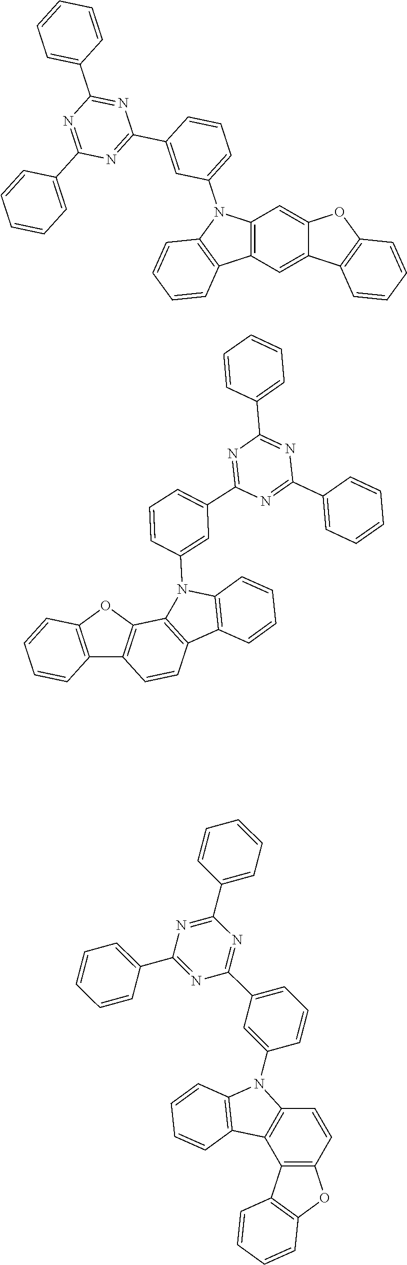 [SMILES: c1ccc(-c2nc(-c3ccccc3)nc(-c3cccc(-n4c5ccccc5c5cc6c(cc54)oc4ccccc46)c3)n2)cc1 ; c1ccc(-c2nc(-c3ccccc3)nc(-c3cccc(-n4c5ccccc5c5ccc6c7ccccc7oc6c54)c3)n2)cc1 ; c1ccc(-c2nc(-c3ccccc3)nc(-c3cccc(-n4c5ccccc5c5c6c(ccc54)oc4ccccc46)c3)n2)cc1]

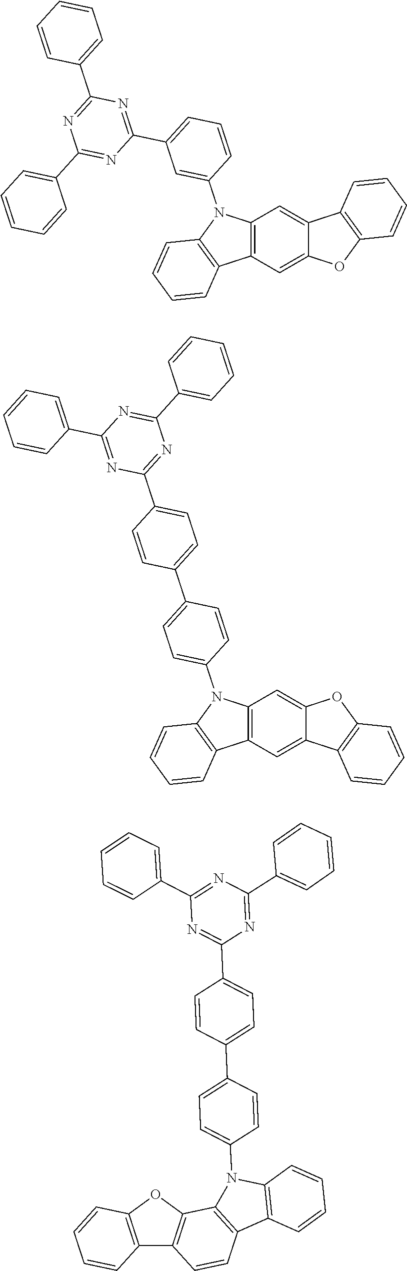

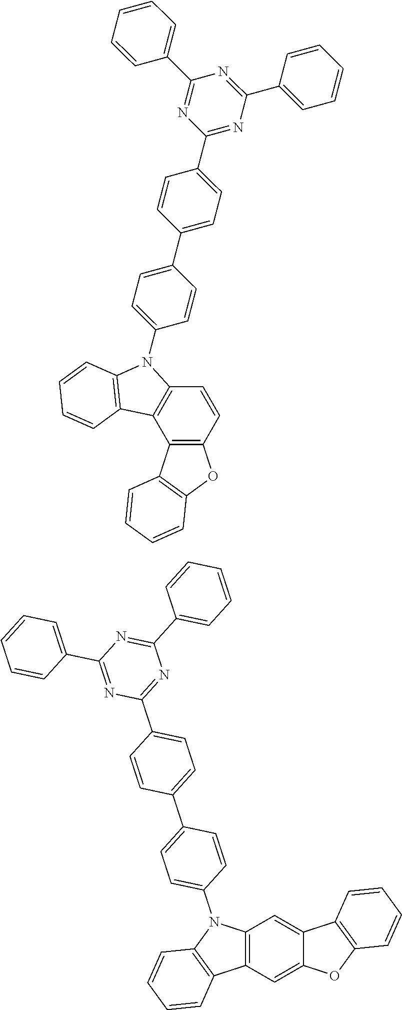

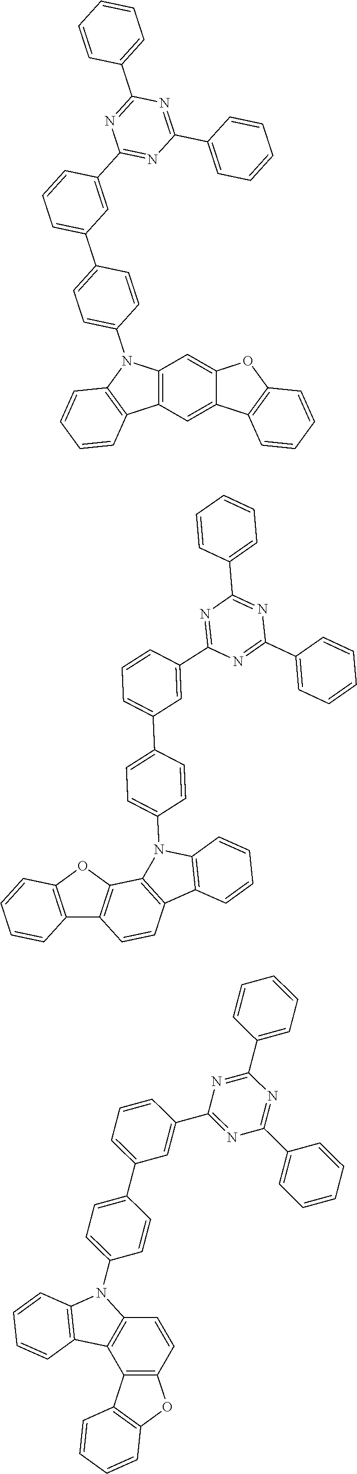 [SMILES: c1ccc(-c2nc(-c3ccccc3)nc(-c3cccc(-c4ccc(-n5c6ccccc6c6cc7c(cc65)oc5ccccc57)cc4)c3)n2)cc1 ; c1ccc(-c2nc(-c3ccccc3)nc(-c3cccc(-c4ccc(-n5c6ccccc6c6ccc7c8ccccc8oc7c65)cc4)c3)n2)cc1 ; c1ccc(-c2nc(-c3ccccc3)nc(-c3cccc(-c4ccc(-n5c6ccccc6c6c7c(ccc65)oc5ccccc57)cc4)c3)n2)cc1]

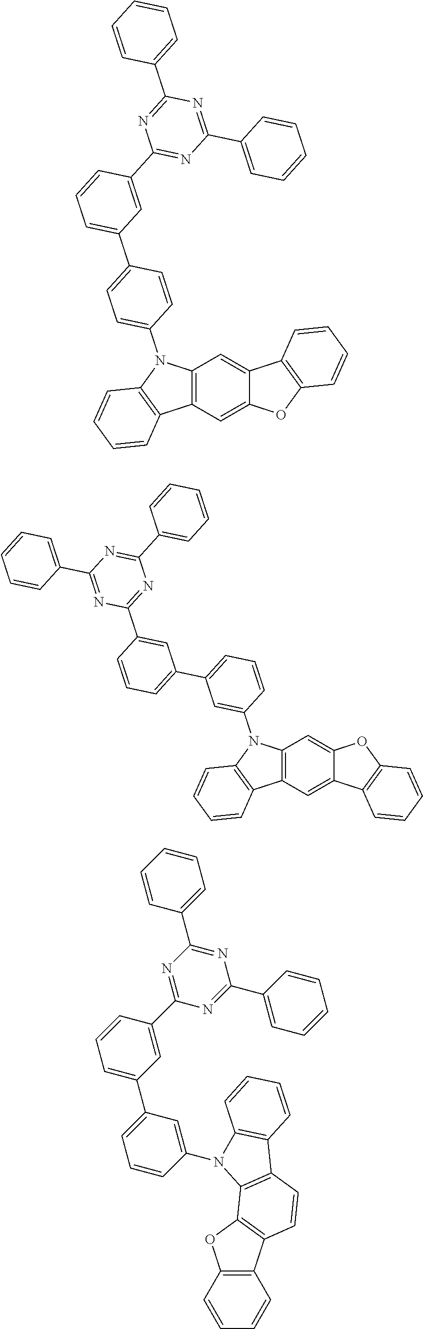

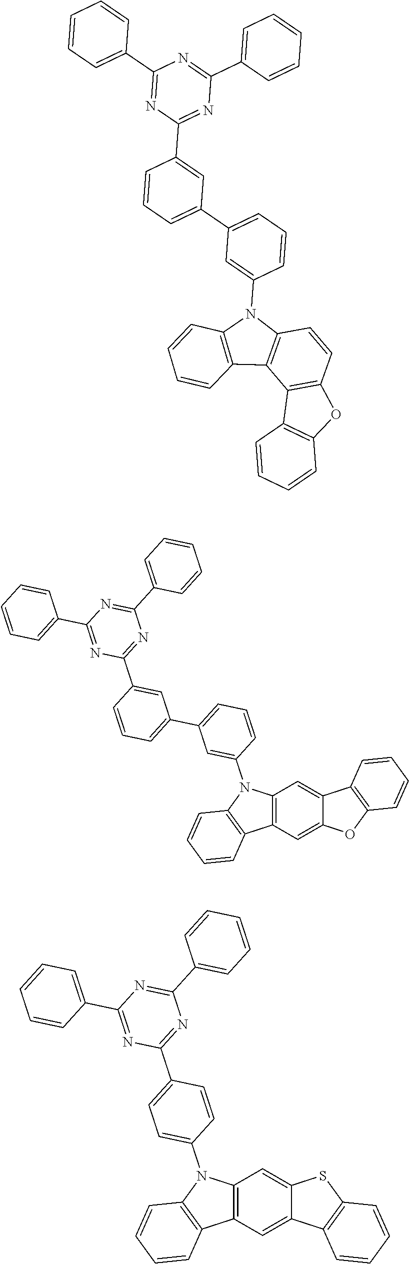 [SMILES: c1ccc(-c2nc(-c3ccccc3)nc(-c3cccc(-c4cccc(-n5c6ccccc6c6c7c(ccc65)oc5ccccc57)c4)c3)n2)cc1 ; c1ccc(-c2nc(-c3ccccc3)nc(-c3cccc(-c4cccc(-n5c6ccccc6c6cc7oc8ccccc8c7cc65)c4)c3)n2)cc1 ; c1ccc(-c2nc(-c3ccccc3)nc(-c3ccc(-n4c5ccccc5c5cc6c(cc54)sc4ccccc46)cc3)n2)cc1]

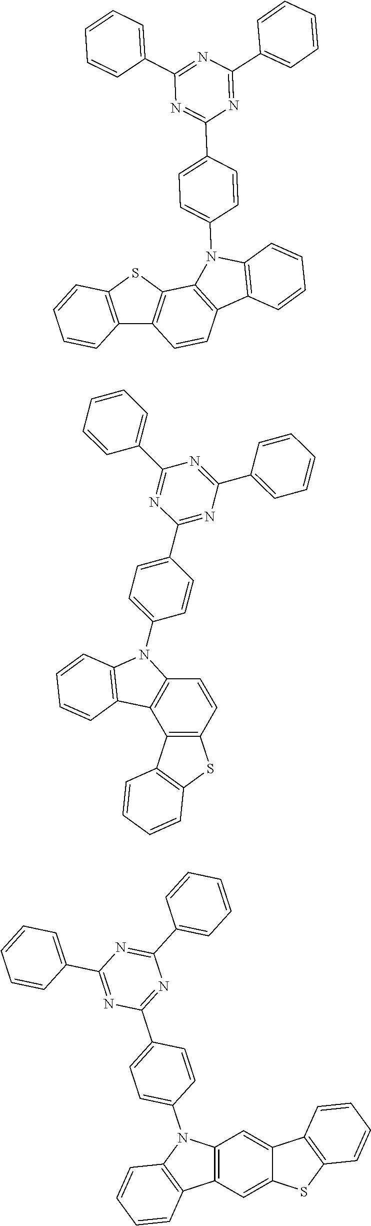 [SMILES: c1ccc(-c2nc(-c3ccccc3)nc(-c3ccc(-n4c5ccccc5c5ccc6c7ccccc7sc6c54)cc3)n2)cc1 ; c1ccc(-c2nc(-c3ccccc3)nc(-c3ccc(-n4c5ccccc5c5c6c(ccc54)sc4ccccc46)cc3)n2)cc1 ; c1ccc(-c2nc(-c3ccccc3)nc(-c3ccc(-n4c5ccccc5c5cc6sc7ccccc7c6cc54)cc3)n2)cc1]

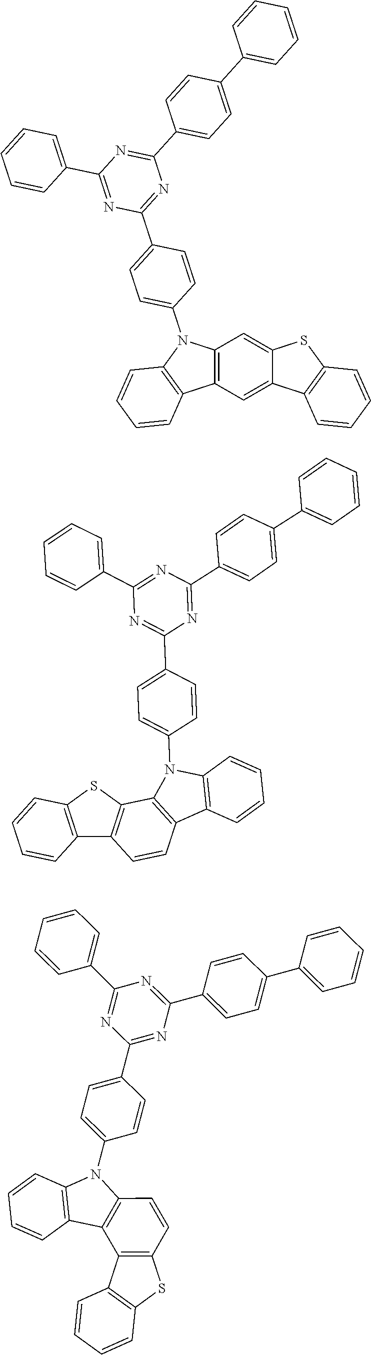

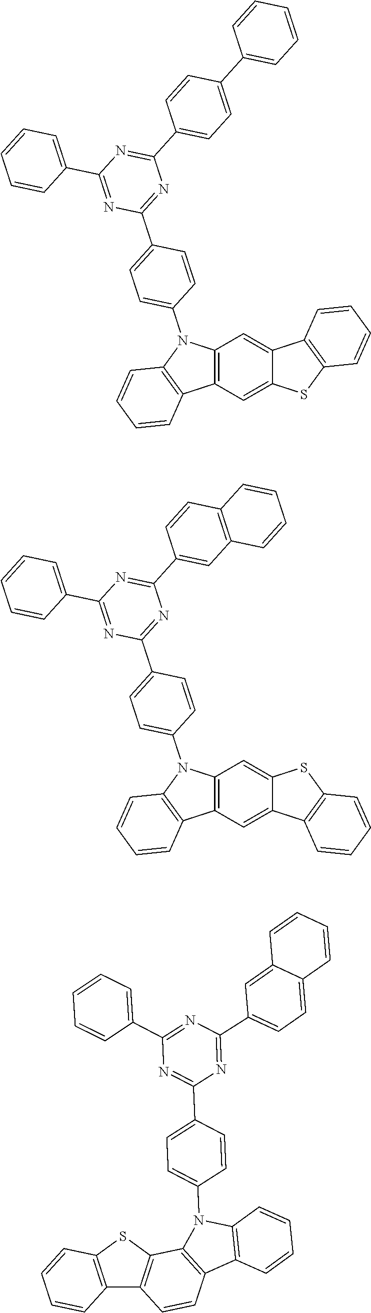 [SMILES: c1ccc(-c2ccc(-c3nc(-c4ccccc4)nc(-c4ccc(-n5c6ccccc6c6cc7sc8ccccc8c7cc65)cc4)n3)cc2)cc1 ; c1ccc(-c2nc(-c3ccc(-n4c5ccccc5c5cc6c(cc54)sc4ccccc46)cc3)nc(-c3ccc4ccccc4c3)n2)cc1 ; c1ccc(-c2nc(-c3ccc(-n4c5ccccc5c5ccc6c7ccccc7sc6c54)cc3)nc(-c3ccc4ccccc4c3)n2)cc1]

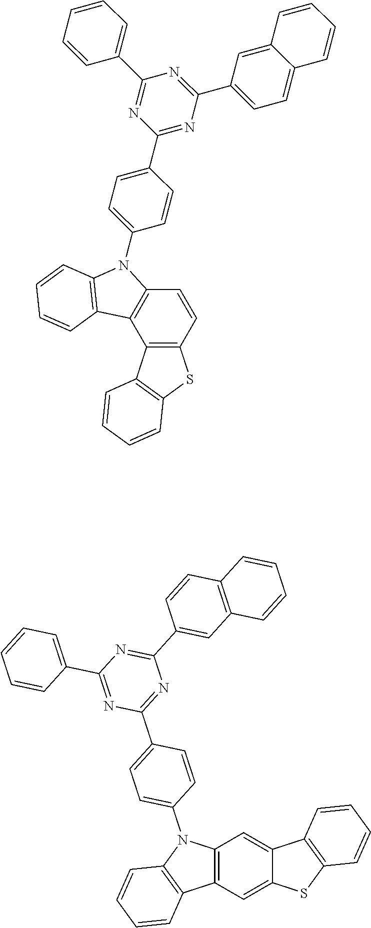 [SMILES: c1ccc(-c2nc(-c3ccc(-n4c5ccccc5c5c6c(ccc54)sc4ccccc46)cc3)nc(-c3ccc4ccccc4c3)n2)cc1 ; c1ccc(-c2nc(-c3ccc(-n4c5ccccc5c5cc6sc7ccccc7c6cc54)cc3)nc(-c3ccc4ccccc4c3)n2)cc1]

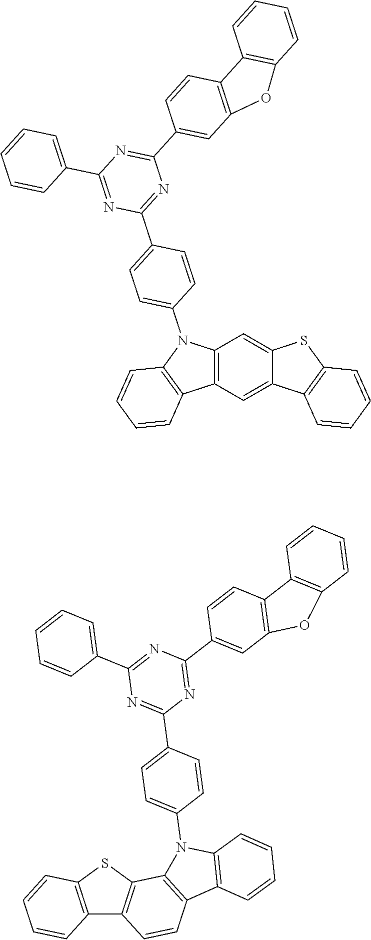 [SMILES: c1ccc(-c2nc(-c3ccc(-n4c5ccccc5c5cc6c(cc54)sc4ccccc46)cc3)nc(-c3ccc4c(c3)oc3ccccc34)n2)cc1 ; c1ccc(-c2nc(-c3ccc(-n4c5ccccc5c5ccc6c7ccccc7sc6c54)cc3)nc(-c3ccc4c(c3)oc3ccccc34)n2)cc1]

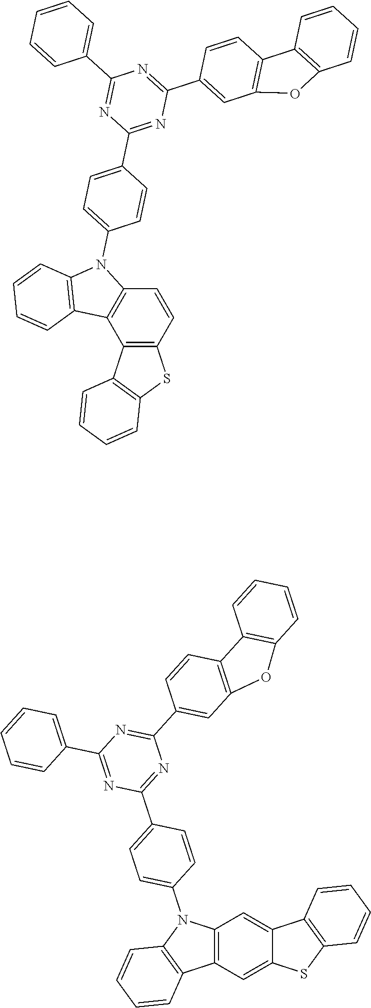 [SMILES: c1ccc(-c2nc(-c3ccc(-n4c5ccccc5c5c6c(ccc54)sc4ccccc46)cc3)nc(-c3ccc4c(c3)oc3ccccc34)n2)cc1 ; c1ccc(-c2nc(-c3ccc(-n4c5ccccc5c5cc6sc7ccccc7c6cc54)cc3)nc(-c3ccc4c(c3)oc3ccccc34)n2)cc1]

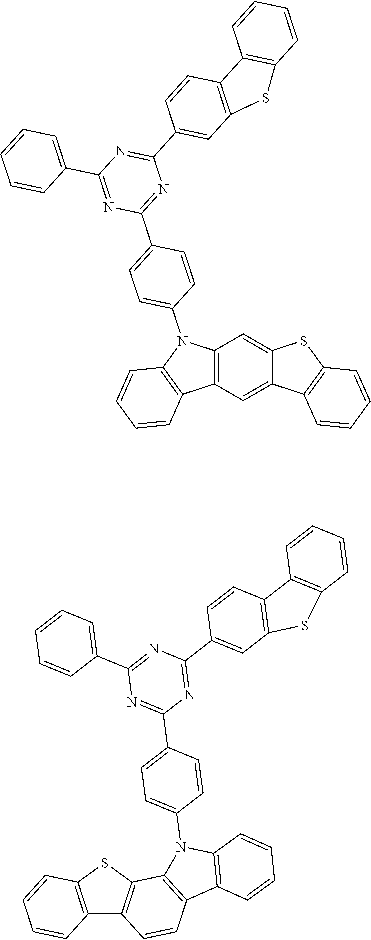 [SMILES: c1ccc(-c2nc(-c3ccc(-n4c5ccccc5c5cc6c(cc54)sc4ccccc46)cc3)nc(-c3ccc4c(c3)sc3ccccc34)n2)cc1 ; c1ccc(-c2nc(-c3ccc(-n4c5ccccc5c5ccc6c7ccccc7sc6c54)cc3)nc(-c3ccc4c(c3)sc3ccccc34)n2)cc1]

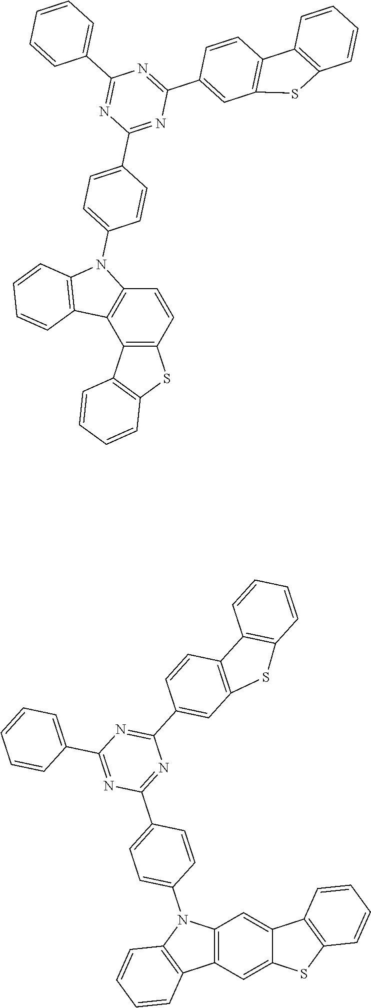 [SMILES: c1ccc(-c2nc(-c3ccc(-n4c5ccccc5c5c6c(ccc54)sc4ccccc46)cc3)nc(-c3ccc4c(c3)sc3ccccc34)n2)cc1 ; c1ccc(-c2nc(-c3ccc(-n4c5ccccc5c5cc6sc7ccccc7c6cc54)cc3)nc(-c3ccc4c(c3)sc3ccccc34)n2)cc1]

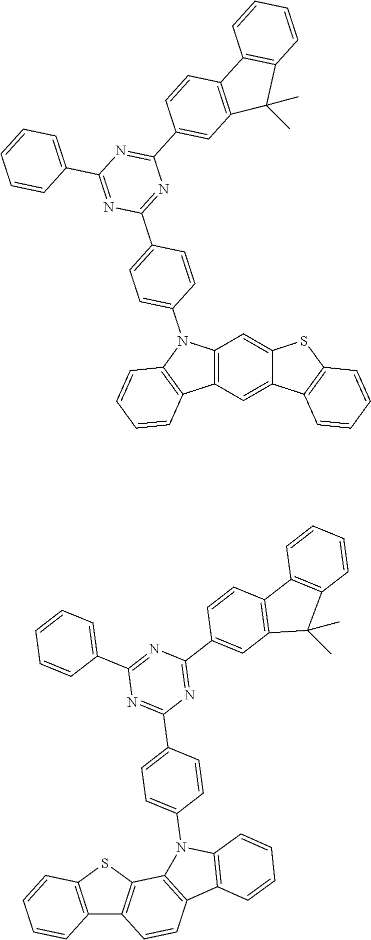 [SMILES: CC1(C)c2ccccc2-c2ccc(-c3nc(-c4ccccc4)nc(-c4ccc(-n5c6ccccc6c6cc7c(cc65)sc5ccccc57)cc4)n3)cc21 ; CC1(C)c2ccccc2-c2ccc(-c3nc(-c4ccccc4)nc(-c4ccc(-n5c6ccccc6c6ccc7c8ccccc8sc7c65)cc4)n3)cc21]

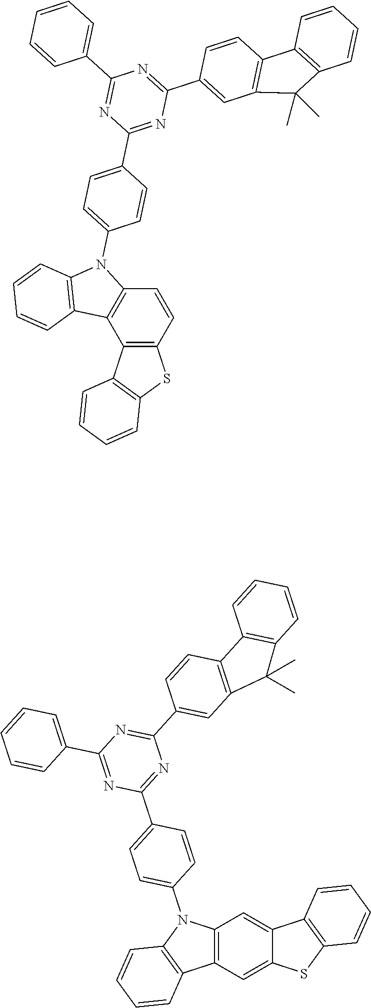 [SMILES: CC1(C)c2ccccc2-c2ccc(-c3nc(-c4ccccc4)nc(-c4ccc(-n5c6ccccc6c6c7c(ccc65)sc5ccccc57)cc4)n3)cc21 ; CC1(C)c2ccccc2-c2ccc(-c3nc(-c4ccccc4)nc(-c4ccc(-n5c6ccccc6c6cc7sc8ccccc8c7cc65)cc4)n3)cc21]

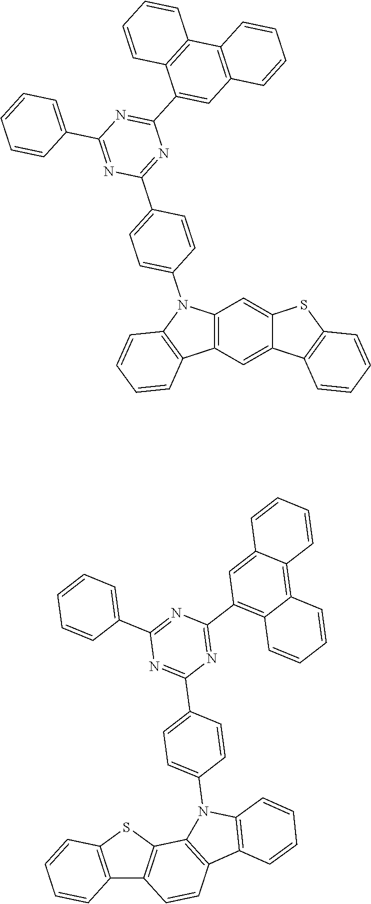 [SMILES: c1ccc(-c2nc(-c3ccc(-n4c5ccccc5c5cc6c(cc54)sc4ccccc46)cc3)nc(-c3cc4ccccc4c4ccccc34)n2)cc1 ; c1ccc(-c2nc(-c3ccc(-n4c5ccccc5c5ccc6c7ccccc7sc6c54)cc3)nc(-c3cc4ccccc4c4ccccc34)n2)cc1]

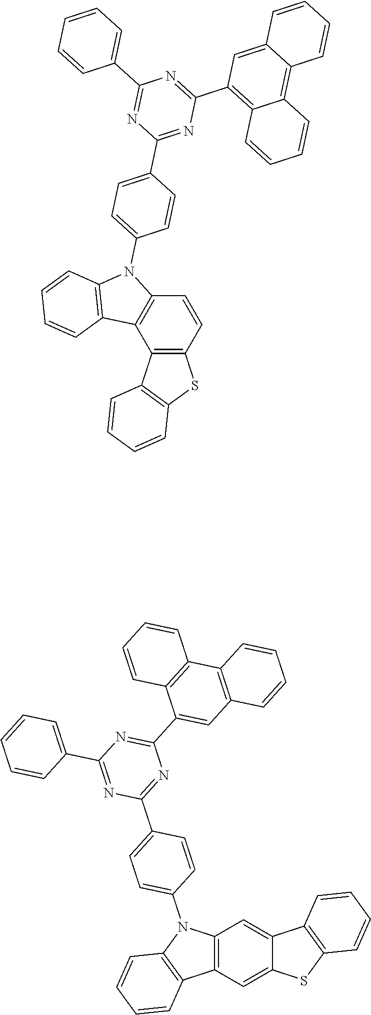 [SMILES: c1ccc(-c2nc(-c3ccc(-n4c5ccccc5c5c6c(ccc54)sc4ccccc46)cc3)nc(-c3cc4ccccc4c4ccccc34)n2)cc1 ; c1ccc(-c2nc(-c3ccc(-n4c5ccccc5c5cc6sc7ccccc7c6cc54)cc3)nc(-c3cc4ccccc4c4ccccc34)n2)cc1]

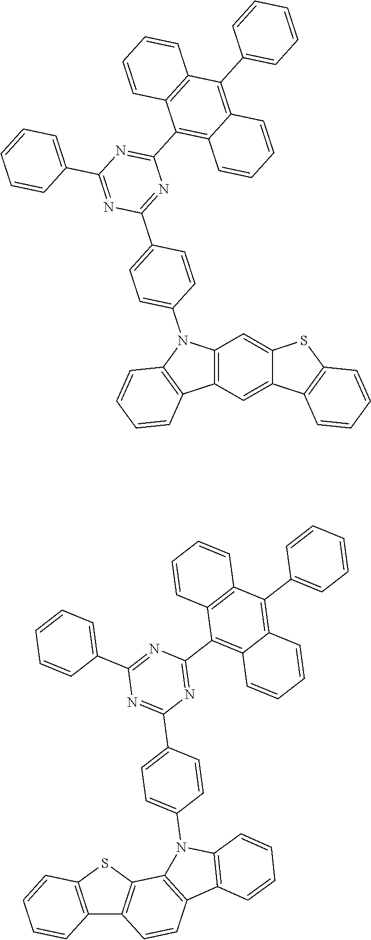 [SMILES: c1ccc(-c2nc(-c3ccc(-n4c5ccccc5c5cc6c(cc54)sc4ccccc46)cc3)nc(-c3c4ccccc4c(-c4ccccc4)c4ccccc34)n2)cc1 ; c1ccc(-c2nc(-c3ccc(-n4c5ccccc5c5ccc6c7ccccc7sc6c54)cc3)nc(-c3c4ccccc4c(-c4ccccc4)c4ccccc34)n2)cc1]

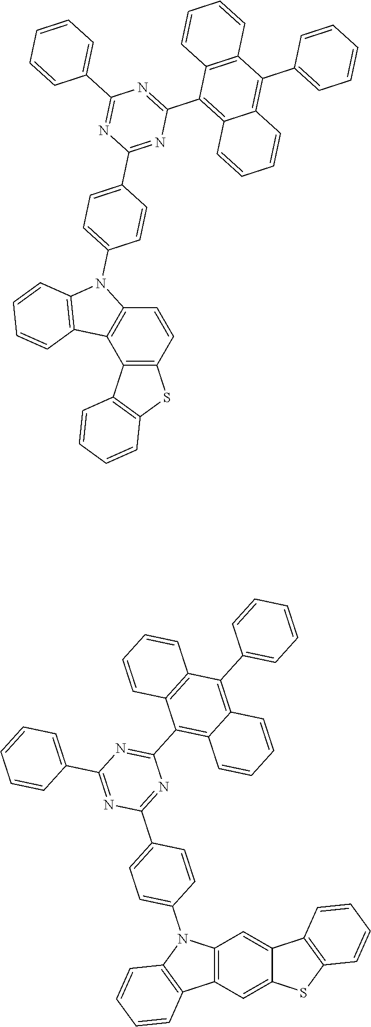 [SMILES: c1ccc(-c2nc(-c3ccc(-n4c5ccccc5c5c6c(ccc54)sc4ccccc46)cc3)nc(-c3c4ccccc4c(-c4ccccc4)c4ccccc34)n2)cc1 ; c1ccc(-c2nc(-c3ccc(-n4c5ccccc5c5cc6sc7ccccc7c6cc54)cc3)nc(-c3c4ccccc4c(-c4ccccc4)c4ccccc34)n2)cc1]

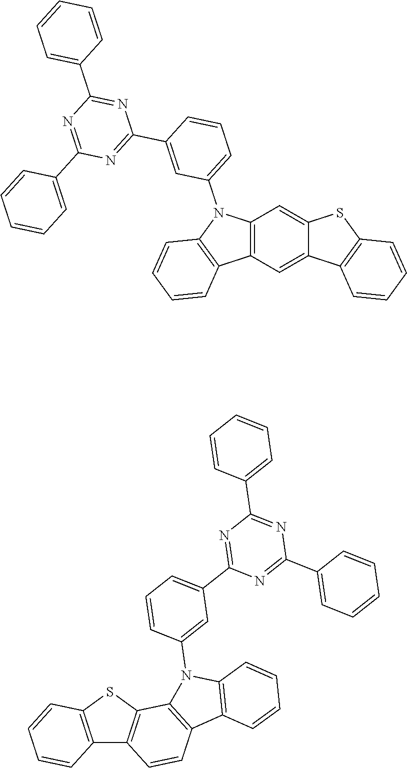 [SMILES: c1ccc(-c2nc(-c3ccccc3)nc(-c3cccc(-n4c5ccccc5c5cc6c(cc54)sc4ccccc46)c3)n2)cc1 ; c1ccc(-c2nc(-c3ccccc3)nc(-c3cccc(-n4c5ccccc5c5ccc6c7ccccc7sc6c54)c3)n2)cc1]

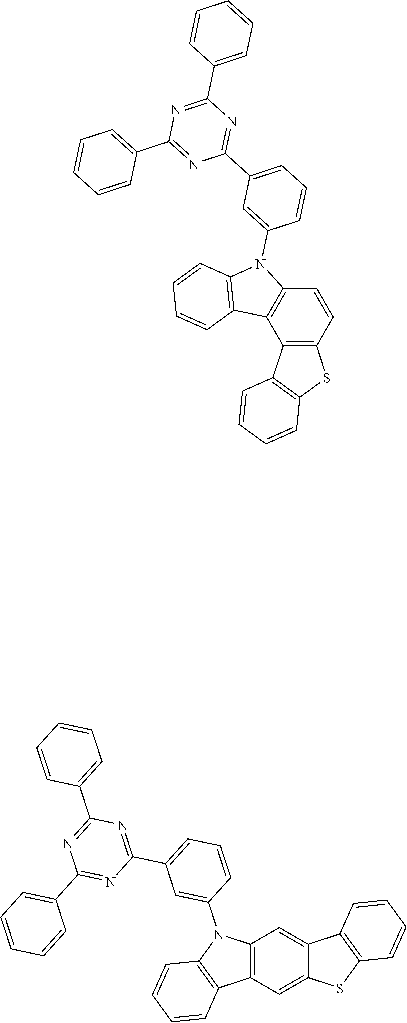 [SMILES: c1ccc(-c2nc(-c3ccccc3)nc(-c3cccc(-n4c5ccccc5c5c6c(ccc54)sc4ccccc46)c3)n2)cc1 ; c1ccc(-c2nc(-c3ccccc3)nc(-c3cccc(-n4c5ccccc5c5cc6sc7ccccc7c6cc54)c3)n2)cc1]

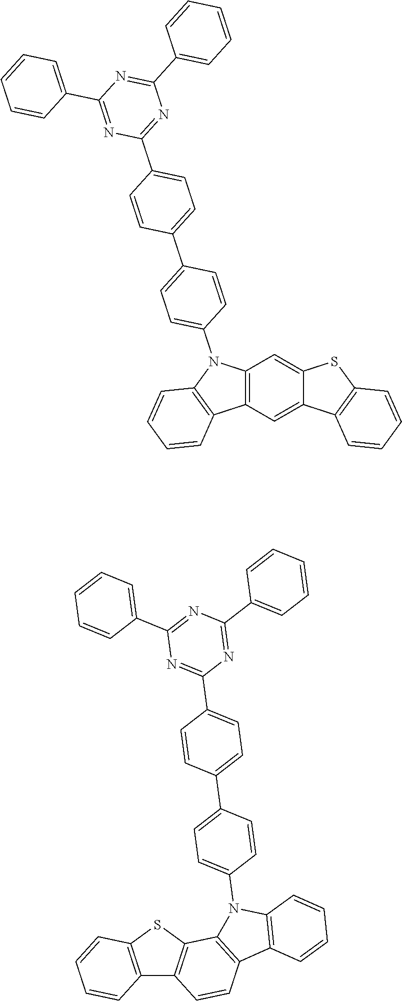 [SMILES: c1ccc(-c2nc(-c3ccccc3)nc(-c3ccc(-c4ccc(-n5c6ccccc6c6cc7c(cc65)sc5ccccc57)cc4)cc3)n2)cc1 ; c1ccc(-c2nc(-c3ccccc3)nc(-c3ccc(-c4ccc(-n5c6ccccc6c6ccc7c8ccccc8sc7c65)cc4)cc3)n2)cc1]

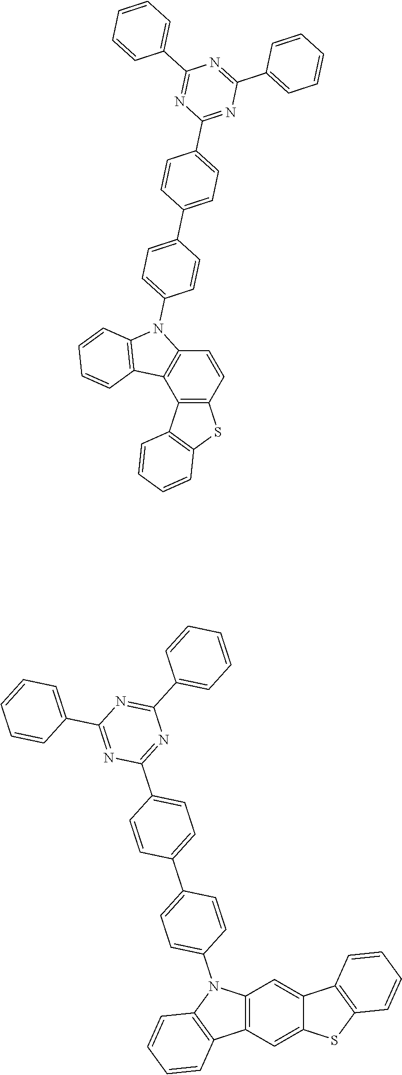 [SMILES: c1ccc(-c2nc(-c3ccccc3)nc(-c3ccc(-c4ccc(-n5c6ccccc6c6c7c(ccc65)sc5ccccc57)cc4)cc3)n2)cc1 ; c1ccc(-c2nc(-c3ccccc3)nc(-c3ccc(-c4ccc(-n5c6ccccc6c6cc7sc8ccccc8c7cc65)cc4)cc3)n2)cc1]

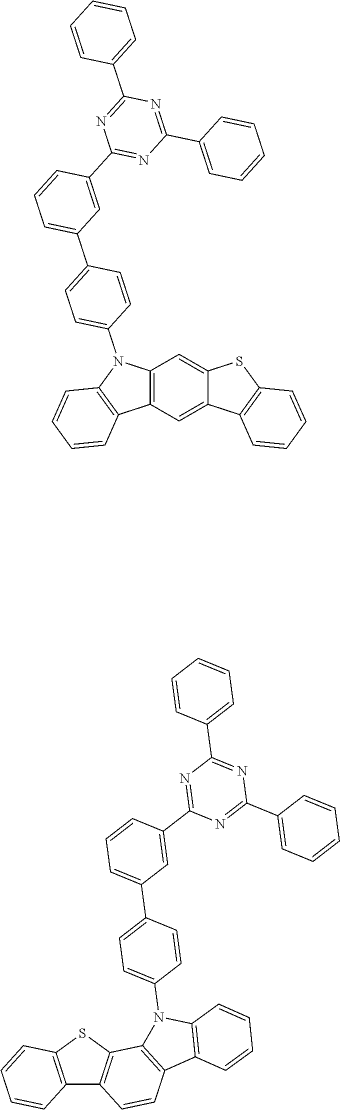 [SMILES: c1ccc(-c2nc(-c3ccccc3)nc(-c3cccc(-c4ccc(-n5c6ccccc6c6cc7c(cc65)sc5ccccc57)cc4)c3)n2)cc1 ; c1ccc(-c2nc(-c3ccccc3)nc(-c3cccc(-c4ccc(-n5c6ccccc6c6ccc7c8ccccc8sc7c65)cc4)c3)n2)cc1]

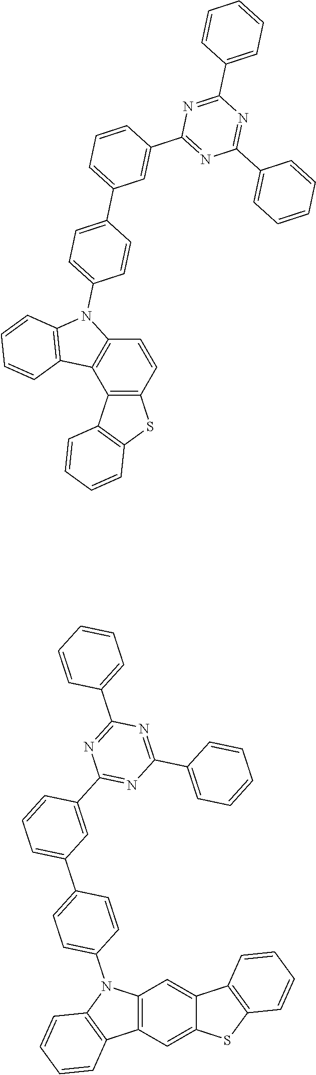 [SMILES: c1ccc(-c2nc(-c3ccccc3)nc(-c3cccc(-c4ccc(-n5c6ccccc6c6c7c(ccc65)sc5ccccc57)cc4)c3)n2)cc1 ; c1ccc(-c2nc(-c3ccccc3)nc(-c3cccc(-c4ccc(-n5c6ccccc6c6cc7sc8ccccc8c7cc65)cc4)c3)n2)cc1]

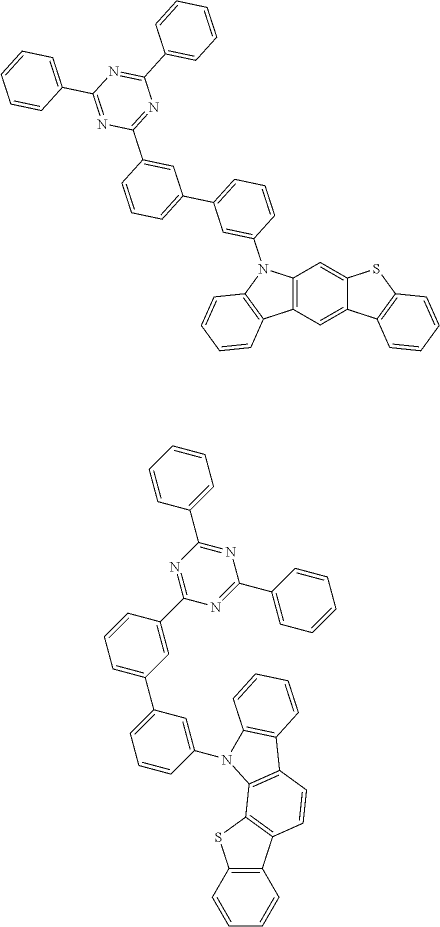 [SMILES: c1ccc(-c2nc(-c3ccccc3)nc(-c3cccc(-c4cccc(-n5c6ccccc6c6cc7c(cc65)sc5ccccc57)c4)c3)n2)cc1 ; c1ccc(-c2nc(-c3ccccc3)nc(-c3cccc(-c4cccc(-n5c6ccccc6c6ccc7c8ccccc8sc7c65)c4)c3)n2)cc1]

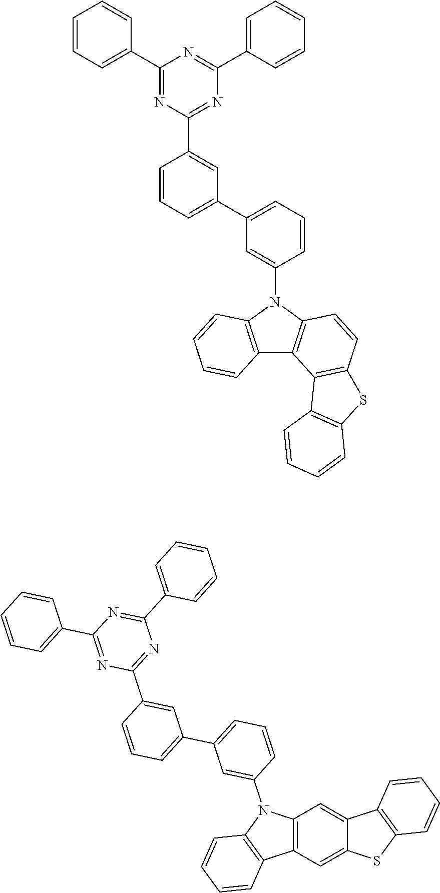 [SMILES: c1ccc(-c2nc(-c3ccccc3)nc(-c3cccc(-c4cccc(-n5c6ccccc6c6c7c(ccc65)sc5ccccc57)c4)c3)n2)cc1 ; c1ccc(-c2nc(-c3ccccc3)nc(-c3cccc(-c4cccc(-n5c6ccccc6c6cc7sc8ccccc8c7cc65)c4)c3)n2)cc1]

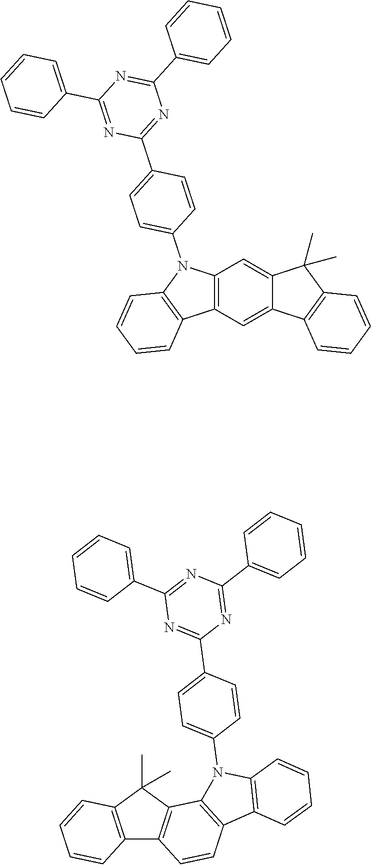 [SMILES: CC1(C)c2ccccc2-c2cc3c4ccccc4n(-c4ccc(-c5nc(-c6ccccc6)nc(-c6ccccc6)n5)cc4)c3cc21 ; CC1(C)c2ccccc2-c2ccc3c4ccccc4n(-c4ccc(-c5nc(-c6ccccc6)nc(-c6ccccc6)n5)cc4)c3c21]

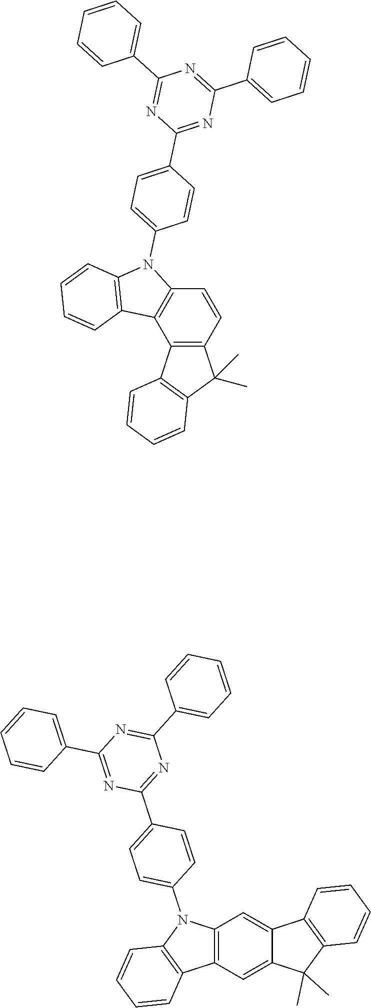 [SMILES: CC1(C)c2ccccc2-c2c1ccc1c2c2ccccc2n1-c1ccc(-c2nc(-c3ccccc3)nc(-c3ccccc3)n2)cc1 ; CC1(C)c2ccccc2-c2cc3c(cc21)c1ccccc1n3-c1ccc(-c2nc(-c3ccccc3)nc(-c3ccccc3)n2)cc1]

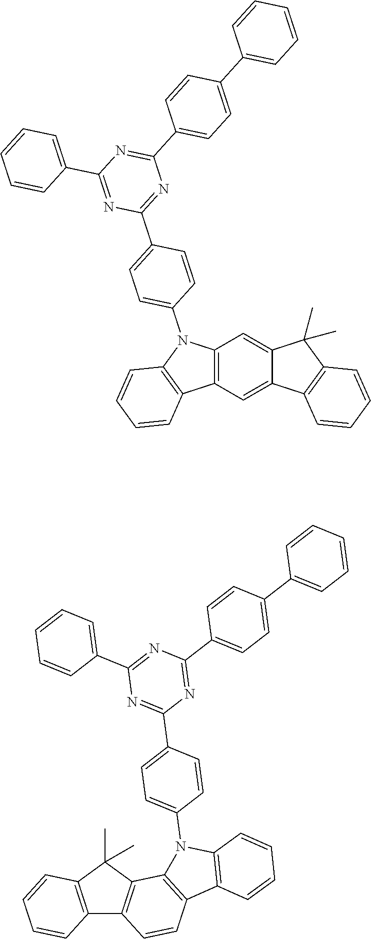 [SMILES: CC1(C)c2ccccc2-c2cc3c4ccccc4n(-c4ccc(-c5nc(-c6ccccc6)nc(-c6ccc(-c7ccccc7)cc6)n5)cc4)c3cc21 ; CC1(C)c2ccccc2-c2ccc3c4ccccc4n(-c4ccc(-c5nc(-c6ccccc6)nc(-c6ccc(-c7ccccc7)cc6)n5)cc4)c3c21]

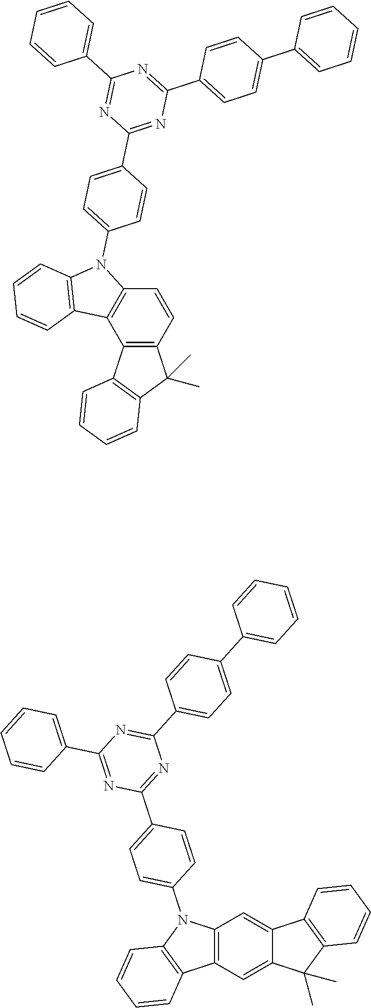 [SMILES: CC1(C)c2ccccc2-c2c1ccc1c2c2ccccc2n1-c1ccc(-c2nc(-c3ccccc3)nc(-c3ccc(-c4ccccc4)cc3)n2)cc1 ; CC1(C)c2ccccc2-c2cc3c(cc21)c1ccccc1n3-c1ccc(-c2nc(-c3ccccc3)nc(-c3ccc(-c4ccccc4)cc3)n2)cc1]

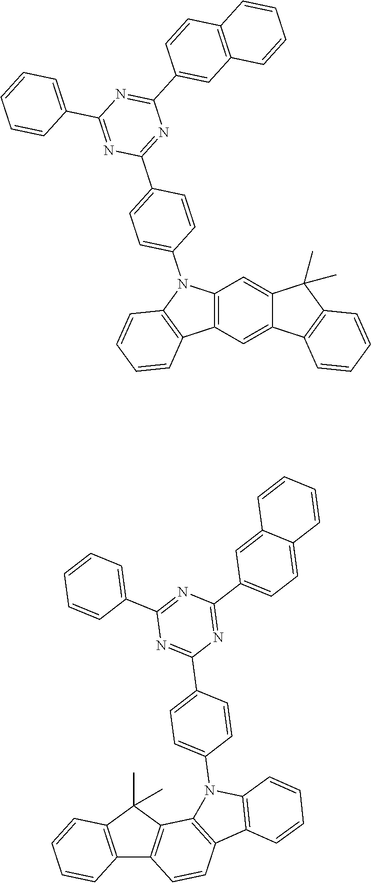 [SMILES: CC1(C)c2ccccc2-c2cc3c4ccccc4n(-c4ccc(-c5nc(-c6ccccc6)nc(-c6ccc7ccccc7c6)n5)cc4)c3cc21 ; CC1(C)c2ccccc2-c2ccc3c4ccccc4n(-c4ccc(-c5nc(-c6ccccc6)nc(-c6ccc7ccccc7c6)n5)cc4)c3c21]

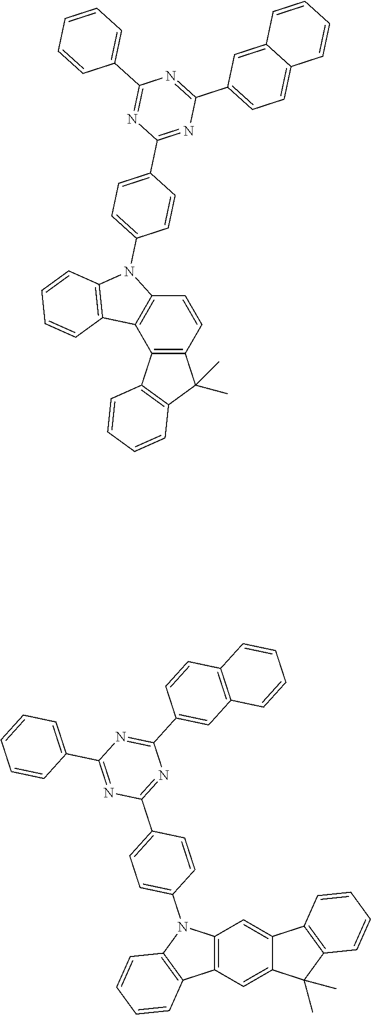 [SMILES: CC1(C)c2ccccc2-c2c1ccc1c2c2ccccc2n1-c1ccc(-c2nc(-c3ccccc3)nc(-c3ccc4ccccc4c3)n2)cc1 ; CC1(C)c2ccccc2-c2cc3c(cc21)c1ccccc1n3-c1ccc(-c2nc(-c3ccccc3)nc(-c3ccc4ccccc4c3)n2)cc1]

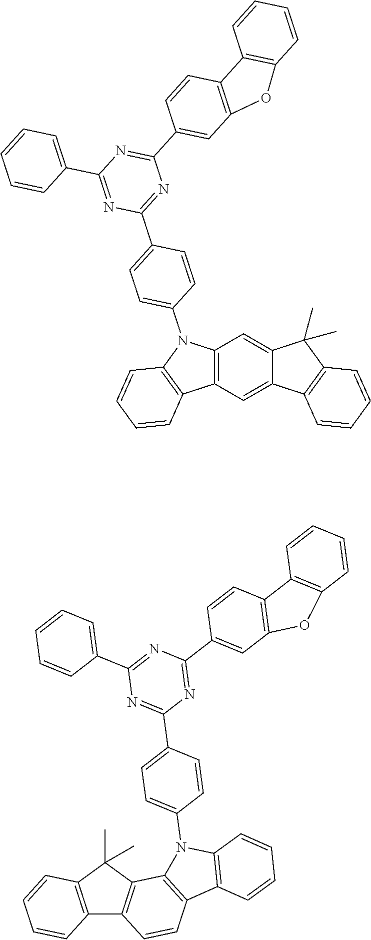 [SMILES: CC1(C)c2ccccc2-c2cc3c4ccccc4n(-c4ccc(-c5nc(-c6ccccc6)nc(-c6ccc7c(c6)oc6ccccc67)n5)cc4)c3cc21 ; CC1(C)c2ccccc2-c2ccc3c4ccccc4n(-c4ccc(-c5nc(-c6ccccc6)nc(-c6ccc7c(c6)oc6ccccc67)n5)cc4)c3c21]

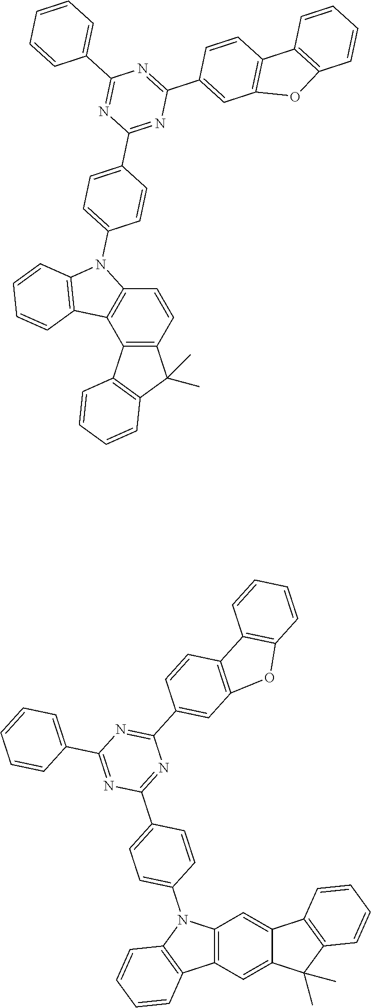 [SMILES: CC1(C)c2ccccc2-c2c1ccc1c2c2ccccc2n1-c1ccc(-c2nc(-c3ccccc3)nc(-c3ccc4c(c3)oc3ccccc34)n2)cc1 ; CC1(C)c2ccccc2-c2cc3c(cc21)c1ccccc1n3-c1ccc(-c2nc(-c3ccccc3)nc(-c3ccc4c(c3)oc3ccccc34)n2)cc1]

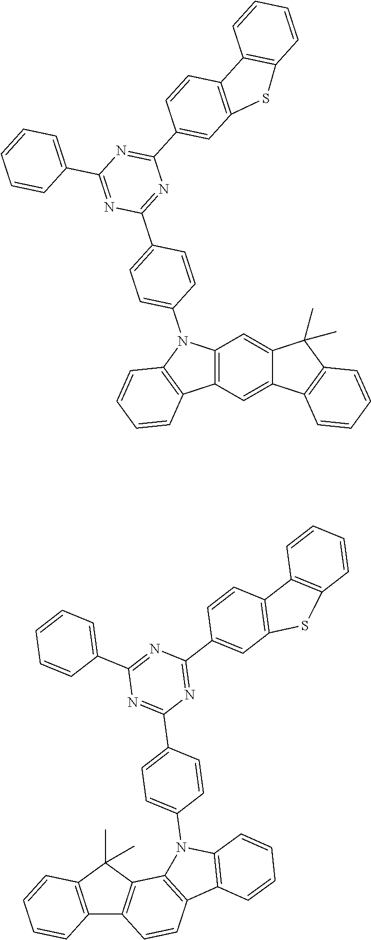 [SMILES: CC1(C)c2ccccc2-c2cc3c4ccccc4n(-c4ccc(-c5nc(-c6ccccc6)nc(-c6ccc7c(c6)sc6ccccc67)n5)cc4)c3cc21 ; CC1(C)c2ccccc2-c2ccc3c4ccccc4n(-c4ccc(-c5nc(-c6ccccc6)nc(-c6ccc7c(c6)sc6ccccc67)n5)cc4)c3c21]

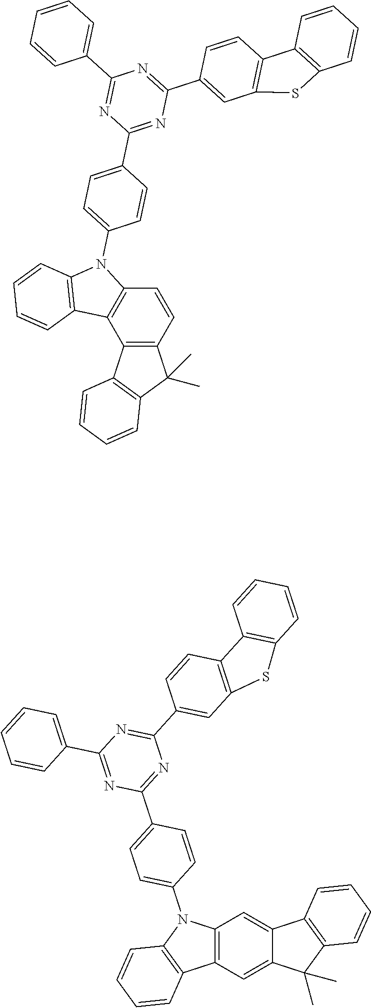 [SMILES: CC1(C)c2ccccc2-c2c1ccc1c2c2ccccc2n1-c1ccc(-c2nc(-c3ccccc3)nc(-c3ccc4c(c3)sc3ccccc34)n2)cc1 ; CC1(C)c2ccccc2-c2cc3c(cc21)c1ccccc1n3-c1ccc(-c2nc(-c3ccccc3)nc(-c3ccc4c(c3)sc3ccccc34)n2)cc1]

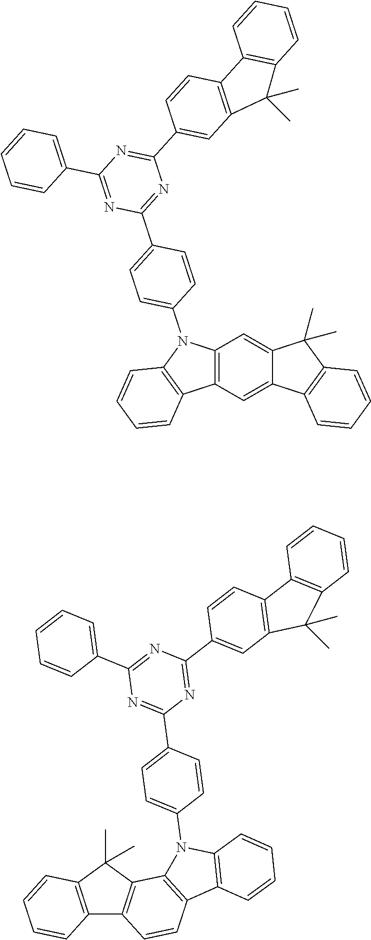 [SMILES: CC1(C)c2ccccc2-c2ccc(-c3nc(-c4ccccc4)nc(-c4ccc(-n5c6ccccc6c6cc7c(cc65)C(C)(C)c5ccccc5-7)cc4)n3)cc21 ; CC1(C)c2ccccc2-c2ccc(-c3nc(-c4ccccc4)nc(-c4ccc(-n5c6ccccc6c6ccc7c(c65)C(C)(C)c5ccccc5-7)cc4)n3)cc21]

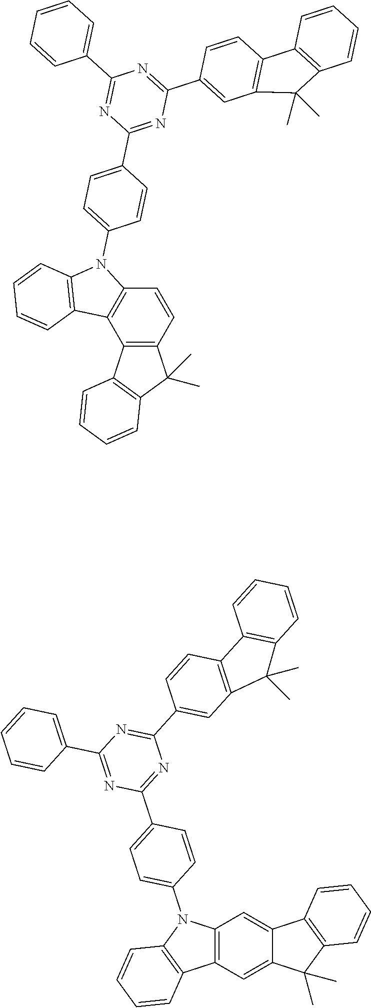 [SMILES: CC1(C)c2ccccc2-c2ccc(-c3nc(-c4ccccc4)nc(-c4ccc(-n5c6ccccc6c6c7c(ccc65)C(C)(C)c5ccccc5-7)cc4)n3)cc21 ; CC1(C)c2ccccc2-c2ccc(-c3nc(-c4ccccc4)nc(-c4ccc(-n5c6ccccc6c6cc7c(cc65)-c5ccccc5C7(C)C)cc4)n3)cc21]

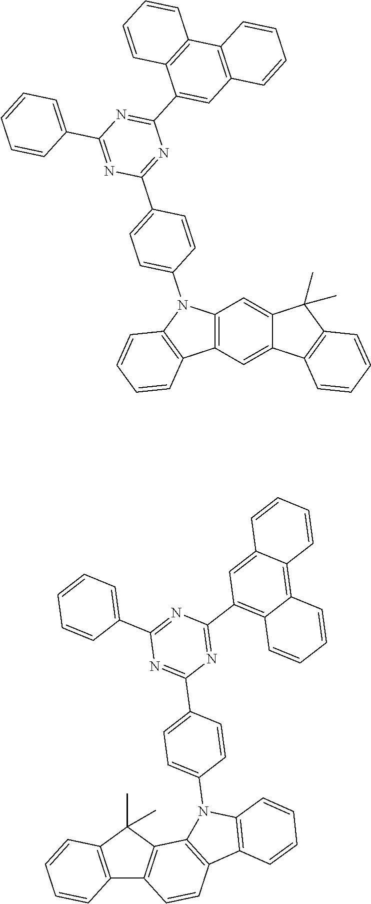 [SMILES: CC1(C)c2ccccc2-c2cc3c4ccccc4n(-c4ccc(-c5nc(-c6ccccc6)nc(-c6cc7ccccc7c7ccccc67)n5)cc4)c3cc21 ; CC1(C)c2ccccc2-c2ccc3c4ccccc4n(-c4ccc(-c5nc(-c6ccccc6)nc(-c6cc7ccccc7c7ccccc67)n5)cc4)c3c21]

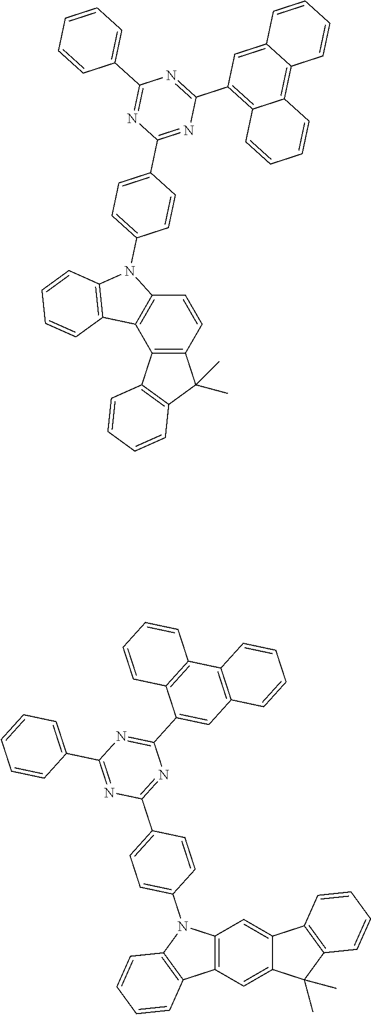 [SMILES: CC1(C)c2ccccc2-c2c1ccc1c2c2ccccc2n1-c1ccc(-c2nc(-c3ccccc3)nc(-c3cc4ccccc4c4ccccc34)n2)cc1 ; CC1(C)c2ccccc2-c2cc3c(cc21)c1ccccc1n3-c1ccc(-c2nc(-c3ccccc3)nc(-c3cc4ccccc4c4ccccc34)n2)cc1]

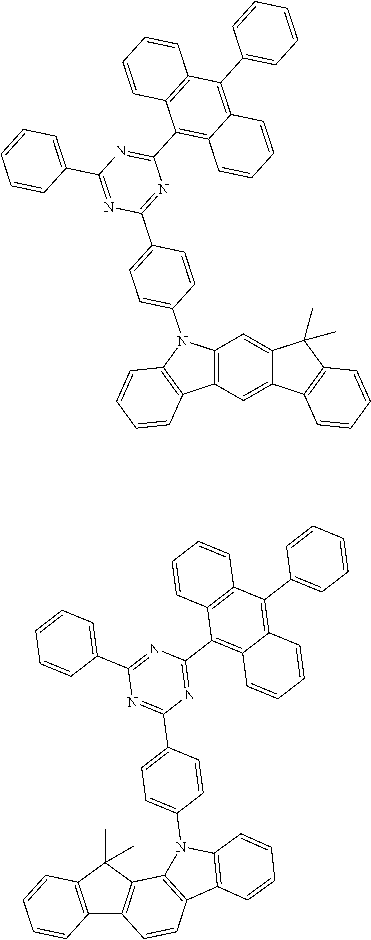 [SMILES: CC1(C)c2ccccc2-c2cc3c4ccccc4n(-c4ccc(-c5nc(-c6ccccc6)nc(-c6c7ccccc7c(-c7ccccc7)c7ccccc67)n5)cc4)c3cc21 ; CC1(C)c2ccccc2-c2ccc3c4ccccc4n(-c4ccc(-c5nc(-c6ccccc6)nc(-c6c7ccccc7c(-c7ccccc7)c7ccccc67)n5)cc4)c3c21]

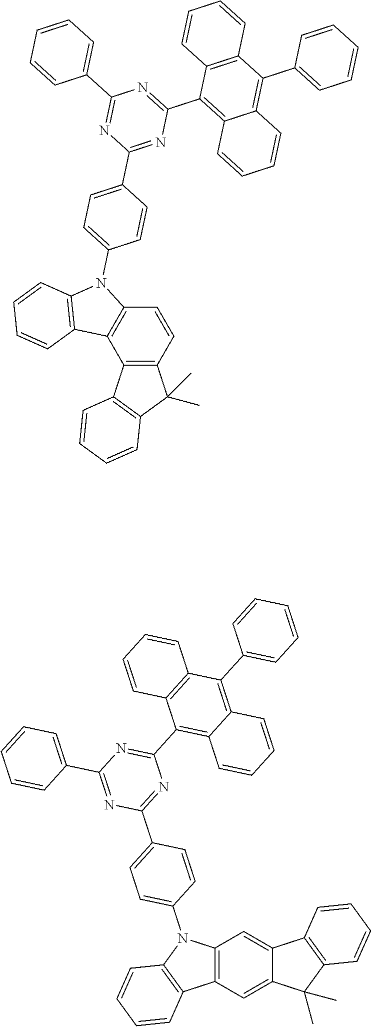 [SMILES: CC1(C)c2ccccc2-c2c1ccc1c2c2ccccc2n1-c1ccc(-c2nc(-c3ccccc3)nc(-c3c4ccccc4c(-c4ccccc4)c4ccccc34)n2)cc1 ; CC1(C)c2ccccc2-c2cc3c(cc21)c1ccccc1n3-c1ccc(-c2nc(-c3ccccc3)nc(-c3c4ccccc4c(-c4ccccc4)c4ccccc34)n2)cc1]

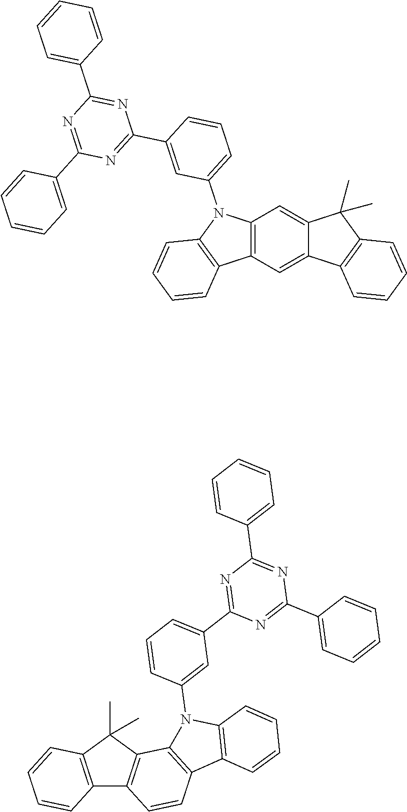 [SMILES: CC1(C)c2ccccc2-c2cc3c4ccccc4n(-c4cccc(-c5nc(-c6ccccc6)nc(-c6ccccc6)n5)c4)c3cc21 ; CC1(C)c2ccccc2-c2ccc3c4ccccc4n(-c4cccc(-c5nc(-c6ccccc6)nc(-c6ccccc6)n5)c4)c3c21]

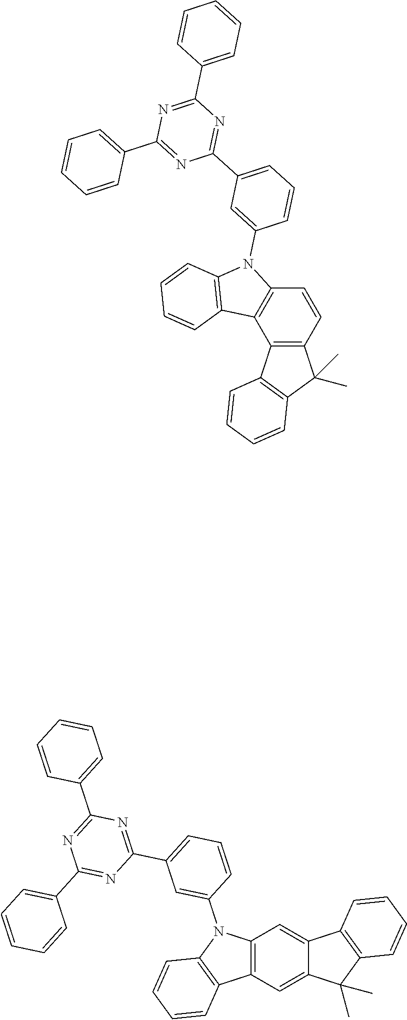 [SMILES: CC1(C)c2ccccc2-c2c1ccc1c2c2ccccc2n1-c1cccc(-c2nc(-c3ccccc3)nc(-c3ccccc3)n2)c1 ; CC1(C)c2ccccc2-c2cc3c(cc21)c1ccccc1n3-c1cccc(-c2nc(-c3ccccc3)nc(-c3ccccc3)n2)c1]

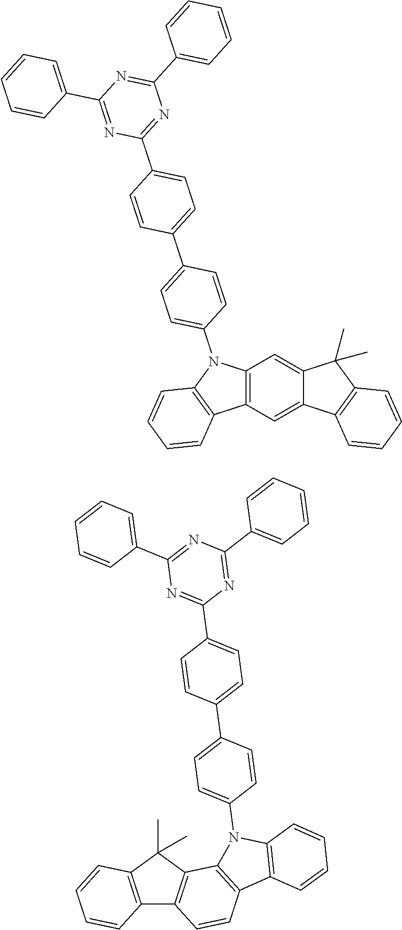 [SMILES: CC1(C)c2ccccc2-c2cc3c4ccccc4n(-c4ccc(-c5ccc(-c6nc(-c7ccccc7)nc(-c7ccccc7)n6)cc5)cc4)c3cc21 ; CC1(C)c2ccccc2-c2ccc3c4ccccc4n(-c4ccc(-c5ccc(-c6nc(-c7ccccc7)nc(-c7ccccc7)n6)cc5)cc4)c3c21]

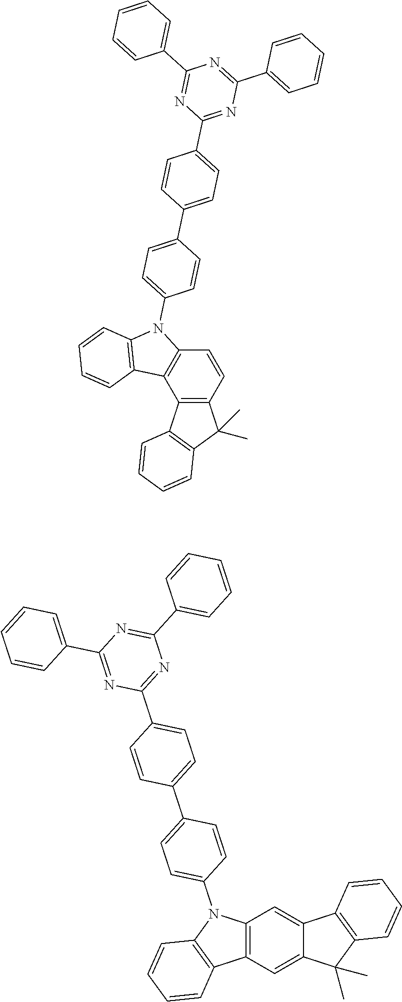 [SMILES: CC1(C)c2ccccc2-c2c1ccc1c2c2ccccc2n1-c1ccc(-c2ccc(-c3nc(-c4ccccc4)nc(-c4ccccc4)n3)cc2)cc1 ; CC1(C)c2ccccc2-c2cc3c(cc21)c1ccccc1n3-c1ccc(-c2ccc(-c3nc(-c4ccccc4)nc(-c4ccccc4)n3)cc2)cc1]

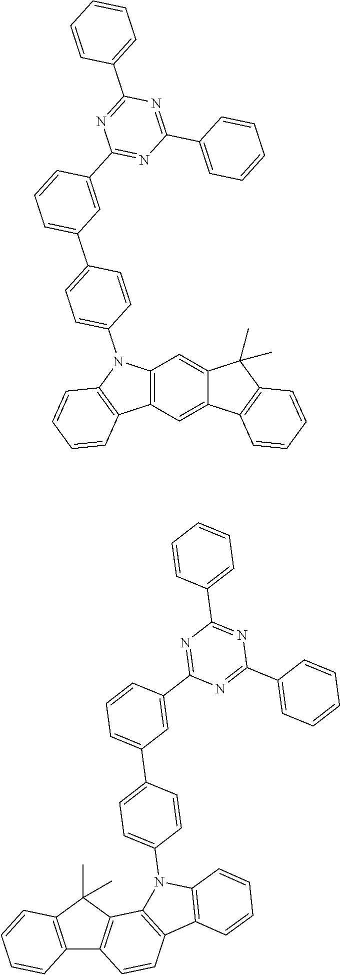 [SMILES: CC1(C)c2ccccc2-c2cc3c4ccccc4n(-c4ccc(-c5cccc(-c6nc(-c7ccccc7)nc(-c7ccccc7)n6)c5)cc4)c3cc21 ; CC1(C)c2ccccc2-c2ccc3c4ccccc4n(-c4ccc(-c5cccc(-c6nc(-c7ccccc7)nc(-c7ccccc7)n6)c5)cc4)c3c21]

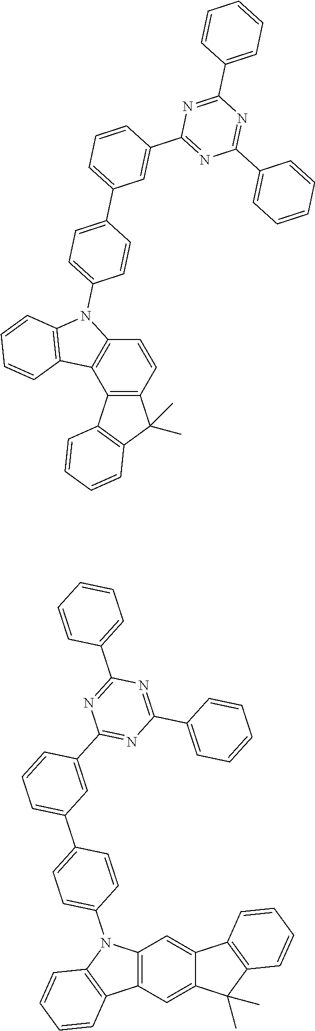 [SMILES: CC1(C)c2ccccc2-c2c1ccc1c2c2ccccc2n1-c1ccc(-c2cccc(-c3nc(-c4ccccc4)nc(-c4ccccc4)n3)c2)cc1 ; CC1(C)c2ccccc2-c2cc3c(cc21)c1ccccc1n3-c1ccc(-c2cccc(-c3nc(-c4ccccc4)nc(-c4ccccc4)n3)c2)cc1]

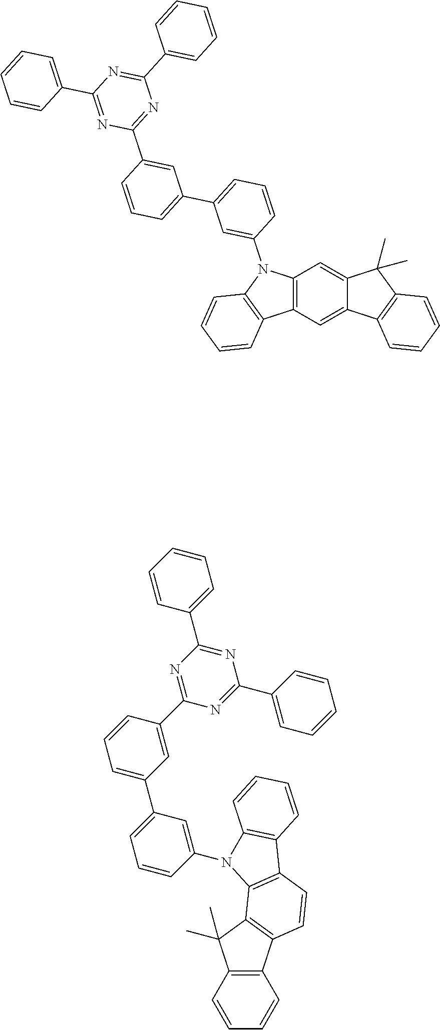 [SMILES: CC1(C)c2ccccc2-c2cc3c4ccccc4n(-c4cccc(-c5cccc(-c6nc(-c7ccccc7)nc(-c7ccccc7)n6)c5)c4)c3cc21 ; CC1(C)c2ccccc2-c2ccc3c4ccccc4n(-c4cccc(-c5cccc(-c6nc(-c7ccccc7)nc(-c7ccccc7)n6)c5)c4)c3c21]

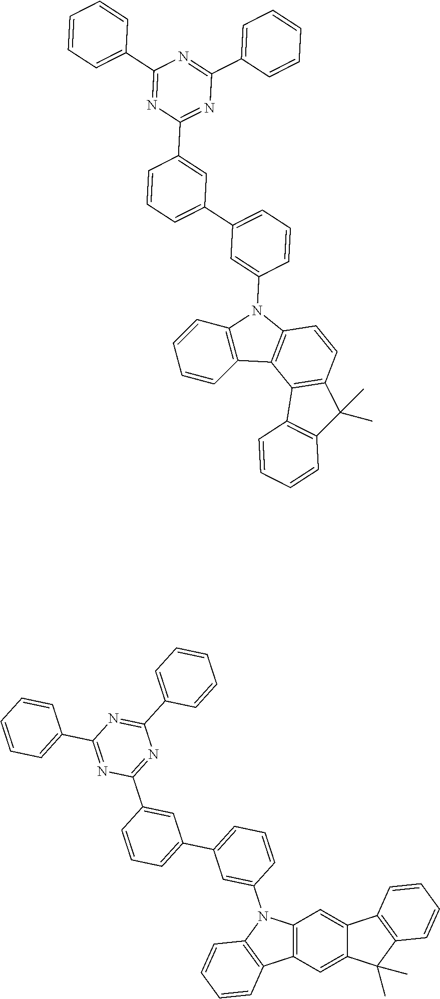 [SMILES: CC1(C)c2ccccc2-c2c1ccc1c2c2ccccc2n1-c1cccc(-c2cccc(-c3nc(-c4ccccc4)nc(-c4ccccc4)n3)c2)c1 ; CC1(C)c2ccccc2-c2cc3c(cc21)c1ccccc1n3-c1cccc(-c2cccc(-c3nc(-c4ccccc4)nc(-c4ccccc4)n3)c2)c1]

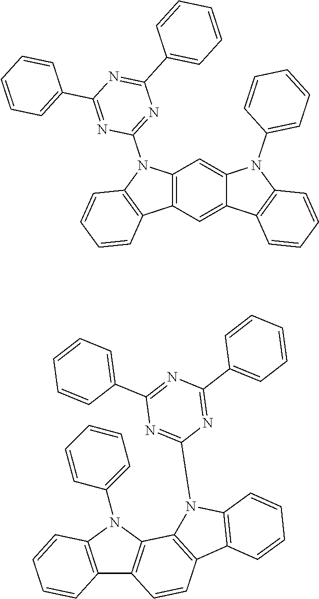 [SMILES: c1ccc(-c2nc(-c3ccccc3)nc(-n3c4ccccc4c4cc5c6ccccc6n(-c6ccccc6)c5cc43)n2)cc1 ; c1ccc(-c2nc(-c3ccccc3)nc(-n3c4ccccc4c4ccc5c6ccccc6n(-c6ccccc6)c5c43)n2)cc1]

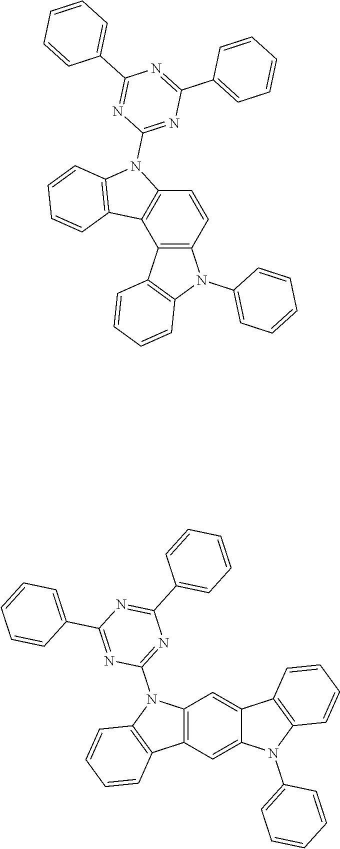 [SMILES: c1ccc(-c2nc(-c3ccccc3)nc(-n3c4ccccc4c4c5c6ccccc6n(-c6ccccc6)c5ccc43)n2)cc1 ; c1ccc(-c2nc(-c3ccccc3)nc(-n3c4ccccc4c4cc5c(cc43)c3ccccc3n5-c3ccccc3)n2)cc1]

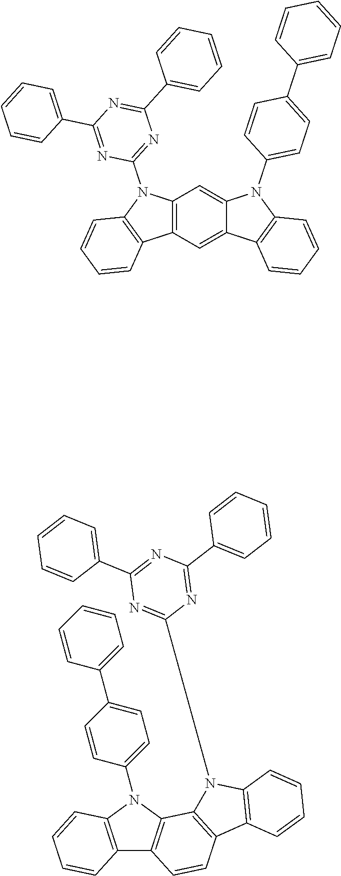 [SMILES: c1ccc(-c2ccc(-n3c4ccccc4c4cc5c6ccccc6n(-c6nc(-c7ccccc7)nc(-c7ccccc7)n6)c5cc43)cc2)cc1 ; c1ccc(-c2ccc(-n3c4ccccc4c4ccc5c6ccccc6n(-c6nc(-c7ccccc7)nc(-c7ccccc7)n6)c5c43)cc2)cc1]

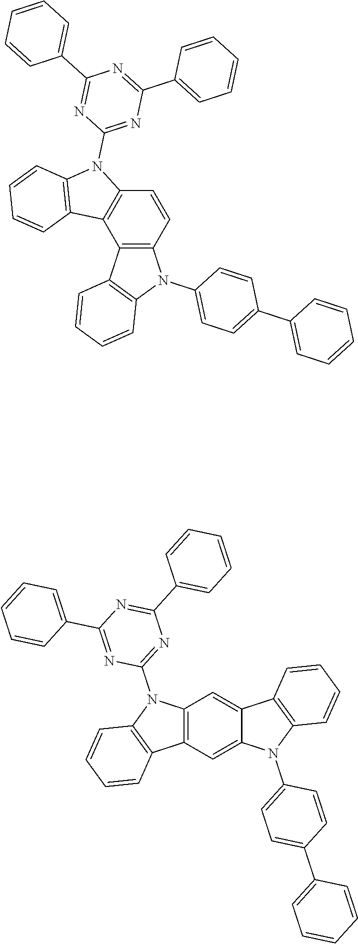 [SMILES: c1ccc(-c2ccc(-n3c4ccccc4c4c5c6ccccc6n(-c6nc(-c7ccccc7)nc(-c7ccccc7)n6)c5ccc43)cc2)cc1 ; c1ccc(-c2ccc(-n3c4ccccc4c4cc5c(cc43)c3ccccc3n5-c3nc(-c4ccccc4)nc(-c4ccccc4)n3)cc2)cc1]

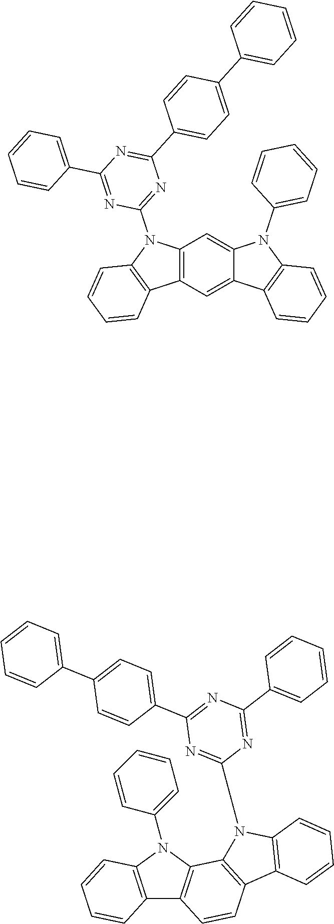 [SMILES: c1ccc(-c2ccc(-c3nc(-c4ccccc4)nc(-n4c5ccccc5c5cc6c7ccccc7n(-c7ccccc7)c6cc54)n3)cc2)cc1 ; c1ccc(-c2ccc(-c3nc(-c4ccccc4)nc(-n4c5ccccc5c5ccc6c7ccccc7n(-c7ccccc7)c6c54)n3)cc2)cc1]

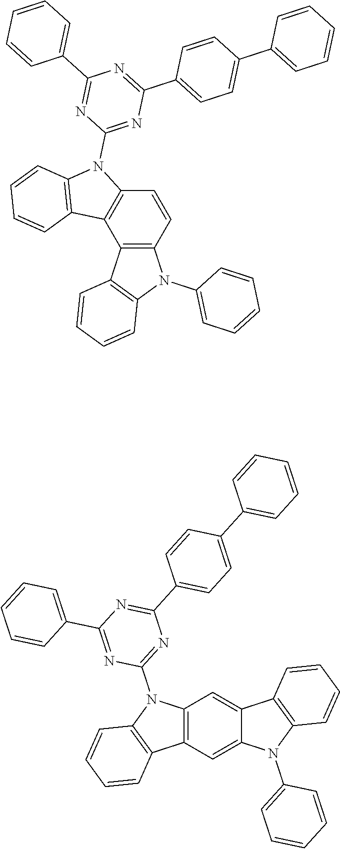 [SMILES: c1ccc(-c2ccc(-c3nc(-c4ccccc4)nc(-n4c5ccccc5c5c6c7ccccc7n(-c7ccccc7)c6ccc54)n3)cc2)cc1 ; c1ccc(-c2ccc(-c3nc(-c4ccccc4)nc(-n4c5ccccc5c5cc6c(cc54)c4ccccc4n6-c4ccccc4)n3)cc2)cc1]

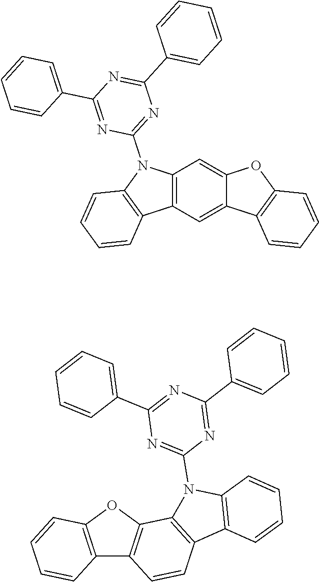 [SMILES: c1ccc(-c2nc(-c3ccccc3)nc(-n3c4ccccc4c4cc5c(cc43)oc3ccccc35)n2)cc1 ; c1ccc(-c2nc(-c3ccccc3)nc(-n3c4ccccc4c4ccc5c6ccccc6oc5c43)n2)cc1]

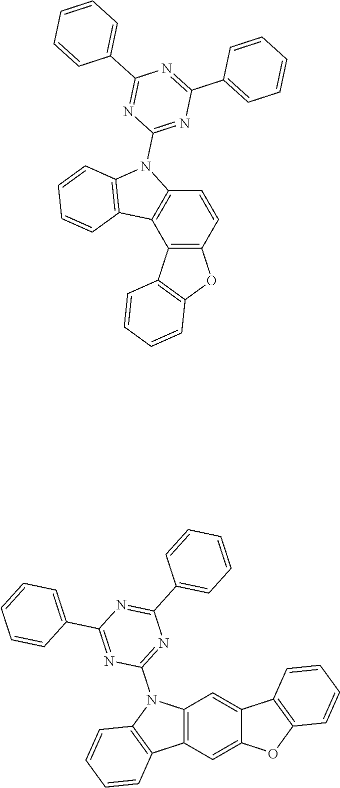 [SMILES: c1ccc(-c2nc(-c3ccccc3)nc(-n3c4ccccc4c4c5c(ccc43)oc3ccccc35)n2)cc1 ; c1ccc(-c2nc(-c3ccccc3)nc(-n3c4ccccc4c4cc5oc6ccccc6c5cc43)n2)cc1]

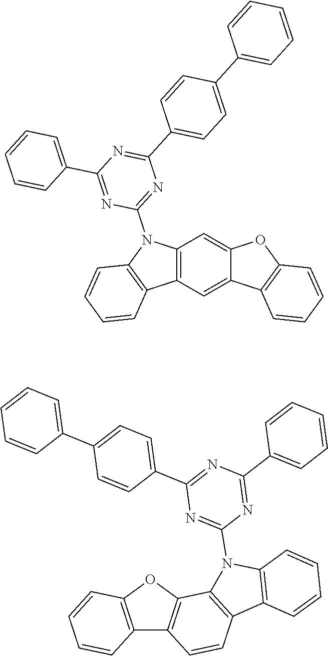 [SMILES: c1ccc(-c2ccc(-c3nc(-c4ccccc4)nc(-n4c5ccccc5c5cc6c(cc54)oc4ccccc46)n3)cc2)cc1 ; c1ccc(-c2ccc(-c3nc(-c4ccccc4)nc(-n4c5ccccc5c5ccc6c7ccccc7oc6c54)n3)cc2)cc1]

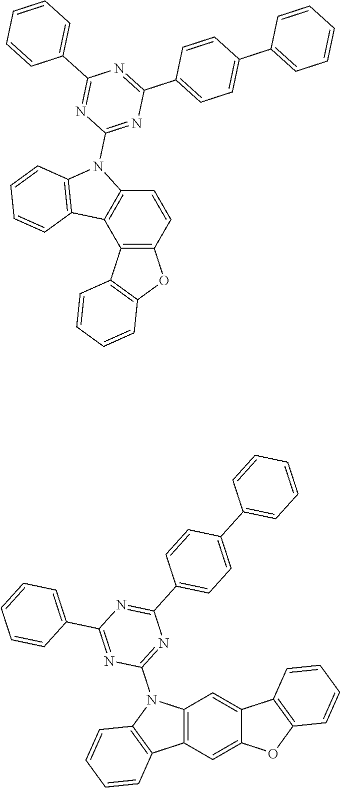 [SMILES: c1ccc(-c2ccc(-c3nc(-c4ccccc4)nc(-n4c5ccccc5c5c6c(ccc54)oc4ccccc46)n3)cc2)cc1 ; c1ccc(-c2ccc(-c3nc(-c4ccccc4)nc(-n4c5ccccc5c5cc6oc7ccccc7c6cc54)n3)cc2)cc1]

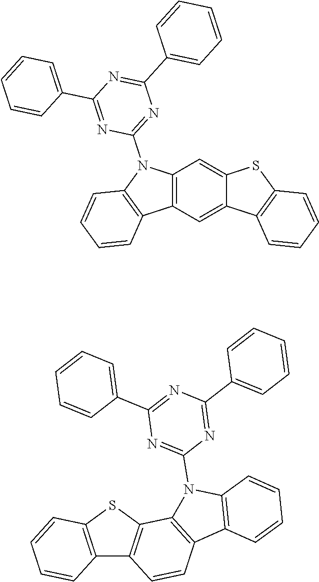 [SMILES: c1ccc(-c2nc(-c3ccccc3)nc(-n3c4ccccc4c4cc5c(cc43)sc3ccccc35)n2)cc1 ; c1ccc(-c2nc(-c3ccccc3)nc(-n3c4ccccc4c4ccc5c6ccccc6sc5c43)n2)cc1]

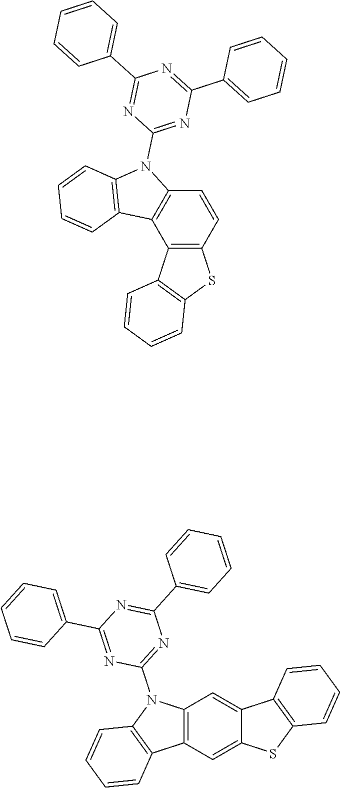 [SMILES: c1ccc(-c2nc(-c3ccccc3)nc(-n3c4ccccc4c4c5c(ccc43)sc3ccccc35)n2)cc1 ; c1ccc(-c2nc(-c3ccccc3)nc(-n3c4ccccc4c4cc5sc6ccccc6c5cc43)n2)cc1]

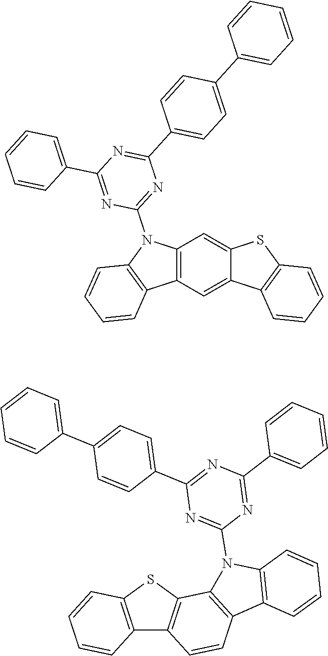 [SMILES: c1ccc(-c2ccc(-c3nc(-c4ccccc4)nc(-n4c5ccccc5c5cc6c(cc54)sc4ccccc46)n3)cc2)cc1 ; c1ccc(-c2ccc(-c3nc(-c4ccccc4)nc(-n4c5ccccc5c5ccc6c7ccccc7sc6c54)n3)cc2)cc1]

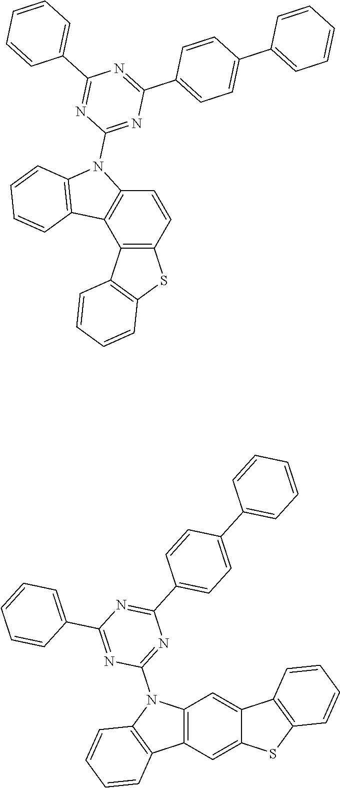 [SMILES: c1ccc(-c2ccc(-c3nc(-c4ccccc4)nc(-n4c5ccccc5c5c6c(ccc54)sc4ccccc46)n3)cc2)cc1 ; c1ccc(-c2ccc(-c3nc(-c4ccccc4)nc(-n4c5ccccc5c5cc6sc7ccccc7c6cc54)n3)cc2)cc1]

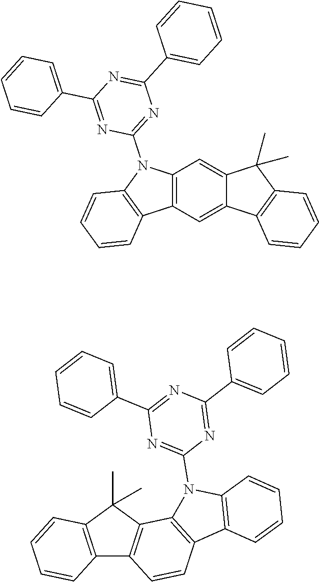 [SMILES: CC1(C)c2ccccc2-c2cc3c4ccccc4n(-c4nc(-c5ccccc5)nc(-c5ccccc5)n4)c3cc21 ; CC1(C)c2ccccc2-c2ccc3c4ccccc4n(-c4nc(-c5ccccc5)nc(-c5ccccc5)n4)c3c21]

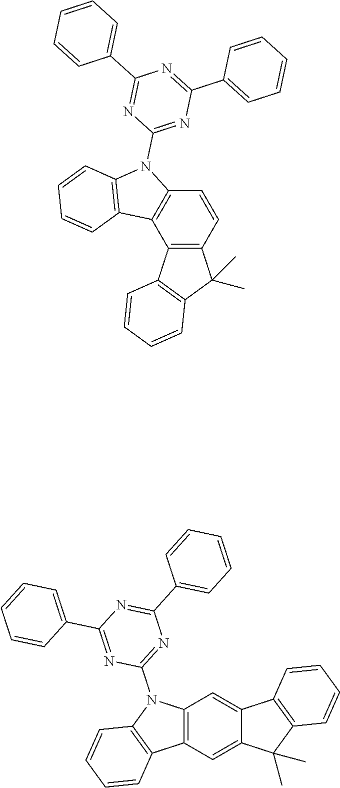 [SMILES: CC1(C)c2ccccc2-c2c1ccc1c2c2ccccc2n1-c1nc(-c2ccccc2)nc(-c2ccccc2)n1 ; CC1(C)c2ccccc2-c2cc3c(cc21)c1ccccc1n3-c1nc(-c2ccccc2)nc(-c2ccccc2)n1]

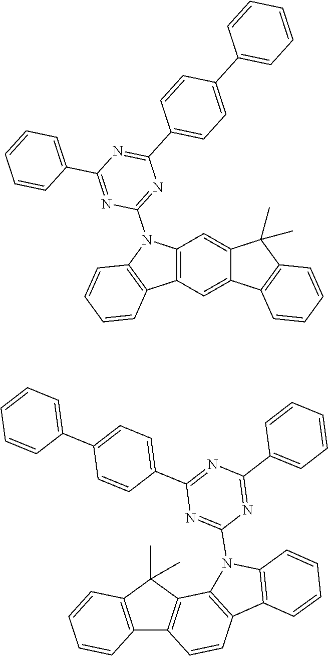 [SMILES: CC1(C)c2ccccc2-c2cc3c4ccccc4n(-c4nc(-c5ccccc5)nc(-c5ccc(-c6ccccc6)cc5)n4)c3cc21 ; CC1(C)c2ccccc2-c2ccc3c4ccccc4n(-c4nc(-c5ccccc5)nc(-c5ccc(-c6ccccc6)cc5)n4)c3c21]

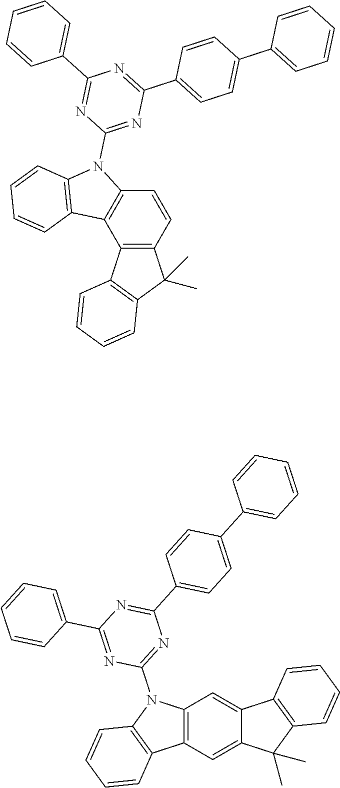 [SMILES: CC1(C)c2ccccc2-c2c1ccc1c2c2ccccc2n1-c1nc(-c2ccccc2)nc(-c2ccc(-c3ccccc3)cc2)n1 ; CC1(C)c2ccccc2-c2cc3c(cc21)c1ccccc1n3-c1nc(-c2ccccc2)nc(-c2ccc(-c3ccccc3)cc2)n1]

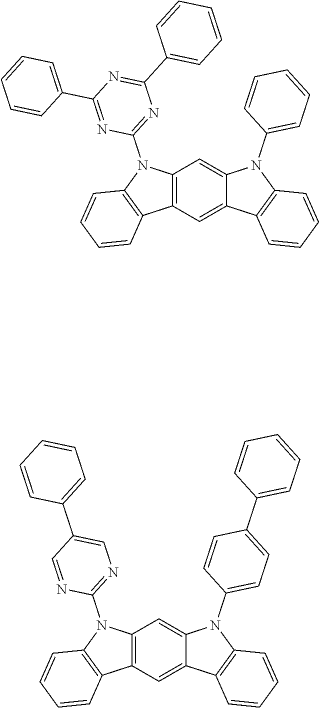 [SMILES: c1ccc(-c2nc(-c3ccccc3)nc(-n3c4ccccc4c4cc5c6ccccc6n(-c6ccccc6)c5cc43)n2)cc1 ; c1ccc(-c2ccc(-n3c4ccccc4c4cc5c6ccccc6n(-c6ncc(-c7ccccc7)cn6)c5cc43)cc2)cc1]

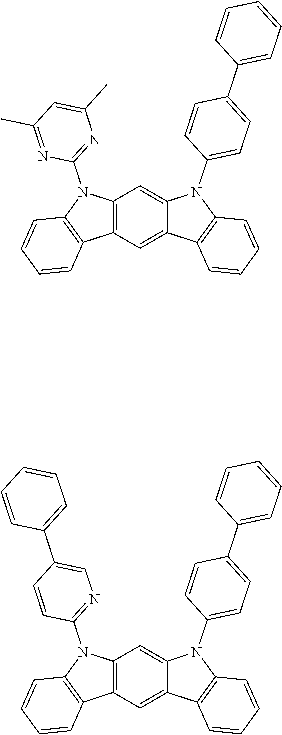 [SMILES: Cc1cc(C)nc(-n2c3ccccc3c3cc4c5ccccc5n(-c5ccc(-c6ccccc6)cc5)c4cc32)n1 ; c1ccc(-c2ccc(-n3c4ccccc4c4cc5c6ccccc6n(-c6ccc(-c7ccccc7)cn6)c5cc43)cc2)cc1]

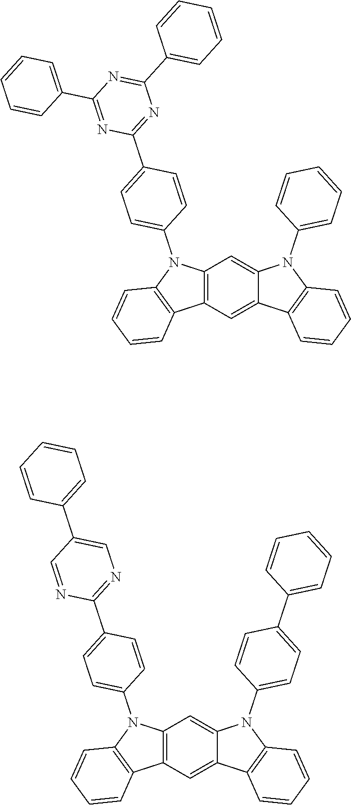 [SMILES: c1ccc(-c2nc(-c3ccccc3)nc(-c3ccc(-n4c5ccccc5c5cc6c7ccccc7n(-c7ccccc7)c6cc54)cc3)n2)cc1 ; c1ccc(-c2ccc(-n3c4ccccc4c4cc5c6ccccc6n(-c6ccc(-c7ncc(-c8ccccc8)cn7)cc6)c5cc43)cc2)cc1]

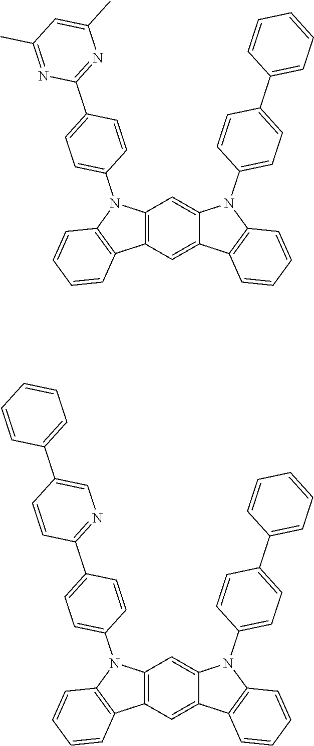 [SMILES: Cc1cc(C)nc(-c2ccc(-n3c4ccccc4c4cc5c6ccccc6n(-c6ccc(-c7ccccc7)cc6)c5cc43)cc2)n1 ; c1ccc(-c2ccc(-n3c4ccccc4c4cc5c6ccccc6n(-c6ccc(-c7ccc(-c8ccccc8)cn7)cc6)c5cc43)cc2)cc1]

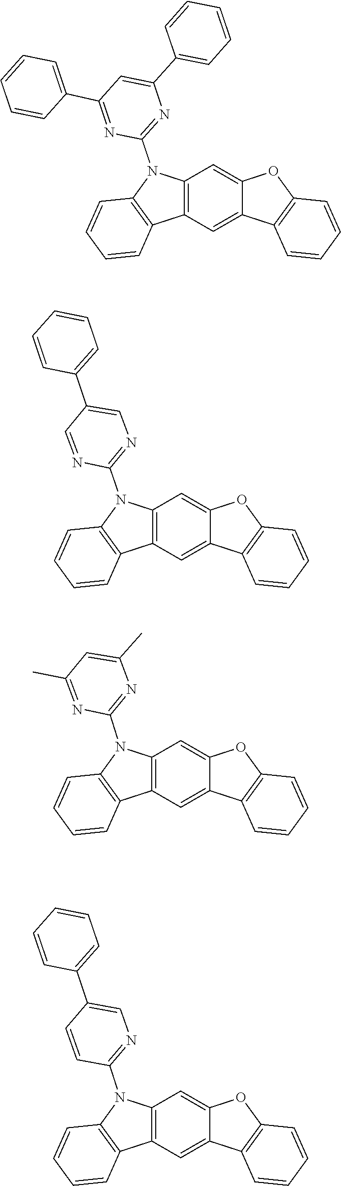 [SMILES: c1ccc(-c2cc(-c3ccccc3)nc(-n3c4ccccc4c4cc5c(cc43)oc3ccccc35)n2)cc1 ; c1ccc(-c2cnc(-n3c4ccccc4c4cc5c(cc43)oc3ccccc35)nc2)cc1 ; Cc1cc(C)nc(-n2c3ccccc3c3cc4c(cc32)oc2ccccc24)n1 ; c1ccc(-c2ccc(-n3c4ccccc4c4cc5c(cc43)oc3ccccc35)nc2)cc1]

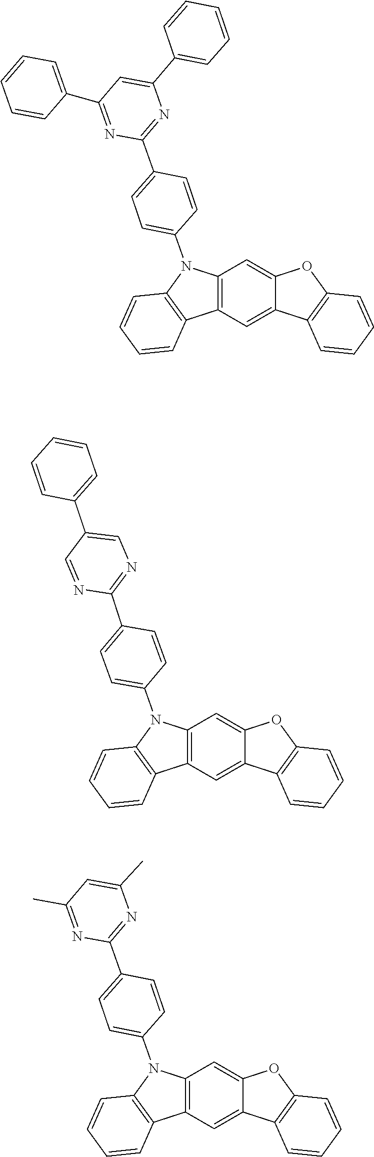 [SMILES: c1ccc(-c2cc(-c3ccccc3)nc(-c3ccc(-n4c5ccccc5c5cc6c(cc54)oc4ccccc46)cc3)n2)cc1 ; c1ccc(-c2cnc(-c3ccc(-n4c5ccccc5c5cc6c(cc54)oc4ccccc46)cc3)nc2)cc1 ; Cc1cc(C)nc(-c2ccc(-n3c4ccccc4c4cc5c(cc43)oc3ccccc35)cc2)n1]

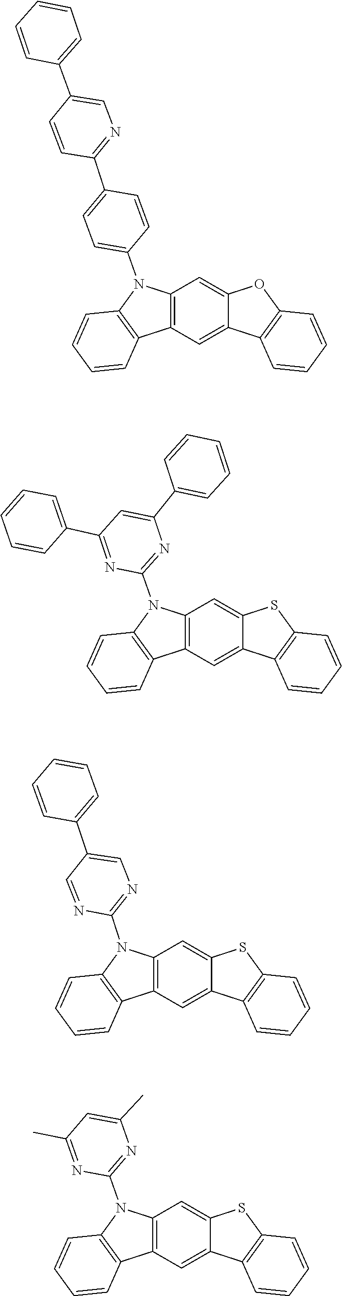 [SMILES: c1ccc(-c2ccc(-c3ccc(-n4c5ccccc5c5cc6c(cc54)oc4ccccc46)cc3)nc2)cc1 ; c1ccc(-c2cc(-c3ccccc3)nc(-n3c4ccccc4c4cc5c(cc43)sc3ccccc35)n2)cc1 ; c1ccc(-c2cnc(-n3c4ccccc4c4cc5c(cc43)sc3ccccc35)nc2)cc1 ; Cc1cc(C)nc(-n2c3ccccc3c3cc4c(cc32)sc2ccccc24)n1]

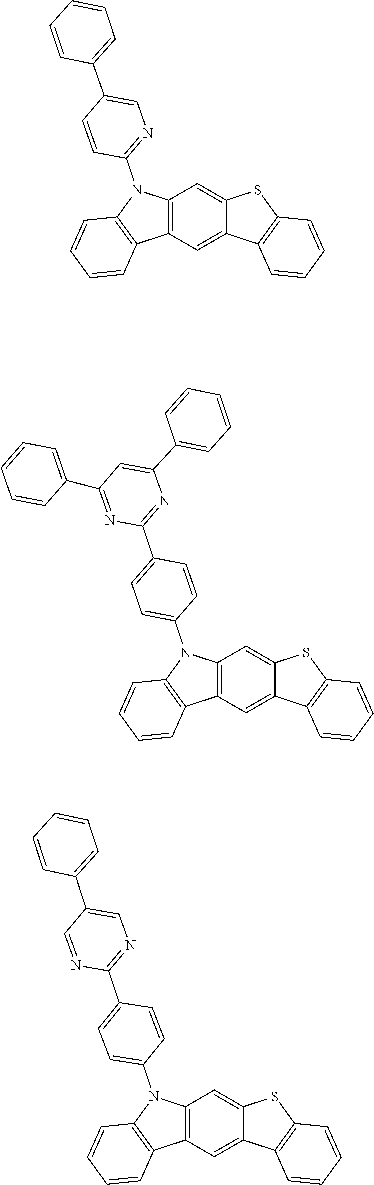 [SMILES: c1ccc(-c2ccc(-n3c4ccccc4c4cc5c(cc43)sc3ccccc35)nc2)cc1 ; c1ccc(-c2cc(-c3ccccc3)nc(-c3ccc(-n4c5ccccc5c5cc6c(cc54)sc4ccccc46)cc3)n2)cc1 ; c1ccc(-c2cnc(-c3ccc(-n4c5ccccc5c5cc6c(cc54)sc4ccccc46)cc3)nc2)cc1]

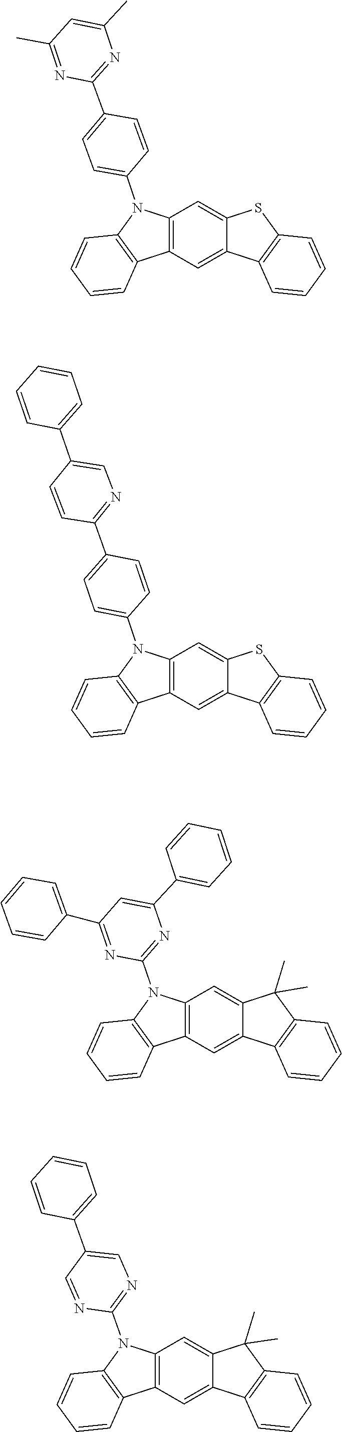 [SMILES: Cc1cc(C)nc(-c2ccc(-n3c4ccccc4c4cc5c(cc43)sc3ccccc35)cc2)n1 ; c1ccc(-c2ccc(-c3ccc(-n4c5ccccc5c5cc6c(cc54)sc4ccccc46)cc3)nc2)cc1 ; CC1(C)c2ccccc2-c2cc3c4ccccc4n(-c4nc(-c5ccccc5)cc(-c5ccccc5)n4)c3cc21 ; CC1(C)c2ccccc2-c2cc3c4ccccc4n(-c4ncc(-c5ccccc5)cn4)c3cc21]

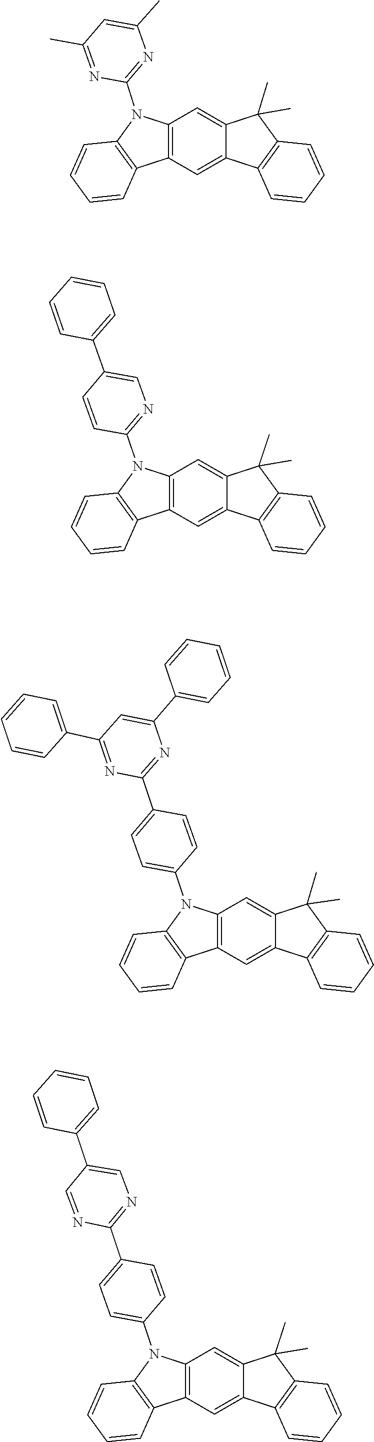 [SMILES: Cc1cc(C)nc(-n2c3ccccc3c3cc4c(cc32)C(C)(C)c2ccccc2-4)n1 ; CC1(C)c2ccccc2-c2cc3c4ccccc4n(-c4ccc(-c5ccccc5)cn4)c3cc21 ; CC1(C)c2ccccc2-c2cc3c4ccccc4n(-c4ccc(-c5nc(-c6ccccc6)cc(-c6ccccc6)n5)cc4)c3cc21 ; CC1(C)c2ccccc2-c2cc3c4ccccc4n(-c4ccc(-c5ncc(-c6ccccc6)cn5)cc4)c3cc21]

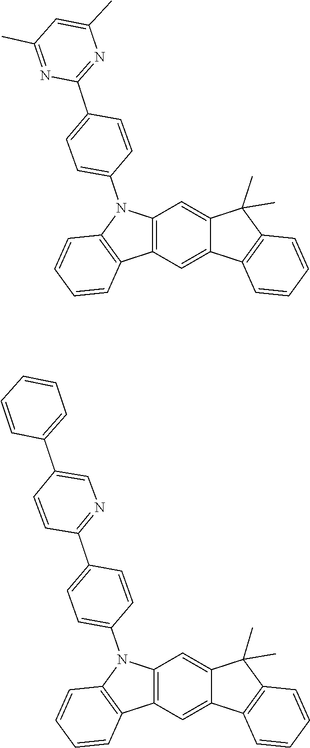 [SMILES: Cc1cc(C)nc(-c2ccc(-n3c4ccccc4c4cc5c(cc43)C(C)(C)c3ccccc3-5)cc2)n1 ; CC1(C)c2ccccc2-c2cc3c4ccccc4n(-c4ccc(-c5ccc(-c6ccccc6)cn5)cc4)c3cc21]

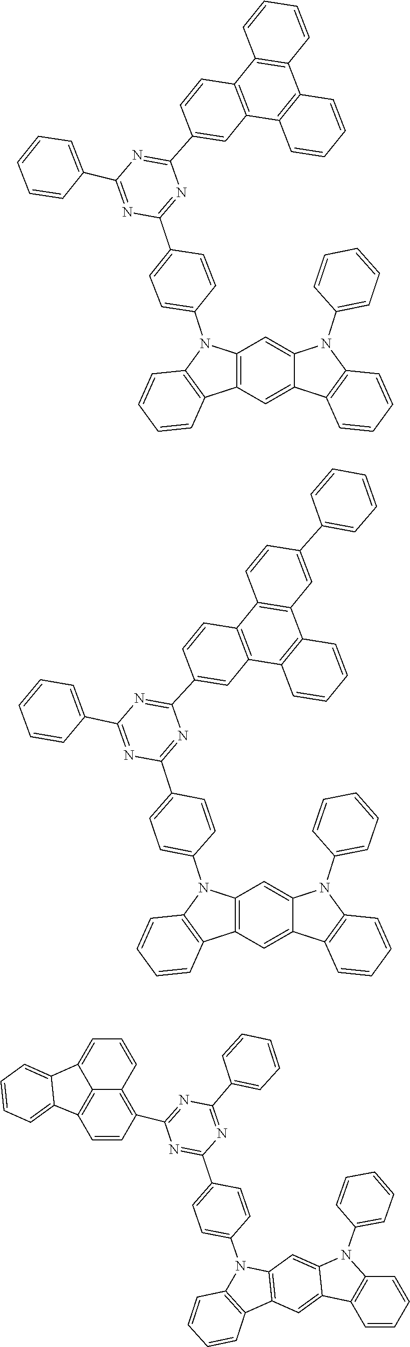

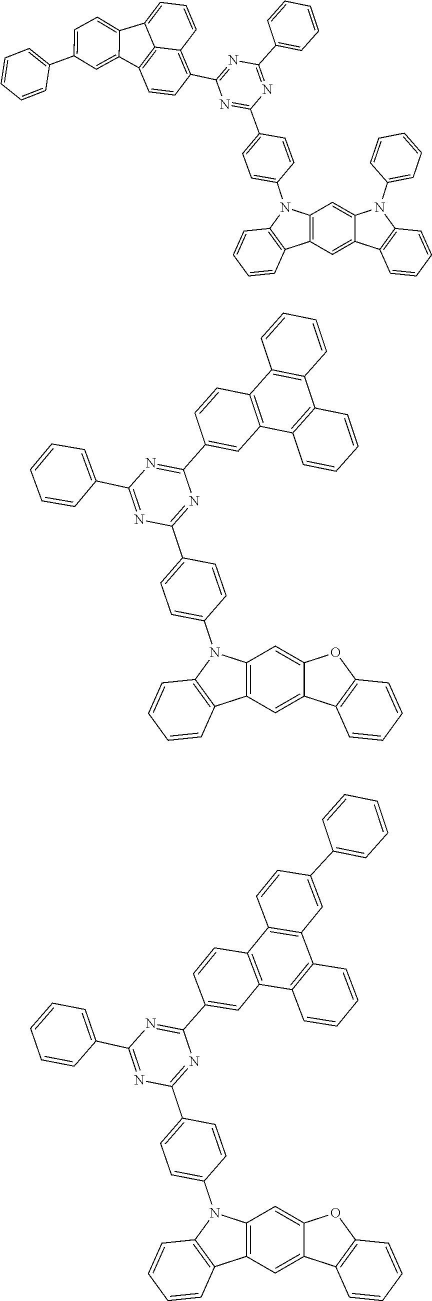

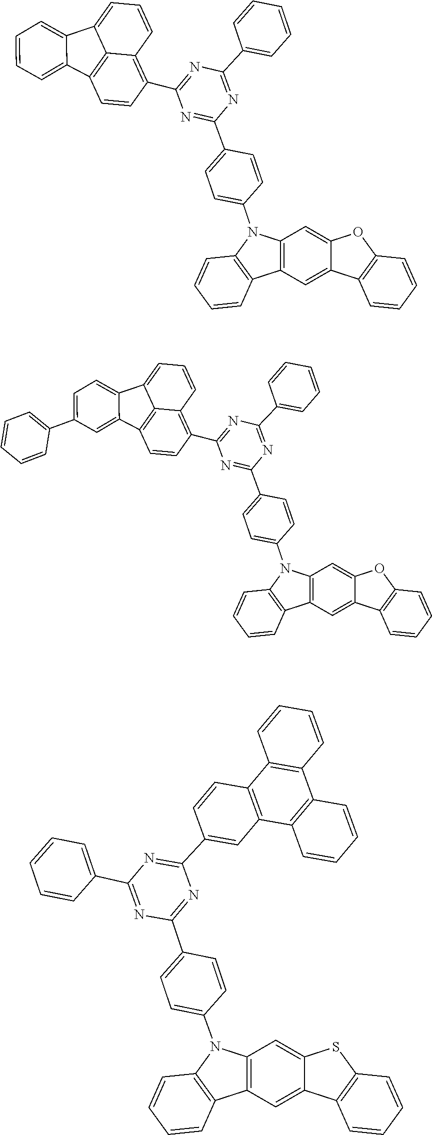 [SMILES: c1ccc(-c2nc(-c3ccc(-n4c5ccccc5c5cc6c(cc54)oc4ccccc46)cc3)nc(-c3ccc4c5c(cccc35)-c3ccccc3-4)n2)cc1 ; c1ccc(-c2ccc3c(c2)-c2ccc(-c4nc(-c5ccccc5)nc(-c5ccc(-n6c7ccccc7c7cc8c(cc76)oc6ccccc68)cc5)n4)c4cccc-3c24)cc1 ; c1ccc(-c2nc(-c3ccc(-n4c5ccccc5c5cc6c(cc54)sc4ccccc46)cc3)nc(-c3ccc4c5ccccc5c5ccccc5c4c3)n2)cc1]

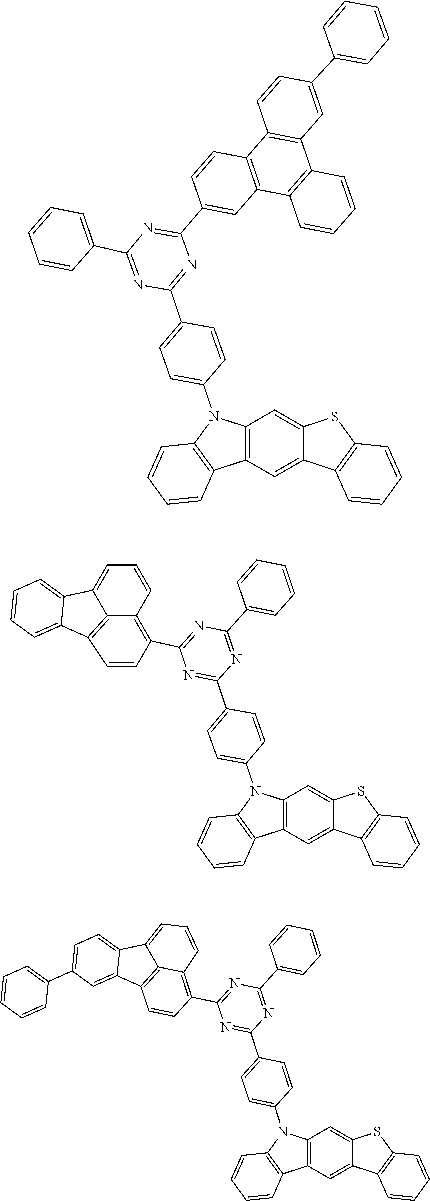 [SMILES: c1ccc(-c2ccc3c4ccc(-c5nc(-c6ccccc6)nc(-c6ccc(-n7c8ccccc8c8cc9c(cc87)sc7ccccc79)cc6)n5)cc4c4ccccc4c3c2)cc1 ; c1ccc(-c2nc(-c3ccc(-n4c5ccccc5c5cc6c(cc54)sc4ccccc46)cc3)nc(-c3ccc4c5c(cccc35)-c3ccccc3-4)n2)cc1 ; c1ccc(-c2ccc3c(c2)-c2ccc(-c4nc(-c5ccccc5)nc(-c5ccc(-n6c7ccccc7c7cc8c(cc76)sc6ccccc68)cc5)n4)c4cccc-3c24)cc1]

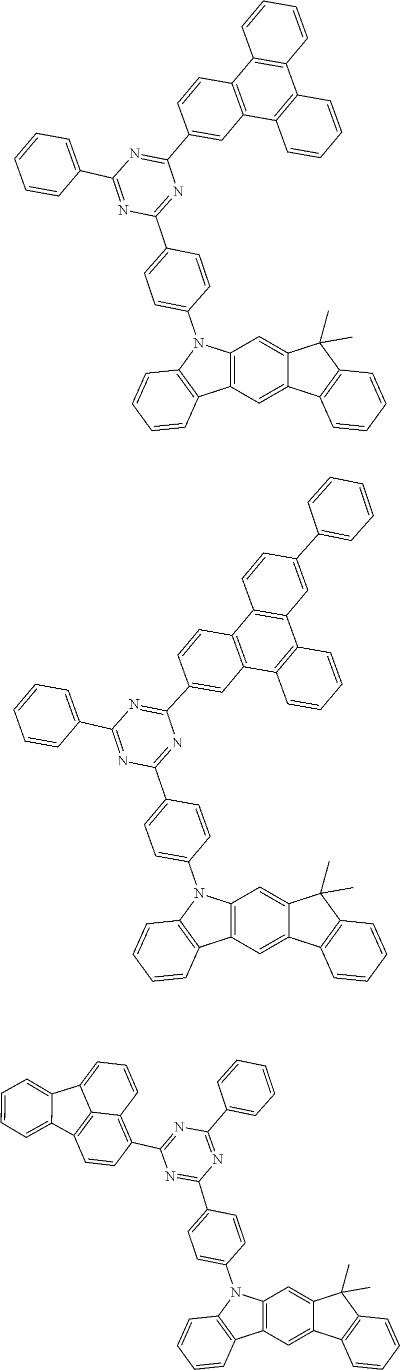 [SMILES: CC1(C)c2ccccc2-c2cc3c4ccccc4n(-c4ccc(-c5nc(-c6ccccc6)nc(-c6ccc7c8ccccc8c8ccccc8c7c6)n5)cc4)c3cc21 ; CC1(C)c2ccccc2-c2cc3c4ccccc4n(-c4ccc(-c5nc(-c6ccccc6)nc(-c6ccc7c8ccc(-c9ccccc9)cc8c8ccccc8c7c6)n5)cc4)c3cc21 ; CC1(C)c2ccccc2-c2cc3c4ccccc4n(-c4ccc(-c5nc(-c6ccccc6)nc(-c6ccc7c8c(cccc68)-c6ccccc6-7)n5)cc4)c3cc21]

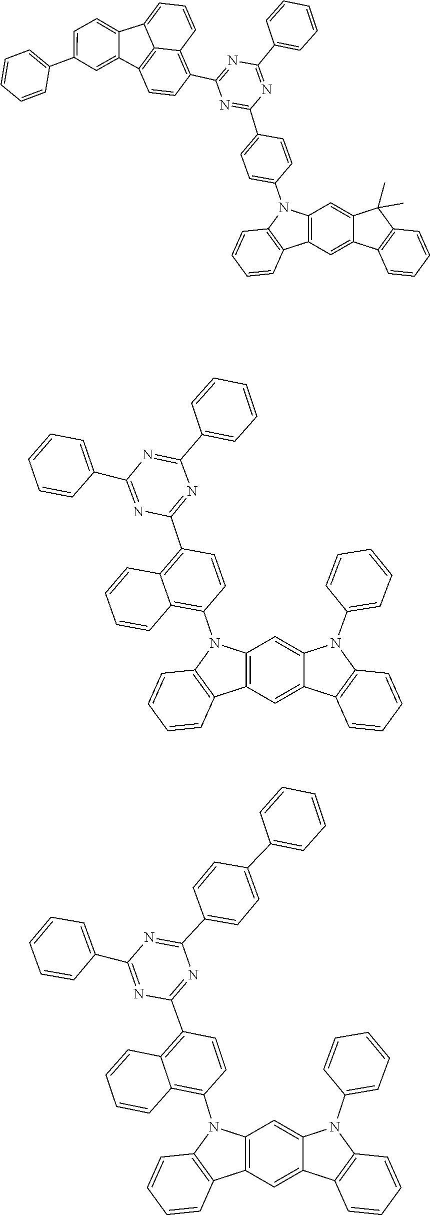 [SMILES: CC1(C)c2ccccc2-c2cc3c4ccccc4n(-c4ccc(-c5nc(-c6ccccc6)nc(-c6ccc7c8c(cccc68)-c6ccc(-c8ccccc8)cc6-7)n5)cc4)c3cc21 ; c1ccc(-c2nc(-c3ccccc3)nc(-c3ccc(-n4c5ccccc5c5cc6c7ccccc7n(-c7ccccc7)c6cc54)c4ccccc34)n2)cc1 ; c1ccc(-c2ccc(-c3nc(-c4ccccc4)nc(-c4ccc(-n5c6ccccc6c6cc7c8ccccc8n(-c8ccccc8)c7cc65)c5ccccc45)n3)cc2)cc1]

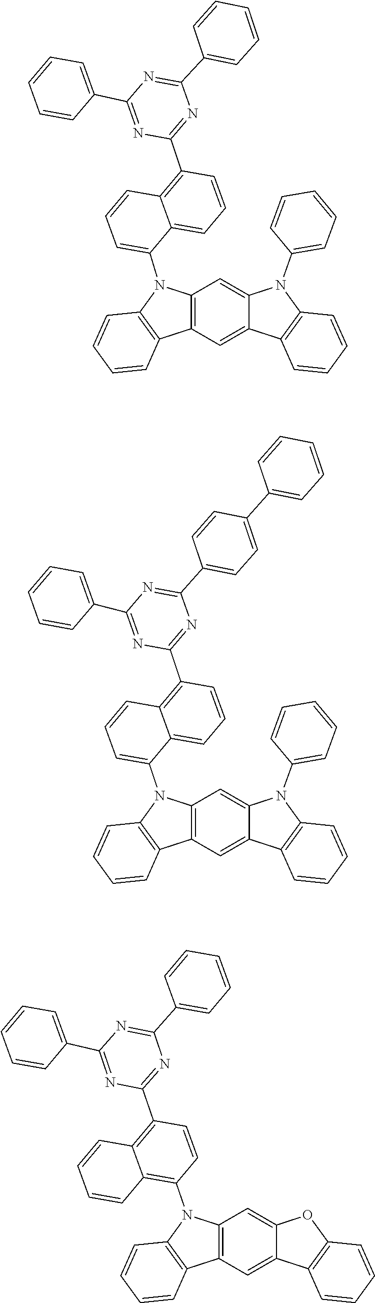 [SMILES: c1ccc(-c2nc(-c3ccccc3)nc(-c3cccc4c(-n5c6ccccc6c6cc7c8ccccc8n(-c8ccccc8)c7cc65)cccc34)n2)cc1 ; c1ccc(-c2ccc(-c3nc(-c4ccccc4)nc(-c4cccc5c(-n6c7ccccc7c7cc8c9ccccc9n(-c9ccccc9)c8cc76)cccc45)n3)cc2)cc1 ; c1ccc(-c2nc(-c3ccccc3)nc(-c3ccc(-n4c5ccccc5c5cc6c(cc54)oc4ccccc46)c4ccccc34)n2)cc1]

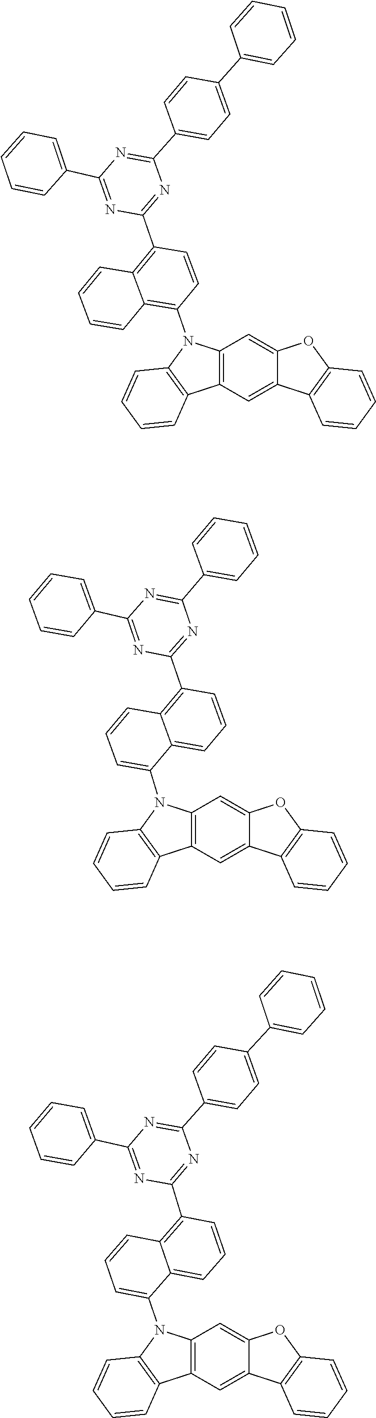 [SMILES: c1ccc(-c2ccc(-c3nc(-c4ccccc4)nc(-c4ccc(-n5c6ccccc6c6cc7c(cc65)oc5ccccc57)c5ccccc45)n3)cc2)cc1 ; c1ccc(-c2nc(-c3ccccc3)nc(-c3cccc4c(-n5c6ccccc6c6cc7c(cc65)oc5ccccc57)cccc34)n2)cc1 ; c1ccc(-c2ccc(-c3nc(-c4ccccc4)nc(-c4cccc5c(-n6c7ccccc7c7cc8c(cc76)oc6ccccc68)cccc45)n3)cc2)cc1]

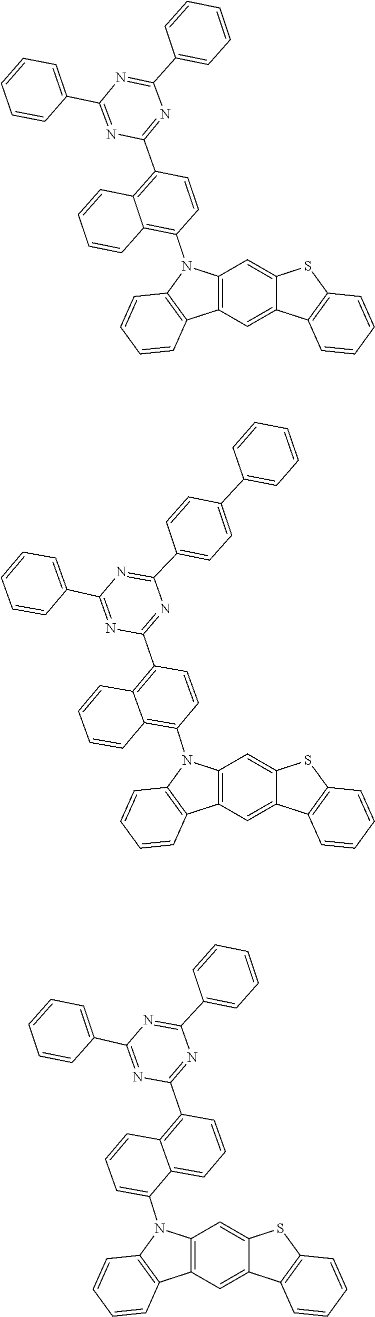 [SMILES: c1ccc(-c2nc(-c3ccccc3)nc(-c3ccc(-n4c5ccccc5c5cc6c(cc54)sc4ccccc46)c4ccccc34)n2)cc1 ; c1ccc(-c2ccc(-c3nc(-c4ccccc4)nc(-c4ccc(-n5c6ccccc6c6cc7c(cc65)sc5ccccc57)c5ccccc45)n3)cc2)cc1 ; c1ccc(-c2nc(-c3ccccc3)nc(-c3cccc4c(-n5c6ccccc6c6cc7c(cc65)sc5ccccc57)cccc34)n2)cc1]

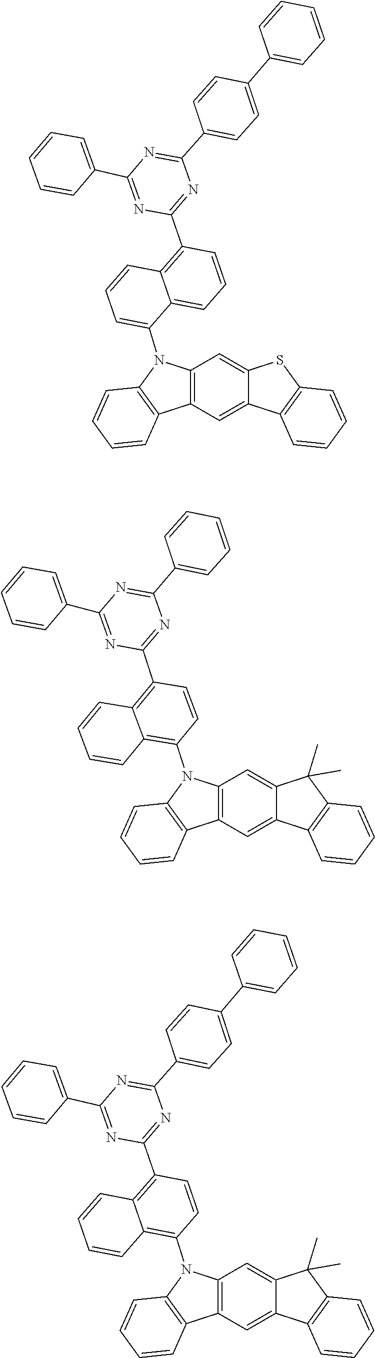 [SMILES: c1ccc(-c2ccc(-c3nc(-c4ccccc4)nc(-c4cccc5c(-n6c7ccccc7c7cc8c(cc76)sc6ccccc68)cccc45)n3)cc2)cc1 ; CC1(C)c2ccccc2-c2cc3c4ccccc4n(-c4ccc(-c5nc(-c6ccccc6)nc(-c6ccccc6)n5)c5ccccc45)c3cc21 ; CC1(C)c2ccccc2-c2cc3c4ccccc4n(-c4ccc(-c5nc(-c6ccccc6)nc(-c6ccc(-c7ccccc7)cc6)n5)c5ccccc45)c3cc21]

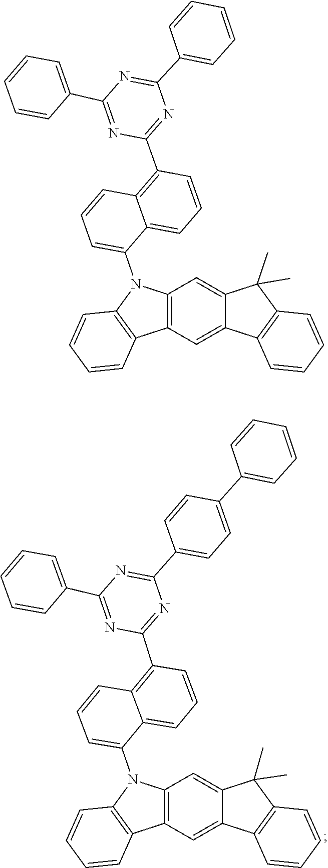 [SMILES: CC1(C)c2ccccc2-c2cc3c4ccccc4n(-c4cccc5c(-c6nc(-c7ccccc7)nc(-c7ccccc7)n6)cccc45)c3cc21 ; CC1(C)c2ccccc2-c2cc3c4ccccc4n(-c4cccc5c(-c6nc(-c7ccccc7)nc(-c7ccc(-c8ccccc8)cc7)n6)cccc45)c3cc21]

the “substituted” means that hydrogen atoms in the above compounds are each independently substituted with deuterium atoms.

In a second aspect, the present application provides a compound, and the compound includes the following compounds:

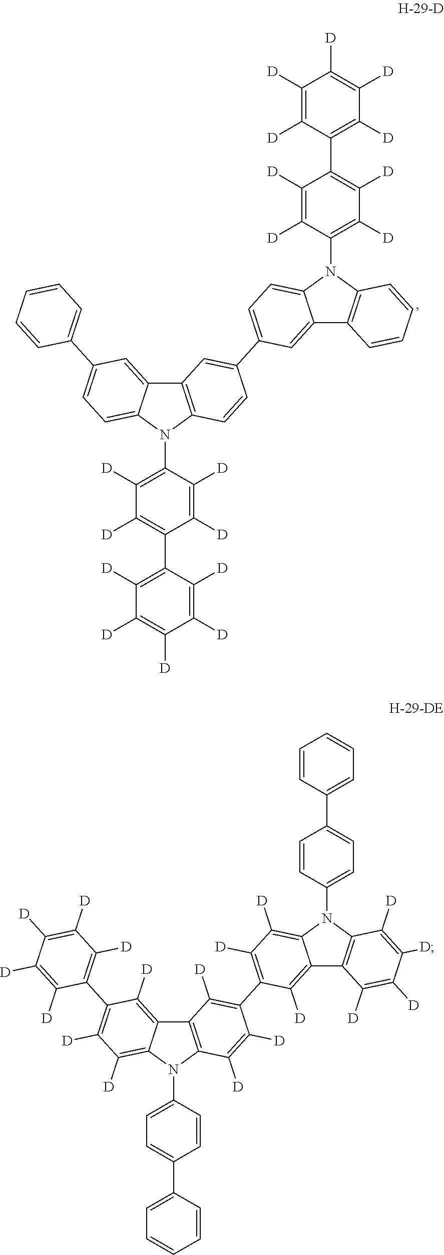

the compound is used for preparing the composition as described in the first aspect.

In a third aspect, the present application provides an intermediate, and the intermediate includes the following compounds:

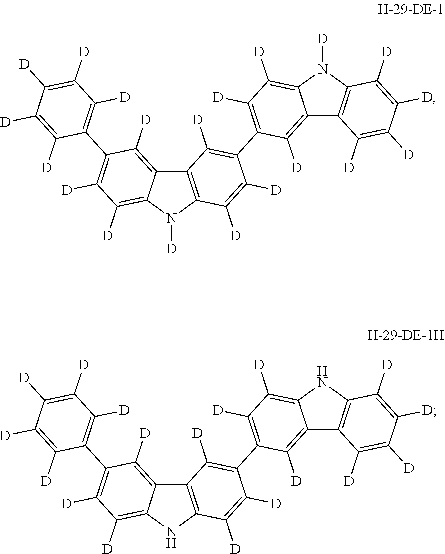

the intermediate is used for preparing the compound represented by Formula I in the composition as described in the first aspect.

It should be noted that in the present application, the preparation methods of the compound represented by Formula I and the compound represented by Formula II are not limited in any particular way, and can be prepared by methods commonly used in the field, exemplarily referring to the preparation methods in CN112996793A, CN112805277A, CN102212066A, and CN114695764A.

It should also be noted that D in the above compounds and intermediates is a deuterium atom.

In a fourth aspect, the present application provides an organic electroluminescent device, and the organic electroluminescent device includes an anode, a cathode, and an organic thin film layer arranged between the anode and the cathode;
wherein a material of the organic thin film layer includes the composition as described in the first aspect.

In one embodiment, the organic thin film layer includes an emissive layer, and a material of the emissive layer includes the composition as described in the first aspect.

In one embodiment, the organic thin film layer includes a hole layer.

In one embodiment, the hole layer includes an electron blocking layer; a material of the electron blocking layer includes a spirofluorene compound.

the spirofluorene compound has a specific structure represented by Formula III below:

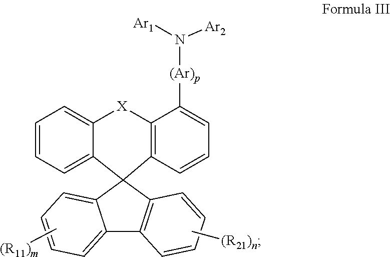

wherein X is selected from O or S;
R₁₁ and R₂₁ are each independently selected from hydrogen, deuterium, fluorine, CN, substituted or unsubstituted C1 to C20 (which may be, for example, C1, C2, C3, C4, C5, C7, C8, C9, C10, C13, C15, C18, or C20, etc.) linear or branched alkyl, substituted or unsubstituted C1 to C20 (which may be, for example, C1, C2, C3, C4, C5, C7, C8, C9, C10, C13, C15, C18, or C20, etc.) alkoxy, or substituted or unsubstituted C6 to C40 (which may be, for example, C6, C8, C10, C12, C15, C18, C24, C30, C36, or C40, etc.) aryl;
Ar is selected from substituted or unsubstituted C6 to C40 (which may be, for example, C6, C8, C10, C12, C15, C18, C24, C30, C36, or C40, etc.) arylene;
Ar₁ and Ar₂ are each independently selected from substituted or unsubstituted C6 to C40 (which may be, for example, C6, C8, C10, C12, C15, C18, C24, C30, C36, or C40, etc.) aryl, substituted or unsubstituted C12 to C40 (C12, C14, C16, C18, C20, C23, C25, C27, C30, C32, C35, C37, C39, or C40, etc.) oxa-aryl, or substituted or unsubstituted C12 to C40 (C12, C14, C16, C18, C20, C23, C25, C27, C30, C32, C35, C37, C39, or C40, etc.) thioaryl, and at least one of Ar₁ or Ar₂ is selected from any one of phenyl, naphthyl, triphenylenyl, or fluoranthenyl; p is selected from 0 or 1; and m and n are each independently selected from an integer from 0 to 4, which may be, for example, 0, 1, 2, 3, or 4.

It should be noted that the oxa-aryl refers to a structure having an oxygen-containing five-membered heterocyclic ring where the two aryl rings connected by a single bond are bridged by an O atom, for example, two benzene rings are connected by a single bond to form a biphenyl, and the carbon atoms on the two benzene rings forming the biphenyl are connected to the O atom at the same time to form a dibenzofuran.

The thioaryl refers to a structure having an sulfur-containing five-membered heterocyclic ring where the two aryl rings connected by a single bond are bridged by an S atom, for example, two benzene rings are connected by a single bond to form a biphenyl, and the carbon atoms on the two benzene rings forming the biphenyl are connected to the S atom at the same time to form a dibenzothiophene.

In one embodiment, the spirofluorene compound is selected from compounds represented by Formula III-1 or compounds represented by Formula III-2:

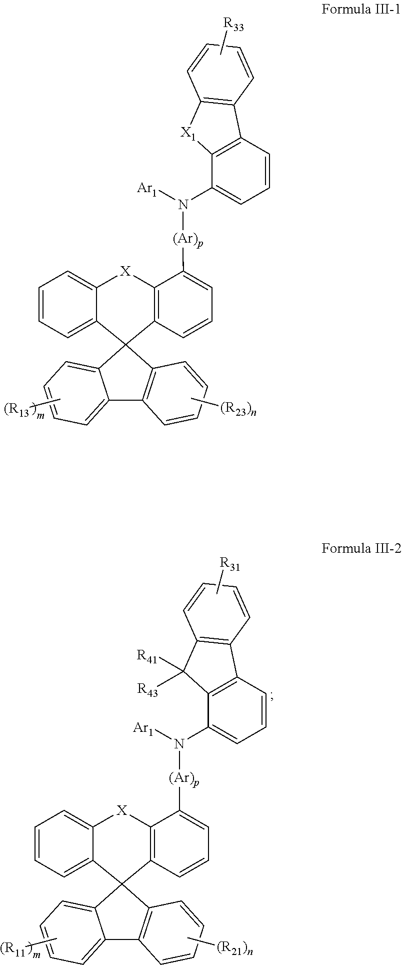

wherein X and X₁ are each independently selected from O or S;
R₁₁, R₂₁, and Ar have the same protection scope as described above;
Ar₁ is selected from any one of phenyl, naphthyl, triphenylenyl, or fluoranthenyl;
R₃₁ is selected from C1 to C20 (which may be, for example, C1, C2, C3, C4, C5, C7, C8, C9, C10, C13, C15, C18, or C20, etc.) linear or branched alkyl, C1 to C20 (which may be, for example, C1, C2, C3, C4, C5, C7, C8, C9, C10, C13, C15, C18, or C20, etc.) alkoxy, or C6 to C40 (which may be, for example, C6, C8, C10, C12, C15, C18, C24, C30, C36, or C40, etc.) aryl;
R₄₁ and R₄₂ are each independently selected from C1 to C20 (which may be, for example, C1, C2, C3, C4, C5, C7, C8, C9, C10, C13, C15, C18, or C20, etc.) linear or branched alkyl, C6 to C40 (which may be, for example, C6, C8, C10, C12, C15, C18, C24, C30, C36, or C40, etc.) aryl, and R₄₁ and R₄₂ are independent with each other or are connected by a single bond to form a ring.

In one embodiment, the spirofluorene compound is selected from any one of the following Compounds 1-140 and Compounds 1S-140S:

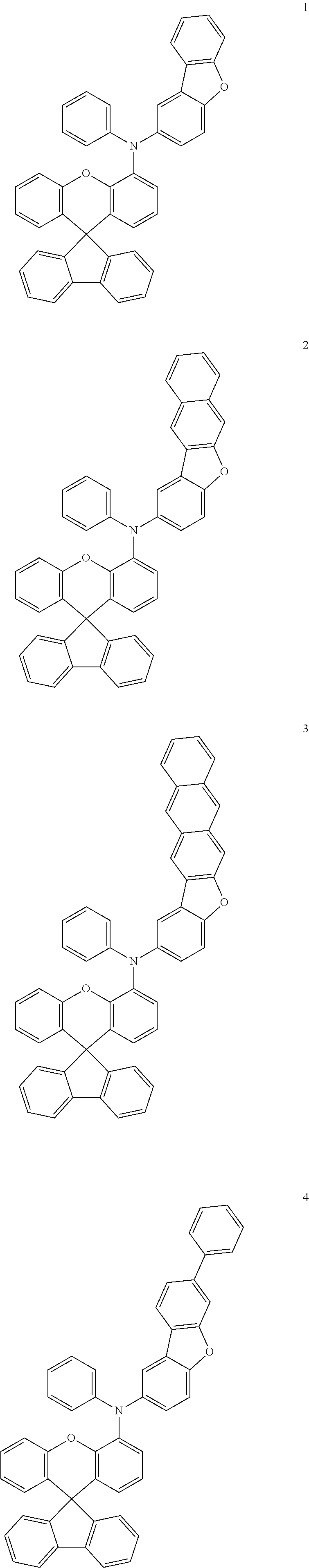 [SMILES: c1ccc(N(c2ccc3oc4ccccc4c3c2)c2cccc3c2Oc2ccccc2C32c3ccccc3-c3ccccc32)cc1 ; c1ccc(N(c2ccc3oc4cc5ccccc5cc4c3c2)c2cccc3c2Oc2ccccc2C32c3ccccc3-c3ccccc32)cc1 ; c1ccc(N(c2ccc3oc4cc5cc6ccccc6cc5cc4c3c2)c2cccc3c2Oc2ccccc2C32c3ccccc3-c3ccccc32)cc1 ; c1ccc(-c2ccc3c(c2)oc2ccc(N(c4ccccc4)c4cccc5c4Oc4ccccc4C54c5ccccc5-c5ccccc54)cc23)cc1]

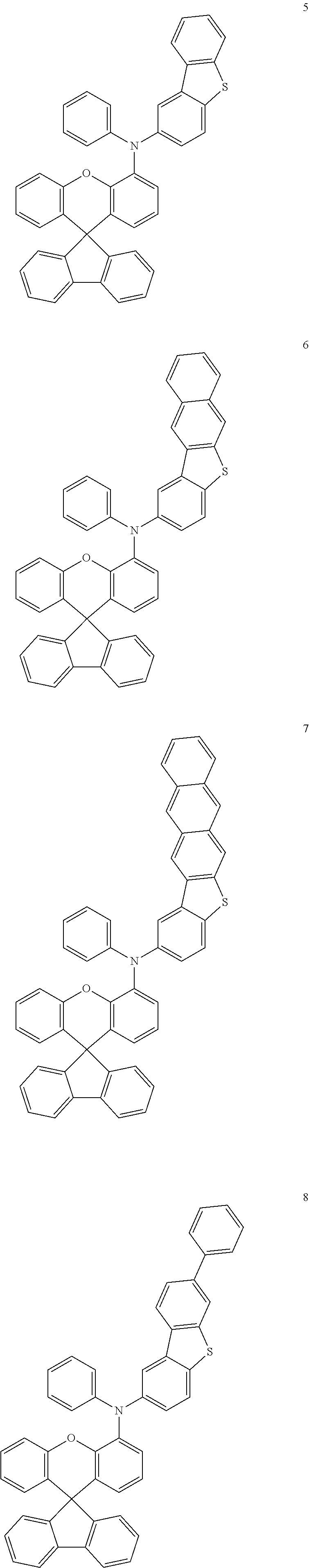 [SMILES: c1ccc(N(c2ccc3sc4ccccc4c3c2)c2cccc3c2Oc2ccccc2C32c3ccccc3-c3ccccc32)cc1 ; c1ccc(N(c2ccc3sc4cc5ccccc5cc4c3c2)c2cccc3c2Oc2ccccc2C32c3ccccc3-c3ccccc32)cc1 ; c1ccc(N(c2ccc3sc4cc5cc6ccccc6cc5cc4c3c2)c2cccc3c2Oc2ccccc2C32c3ccccc3-c3ccccc32)cc1 ; c1ccc(-c2ccc3c(c2)sc2ccc(N(c4ccccc4)c4cccc5c4Oc4ccccc4C54c5ccccc5-c5ccccc54)cc23)cc1]

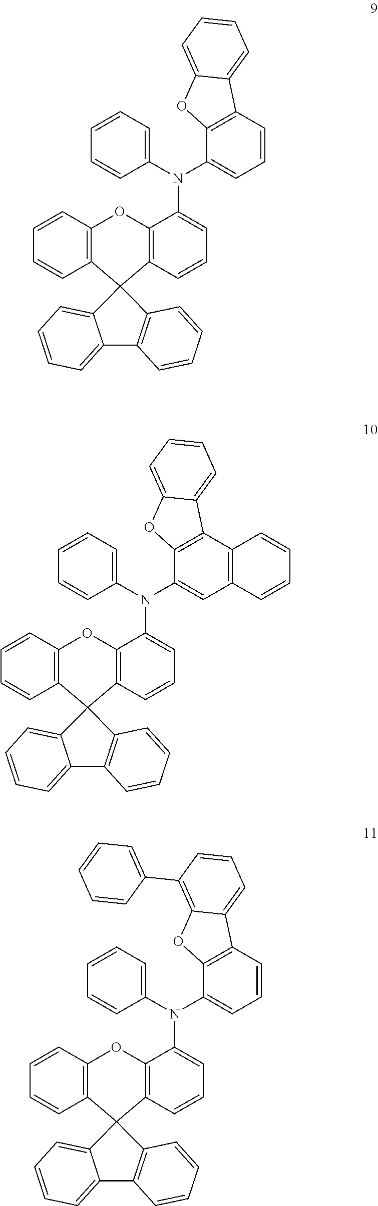 [SMILES: c1ccc(N(c2cccc3c2Oc2ccccc2C32c3ccccc3-c3ccccc32)c2cccc3c2oc2ccccc23)cc1 ; c1ccc(N(c2cccc3c2Oc2ccccc2C32c3ccccc3-c3ccccc32)c2cc3ccccc3c3c2oc2ccccc23)cc1 ; c1ccc(-c2cccc3c2oc2c(N(c4ccccc4)c4cccc5c4Oc4ccccc4C54c5ccccc5-c5ccccc54)cccc23)cc1]

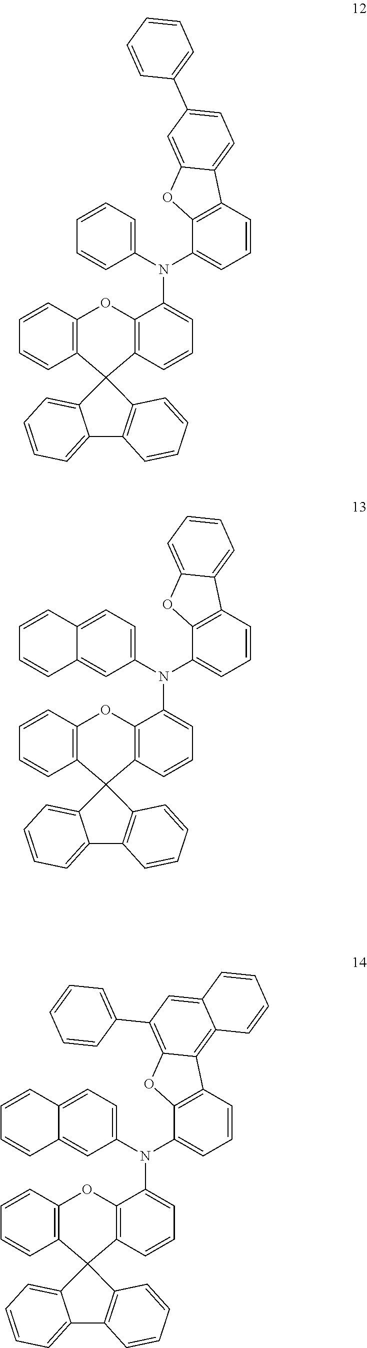 [SMILES: c1ccc(-c2ccc3c(c2)oc2c(N(c4ccccc4)c4cccc5c4Oc4ccccc4C54c5ccccc5-c5ccccc54)cccc23)cc1 ; c1ccc2c(c1)Oc1c(N(c3ccc4ccccc4c3)c3cccc4c3oc3ccccc34)cccc1C21c2ccccc2-c2ccccc21 ; c1ccc(-c2cc3ccccc3c3c2oc2c(N(c4ccc5ccccc5c4)c4cccc5c4Oc4ccccc4C54c5ccccc5-c5ccccc54)cccc23)cc1]

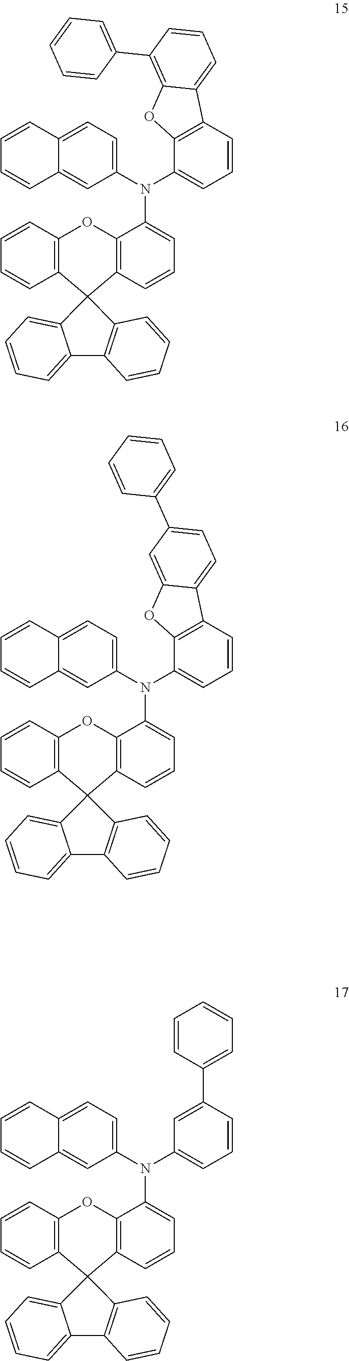 [SMILES: c1ccc(-c2cccc3c2oc2c(N(c4ccc5ccccc5c4)c4cccc5c4Oc4ccccc4C54c5ccccc5-c5ccccc54)cccc23)cc1 ; c1ccc(-c2ccc3c(c2)oc2c(N(c4ccc5ccccc5c4)c4cccc5c4Oc4ccccc4C54c5ccccc5-c5ccccc54)cccc23)cc1 ; c1ccc(-c2cccc(N(c3ccc4ccccc4c3)c3cccc4c3Oc3ccccc3C43c4ccccc4-c4ccccc43)c2)cc1]

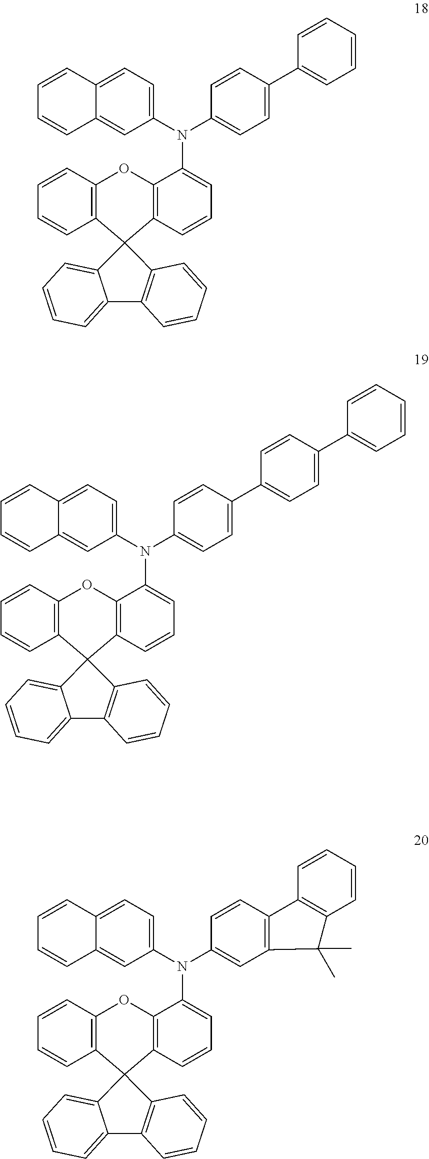 [SMILES: c1ccc(-c2ccc(N(c3ccc4ccccc4c3)c3cccc4c3Oc3ccccc3C43c4ccccc4-c4ccccc43)cc2)cc1 ; c1ccc(-c2ccc(-c3ccc(N(c4ccc5ccccc5c4)c4cccc5c4Oc4ccccc4C54c5ccccc5-c5ccccc54)cc3)cc2)cc1 ; CC1(C)c2ccccc2-c2ccc(N(c3ccc4ccccc4c3)c3cccc4c3Oc3ccccc3C43c4ccccc4-c4ccccc43)cc21]

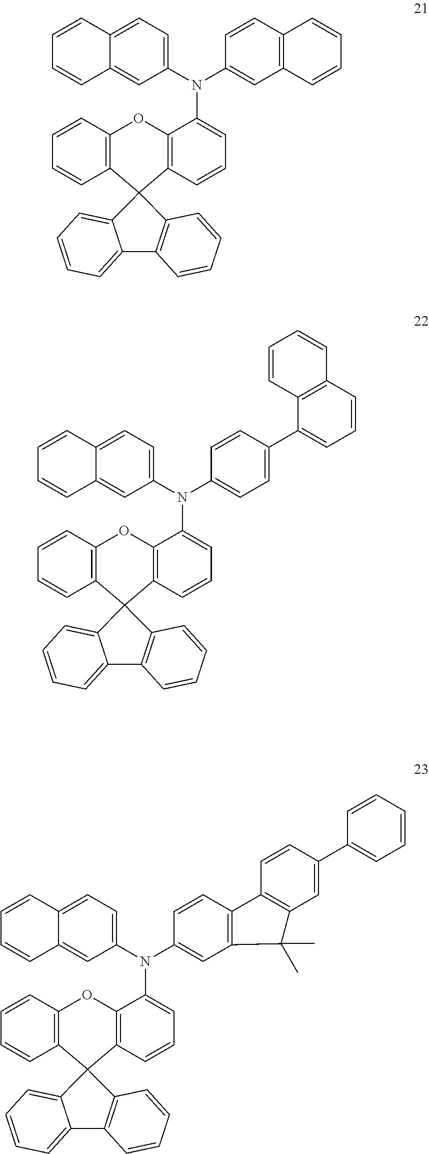 [SMILES: c1ccc2c(c1)Oc1c(N(c3ccc4ccccc4c3)c3ccc4ccccc4c3)cccc1C21c2ccccc2-c2ccccc21 ; c1ccc2c(c1)Oc1c(N(c3ccc(-c4cccc5ccccc45)cc3)c3ccc4ccccc4c3)cccc1C21c2ccccc2-c2ccccc21 ; CC1(C)c2cc(-c3ccccc3)ccc2-c2ccc(N(c3ccc4ccccc4c3)c3cccc4c3Oc3ccccc3C43c4ccccc4-c4ccccc43)cc21]

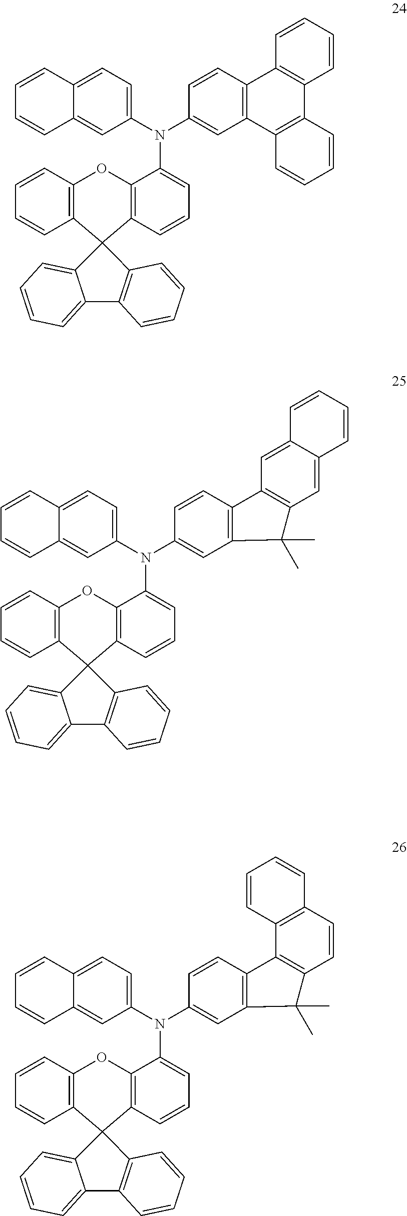 [SMILES: c1ccc2c(c1)Oc1c(N(c3ccc4ccccc4c3)c3ccc4c5ccccc5c5ccccc5c4c3)cccc1C21c2ccccc2-c2ccccc21 ; CC1(C)c2cc(N(c3ccc4ccccc4c3)c3cccc4c3Oc3ccccc3C43c4ccccc4-c4ccccc43)ccc2-c2cc3ccccc3cc21 ; CC1(C)c2cc(N(c3ccc4ccccc4c3)c3cccc4c3Oc3ccccc3C43c4ccccc4-c4ccccc43)ccc2-c2c1ccc1ccccc21]

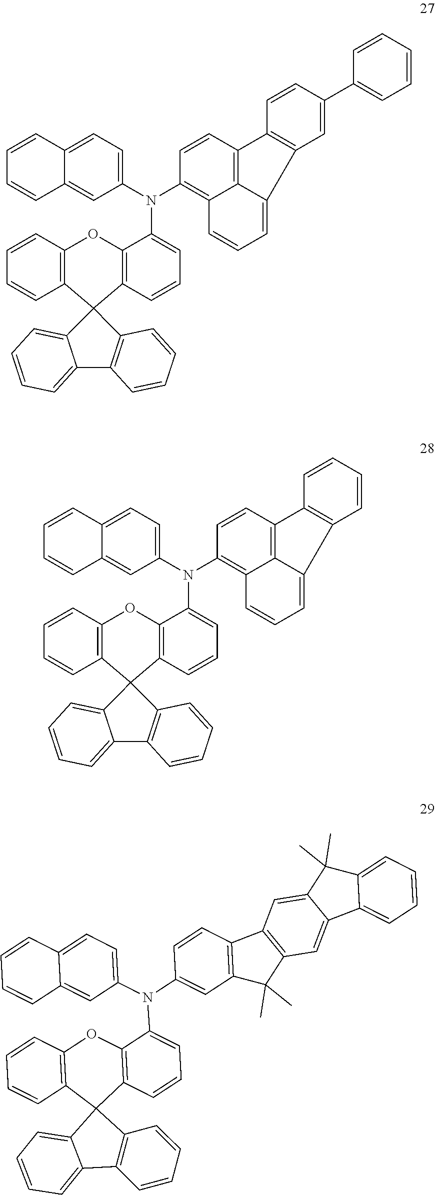 [SMILES: c1ccc(-c2ccc3c(c2)-c2cccc4c(N(c5ccc6ccccc6c5)c5cccc6c5Oc5ccccc5C65c6ccccc6-c6ccccc65)ccc-3c24)cc1 ; c1ccc2c(c1)Oc1c(N(c3ccc4ccccc4c3)c3ccc4c5c(cccc35)-c3ccccc3-4)cccc1C21c2ccccc2-c2ccccc21 ; CC1(C)c2ccccc2-c2cc3c(cc21)-c1ccc(N(c2ccc4ccccc4c2)c2cccc4c2Oc2ccccc2C42c4ccccc4-c4ccccc42)cc1C3(C)C]

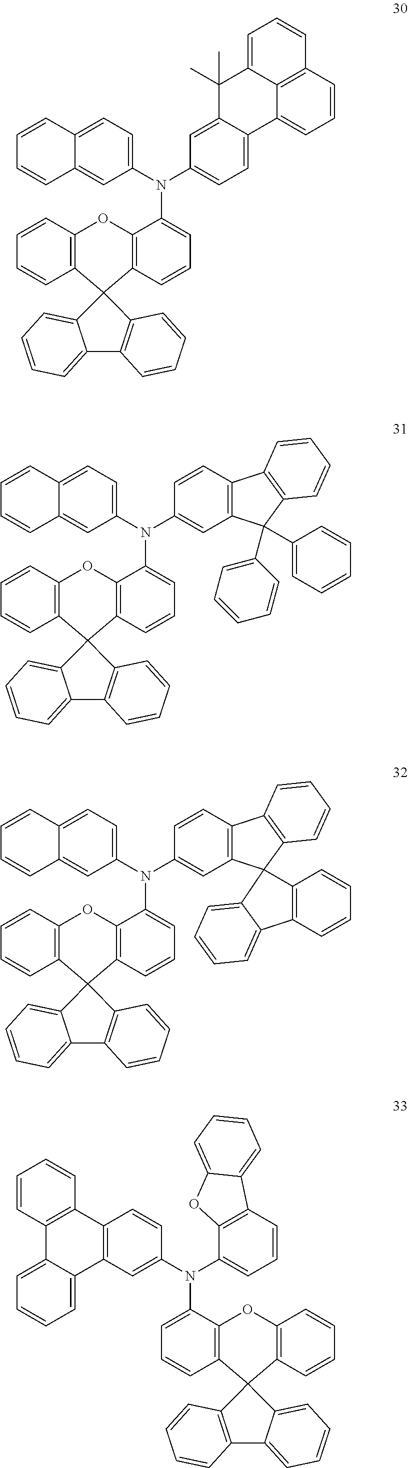 [SMILES: CC1(C)c2cc(N(c3ccc4ccccc4c3)c3cccc4c3Oc3ccccc3C43c4ccccc4-c4ccccc43)ccc2-c2cccc3cccc1c23 ; c1ccc(C2(c3ccccc3)c3ccccc3-c3ccc(N(c4ccc5ccccc5c4)c4cccc5c4Oc4ccccc4C54c5ccccc5-c5ccccc54)cc32)cc1 ; c1ccc2c(c1)Oc1c(N(c3ccc4c(c3)C3(c5ccccc5-c5ccccc53)c3ccccc3-4)c3ccc4ccccc4c3)cccc1C21c2ccccc2-c2ccccc21 ; c1ccc2c(c1)Oc1c(N(c3ccc4c5ccccc5c5ccccc5c4c3)c3cccc4c3oc3ccccc34)cccc1C21c2ccccc2-c2ccccc21]

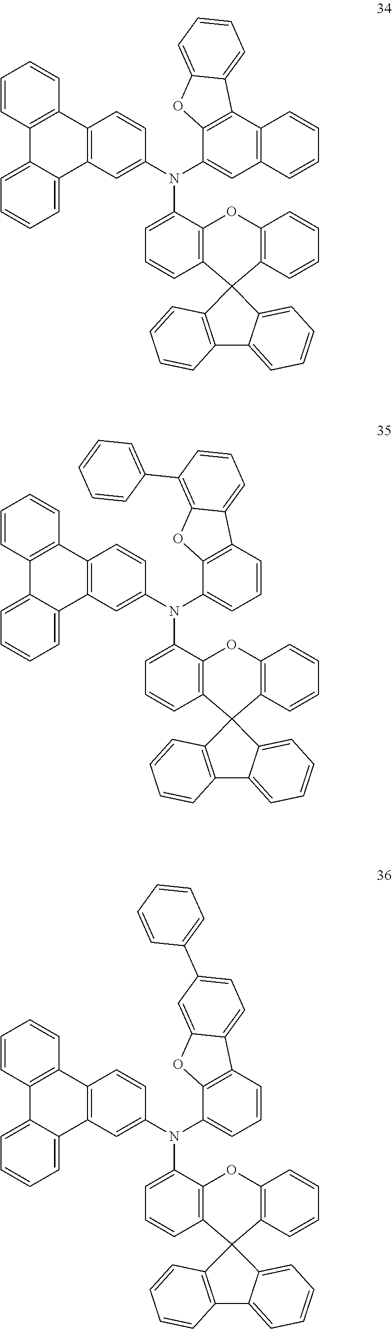 [SMILES: c1ccc2c(c1)Oc1c(N(c3ccc4c5ccccc5c5ccccc5c4c3)c3cc4ccccc4c4c3oc3ccccc34)cccc1C21c2ccccc2-c2ccccc21 ; c1ccc(-c2cccc3c2oc2c(N(c4ccc5c6ccccc6c6ccccc6c5c4)c4cccc5c4Oc4ccccc4C54c5ccccc5-c5ccccc54)cccc23)cc1 ; c1ccc(-c2ccc3c(c2)oc2c(N(c4ccc5c6ccccc6c6ccccc6c5c4)c4cccc5c4Oc4ccccc4C54c5ccccc5-c5ccccc54)cccc23)cc1]

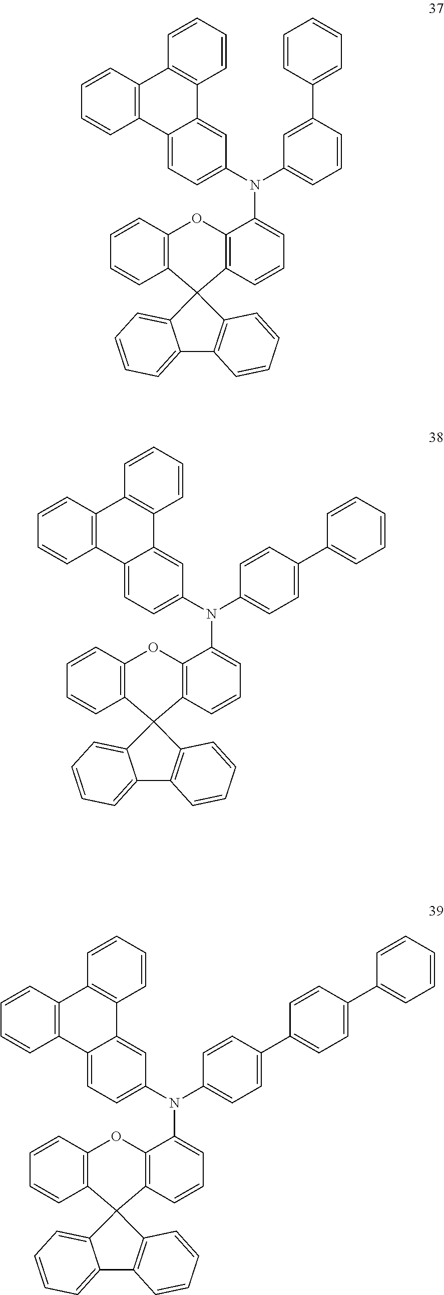 [SMILES: c1ccc(-c2cccc(N(c3ccc4c5ccccc5c5ccccc5c4c3)c3cccc4c3Oc3ccccc3C43c4ccccc4-c4ccccc43)c2)cc1 ; c1ccc(-c2ccc(N(c3ccc4c5ccccc5c5ccccc5c4c3)c3cccc4c3Oc3ccccc3C43c4ccccc4-c4ccccc43)cc2)cc1 ; c1ccc(-c2ccc(-c3ccc(N(c4ccc5c6ccccc6c6ccccc6c5c4)c4cccc5c4Oc4ccccc4C54c5ccccc5-c5ccccc54)cc3)cc2)cc1]

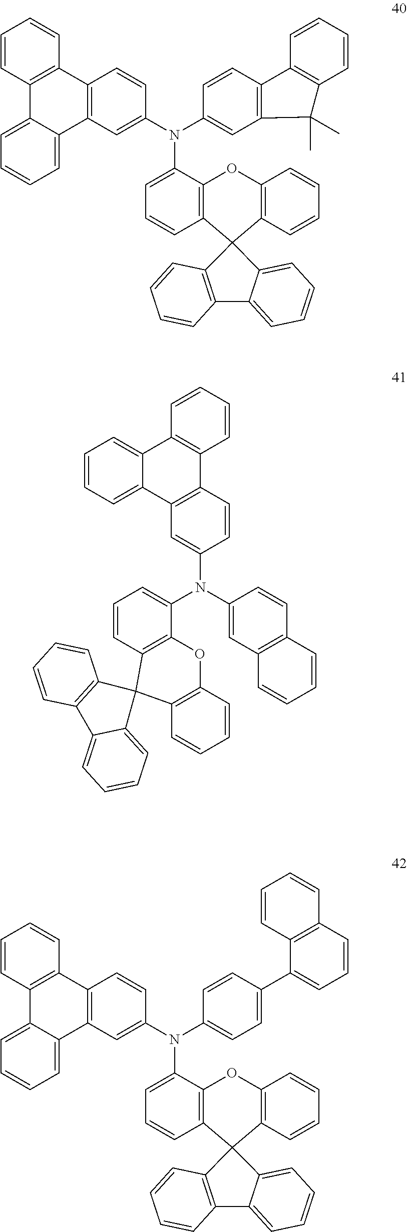 [SMILES: CC1(C)c2ccccc2-c2ccc(N(c3ccc4c5ccccc5c5ccccc5c4c3)c3cccc4c3Oc3ccccc3C43c4ccccc4-c4ccccc43)cc21 ; c1ccc2c(c1)Oc1c(N(c3ccc4ccccc4c3)c3ccc4c5ccccc5c5ccccc5c4c3)cccc1C21c2ccccc2-c2ccccc21 ; c1ccc2c(c1)Oc1c(N(c3ccc(-c4cccc5ccccc45)cc3)c3ccc4c5ccccc5c5ccccc5c4c3)cccc1C21c2ccccc2-c2ccccc21]

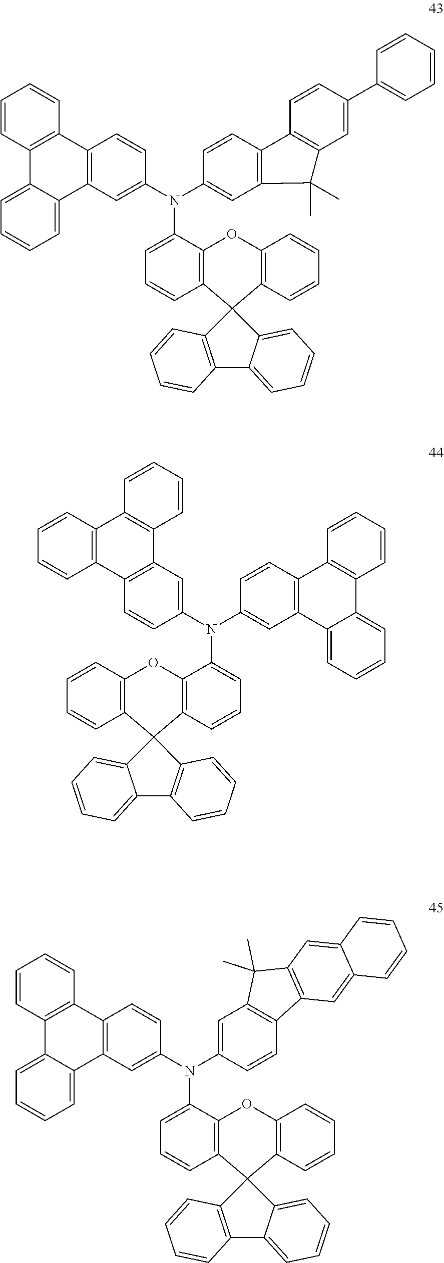 [SMILES: CC1(C)c2cc(-c3ccccc3)ccc2-c2ccc(N(c3ccc4c5ccccc5c5ccccc5c4c3)c3cccc4c3Oc3ccccc3C43c4ccccc4-c4ccccc43)cc21 ; c1ccc2c(c1)Oc1c(N(c3ccc4c5ccccc5c5ccccc5c4c3)c3ccc4c5ccccc5c5ccccc5c4c3)cccc1C21c2ccccc2-c2ccccc21 ; CC1(C)c2cc(N(c3ccc4c5ccccc5c5ccccc5c4c3)c3cccc4c3Oc3ccccc3C43c4ccccc4-c4ccccc43)ccc2-c2cc3ccccc3cc21]

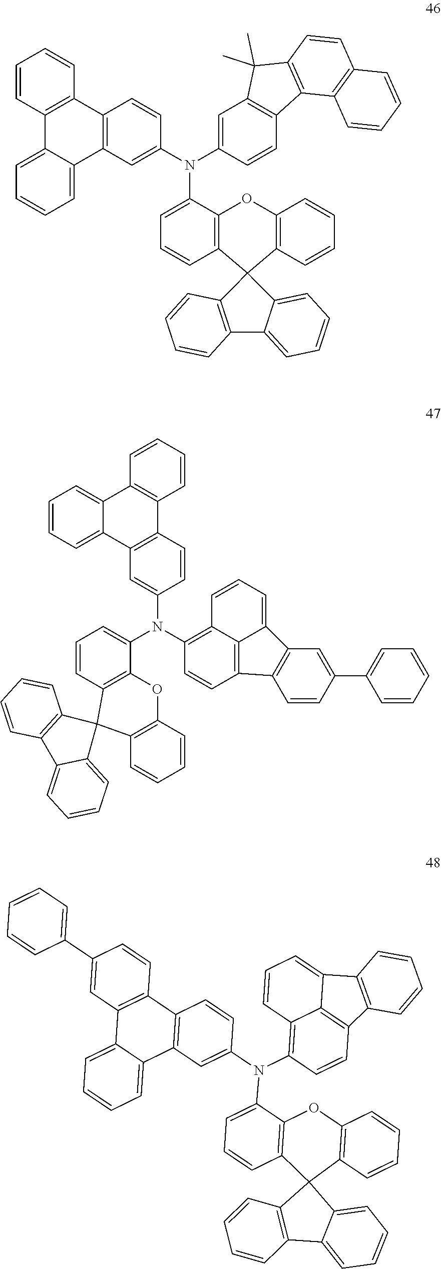 [SMILES: CC1(C)c2cc(N(c3ccc4c5ccccc5c5ccccc5c4c3)c3cccc4c3Oc3ccccc3C43c4ccccc4-c4ccccc43)ccc2-c2c1ccc1ccccc21 ; c1ccc(-c2ccc3c(c2)-c2cccc4c(N(c5ccc6c7ccccc7c7ccccc7c6c5)c5cccc6c5Oc5ccccc5C65c6ccccc6-c6ccccc65)ccc-3c24)cc1 ; c1ccc(-c2ccc3c4ccc(N(c5cccc6c5Oc5ccccc5C65c6ccccc6-c6ccccc65)c5ccc6c7c(cccc57)-c5ccccc5-6)cc4c4ccccc4c3c2)cc1]

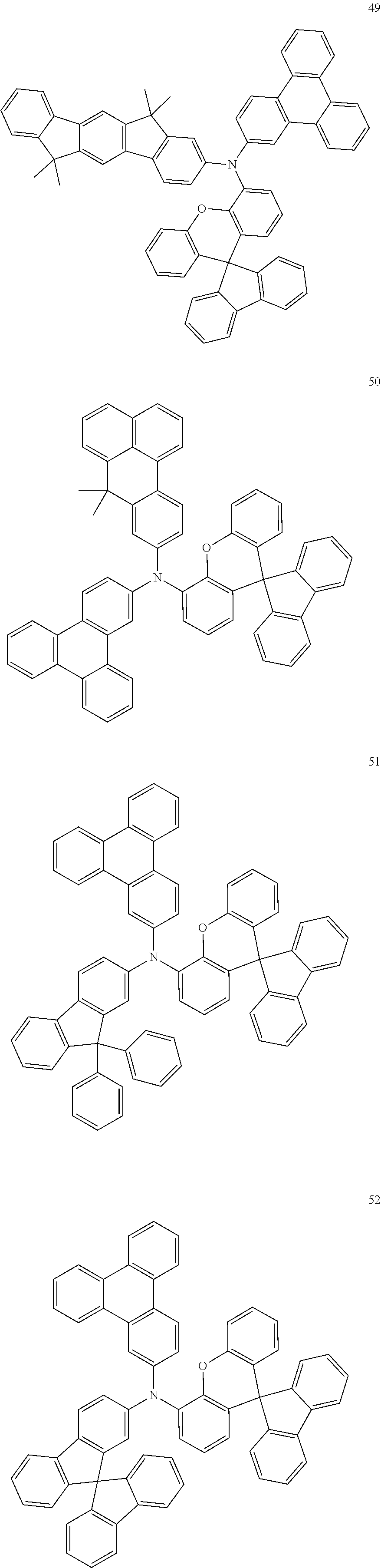 [SMILES: CC1(C)c2ccccc2-c2cc3c(cc21)-c1ccc(N(c2ccc4c5ccccc5c5ccccc5c4c2)c2cccc4c2Oc2ccccc2C42c4ccccc4-c4ccccc42)cc1C3(C)C ; CC1(C)c2cc(N(c3ccc4c5ccccc5c5ccccc5c4c3)c3cccc4c3Oc3ccccc3C43c4ccccc4-c4ccccc43)ccc2-c2cccc3cccc1c23 ; c1ccc(C2(c3ccccc3)c3ccccc3-c3ccc(N(c4ccc5c6ccccc6c6ccccc6c5c4)c4cccc5c4Oc4ccccc4C54c5ccccc5-c5ccccc54)cc32)cc1 ; c1ccc2c(c1)Oc1c(N(c3ccc4c(c3)C3(c5ccccc5-c5ccccc53)c3ccccc3-4)c3ccc4c5ccccc5c5ccccc5c4c3)cccc1C21c2ccccc2-c2ccccc21]

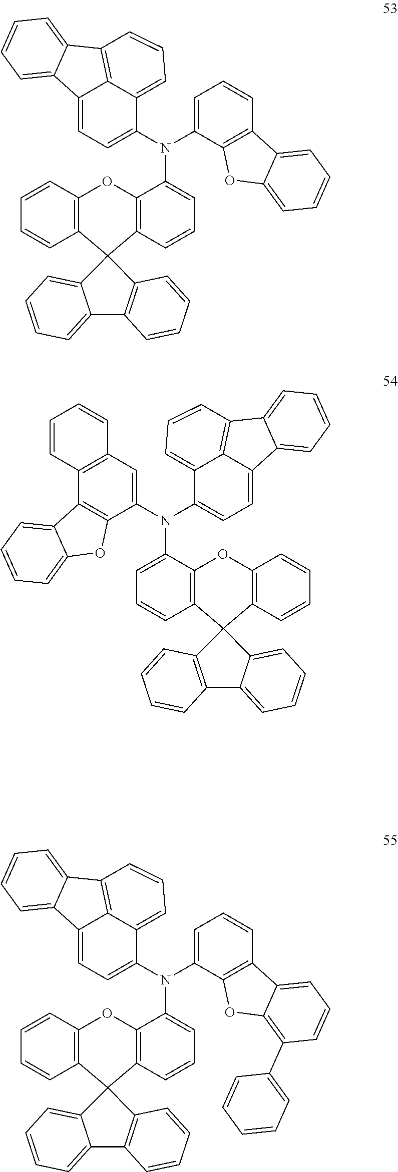 [SMILES: c1ccc2c(c1)Oc1c(N(c3ccc4c5c(cccc35)-c3ccccc3-4)c3cccc4c3oc3ccccc34)cccc1C21c2ccccc2-c2ccccc21 ; c1ccc2c(c1)Oc1c(N(c3ccc4c5c(cccc35)-c3ccccc3-4)c3cc4ccccc4c4c3oc3ccccc34)cccc1C21c2ccccc2-c2ccccc21 ; c1ccc(-c2cccc3c2oc2c(N(c4cccc5c4Oc4ccccc4C54c5ccccc5-c5ccccc54)c4ccc5c6c(cccc46)-c4ccccc4-5)cccc23)cc1]

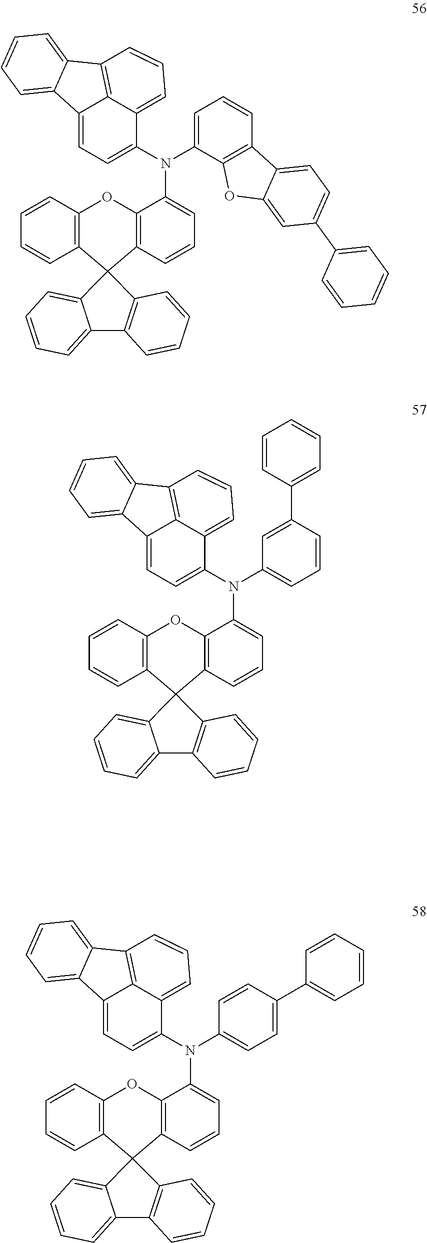 [SMILES: c1ccc(-c2ccc3c(c2)oc2c(N(c4cccc5c4Oc4ccccc4C54c5ccccc5-c5ccccc54)c4ccc5c6c(cccc46)-c4ccccc4-5)cccc23)cc1 ; c1ccc(-c2cccc(N(c3cccc4c3Oc3ccccc3C43c4ccccc4-c4ccccc43)c3ccc4c5c(cccc35)-c3ccccc3-4)c2)cc1 ; c1ccc(-c2ccc(N(c3cccc4c3Oc3ccccc3C43c4ccccc4-c4ccccc43)c3ccc4c5c(cccc35)-c3ccccc3-4)cc2)cc1]

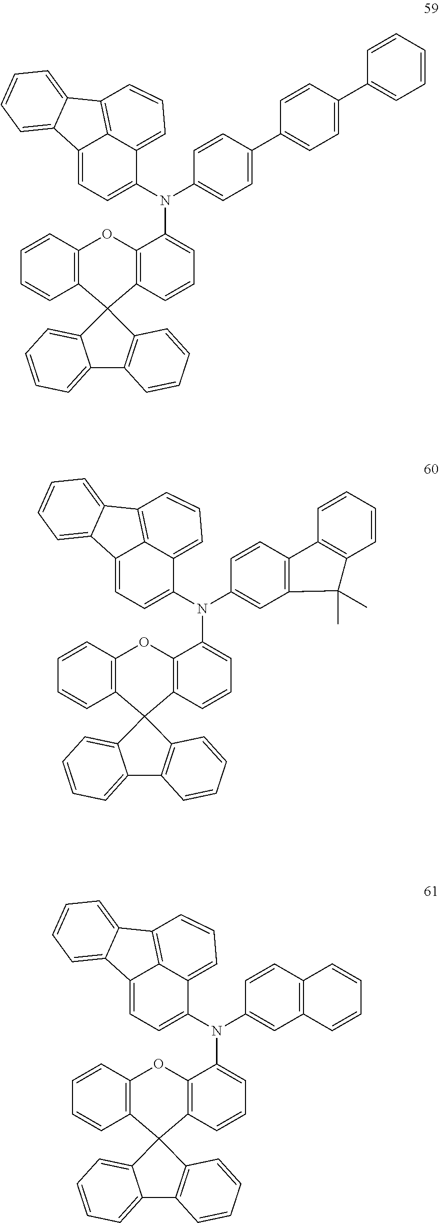 [SMILES: c1ccc(-c2ccc(-c3ccc(N(c4cccc5c4Oc4ccccc4C54c5ccccc5-c5ccccc54)c4ccc5c6c(cccc46)-c4ccccc4-5)cc3)cc2)cc1 ; CC1(C)c2ccccc2-c2ccc(N(c3cccc4c3Oc3ccccc3C43c4ccccc4-c4ccccc43)c3ccc4c5c(cccc35)-c3ccccc3-4)cc21 ; c1ccc2c(c1)Oc1c(N(c3ccc4ccccc4c3)c3ccc4c5c(cccc35)-c3ccccc3-4)cccc1C21c2ccccc2-c2ccccc21]

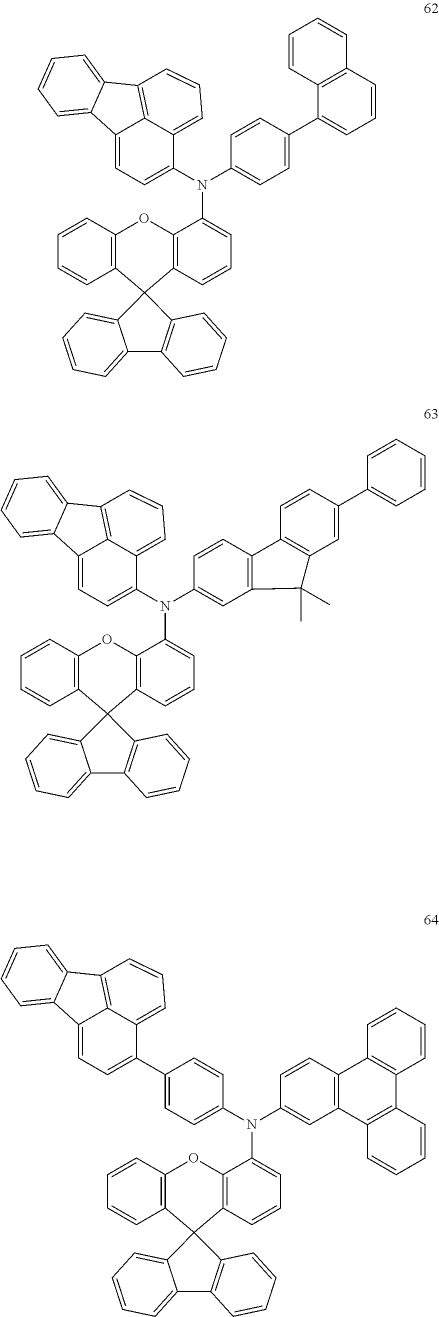 [SMILES: c1ccc2c(c1)Oc1c(N(c3ccc(-c4cccc5ccccc45)cc3)c3ccc4c5c(cccc35)-c3ccccc3-4)cccc1C21c2ccccc2-c2ccccc21 ; CC1(C)c2cc(-c3ccccc3)ccc2-c2ccc(N(c3cccc4c3Oc3ccccc3C43c4ccccc4-c4ccccc43)c3ccc4c5c(cccc35)-c3ccccc3-4)cc21 ; c1ccc2c(c1)Oc1c(N(c3ccc(-c4ccc5c6c(cccc46)-c4ccccc4-5)cc3)c3ccc4c5ccccc5c5ccccc5c4c3)cccc1C21c2ccccc2-c2ccccc21]

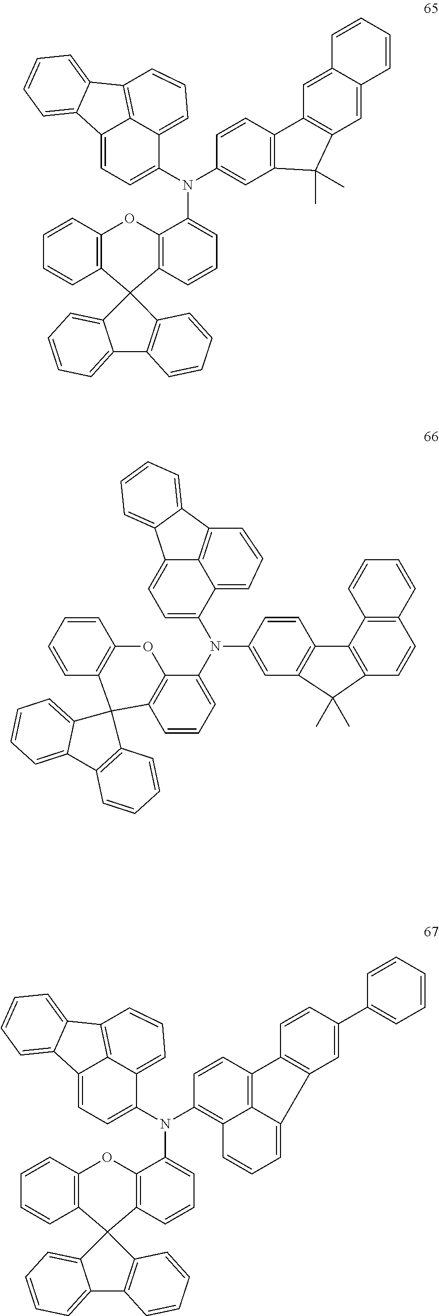 [SMILES: CC1(C)c2cc(N(c3cccc4c3Oc3ccccc3C43c4ccccc4-c4ccccc43)c3ccc4c5c(cccc35)-c3ccccc3-4)ccc2-c2cc3ccccc3cc21 ; CC1(C)c2cc(N(c3cccc4c3Oc3ccccc3C43c4ccccc4-c4ccccc43)c3ccc4c5c(cccc35)-c3ccccc3-4)ccc2-c2c1ccc1ccccc21 ; c1ccc(-c2ccc3c(c2)-c2cccc4c(N(c5cccc6c5Oc5ccccc5C65c6ccccc6-c6ccccc65)c5ccc6c7c(cccc57)-c5ccccc5-6)ccc-3c24)cc1]

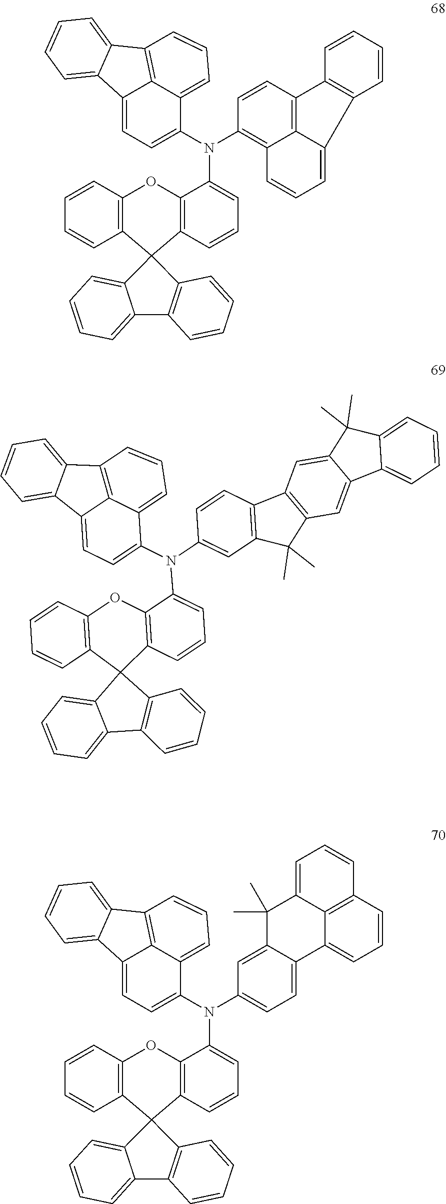 [SMILES: c1ccc2c(c1)Oc1c(N(c3ccc4c5c(cccc35)-c3ccccc3-4)c3ccc4c5c(cccc35)-c3ccccc3-4)cccc1C21c2ccccc2-c2ccccc21 ; CC1(C)c2ccccc2-c2cc3c(cc21)-c1ccc(N(c2cccc4c2Oc2ccccc2C42c4ccccc4-c4ccccc42)c2ccc4c5c(cccc25)-c2ccccc2-4)cc1C3(C)C ; CC1(C)c2cc(N(c3cccc4c3Oc3ccccc3C43c4ccccc4-c4ccccc43)c3ccc4c5c(cccc35)-c3ccccc3-4)ccc2-c2cccc3cccc1c23]

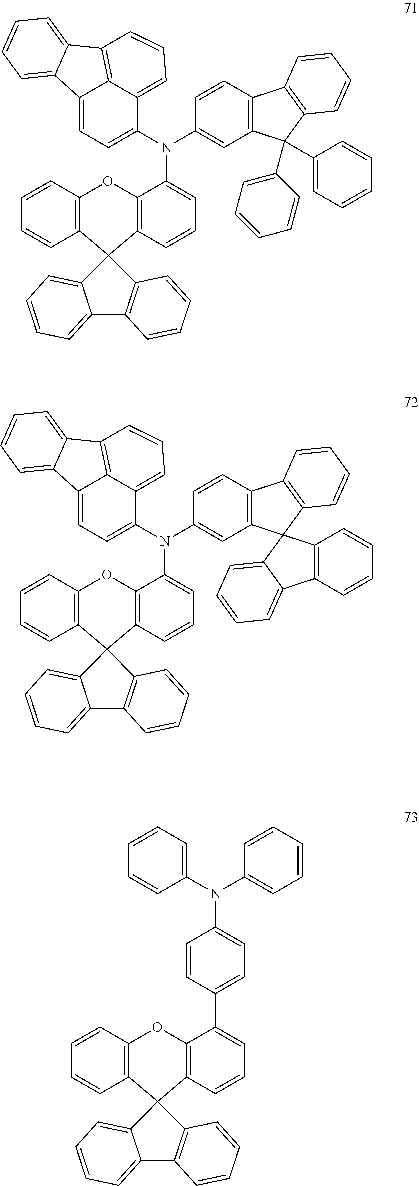 [SMILES: c1ccc(C2(c3ccccc3)c3ccccc3-c3ccc(N(c4cccc5c4Oc4ccccc4C54c5ccccc5-c5ccccc54)c4ccc5c6c(cccc46)-c4ccccc4-5)cc32)cc1 ; c1ccc2c(c1)Oc1c(N(c3ccc4c(c3)C3(c5ccccc5-c5ccccc53)c3ccccc3-4)c3ccc4c5c(cccc35)-c3ccccc3-4)cccc1C21c2ccccc2-c2ccccc21 ; c1ccc(N(c2ccccc2)c2ccc(-c3cccc4c3Oc3ccccc3C43c4ccccc4-c4ccccc43)cc2)cc1]

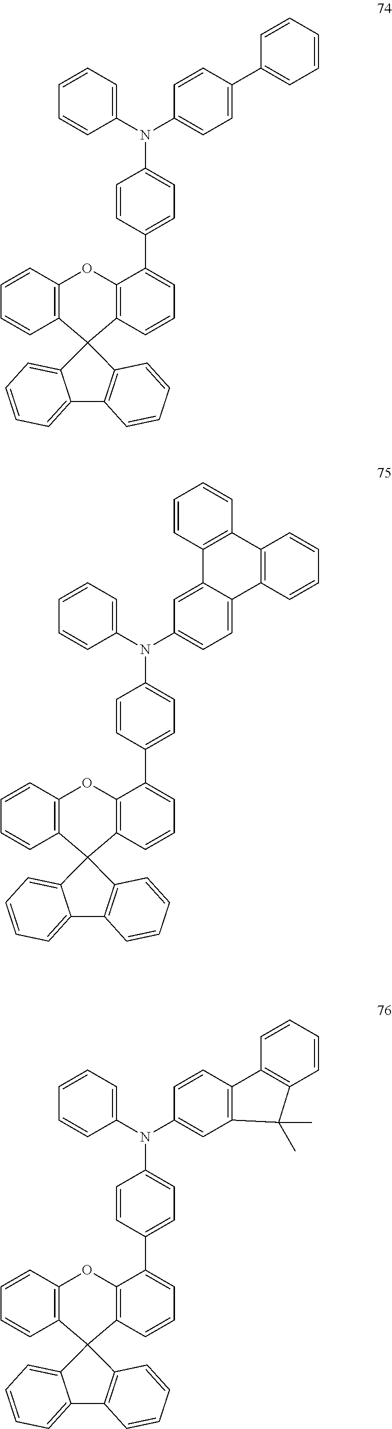 [SMILES: c1ccc(-c2ccc(N(c3ccccc3)c3ccc(-c4cccc5c4Oc4ccccc4C54c5ccccc5-c5ccccc54)cc3)cc2)cc1 ; c1ccc(N(c2ccc(-c3cccc4c3Oc3ccccc3C43c4ccccc4-c4ccccc43)cc2)c2ccc3c4ccccc4c4ccccc4c3c2)cc1 ; CC1(C)c2ccccc2-c2ccc(N(c3ccccc3)c3ccc(-c4cccc5c4Oc4ccccc4C54c5ccccc5-c5ccccc54)cc3)cc21]

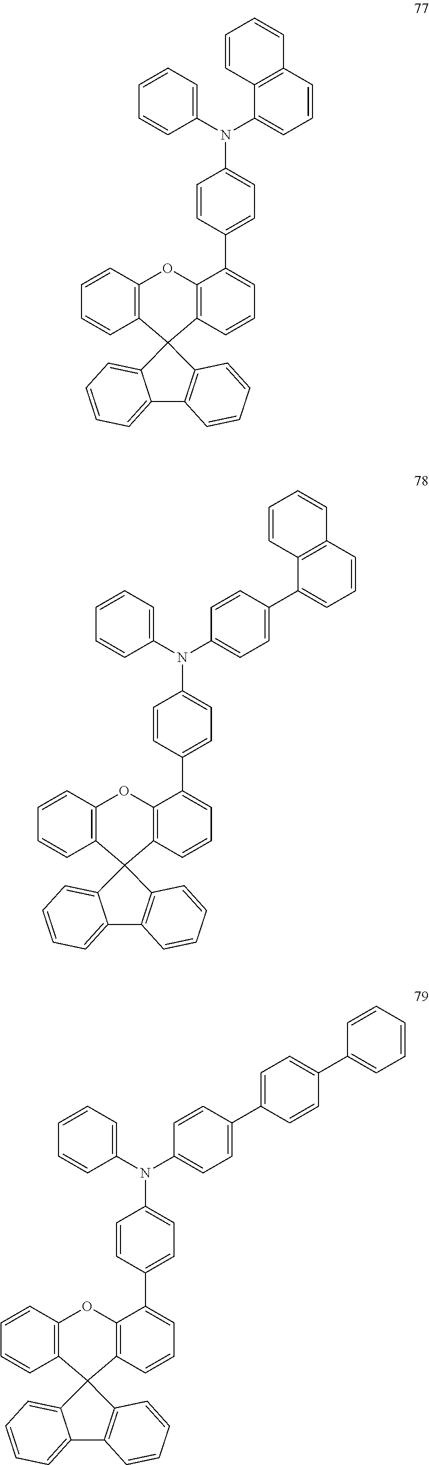 [SMILES: c1ccc(N(c2ccc(-c3cccc4c3Oc3ccccc3C43c4ccccc4-c4ccccc43)cc2)c2cccc3ccccc23)cc1 ; c1ccc(N(c2ccc(-c3cccc4c3Oc3ccccc3C43c4ccccc4-c4ccccc43)cc2)c2ccc(-c3cccc4ccccc34)cc2)cc1 ; c1ccc(-c2ccc(-c3ccc(N(c4ccccc4)c4ccc(-c5cccc6c5Oc5ccccc5C65c6ccccc6-c6ccccc65)cc4)cc3)cc2)cc1]

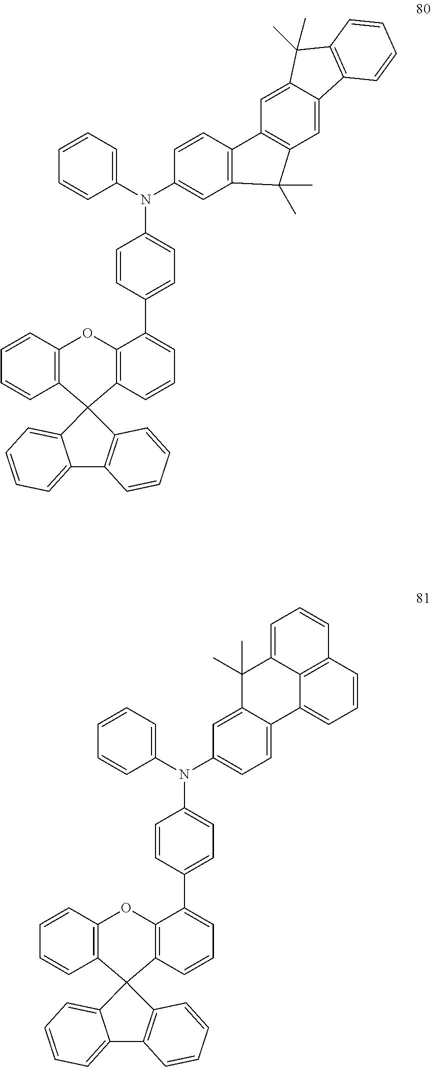 [SMILES: CC1(C)c2ccccc2-c2cc3c(cc21)-c1ccc(N(c2ccccc2)c2ccc(-c4cccc5c4Oc4ccccc4C54c5ccccc5-c5ccccc54)cc2)cc1C3(C)C ; CC1(C)c2cc(N(c3ccccc3)c3ccc(-c4cccc5c4Oc4ccccc4C54c5ccccc5-c5ccccc54)cc3)ccc2-c2cccc3cccc1c23]

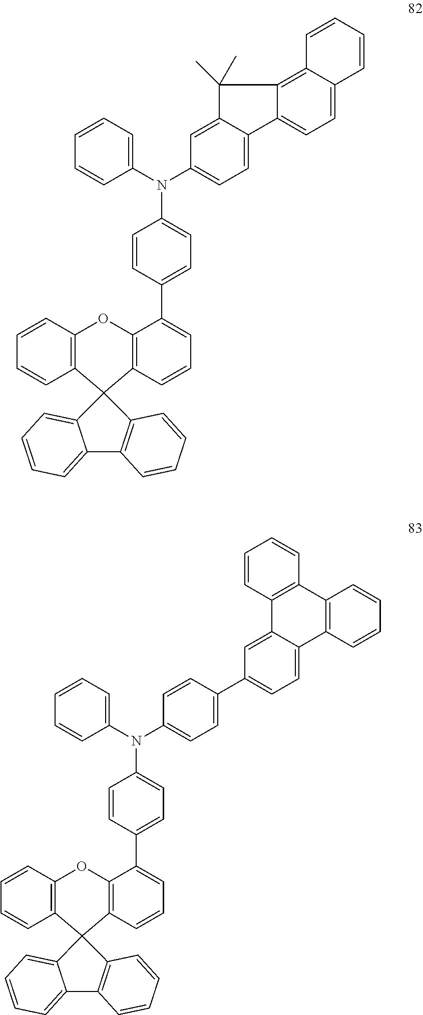 [SMILES: CC1(C)c2cc(N(c3ccccc3)c3ccc(-c4cccc5c4Oc4ccccc4C54c5ccccc5-c5ccccc54)cc3)ccc2-c2ccc3ccccc3c21 ; c1ccc(N(c2ccc(-c3ccc4c5ccccc5c5ccccc5c4c3)cc2)c2ccc(-c3cccc4c3Oc3ccccc3C43c4ccccc4-c4ccccc43)cc2)cc1]

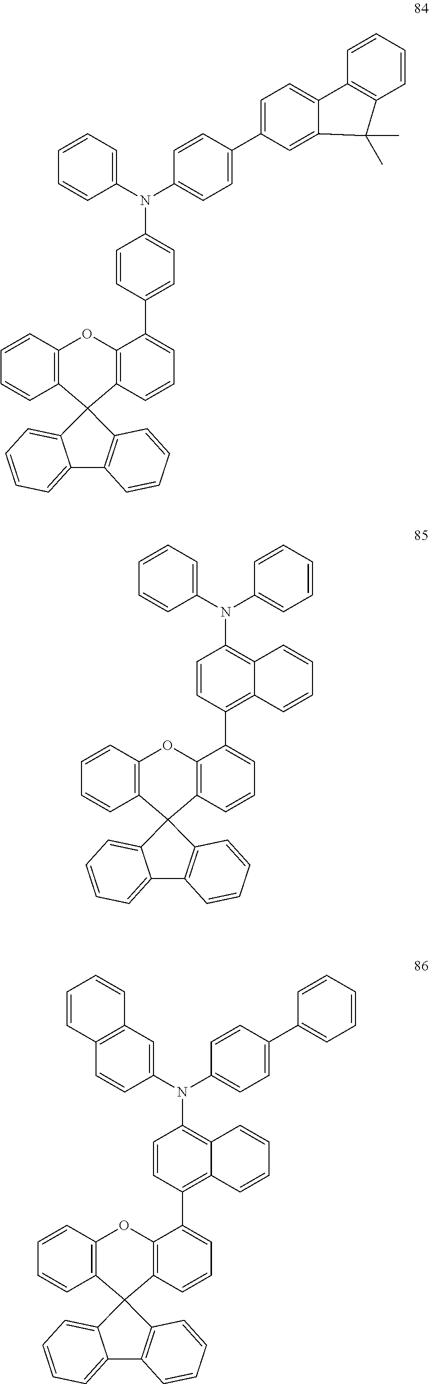 [SMILES: CC1(C)c2ccccc2-c2ccc(-c3ccc(N(c4ccccc4)c4ccc(-c5cccc6c5Oc5ccccc5C65c6ccccc6-c6ccccc65)cc4)cc3)cc21 ; c1ccc(N(c2ccccc2)c2ccc(-c3cccc4c3Oc3ccccc3C43c4ccccc4-c4ccccc43)c3ccccc23)cc1 ; c1ccc(-c2ccc(N(c3ccc4ccccc4c3)c3ccc(-c4cccc5c4Oc4ccccc4C54c5ccccc5-c5ccccc54)c4ccccc34)cc2)cc1]

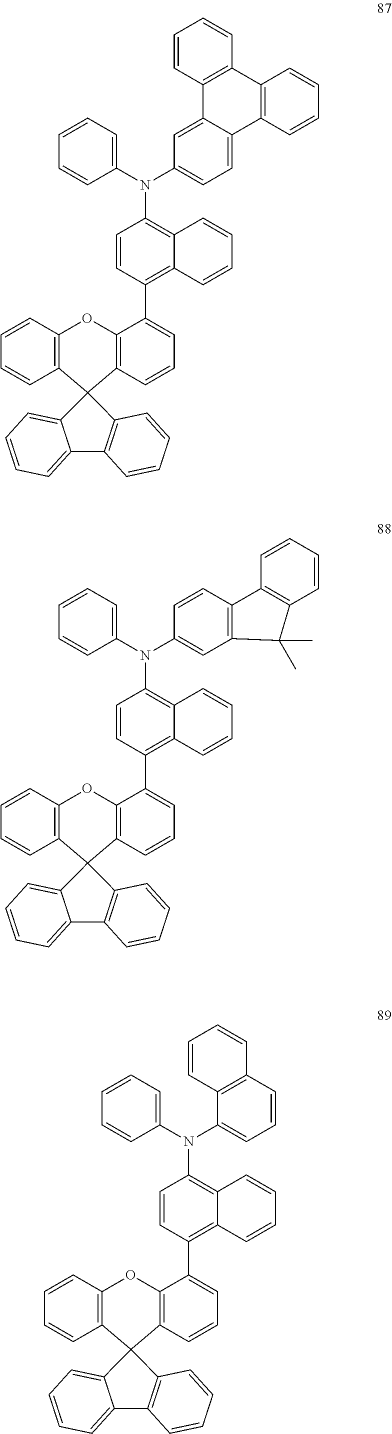 [SMILES: c1ccc(N(c2ccc3c4ccccc4c4ccccc4c3c2)c2ccc(-c3cccc4c3Oc3ccccc3C43c4ccccc4-c4ccccc43)c3ccccc23)cc1 ; CC1(C)c2ccccc2-c2ccc(N(c3ccccc3)c3ccc(-c4cccc5c4Oc4ccccc4C54c5ccccc5-c5ccccc54)c4ccccc34)cc21 ; c1ccc(N(c2cccc3ccccc23)c2ccc(-c3cccc4c3Oc3ccccc3C43c4ccccc4-c4ccccc43)c3ccccc23)cc1]

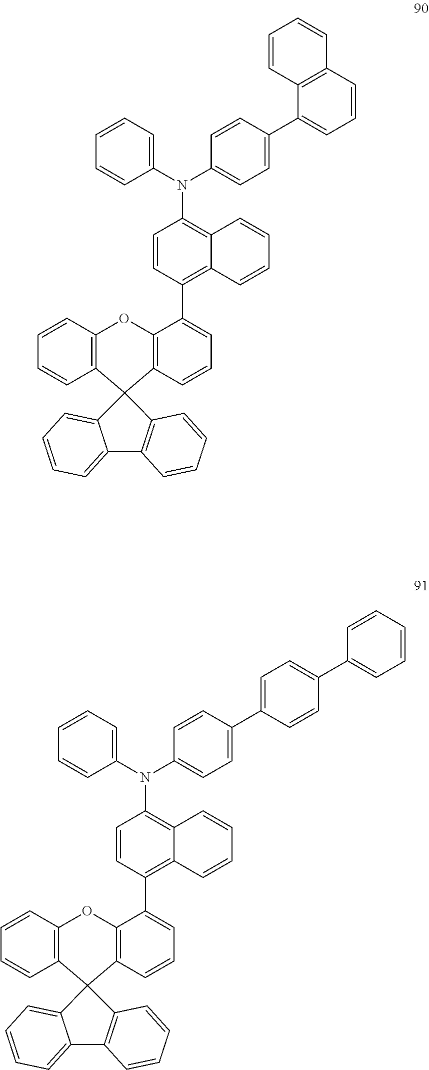 [SMILES: c1ccc(N(c2ccc(-c3cccc4ccccc34)cc2)c2ccc(-c3cccc4c3Oc3ccccc3C43c4ccccc4-c4ccccc43)c3ccccc23)cc1 ; c1ccc(-c2ccc(-c3ccc(N(c4ccccc4)c4ccc(-c5cccc6c5Oc5ccccc5C65c6ccccc6-c6ccccc65)c5ccccc45)cc3)cc2)cc1]

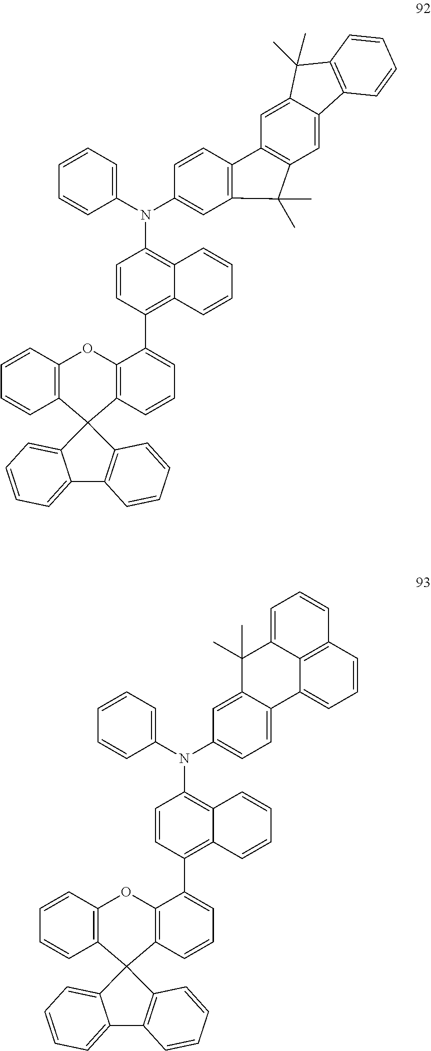 [SMILES: CC1(C)c2ccccc2-c2cc3c(cc21)-c1ccc(N(c2ccccc2)c2ccc(-c4cccc5c4Oc4ccccc4C54c5ccccc5-c5ccccc54)c4ccccc24)cc1C3(C)C ; CC1(C)c2cc(N(c3ccccc3)c3ccc(-c4cccc5c4Oc4ccccc4C54c5ccccc5-c5ccccc54)c4ccccc34)ccc2-c2cccc3cccc1c23]

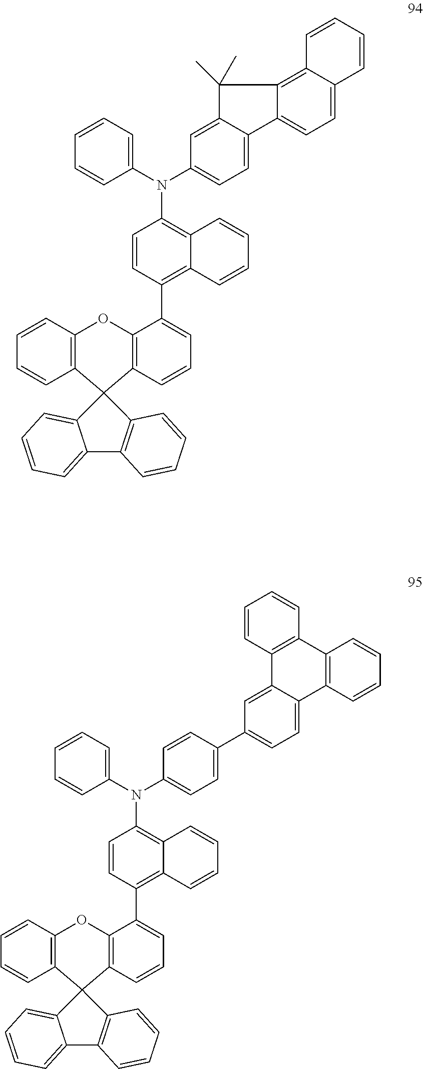 [SMILES: CC1(C)c2cc(N(c3ccccc3)c3ccc(-c4cccc5c4Oc4ccccc4C54c5ccccc5-c5ccccc54)c4ccccc34)ccc2-c2ccc3ccccc3c21 ; c1ccc(N(c2ccc(-c3ccc4c5ccccc5c5ccccc5c4c3)cc2)c2ccc(-c3cccc4c3Oc3ccccc3C43c4ccccc4-c4ccccc43)c3ccccc23)cc1]

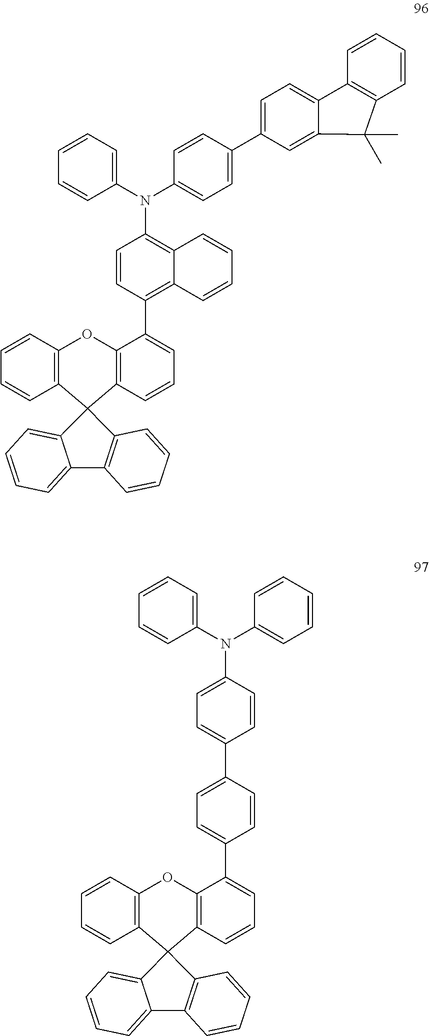 [SMILES: CC1(C)c2ccccc2-c2ccc(-c3ccc(N(c4ccccc4)c4ccc(-c5cccc6c5Oc5ccccc5C65c6ccccc6-c6ccccc65)c5ccccc45)cc3)cc21 ; c1ccc(N(c2ccccc2)c2ccc(-c3ccc(-c4cccc5c4Oc4ccccc4C54c5ccccc5-c5ccccc54)cc3)cc2)cc1]

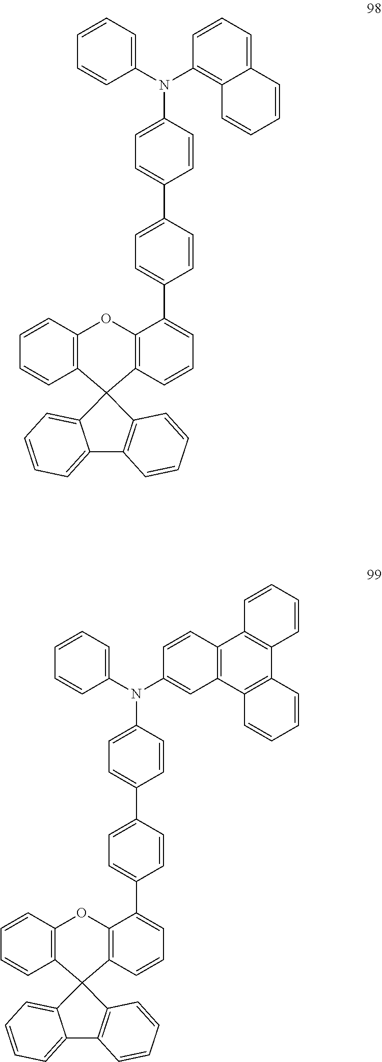 [SMILES: c1ccc(N(c2ccc(-c3ccc(-c4cccc5c4Oc4ccccc4C54c5ccccc5-c5ccccc54)cc3)cc2)c2cccc3ccccc23)cc1 ; c1ccc(N(c2ccc(-c3ccc(-c4cccc5c4Oc4ccccc4C54c5ccccc5-c5ccccc54)cc3)cc2)c2ccc3c4ccccc4c4ccccc4c3c2)cc1]

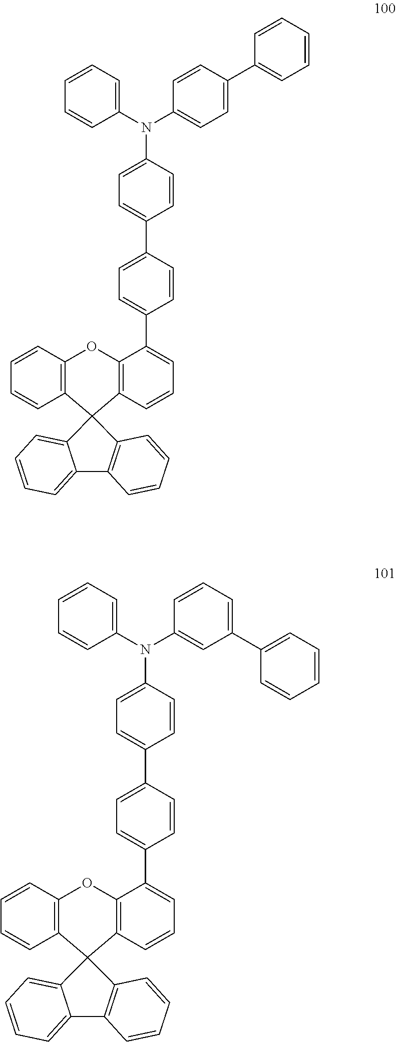 [SMILES: c1ccc(-c2ccc(N(c3ccccc3)c3ccc(-c4ccc(-c5cccc6c5Oc5ccccc5C65c6ccccc6-c6ccccc65)cc4)cc3)cc2)cc1 ; c1ccc(-c2cccc(N(c3ccccc3)c3ccc(-c4ccc(-c5cccc6c5Oc5ccccc5C65c6ccccc6-c6ccccc65)cc4)cc3)c2)cc1]

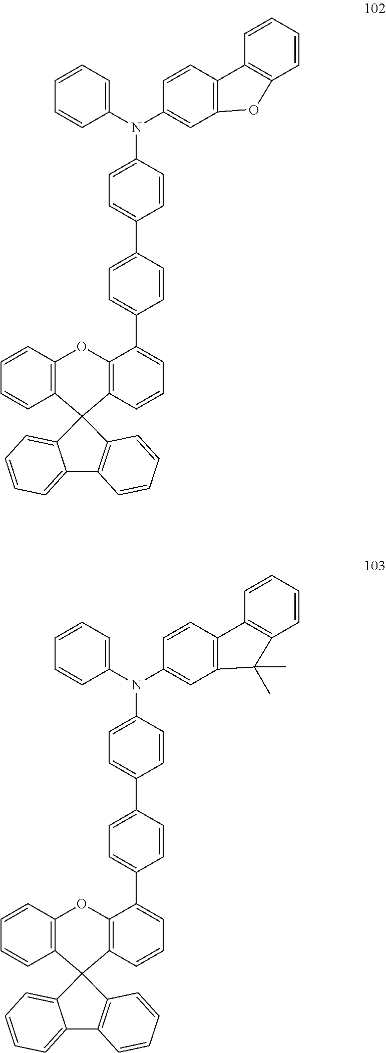 [SMILES: c1ccc(N(c2ccc(-c3ccc(-c4cccc5c4Oc4ccccc4C54c5ccccc5-c5ccccc54)cc3)cc2)c2ccc3c(c2)oc2ccccc23)cc1 ; CC1(C)c2ccccc2-c2ccc(N(c3ccccc3)c3ccc(-c4ccc(-c5cccc6c5Oc5ccccc5C65c6ccccc6-c6ccccc65)cc4)cc3)cc21]

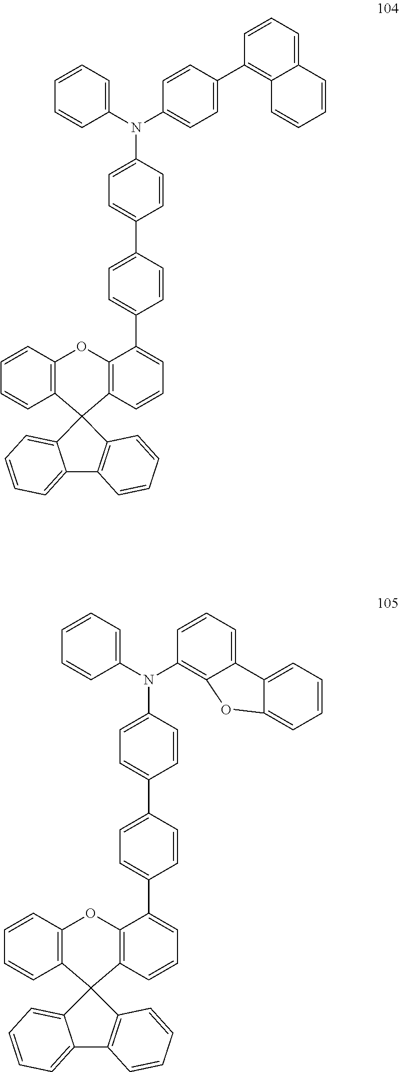 [SMILES: c1ccc(N(c2ccc(-c3ccc(-c4cccc5c4Oc4ccccc4C54c5ccccc5-c5ccccc54)cc3)cc2)c2ccc(-c3cccc4ccccc34)cc2)cc1 ; c1ccc(N(c2ccc(-c3ccc(-c4cccc5c4Oc4ccccc4C54c5ccccc5-c5ccccc54)cc3)cc2)c2cccc3c2oc2ccccc23)cc1]

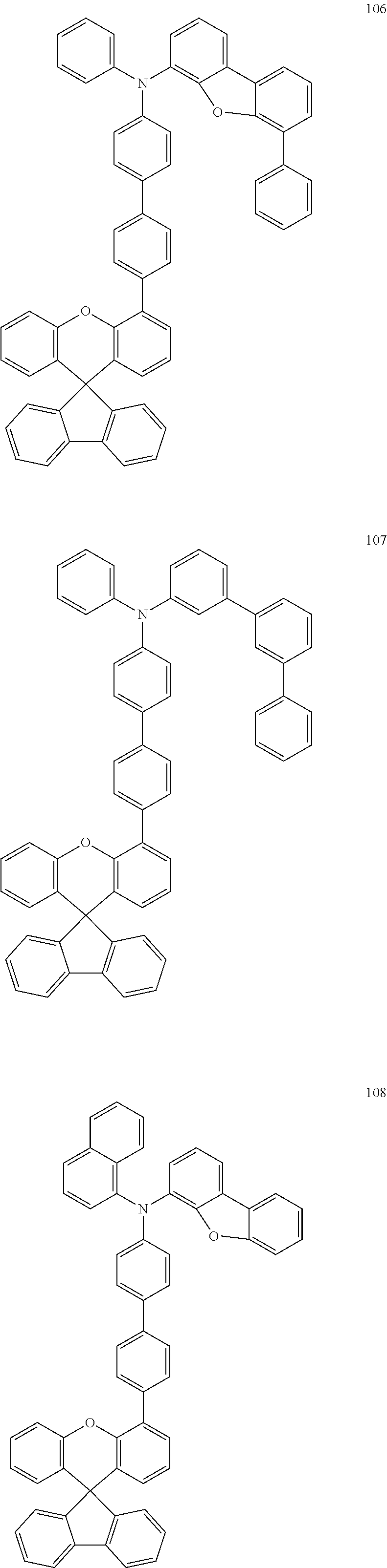 [SMILES: c1ccc(-c2cccc3c2oc2c(N(c4ccccc4)c4ccc(-c5ccc(-c6cccc7c6Oc6ccccc6C76c7ccccc7-c7ccccc76)cc5)cc4)cccc23)cc1 ; c1ccc(-c2cccc(-c3cccc(N(c4ccccc4)c4ccc(-c5ccc(-c6cccc7c6Oc6ccccc6C76c7ccccc7-c7ccccc76)cc5)cc4)c3)c2)cc1 ; c1ccc2c(c1)Oc1c(-c3ccc(-c4ccc(N(c5cccc6ccccc56)c5cccc6c5oc5ccccc56)cc4)cc3)cccc1C21c2ccccc2-c2ccccc21]

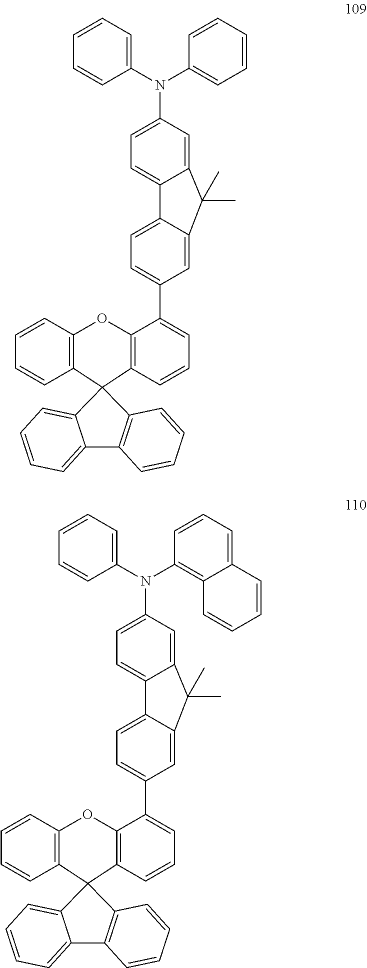 [SMILES: CC1(C)c2cc(-c3cccc4c3Oc3ccccc3C43c4ccccc4-c4ccccc43)ccc2-c2ccc(N(c3ccccc3)c3ccccc3)cc21 ; CC1(C)c2cc(-c3cccc4c3Oc3ccccc3C43c4ccccc4-c4ccccc43)ccc2-c2ccc(N(c3ccccc3)c3cccc4ccccc34)cc21]

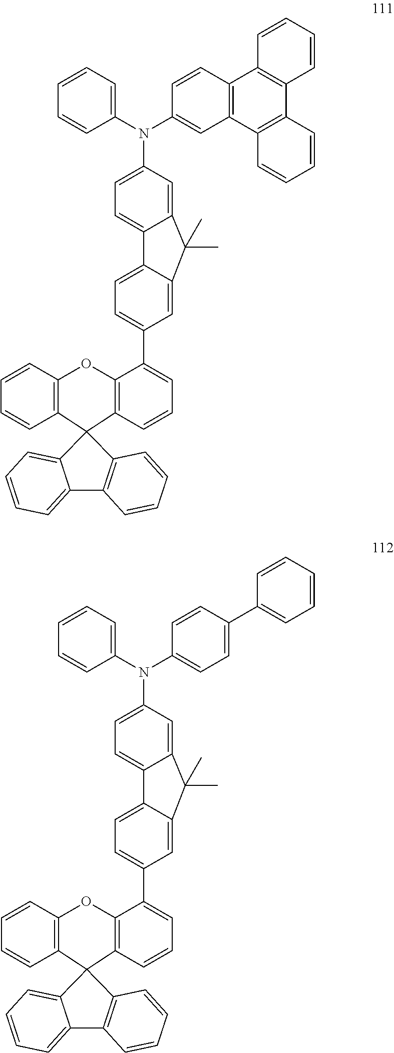 [SMILES: CC1(C)c2cc(-c3cccc4c3Oc3ccccc3C43c4ccccc4-c4ccccc43)ccc2-c2ccc(N(c3ccccc3)c3ccc4c5ccccc5c5ccccc5c4c3)cc21 ; CC1(C)c2cc(-c3cccc4c3Oc3ccccc3C43c4ccccc4-c4ccccc43)ccc2-c2ccc(N(c3ccccc3)c3ccc(-c4ccccc4)cc3)cc21]

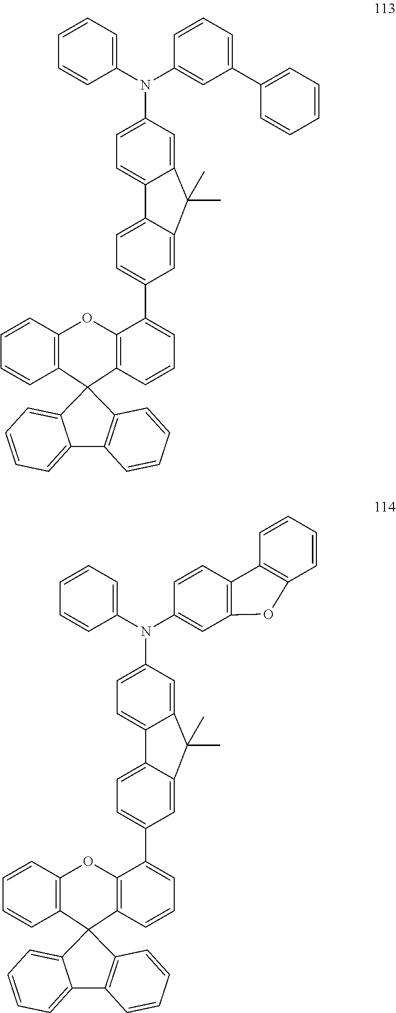 [SMILES: CC1(C)c2cc(-c3cccc4c3Oc3ccccc3C43c4ccccc4-c4ccccc43)ccc2-c2ccc(N(c3ccccc3)c3cccc(-c4ccccc4)c3)cc21 ; CC1(C)c2cc(-c3cccc4c3Oc3ccccc3C43c4ccccc4-c4ccccc43)ccc2-c2ccc(N(c3ccccc3)c3ccc4c(c3)oc3ccccc34)cc21]

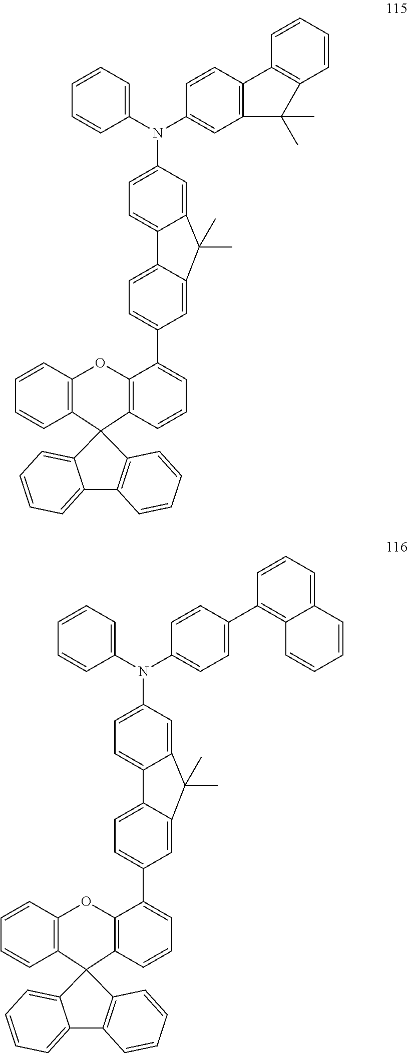 [SMILES: CC1(C)c2ccccc2-c2ccc(N(c3ccccc3)c3ccc4c(c3)C(C)(C)c3cc(-c5cccc6c5Oc5ccccc5C65c6ccccc6-c6ccccc65)ccc3-4)cc21 ; CC1(C)c2cc(-c3cccc4c3Oc3ccccc3C43c4ccccc4-c4ccccc43)ccc2-c2ccc(N(c3ccccc3)c3ccc(-c4cccc5ccccc45)cc3)cc21]

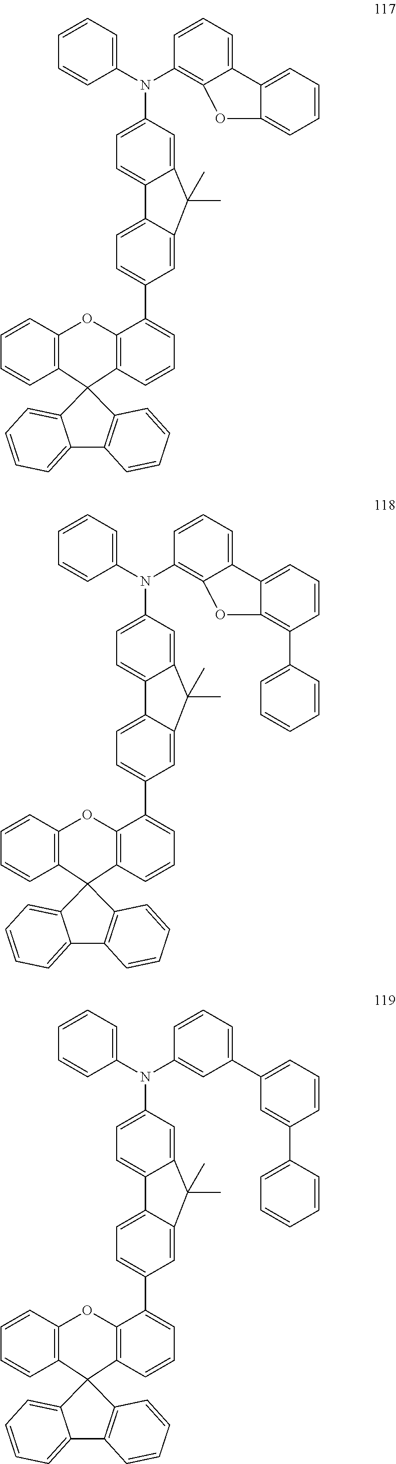 [SMILES: CC1(C)c2cc(-c3cccc4c3Oc3ccccc3C43c4ccccc4-c4ccccc43)ccc2-c2ccc(N(c3ccccc3)c3cccc4c3oc3ccccc34)cc21 ; CC1(C)c2cc(-c3cccc4c3Oc3ccccc3C43c4ccccc4-c4ccccc43)ccc2-c2ccc(N(c3ccccc3)c3cccc4c3oc3c(-c5ccccc5)cccc34)cc21 ; CC1(C)c2cc(-c3cccc4c3Oc3ccccc3C43c4ccccc4-c4ccccc43)ccc2-c2ccc(N(c3ccccc3)c3cccc(-c4cccc(-c5ccccc5)c4)c3)cc21]

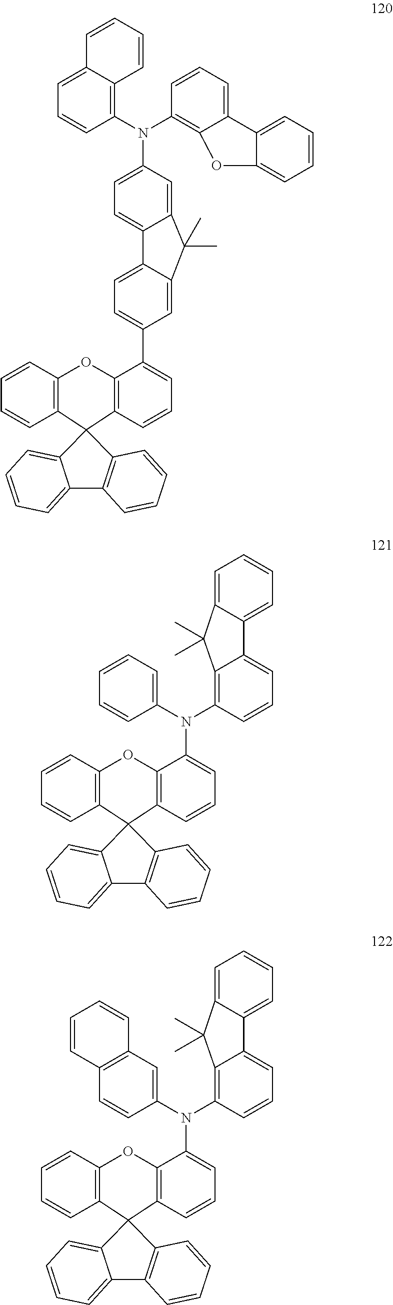 [SMILES: CC1(C)c2cc(-c3cccc4c3Oc3ccccc3C43c4ccccc4-c4ccccc43)ccc2-c2ccc(N(c3cccc4ccccc34)c3cccc4c3oc3ccccc34)cc21 ; CC1(C)c2ccccc2-c2cccc(N(c3ccccc3)c3cccc4c3Oc3ccccc3C43c4ccccc4-c4ccccc43)c21 ; CC1(C)c2ccccc2-c2cccc(N(c3ccc4ccccc4c3)c3cccc4c3Oc3ccccc3C43c4ccccc4-c4ccccc43)c21]

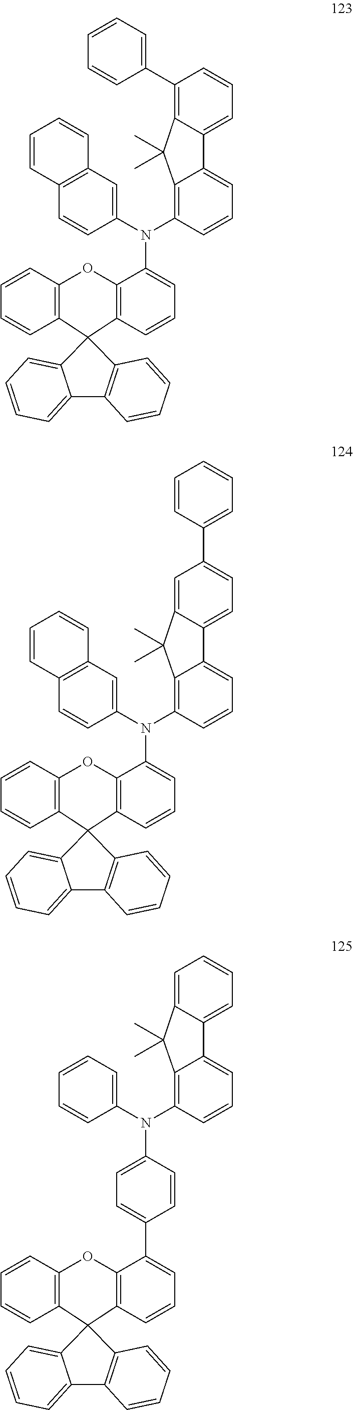 [SMILES: CC1(C)c2c(-c3ccccc3)cccc2-c2cccc(N(c3ccc4ccccc4c3)c3cccc4c3Oc3ccccc3C43c4ccccc4-c4ccccc43)c21 ; CC1(C)c2cc(-c3ccccc3)ccc2-c2cccc(N(c3ccc4ccccc4c3)c3cccc4c3Oc3ccccc3C43c4ccccc4-c4ccccc43)c21 ; CC1(C)c2ccccc2-c2cccc(N(c3ccccc3)c3ccc(-c4cccc5c4Oc4ccccc4C54c5ccccc5-c5ccccc54)cc3)c21]

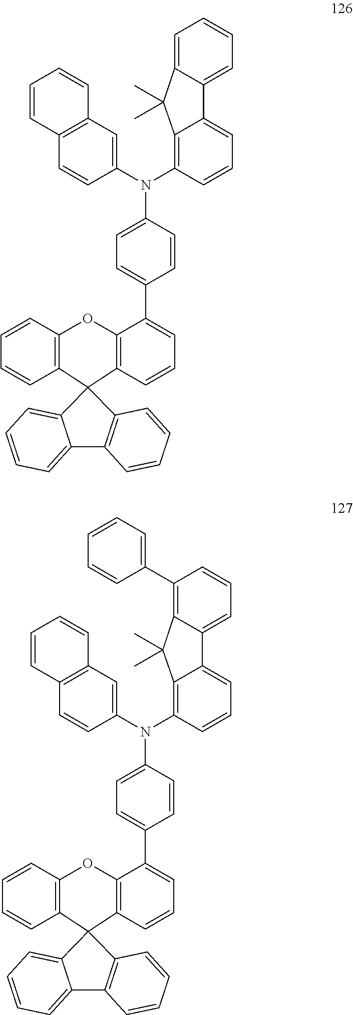 [SMILES: CC1(C)c2ccccc2-c2cccc(N(c3ccc(-c4cccc5c4Oc4ccccc4C54c5ccccc5-c5ccccc54)cc3)c3ccc4ccccc4c3)c21 ; CC1(C)c2c(-c3ccccc3)cccc2-c2cccc(N(c3ccc(-c4cccc5c4Oc4ccccc4C54c5ccccc5-c5ccccc54)cc3)c3ccc4ccccc4c3)c21]

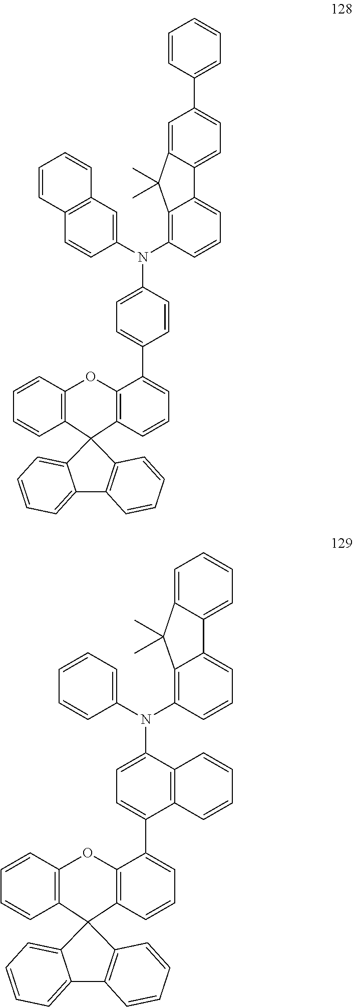 [SMILES: CC1(C)c2cc(-c3ccccc3)ccc2-c2cccc(N(c3ccc(-c4cccc5c4Oc4ccccc4C54c5ccccc5-c5ccccc54)cc3)c3ccc4ccccc4c3)c21 ; CC1(C)c2ccccc2-c2cccc(N(c3ccccc3)c3ccc(-c4cccc5c4Oc4ccccc4C54c5ccccc5-c5ccccc54)c4ccccc34)c21]

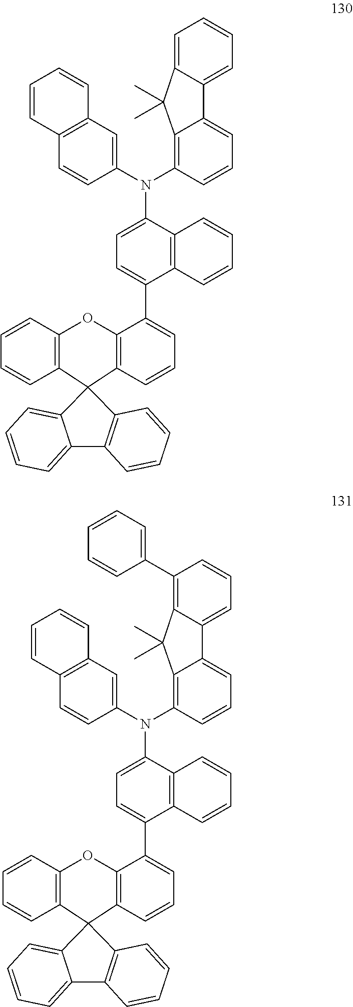

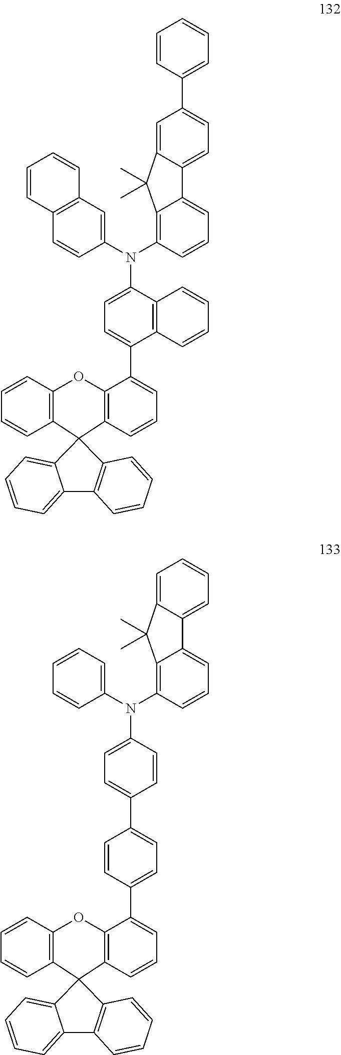 [SMILES: CC1(C)c2cc(-c3ccccc3)ccc2-c2cccc(N(c3ccc4ccccc4c3)c3ccc(-c4cccc5c4Oc4ccccc4C54c5ccccc5-c5ccccc54)c4ccccc34)c21 ; CC1(C)c2ccccc2-c2cccc(N(c3ccccc3)c3ccc(-c4ccc(-c5cccc6c5Oc5ccccc5C65c6ccccc6-c6ccccc65)cc4)cc3)c21]

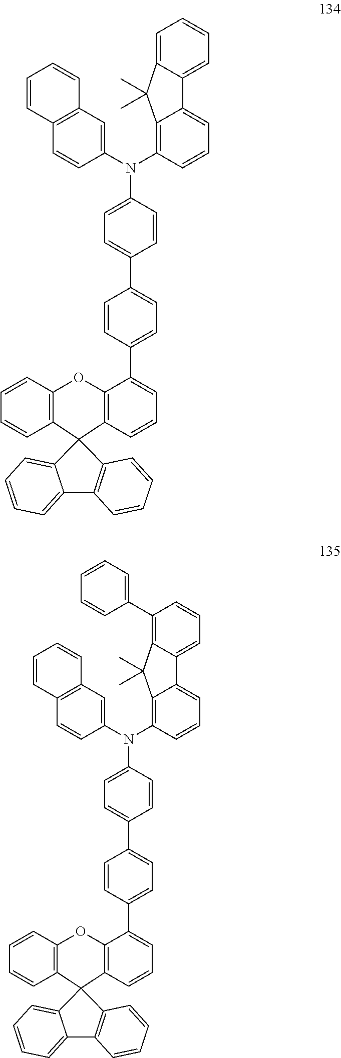 [SMILES: CC1(C)c2ccccc2-c2cccc(N(c3ccc(-c4ccc(-c5cccc6c5Oc5ccccc5C65c6ccccc6-c6ccccc65)cc4)cc3)c3ccc4ccccc4c3)c21 ; CC1(C)c2c(-c3ccccc3)cccc2-c2cccc(N(c3ccc(-c4ccc(-c5cccc6c5Oc5ccccc5C65c6ccccc6-c6ccccc65)cc4)cc3)c3ccc4ccccc4c3)c21]

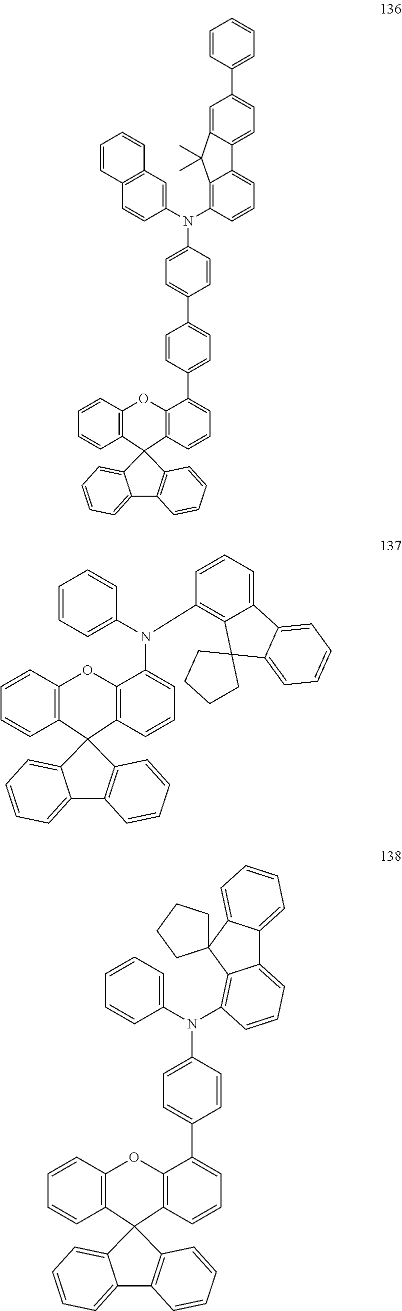 [SMILES: CC1(C)c2cc(-c3ccccc3)ccc2-c2cccc(N(c3ccc(-c4ccc(-c5cccc6c5Oc5ccccc5C65c6ccccc6-c6ccccc65)cc4)cc3)c3ccc4ccccc4c3)c21 ; c1ccc(N(c2cccc3c2Oc2ccccc2C32c3ccccc3-c3ccccc32)c2cccc3c2C2(CCCC2)c2ccccc2-3)cc1 ; c1ccc(N(c2ccc(-c3cccc4c3Oc3ccccc3C43c4ccccc4-c4ccccc43)cc2)c2cccc3c2C2(CCCC2)c2ccccc2-3)cc1]

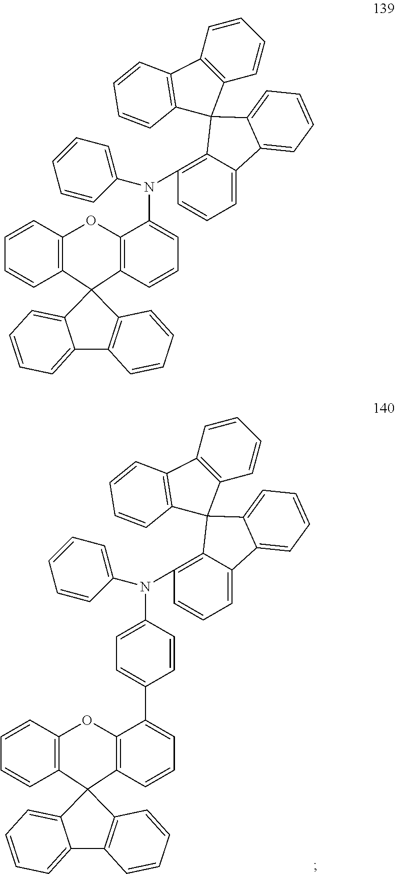 [SMILES: c1ccc(N(c2cccc3c2Oc2ccccc2C32c3ccccc3-c3ccccc32)c2cccc3c2C2(c4ccccc4-c4ccccc42)c2ccccc2-3)cc1 ; c1ccc(N(c2ccc(-c3cccc4c3Oc3ccccc3C43c4ccccc4-c4ccccc43)cc2)c2cccc3c2C2(c4ccccc4-c4ccccc42)c2ccccc2-3)cc1]

the compounds 1S-140S are obtained by replacing

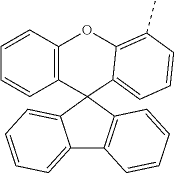

in Compounds 1-140 with

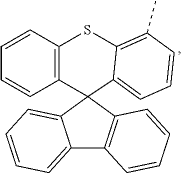

wherein the dashed line represents the linkage site.

For example, the structure of Compound 2 is

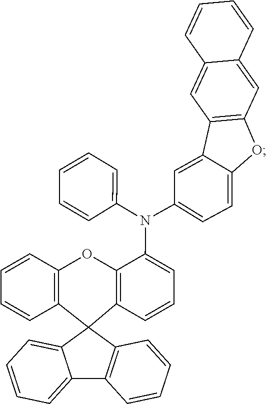 [SMILES: c1ccc(N(c2ccc3oc4cc5ccccc5cc4c3c2)c2cccc3c2Oc2ccccc2C32c3ccccc3-c3ccccc32)cc1]

and the structure of Compound 2S is

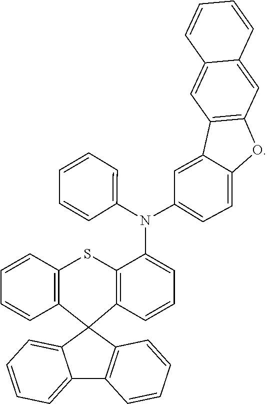 [SMILES: c1ccc(N(c2ccc3oc4cc5ccccc5cc4c3c2)c2cccc3c2Sc2ccccc2C32c3ccccc3-c3ccccc32)cc1]

In a fifth aspect, the present application provides a display apparatus, and the display apparatus includes the organic electroluminescent device as described in the fourth aspect.

Compared with the prior art, the present application has the following beneficial effects.

A composition with specific component is obtained by designing a specific component of the composition, and an organic electroluminescent device with a lower driving voltage, a higher current efficiency and a longer lifetime can be fabricated by using this composition as the material of the emissive layer of the organic electroluminescent device.

Other aspects can be understood after reading and understanding the detailed description.

### DETAILED DESCRIPTION

In order to facilitate the understanding of the present application, embodiments of the present application are provided below. It should be understood by those skilled in the art that the embodiments are merely an aid to understanding the present application and should not be regarded as a specific limitation of the present application.

### Synthesis Example 1

This synthesis example provides Compound H-29-D and a synthesis method thereof, and the synthesis method is as follows.

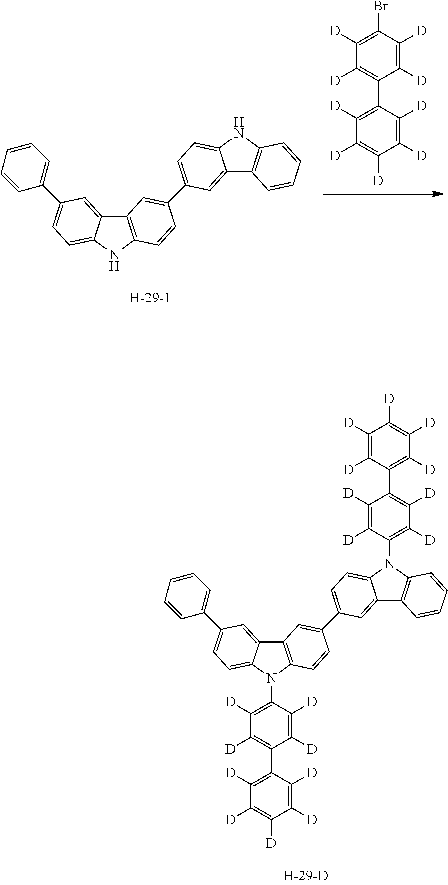 [SMILES: c1ccc(-c2ccc3[nH]c4ccc(-c5ccc6[nH]c7ccccc7c6c5)cc4c3c2)cc1 ; [2H]c1c([2H])c([2H])c(-c2c([2H])c([2H])c(Br)c([2H])c2[2H])c([2H])c1[2H] ; [2H]c1c([2H])c([2H])c(-c2c([2H])c([2H])c(-n3c4ccccc4c4cc(-c5ccc6c(c5)c5cc(-c7ccccc7)ccc5n6-c5c([2H])c([2H])c(-c6c([2H])c([2H])c([2H])c([2H])c6[2H])c([2H])c5[2H])ccc43)c([2H])c2[2H])c([2H])c1[2H]]

Under the nitrogen protection, 100 mL of dry toluene, the intermediate H-29-1 (4.1 g), brominated deuterated biphenyl (5.2 g), Pd(dba)₂ (bis(dibenzylideneacetone)palladium, 0.1 g), a toluene solution of tri-tert-butylphosphine with a mass percent of 10% (0.8 g), and sodium tert-butoxide (3.1 g) were added to a 250 mL three-necked flask. The mixture was heated slowly to reflux and reacted for 8 hours, cooled down to room temperature and added with water for phase separation. The organic layer was washed with water until neutral, dried with magnesium sulfate, filtered to remove magnesium sulfate and concentrated to dryness. Purification was performed by silica gel column chromatography using a petroleum ether:dichloromethane mixture (10:1, v/v) as a eluent, yielding 6.0 g of Compound H-29-D.

Mass spectrometry analysis of Compound H-29-D: A mass-to-charge ratio (m/z) of 730.40 was detected.

### Synthesis Example 2

This synthesis example provides Compound H-29-DE and a synthesis method thereof, and the synthesis method is as follows.

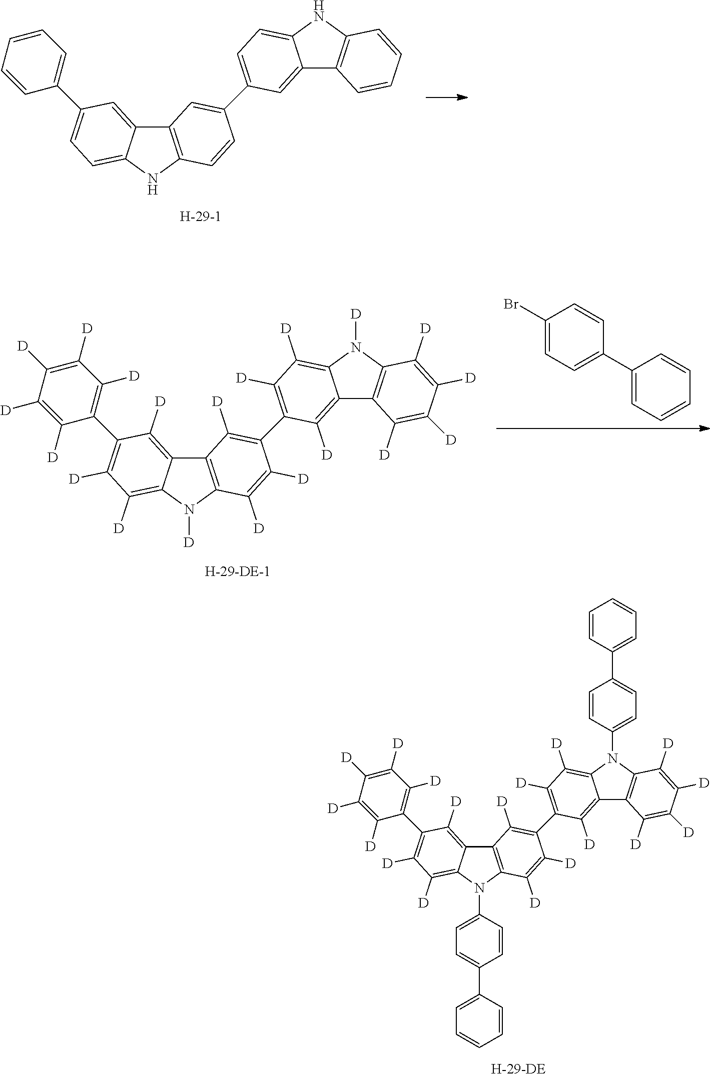

### (1) Synthesis of the Intermediate H-29-DE-1

Compound H-29-1 (3.9 g), palladium chloride (0.026 g), anhydrous nickel chloride (0.018 g), activated charcoal (0.3 g), D₂O (48 mL) and C₆D₆ (120 mL) were added to a 500 mL autoclave at 25° C. and introduced with hydrogen to a pressure of 0.02 MPa, and then the reaction was heated to 90° C. for 60 h, and cooled down to room temperature, and subjected to filtration and phase separation, and the organic layer after the phase separation was dried with magnesium sulfate, decolorized with a short silica gel column, concentrated to dryness. Purification was performed by silica gel column chromatography using a petroleum ether:ethyl acetate mixture (10:1, v/v), yielding 3.2 g of intermediate H-29-DE-1.

Mass spectrometry analysis of intermediate H-29-D1-1: A mass-to-charge ratio (m/z) of 428.29 was detected.

### (2) Synthesis of Compound H-29-DE

The procedure was carried out similarly to the synthesis of Compound H-29-D, except that the Compound H-29-D was substituted with an equimolar amount of intermediate H-29-DE-1, and brominated deuterated biphenyl was substituted with an equimolar amount of brominated biphenyl. Compound H-29-DE was obtained.

Mass spectrometry analysis of Compound H-29-DE: A mass-to-charge ratio (m/z) of 730.40 was detected.

This synthesis example provides another synthesis method of Compound H-29-DE, and the synthesis method is as follows.

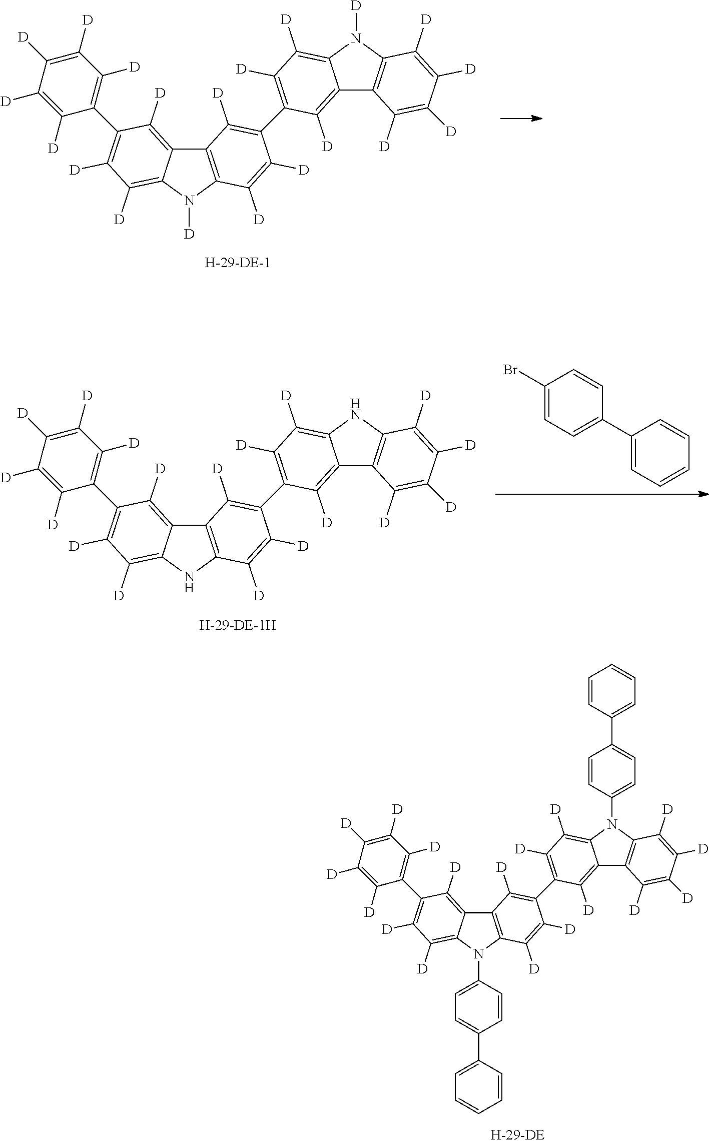

### (1) Synthesis of the Intermediate H-29-DE-1H

An intermediate H-29-DE-1 (4.3 g) and 50 mL of dry tetrahydrofuran were added to a 250 mL three-necked flask, and the reaction was slowly cool down to −50° C., slowly dropwise added with 14 mL of the 1.6 M butyl lithium in n-hexane, held at −50° C. for 30 min after the addition, slowly added with 2 g of methanol, heated to room temperature, added with water and dichloromethane for phase separation, then the organic layer was washed with water, dried with magnesium sulfate, separated by silica gel column chromatography to obtain the intermediate H-29-DE-1H (3.6 g).

Mass spectrometry analysis of Compound H-29-DE-1H: A mass-to-charge ratio (m/z) of 426.28 was detected.

### (2) Synthesis of the Intermediate H-29-DE

The procedure was carried out similarly to the synthesis of Compound H-29-D, except that the Compound H-29-D was substituted with an equimolar amount of intermediate H-29-DE-1H, and brominated deuterated biphenyl was substituted with an equimolar amount of brominated biphenyl. Compound H-29-DE was obtained.

Mass spectrometry analysis of Compound H-29-DE: A mass-to-charge ratio (m/z) of 730.40 was detected.

Other compounds with no specific synthesis steps listed can be prepared by common knowledge in the field in conjunction with the above examples.

The specific structures of the compounds used in the following Device Examples and Device Comparative Examples are as follows.

The specific structures of the compounds adopted in the following examples are shown below:

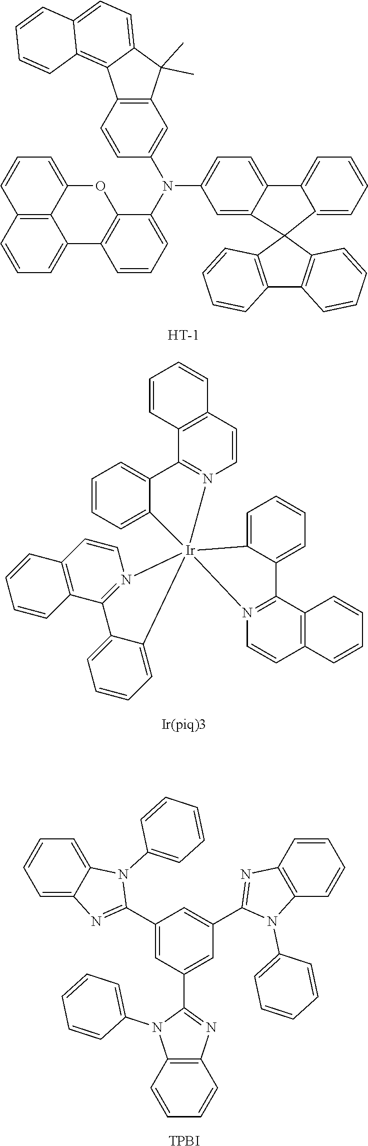

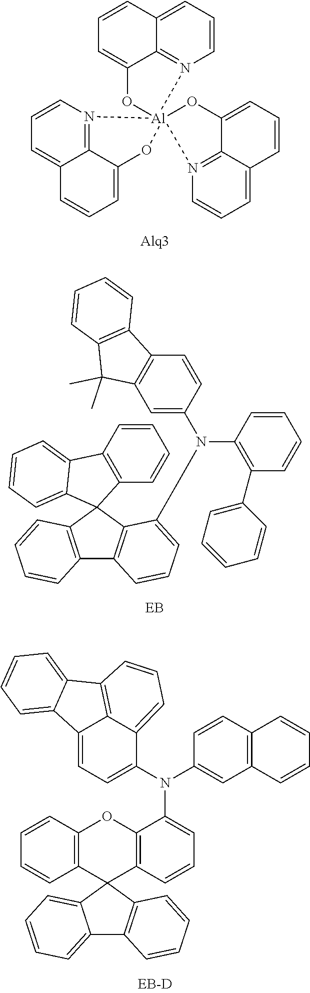

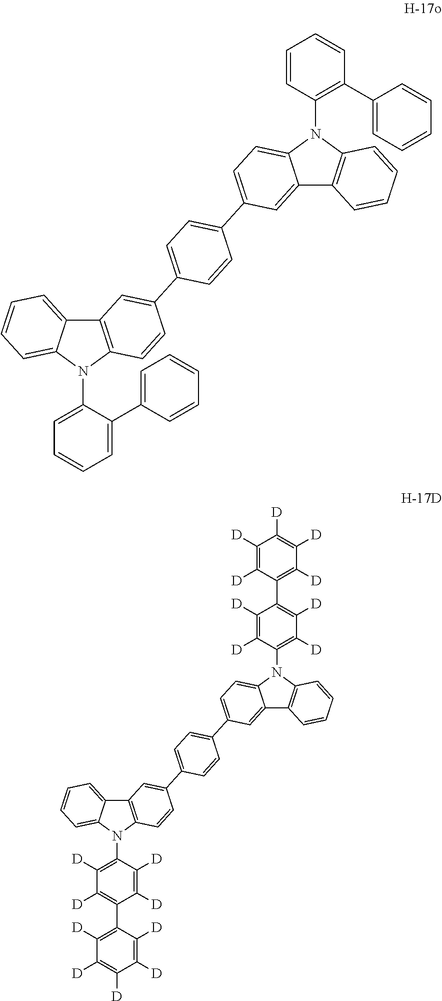 [SMILES: c1ccc(-c2ccccc2-n2c3ccccc3c3cc(-c4ccc(-c5ccc6c(c5)c5ccccc5n6-c5ccccc5-c5ccccc5)cc4)ccc32)cc1 ; [2H]c1c([2H])c([2H])c(-c2c([2H])c([2H])c(-n3c4ccccc4c4cc(-c5ccc(-c6ccc7c(c6)c6ccccc6n7-c6c([2H])c([2H])c(-c7c([2H])c([2H])c([2H])c([2H])c7[2H])c([2H])c6[2H])cc5)ccc43)c([2H])c2[2H])c([2H])c1[2H]]

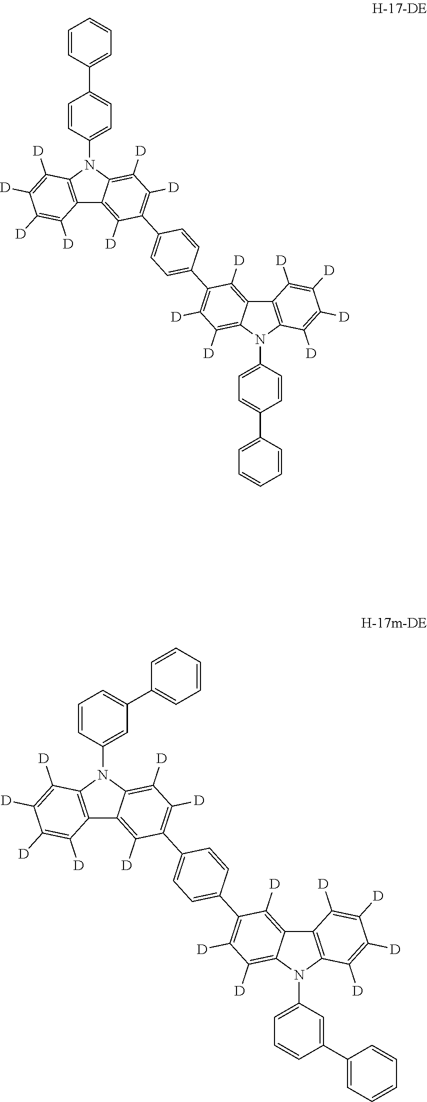 [SMILES: [2H]c1c([2H])c([2H])c2c(c1[2H])c1c([2H])c(-c3ccc(-c4c([2H])c([2H])c5c(c4[2H])c4c([2H])c([2H])c([2H])c([2H])c4n5-c4ccc(-c5ccccc5)cc4)cc3)c([2H])c([2H])c1n2-c1ccc(-c2ccccc2)cc1 ; [2H]c1c([2H])c([2H])c2c(c1[2H])c1c([2H])c(-c3ccc(-c4c([2H])c([2H])c5c(c4[2H])c4c([2H])c([2H])c([2H])c([2H])c4n5-c4cccc(-c5ccccc5)c4)cc3)c([2H])c([2H])c1n2-c1cccc(-c2ccccc2)c1]

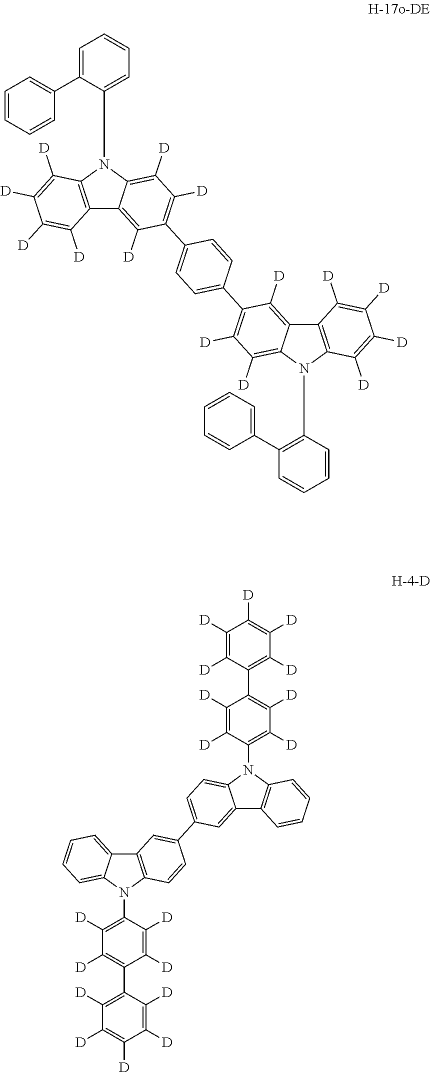 [SMILES: [2H]c1c([2H])c([2H])c2c(c1[2H])c1c([2H])c(-c3ccc(-c4c([2H])c([2H])c5c(c4[2H])c4c([2H])c([2H])c([2H])c([2H])c4n5-c4ccccc4-c4ccccc4)cc3)c([2H])c([2H])c1n2-c1ccccc1-c1ccccc1 ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c([2H])c6[2H])c([2H])c5[2H])ccc43)c([2H])c2[2H])c([2H])c1[2H]]

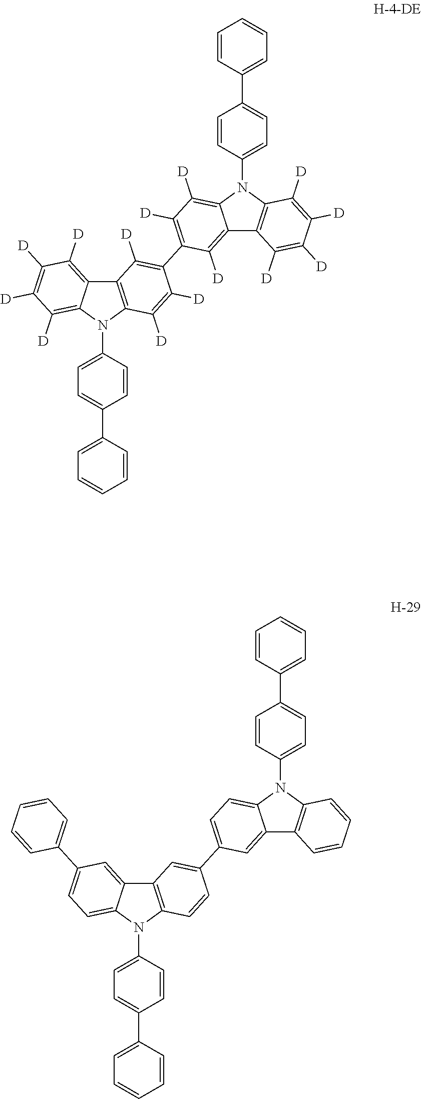 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1 ; c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1]

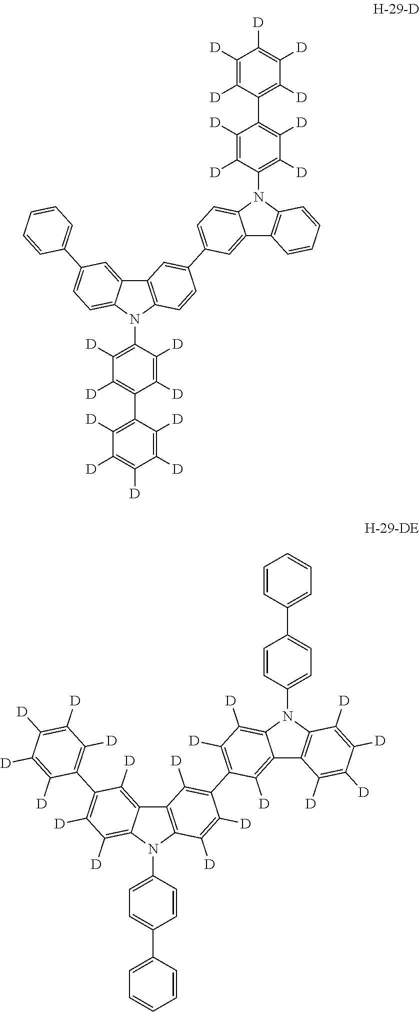 [SMILES: [2H]c1c([2H])c([2H])c(-c2c([2H])c([2H])c(-n3c4ccccc4c4cc(-c5ccc6c(c5)c5cc(-c7ccccc7)ccc5n6-c5c([2H])c([2H])c(-c6c([2H])c([2H])c([2H])c([2H])c6[2H])c([2H])c5[2H])ccc43)c([2H])c2[2H])c([2H])c1[2H] ; [2H]c1c([2H])c([2H])c(-c2c([2H])c([2H])c3c(c2[2H])c2c([2H])c(-c4c([2H])c([2H])c5c(c4[2H])c4c([2H])c([2H])c([2H])c([2H])c4n5-c4ccc(-c5ccccc5)cc4)c([2H])c([2H])c2n3-c2ccc(-c3ccccc3)cc2)c([2H])c1[2H]]

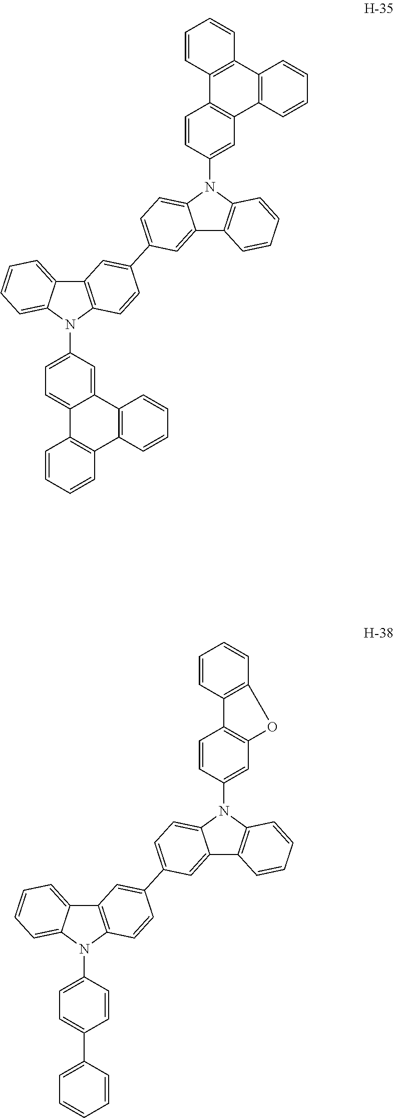 [SMILES: c1ccc2c(c1)c1ccccc1c1cc(-n3c4ccccc4c4cc(-c5ccc6c(c5)c5ccccc5n6-c5ccc6c7ccccc7c7ccccc7c6c5)ccc43)ccc21 ; c1ccc(-c2ccc(-n3c4ccccc4c4cc(-c5ccc6c(c5)c5ccccc5n6-c5ccc6c(c5)oc5ccccc56)ccc43)cc2)cc1]

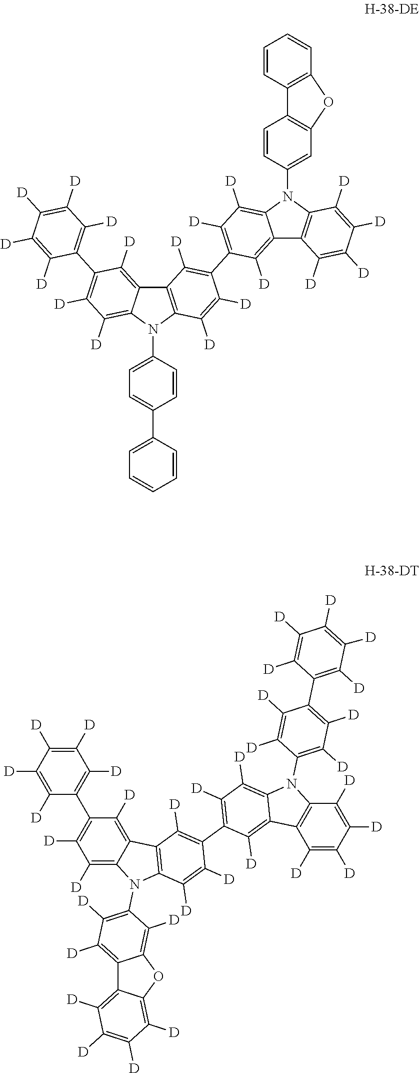 [SMILES: [2H]c1c([2H])c([2H])c(-c2c([2H])c([2H])c3c(c2[2H])c2c([2H])c(-c4c([2H])c([2H])c5c(c4[2H])c4c([2H])c([2H])c([2H])c([2H])c4n5-c4ccc5c(c4)oc4ccccc45)c([2H])c([2H])c2n3-c2ccc(-c3ccccc3)cc2)c([2H])c1[2H] ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c(-c7c([2H])c([2H])c([2H])c([2H])c7[2H])c([2H])c([2H])c5n6-c5c([2H])c([2H])c6c(oc7c([2H])c([2H])c([2H])c([2H])c76)c5[2H])c([2H])c([2H])c43)c([2H])c2[2H])c([2H])c1[2H]]

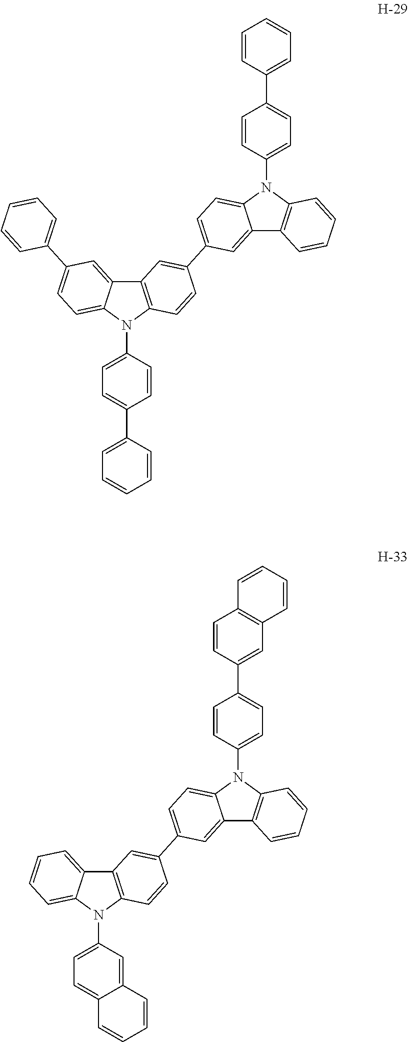 [SMILES: c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1 ; c1ccc2cc(-c3ccc(-n4c5ccccc5c5cc(-c6ccc7c(c6)c6ccccc6n7-c6ccc7ccccc7c6)ccc54)cc3)ccc2c1]

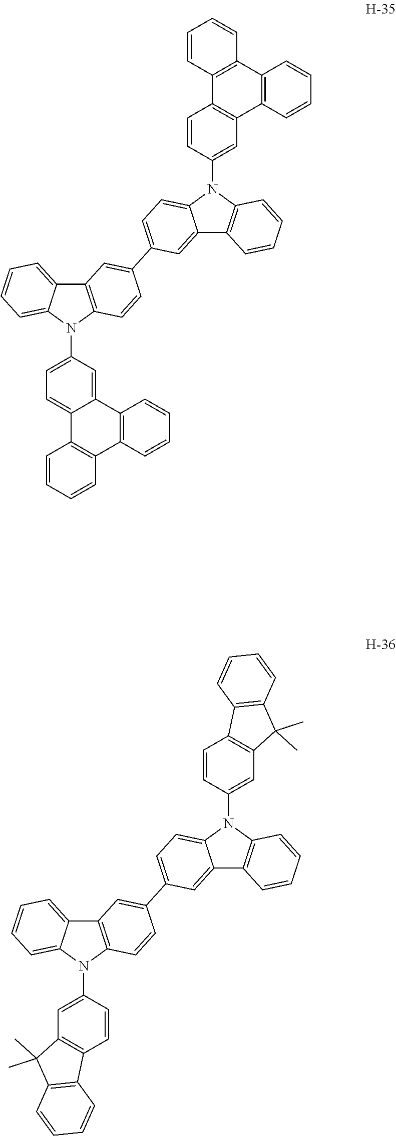

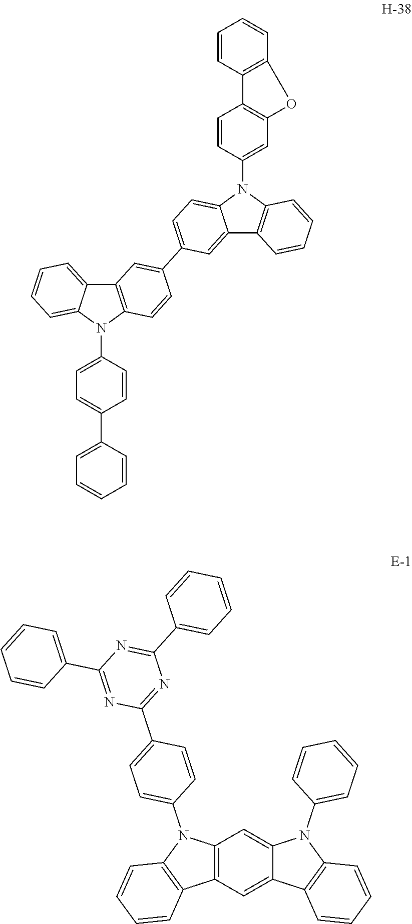 [SMILES: c1ccc(-c2ccc(-n3c4ccccc4c4cc(-c5ccc6c(c5)c5ccccc5n6-c5ccc6c(c5)oc5ccccc56)ccc43)cc2)cc1 ; c1ccc(-c2nc(-c3ccccc3)nc(-c3ccc(-n4c5ccccc5c5cc6c7ccccc7n(-c7ccccc7)c6cc54)cc3)n2)cc1]

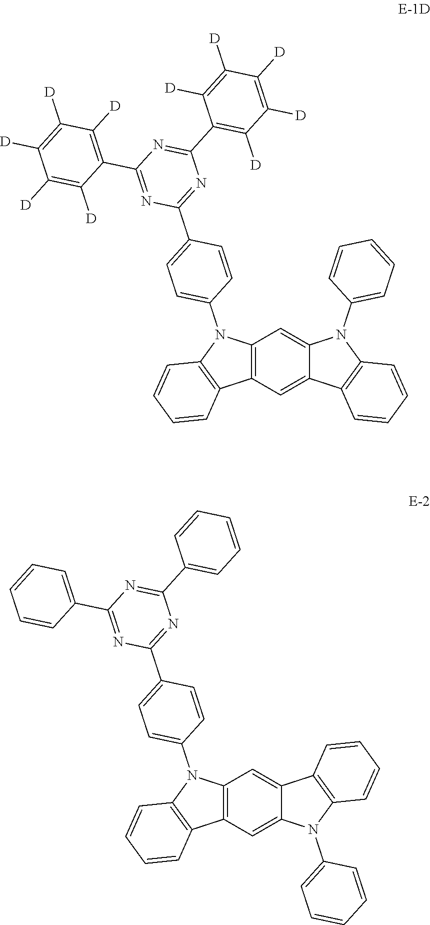 [SMILES: [2H]c1c([2H])c([2H])c(-c2nc(-c3ccc(-n4c5ccccc5c5cc6c7ccccc7n(-c7ccccc7)c6cc54)cc3)nc(-c3c([2H])c([2H])c([2H])c([2H])c3[2H])n2)c([2H])c1[2H] ; c1ccc(-c2nc(-c3ccccc3)nc(-c3ccc(-n4c5ccccc5c5cc6c(cc54)c4ccccc4n6-c4ccccc4)cc3)n2)cc1]

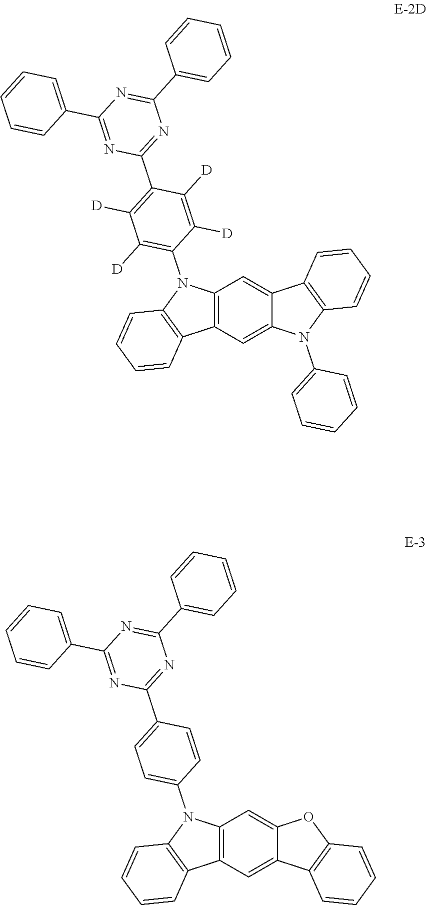 [SMILES: [2H]c1c([2H])c(-n2c3ccccc3c3cc4c(cc32)c2ccccc2n4-c2ccccc2)c([2H])c([2H])c1-c1nc(-c2ccccc2)nc(-c2ccccc2)n1 ; c1ccc(-c2nc(-c3ccccc3)nc(-c3ccc(-n4c5ccccc5c5cc6c(cc54)oc4ccccc46)cc3)n2)cc1]

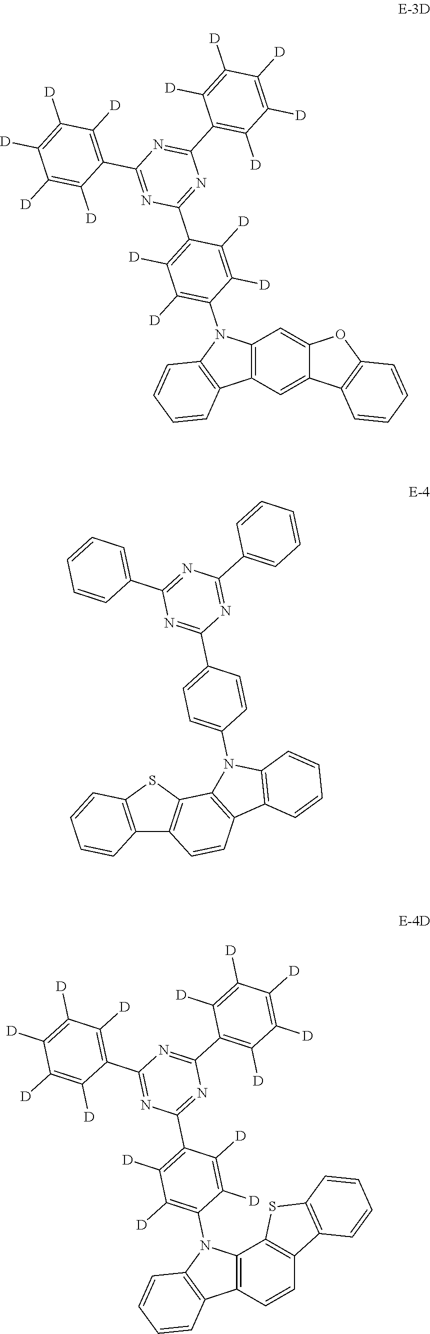 [SMILES: [2H]c1c([2H])c([2H])c(-c2nc(-c3c([2H])c([2H])c([2H])c([2H])c3[2H])nc(-c3c([2H])c([2H])c(-n4c5ccccc5c5cc6c(cc54)oc4ccccc46)c([2H])c3[2H])n2)c([2H])c1[2H] ; c1ccc(-c2nc(-c3ccccc3)nc(-c3ccc(-n4c5ccccc5c5ccc6c7ccccc7sc6c54)cc3)n2)cc1 ; [2H]c1c([2H])c([2H])c(-c2nc(-c3c([2H])c([2H])c([2H])c([2H])c3[2H])nc(-c3c([2H])c([2H])c(-n4c5ccccc5c5ccc6c7ccccc7sc6c54)c([2H])c3[2H])n2)c([2H])c1[2H]]

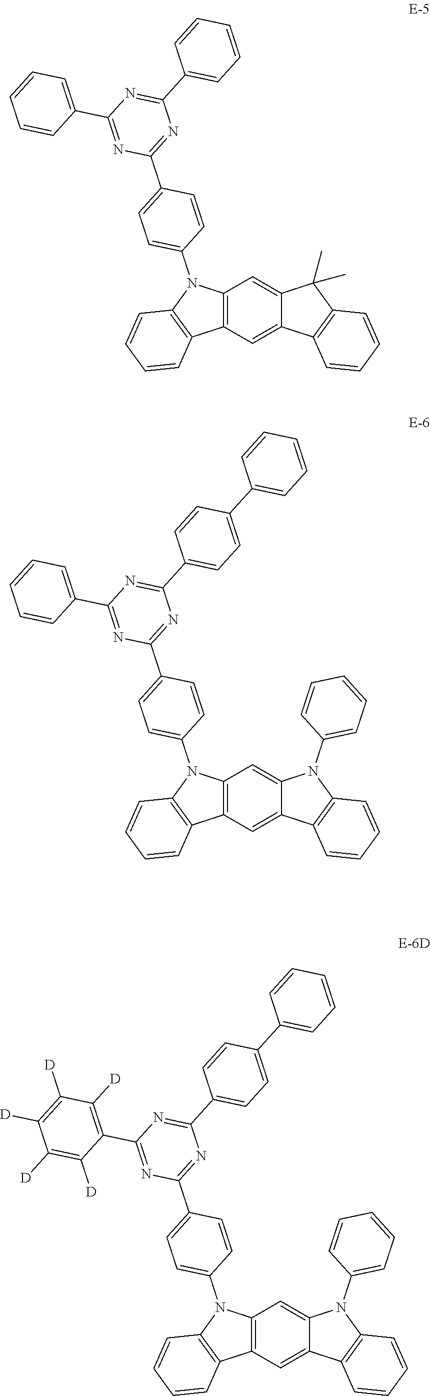 [SMILES: CC1(C)c2ccccc2-c2cc3c4ccccc4n(-c4ccc(-c5nc(-c6ccccc6)nc(-c6ccccc6)n5)cc4)c3cc21 ; c1ccc(-c2ccc(-c3nc(-c4ccccc4)nc(-c4ccc(-n5c6ccccc6c6cc7c8ccccc8n(-c8ccccc8)c7cc65)cc4)n3)cc2)cc1 ; [2H]c1c([2H])c([2H])c(-c2nc(-c3ccc(-c4ccccc4)cc3)nc(-c3ccc(-n4c5ccccc5c5cc6c7ccccc7n(-c7ccccc7)c6cc54)cc3)n2)c([2H])c1[2H]]

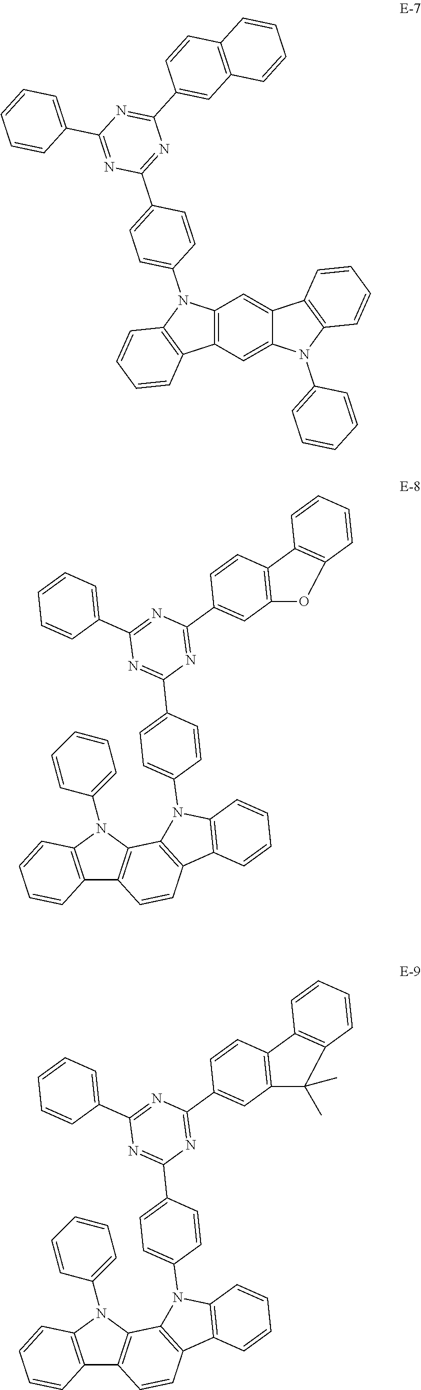 [SMILES: c1ccc(-c2nc(-c3ccc(-n4c5ccccc5c5cc6c(cc54)c4ccccc4n6-c4ccccc4)cc3)nc(-c3ccc4ccccc4c3)n2)cc1 ; c1ccc(-c2nc(-c3ccc(-n4c5ccccc5c5ccc6c7ccccc7n(-c7ccccc7)c6c54)cc3)nc(-c3ccc4c(c3)oc3ccccc34)n2)cc1 ; CC1(C)c2ccccc2-c2ccc(-c3nc(-c4ccccc4)nc(-c4ccc(-n5c6ccccc6c6ccc7c8ccccc8n(-c8ccccc8)c7c65)cc4)n3)cc21]

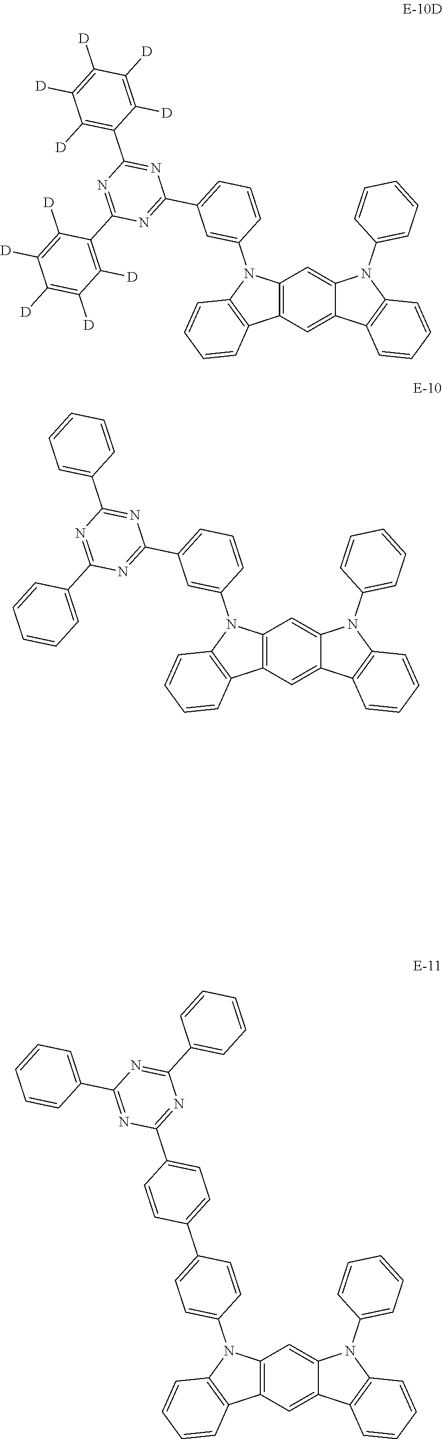 [SMILES: [2H]c1c([2H])c([2H])c(-c2nc(-c3cccc(-n4c5ccccc5c5cc6c7ccccc7n(-c7ccccc7)c6cc54)c3)nc(-c3c([2H])c([2H])c([2H])c([2H])c3[2H])n2)c([2H])c1[2H] ; c1ccc(-c2nc(-c3ccccc3)nc(-c3cccc(-n4c5ccccc5c5cc6c7ccccc7n(-c7ccccc7)c6cc54)c3)n2)cc1 ; c1ccc(-c2nc(-c3ccccc3)nc(-c3ccc(-c4ccc(-n5c6ccccc6c6cc7c8ccccc8n(-c8ccccc8)c7cc65)cc4)cc3)n2)cc1]

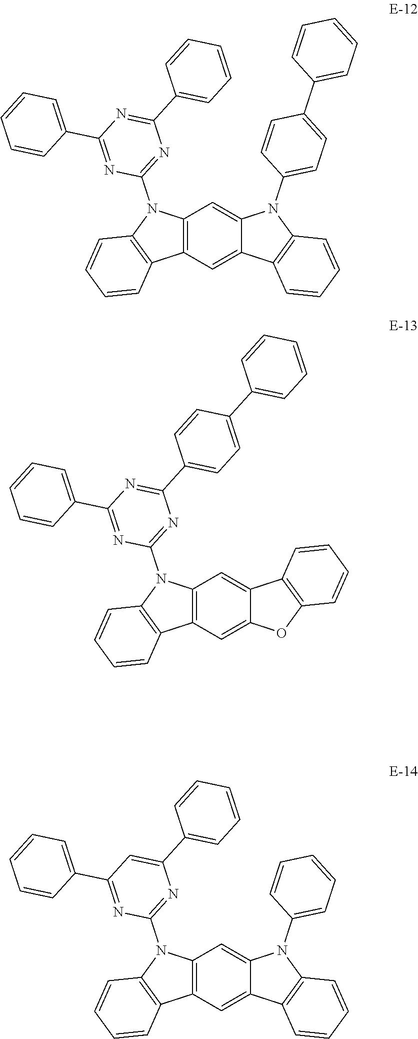 [SMILES: c1ccc(-c2ccc(-n3c4ccccc4c4cc5c6ccccc6n(-c6nc(-c7ccccc7)nc(-c7ccccc7)n6)c5cc43)cc2)cc1 ; c1ccc(-c2ccc(-c3nc(-c4ccccc4)nc(-n4c5ccccc5c5cc6oc7ccccc7c6cc54)n3)cc2)cc1 ; c1ccc(-c2cc(-c3ccccc3)nc(-n3c4ccccc4c4cc5c6ccccc6n(-c6ccccc6)c5cc43)n2)cc1]

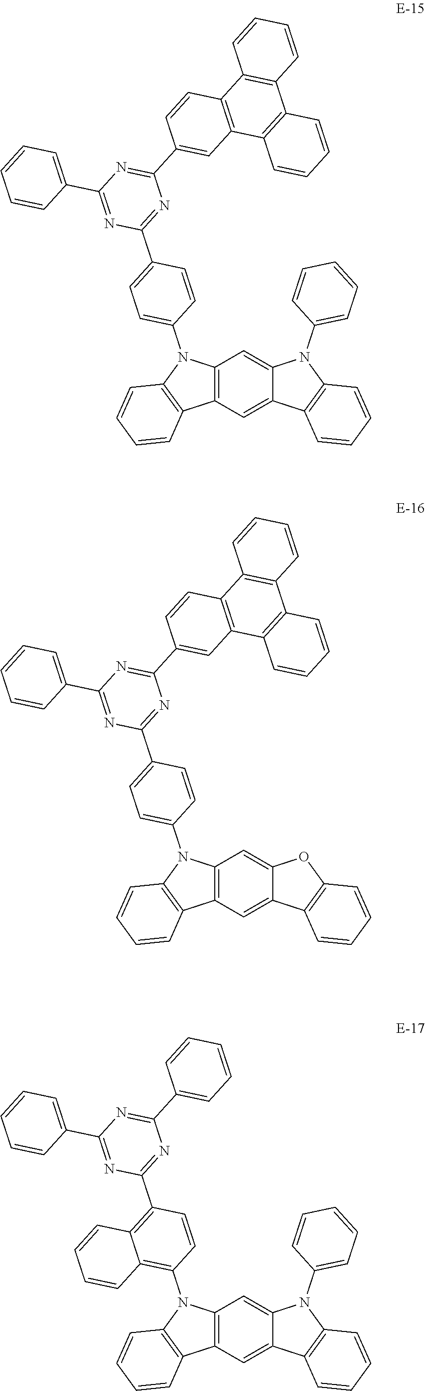 [SMILES: c1ccc(-c2nc(-c3ccc(-n4c5ccccc5c5cc6c7ccccc7n(-c7ccccc7)c6cc54)cc3)nc(-c3ccc4c5ccccc5c5ccccc5c4c3)n2)cc1 ; c1ccc(-c2nc(-c3ccc(-n4c5ccccc5c5cc6c(cc54)oc4ccccc46)cc3)nc(-c3ccc4c5ccccc5c5ccccc5c4c3)n2)cc1 ; c1ccc(-c2nc(-c3ccccc3)nc(-c3ccc(-n4c5ccccc5c5cc6c7ccccc7n(-c7ccccc7)c6cc54)c4ccccc34)n2)cc1]

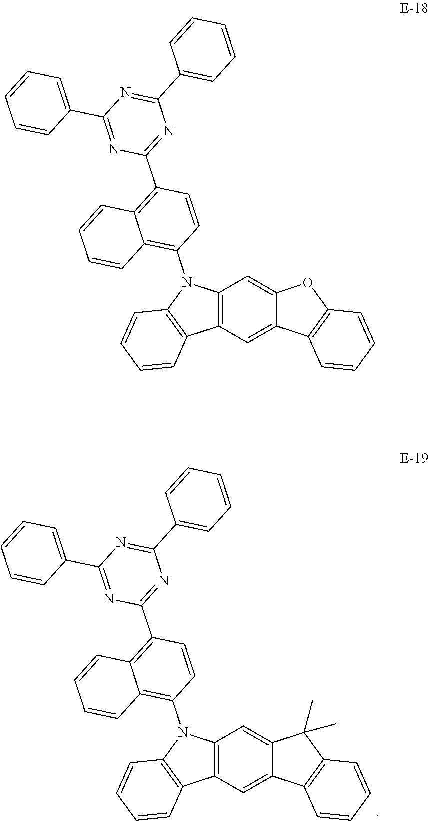 [SMILES: c1ccc(-c2nc(-c3ccccc3)nc(-c3ccc(-n4c5ccccc5c5cc6c(cc54)oc4ccccc46)c4ccccc34)n2)cc1 ; CC1(C)c2ccccc2-c2cc3c4ccccc4n(-c4ccc(-c5nc(-c6ccccc6)nc(-c6ccccc6)n5)c5ccccc45)c3cc21]

### Device Example 1

This device example provides an organic electroluminescent device, wherein the composition provided in the present application is selected as a red-emitting host material.

The structure of the organic electroluminescent device is as follows: ITO/HT-1 (20 nm)/red-emitting host material (35 nm): Ir(piq)₃[10%]/TPBI (10 nm)/Alq3 (15 nm)/LiF (0.5 nm)/Al (150 nm); wherein ‘Ir(piq)₃[10%]’ refers to a doping ratio of the red dopant, that is, a volume ratio of the red-emitting host material to Ir(piq)₃ is 90:10.

The fabrication process of the organic electroluminescent device is as follows:
a glass substrate coated with an ITO transparent conductive layer was ultrasonically cleaned in a commercial detergent, rinsed with deionized water, degreased in an acetone:ethanol mixed solvent via ultrasonication, baked in a clean environment to remove moisture completely, treated with UV-ozone cleaning, and the surface bombarded with a low-energy cation beam;
the above glass substrate with an anode was placed in a vacuum chamber, and the chamber was evacuated to 1×10⁻⁵-9×10⁻⁴ Pa, and a hole transport layer (HT-1) was vacuum-evaporated onto the anode layer at a evaporation rate of 0.1 nm/s, forming a 20 nm-thick film;
the red-emitting host material and dopant Ir(piq)₃ were co-evaporated on the hole transporting layer as an emissive layer of the organic electroluminescent device with an evaporation rate of 0.1 nm/s, and a thickness of the total evaporation film was 35 nm; in this example, if the red-emitting host material is two or more compounds, each of compound is placed in different evaporation sources to be heated, and the heating rate is controlled so that the ratio of each volume that is evaporated onto the substrate is in the same proportion, as a red-emitting host material;
an electron transport layer (TPBI) and Alq3 were sequentially evaporated on the emissive layer at a rate of 0.1 nm/s, with a thicknesses of 10 nm and 15 nm, respectively; and
a 0.5 nm-thick LiF layer and a 150 nm-thick Al layer were vacuum-evaporated as an electron injection layer and cathode on the electron transport layer, respectively.

### Device Examples 2-15

Device Examples 2-15 each provide an organic electroluminescent device, which differs from Device Example 1 only in that the red-emitting host material is different, and if the red-emitting host material is two or more compounds, each of compounds is placed in different evaporation sources for heating, and the heating rate is controlled so that the ratio of each volume that is evaporated onto the substrate is in the same proportion, as a red-emitting host material (see Table 1 below in detail), and other preparation steps and conditions are the same as in Device Example 1.

### Device Comparative Examples 1-3

Device Comparative Examples 1-3 each provide an organic electroluminescent device, which differs from Device Example 1 only in that the red-emitting host material is different, and if the red-emitting host material is two or more compounds, each of compound is placed in different evaporation sources for heating, and the heating rate is controlled so that the ratio of each volume that is evaporated onto the substrate is in the same proportion, as a red-emitting host material (see Table 1), and other preparation steps and conditions are the same as in Device Example 1.

### Performance Test

OLED-1000 Multi-channel Accelerated Lifetime & Optical Property Test System for OLED manufactured by Hangzhou EVERFINE was used to test and measure the luminance, driving voltage, current efficiency and the lifetime LT90 of the fabricated organic electroluminescent devices. The lifetime test LT90 is the time that the luminance is reduced to 90% of the initial luminance at room temperature (25-27° C.), while the current density is kept constant at the initial luminance (1000 cd/m²). In the following tables, driving voltage, current efficiency, and LT90 lifetime are relative values. The test results are detailed in Table 1 below.

TABLE 1

| | Red-emitting host material | | | | Required | | | |
|---|---|---|---|---|---|---|---|---|
| | Component | Component | Component | Component | luminance/ | Driving | Current | LT90 |
| | A | B | C | D | (cd/m²) | voltage | efficiency | lifetime |
| Device | H-17 | H-17D | E-1 | E-1D | 1000 | 1.02 | 1.11 | 1.06 |
| Example 1 | | | | | | | | |
| Device | H-17 | H-17- | E-1 | E-1D | 1000 | 1.08 | 1.29 | 1.30 |
| Example 2 | | DE | | | | | | |
| Device | H-4 | H-4-D | E-1 | E-1D | 1000 | 0.96 | 1.13 | 2.02 |
| Example 3 | | | | | | | | |
| Device | H-4 | H-4- | E-1 | E-1D | 1000 | 0.91 | 1.15 | 2.58 |
| Example 4 | | DE | | | | | | |
| Device | H-4 | H-4- | E-3 | E-3D | 1000 | 0.88 | 1.09 | 1.91 |
| Example 5 | | DE | | | | | | |
| Device | H-4 | H-4- | E-1 | E-3 | 1000 | 0.95 | 1.11 | 1.69 |
| Example 6 | | DE | | | | | | |
| Device | H-4 | H-33 | E-1 | E-3 | 1000 | 0.78 | 1.11 | 1.26 |
| Example 7 | | | | | | | | |
| Device | H-4 | H-35 | E-5 | E-6D | 1000 | 0.82 | 1.17 | 1.51 |
| Example 8 | | | | | | | | |
| Device | H-4 | H-36 | E-10D | / | 1000 | 0.65 | 1.31 | 2.86 |
| Example 9 | | | | | | | | |
| Device | H-4 | H-36 | E-10D | E-10 | 1000 | 0.66 | 1.43 | 3.11 |
| Example 10 | | | | | | | | |
| Device | H-29 | H-29-D | E-1 | E-1D | 1000 | 0.86 | 2.02 | 2.25 |
| Example 11 | | | | | | | | |
| Device | H-29 | H-29- | E-1 | E-1D | 1000 | 0.88 | 2.31 | 3.01 |
| Example 12 | | DE | | | | | | |
| Device | H-29- | H-29-D | E-1 | E-1D | 1000 | 0.89 | 1.98 | 2.15 |
| Example 13 | DE | | | | | | | |
| Device | H-4 | H-33 | E-13 | E-14 | 1000 | 0.39 | 1.19 | 1.21 |
| Example 14 | | | | | | | | |
| Device | / | H-4- | E-1 | E-1D | 1000 | 0.90 | 1.12 | 2.02 |
| Example 15 | | DE | | | | | | |
| Device | H-4 | / | E-1 | / | 1000 | 1.12 | 0.79 | 0.71 |
| Comparative | | | | | | | | |
| Example 1 | | | | | | | | |
| Device | / | H-4- | E-3D | / | 1000 | 1 | 1 | 1 |
| Comparative | | DE | | | | | | |
| Example 2 | | | | | | | | |
| Device | / | H-4- | E-3 | / | 1000 | 1.02 | 0.92 | 0.89 |
| Comparative | | DE | | | | | | |
| Example 3 | | | | | | | | |

Note:
“/” in Table 1 represents the absence of the compound.

As can be seen from Table 1, in the present application, a composition with specific component is obtained by designing the specific component of the composition and further using the specific composition with multiple component, and an organic electroluminescent device with a lower driving voltage, a higher current efficiency and a longer lifetime can be fabricated by using this composition as the material of the emissive layer of the organic electroluminescent device.

It can be seen by comparing Device Example 3 with Device Comparative Example 1, as well as Device Example 5 with Device Comparative Example 2 and Device Comparative Example 3, that the organic electroluminescent device fabricated by using a multi-component composition in the present application has excellent comprehensive performance.

It can be seen from Device Example 4 with Device Example 15 that the organic electroluminescent device prepared by combining four components has more excellent comprehensive performance.

It can be seen from Device Example 3 and Device Example 4 that, in the present application, the lifetime of the OLED can be further improved by using the compound represented by Formula I in which hydrogen atoms on the carbazole group are substituted with deuterium atoms as one of the components of the composition.

It can be seen from Device Example 9 and Device Example 10 that in the present application, the substituent at the corresponding position of Ar₁₁ in the compound represented by Formula II is meta-disubstituent phenylene, which can further improve the performance of the OLED. Moreover, it can be seen from Device Example 9 and Device Example 10 that the OLED device obtained by using the four-component composition has a higher current efficiency and a longer lifetime.

It can be seen from Device Examples 11-13 that the compound represented by Formula I in which the hydrogen atom on the carbazole group is substituted with a deuterium atom and no hydrogen atom is contained on the benzene ring (substituents at the corresponding positions of Ar₂₁ and Ar₂₂) is selected, the performance of the OLED device prepared is superior. Moreover, the two compounds conforming to the structure in Formula I, one of which contains no D atom, the prepared OLED device has better performance.

### Device Example 16

This device example provides an organic electroluminescent device, wherein the composition provided in the present application is selected as a red-emitting host material.

The structure of the organic electroluminescent device is as follows: ITO/HT-1 (20 nm)/electron blocking layer (5 nm)/red-emitting host material (35 nm): Ir(piq)₃[10%]/TPBI (10 nm)/Alq3 (15 nm)/LiF (0.5 nm)/Al (150 nm); where “Ir(piq)₃[10%]” refers to a doping ratio of the red dopant, that is, a volume ratio of the red-emitting host material to Ir(piq)₃ is 90:10.

The fabrication process of the organic electroluminescent device is as follows:
a glass substrate coated with an ITO transparent conductive layer was ultrasonically cleaned in a commercial detergent, rinsed with deionized water, degreased in an acetone:ethanol mixed solvent via ultrasonication, baked in a clean environment to remove moisture completely, treated with UV-ozone cleaning, and the surface bombarded with a low-energy cation beam;
the above glass substrate with an anode was placed in a vacuum chamber, and the chamber was evacuated to 1×10⁻⁵-9×10⁻⁴ Pa, and a hole transport layer (HT-1) was vacuum-evaporated onto the anode layer at a evaporation rate of 0.1 nm/s, forming a 20 nm-thick film;
EB was evaporated as an electron blocking layer on the hole transport layer with an evaporation rate of 0.1 nm/s, and a thickness of the evaporation film was 5 nm;
the red-emitting host material and dopant Ir(piq)₃ were co-evaporated on the electron blocking layer as an emissive layer of the organic electroluminescent device with an evaporation rate of 0.1 nm/s, and a thickness of the total evaporation film was 35 nm; in this example, and if the red-emitting host material is H-3, H-3-D-E, E-1, and E-1D, each compounds are placed in different evaporation sources for heating, and the heating rate is controlled so that the ratio of each volume that is evaporated onto the substrate is in the same proportion, as a red-emitting host material;
an electron transport layer (TPBI) and Alq3 were sequentially evaporated on the emissive layer at a rate of 0.1 nm/s, with a thicknesses of 10 nm and 15 nm, respectively; and
a 0.5 nm-thick LiF layer and a 150 nm-thick Al layer were vacuum-evaporated as an electron injection layer and cathode on the electron transport layer, respectively.

### Device Example 17

Device Example 17 provide an organic electroluminescent device, which differs from Device Example 16 only in that the material of the electron blocking layer was different (see Table 2 below in detail), and other preparation steps and conditions were the same as those of Device Example 16.

The performance of the organic electroluminescent devices provided by Device Examples 16-17 was tested in the same manner as above. The test results are detailed in Table 2 below.

TABLE 2

| | Electron | | | | |
|---|---|---|---|---|---|
| | blocking | Required | | | |
| | layer | luminance/ | Driving | Current | LT90 |
| | material | (cd/m²) | voltage | efficiency | lifetime |
| Device | EB | 1000 | 1 | 1 | 1 |
| Example 16 | | | | | |
| Device | EB-D | 1000 | 0.88 | 1.26 | 1.31 |
| Example 17 | | | | | |

As can be seen from Table 2, in the present application, the organic electroluminescent devices fabricated by employing spirofluorene compounds with specific structures as electron blocking layer materials, combined with the composition provided herein as the emissive layer material, exhibit enhanced current efficiency and prolonged operational lifetime.

The applicant declares that the detailed process flow of the present application are illustrated by the above examples in the present application, but the present application is not limited to the above detailed process flow, that is, the present application does not necessarily rely on the above detailed process flow to be implemented. Those skilled in the art should understand that any improvement of the present application, the equivalent substitution of each raw material of the product, the addition of auxiliary ingredients, and the selection of specific methods in the present application shall fall within the protection scope and disclosure scope of the present application.

1. A composition, comprising two compounds represented by Formula I and one compound represented by Formula II;
or the composition comprises one compound represented by Formula I and two compounds represented by Formula II;
a structural formula of the compound represented by the Formula I is as follows: 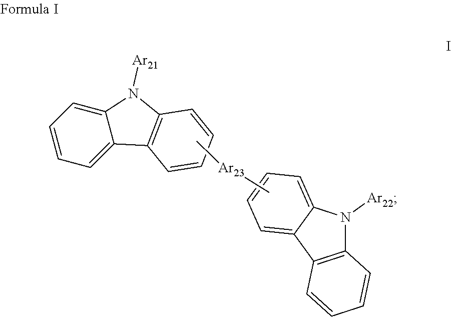
wherein Ar₂₁ and Ar₂₂ are each independently selected from C6 to C30 aryl or C6 to C20 heteroaryl;
Ar₂₃ is selected from any one of a single bond, phenylene, naphthylene, or biphenylene;
the hydrogen atom in the compound represented by Formula I can be substituted with at least one of —F, —CN, C6 to C20 aryl, C1 to C6 alkyl, or C1 to C6 alkoxy;
the compound represented by Formula I meets at least one of the following conditions:
(1) the compound represented by Formula I contains no deuterium atom;
(2) hydrogen atoms attached to two carbazolyl in the compound represented by the Formula I are all substituted with deuterium atoms;
(3) at least one hydrogen atom in Ar₂₁ of the compound represented by Formula I is substituted with a deuterium atom;
(4) at least one hydrogen atom in Ar₂₂ of the compound represented by Formula I is substituted with a deuterium atom;
(5) Ar₂₃ in the compound represented by Formula I is phenylene and/or biphenylene, and at least one hydrogen atom in the Ar₂₃ is substituted with a deuterium atom;
(6) a hydrogen atom in the compound represented by Formula I is substituted with C6 to C20 aryl, and at least one hydrogen atom in the C6 to C20 aryl is substituted with a deuterium atom; and
(7) a hydrogen atom in the compound represented by Formula I is substituted with C1 to C6 alkyl and/or C1 to C6 alkoxy, and all hydrogen atoms in C1 to C6 alkyl and/or C1 to C6 alkoxy are substituted with deuterium atoms;
the compound represented by Formula II is obtained by fusing a group represented by Formula I-C with any two adjacent carbon atoms on ring E in the group represented by Formula I-D; 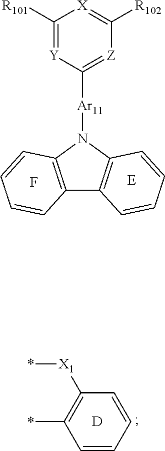
wherein “*” represents a fusion site;
Ar₁₁ is selected from any one of a single bond, phenylene, naphthylene, or biphenylene;
R₁₀₁ and R₁₀₂ are each independently selected from H, C6 to C30 aryl, or C6 to C20 heteroaryl;
X, Y, and Z are each independently selected from N or CR₃₀₄, wherein R₃₀₄ is selected from any one of H, phenyl, biphenyl, naphthyl, 9,9-dimethylfluorenyl, dibenzofuranyl, or dibenzothienyl, and at least one of X, Y, and Z is N;
X₁ is selected from O, S, 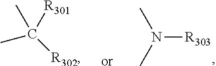
wherein R₃₀₁ and R₃₀₂ are each independently selected from C1 to C5 alkyl or phenyl, and R₃₀₃ is selected from phenyl or biphenyl, and the dashed line represents a linkage site;
the hydrogen atom in the compound represented by Formula II may be substituted with at least one of —F, —CN, C6 to C20 aryl, C1 to C6 alkyl, or C1 to C6 alkoxy;
the compound represented by Formula II meets at least one of the following conditions:
(a) the compound represented by Formula II contains no deuterium atom;
(b) hydrogen atoms on at least one of the ring F, ring D, and ring E in the compound represented by Formula II are all substituted with deuterium atoms;
(c) at least one hydrogen atom in R₁₀₁ of the compound represented by Formula II is substituted with a deuterium atom;
(d) at least one hydrogen atom in R₁₀₂ of the compound represented by Formula II is substituted with a deuterium atom;
(e) at least one hydrogen atom in Ar₁₁ of the compound represented by Formula II is substituted with a deuterium atom;
(f) hydrogen atoms in R₃₀₁ and R₃₀₂ in the compound represented by Formula II are all substituted with deuterium atoms;
(g) at least one hydrogen atom in R₃₀₃ of the compound represented by Formula II is substituted with a deuterium atom;
(h) R₃₀₄ in the compound represented by Formula II is a deuterium atom;
(i) in the compound represented by Formula II, when R₃₀₄ is selected from any one of phenyl, biphenyl, naphthyl, 9,9-dimethylfluorenyl, dibenzofuranyl, or dibenzothienyl, at least one hydrogen atom in the phenyl, biphenyl, naphthyl, 9,9-dimethylfluorenyl, dibenzofuranyl, or dibenzothienyl is substituted with a deuterium atom;
(j) a hydrogen atom in the compound represented by Formula II is substituted with C6 to C20 aryl, and at least one hydrogen atom in the C6 to C20 aryl is substituted with a deuterium atom; and
(k) a hydrogen atom in the compound represented by Formula II is substituted with C1 to C6 alkyl and/or C1 to C6 alkoxy, and all hydrogen atoms in C1 to C6 alkyl and/or C1 to C6 alkoxy are substituted with deuterium atoms.

2. The composition according to claim 1, wherein the compound represented by Formula I has a structure as represented by Formula I-1 or Formula I-2: 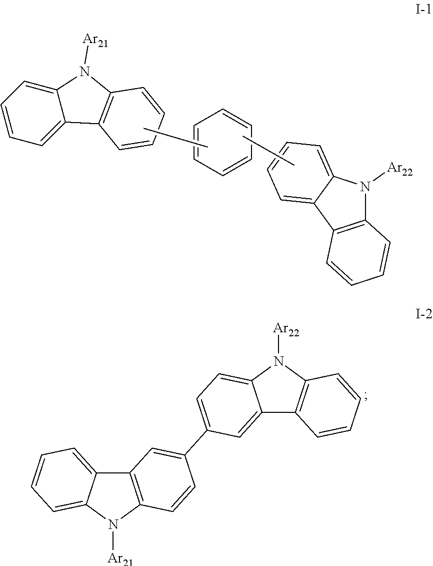
wherein Ar₂₁ and Ar₂₂ are defined the same as in claim 1.

3. The composition according to claim 1, wherein the compound represented by Formula I meets condition (3) and/or condition (4);
optionally, the compound represented by Formula I has a structure as represented by Formula I-1-D or Formula I-2-D: 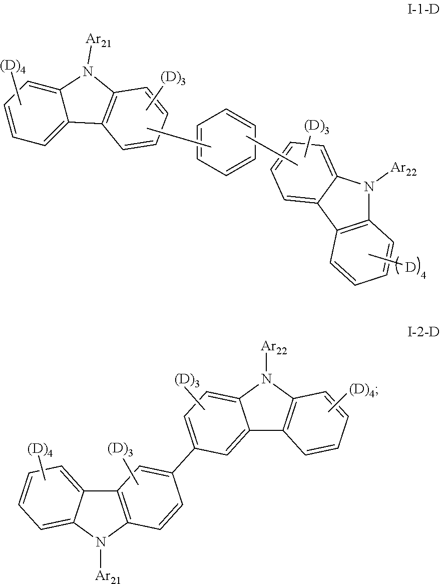
wherein Ar₂₁ and Ar₂₂ are defined the same as in claim 1, and Ar₂₁ and Ar₂₂ contain no deuterium atom.

4. The composition according to claim 1, wherein the Ar₂₁ and the Ar₂₂ are each independently selected from any one or a combination of at least two of phenyl, biphenyl, naphthyl, fluorenyl, triphenylenyl, fluorenyl, dibenzofuranyl, dibenzothienyl, terphenyl, or quaterphenyl;
optionally, the R₁₀₁ and the R₁₀₂ are each independently selected from any one of H, phenyl, naphthyl, triphenylenyl, fluoranthenyl, fluorenyl, anthryl, phenanthryl, biphenyl, naphthyl, dibenzofuranyl, or dibenzothienyl.

5. The composition according to claim 1, wherein the compound represented by Formula I is selected from any one of the following substituted or unsubstituted compounds: 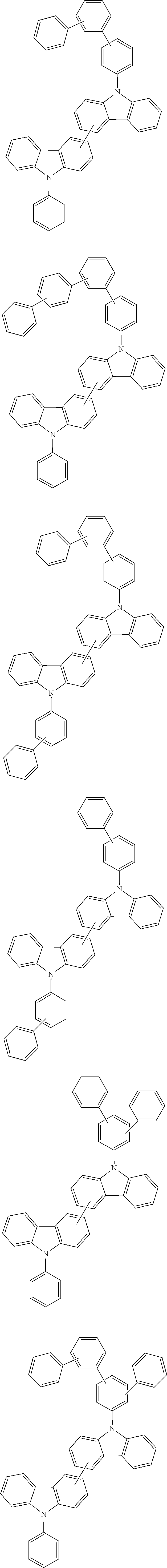 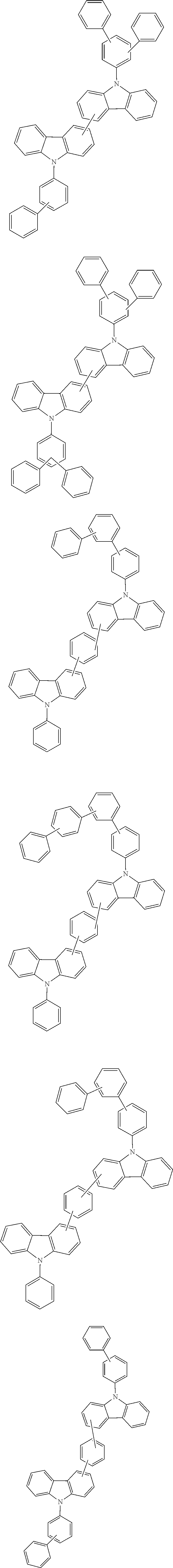 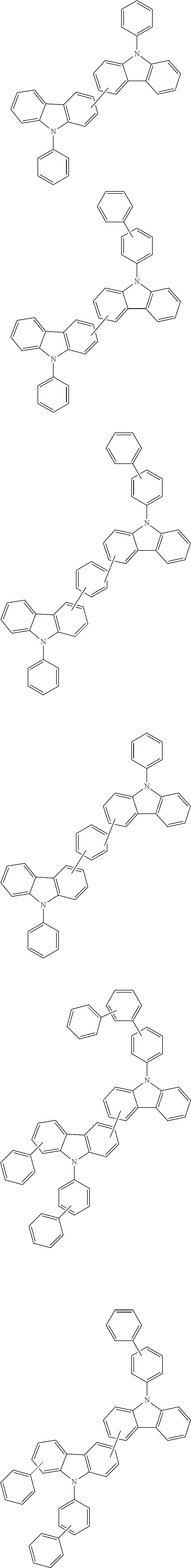 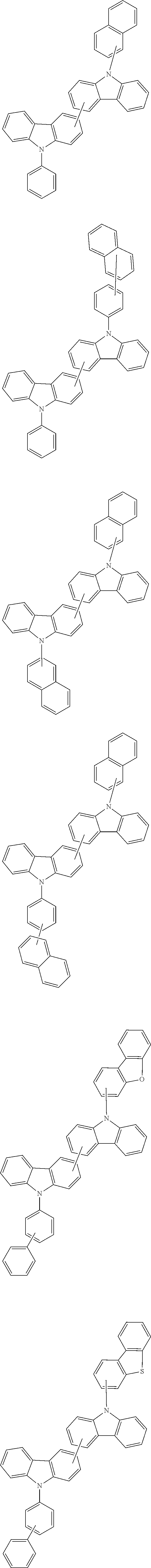 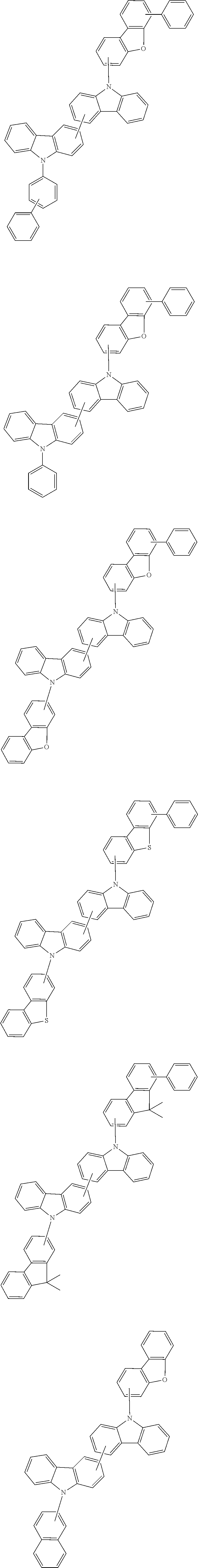 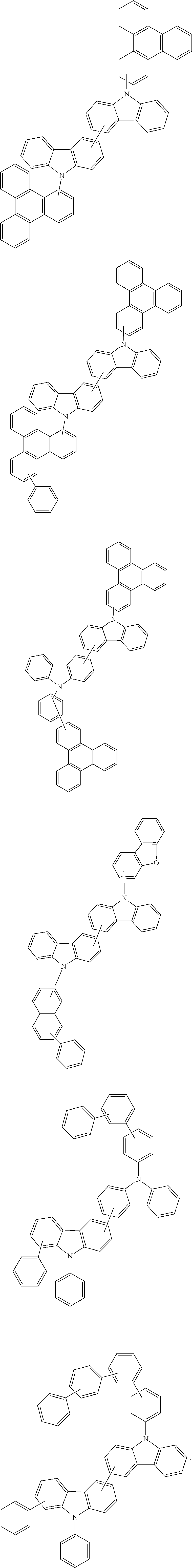
the “substituted” means that hydrogen atoms in the above compounds are each independently substituted with deuterium atoms;
optionally, the compound represented by Formula I is selected from any one of the following substituted or unsubstituted compounds H-1 to H-40, H-170, or H-17m: 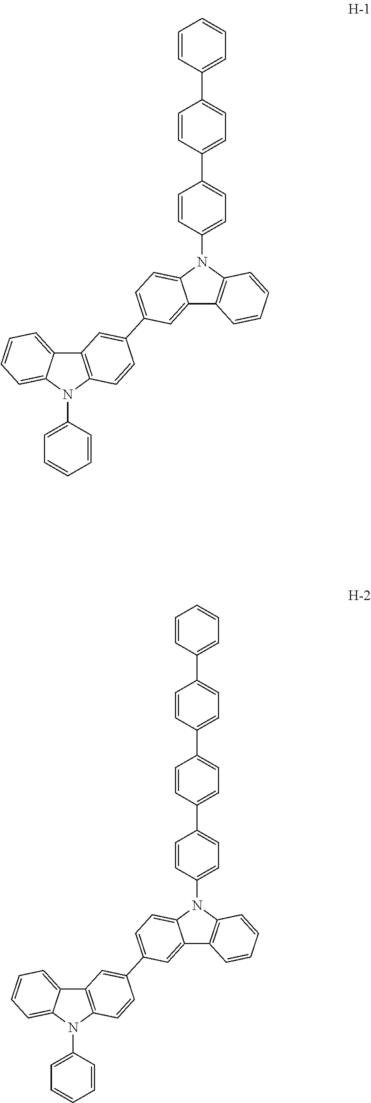 [SMILES: c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-c2ccc(-c3ccc(-c4ccc(-n5c6ccccc6c6cc(-c7ccc8c(c7)c7ccccc7n8-c7ccccc7)ccc65)cc4)cc3)cc2)cc1] 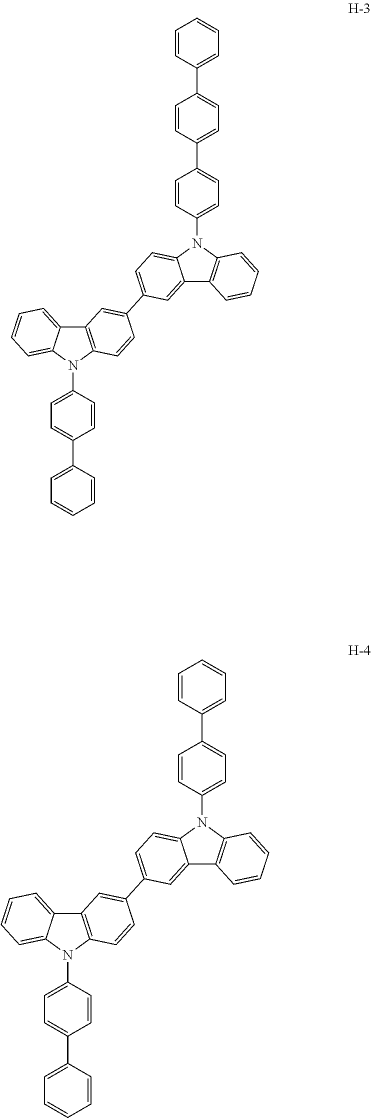 [SMILES: c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1] 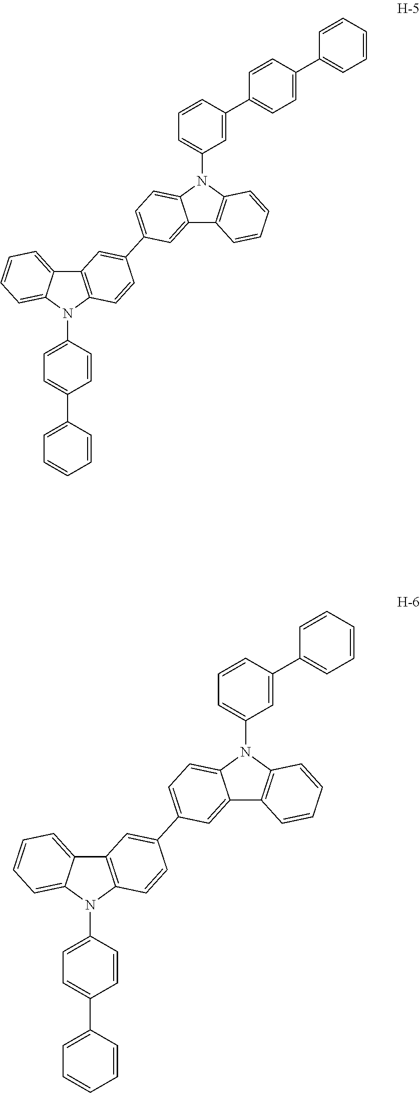 [SMILES: c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1] 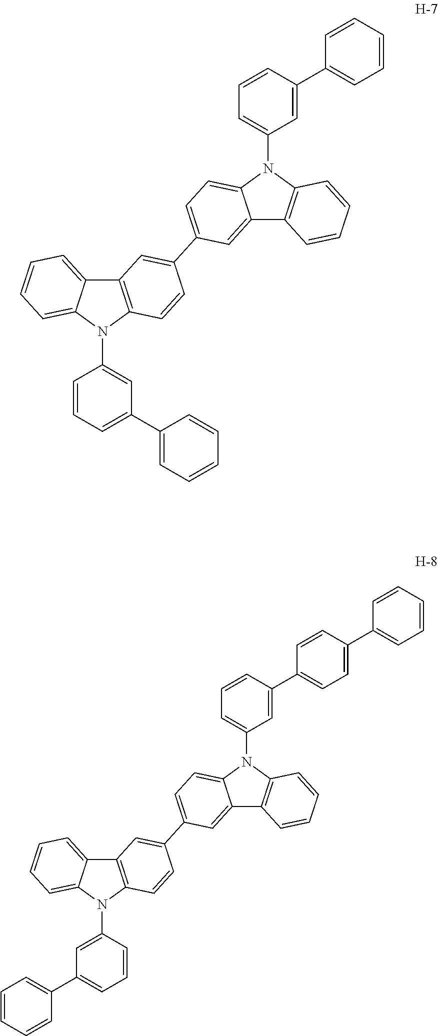 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1] 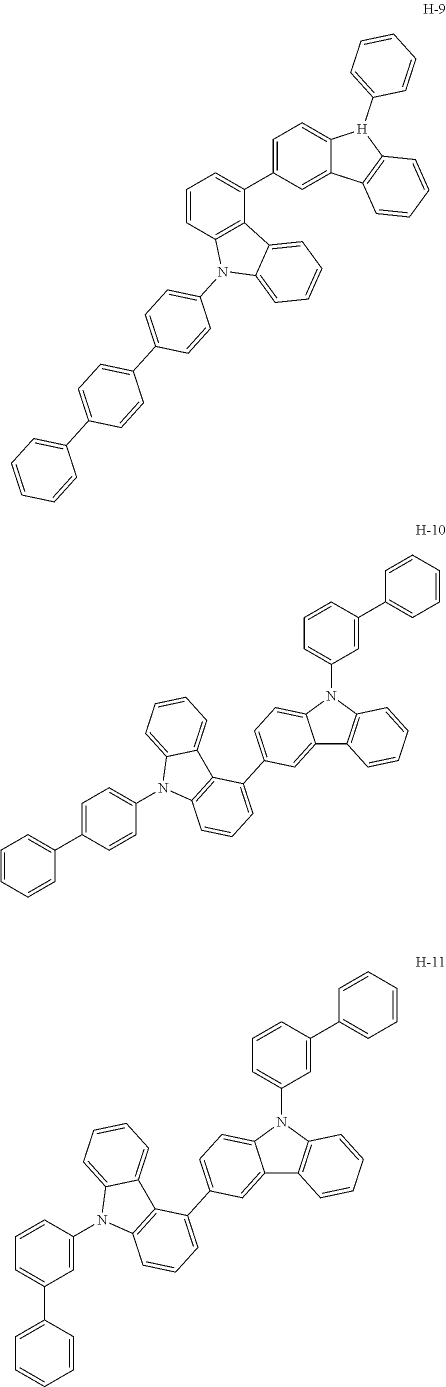 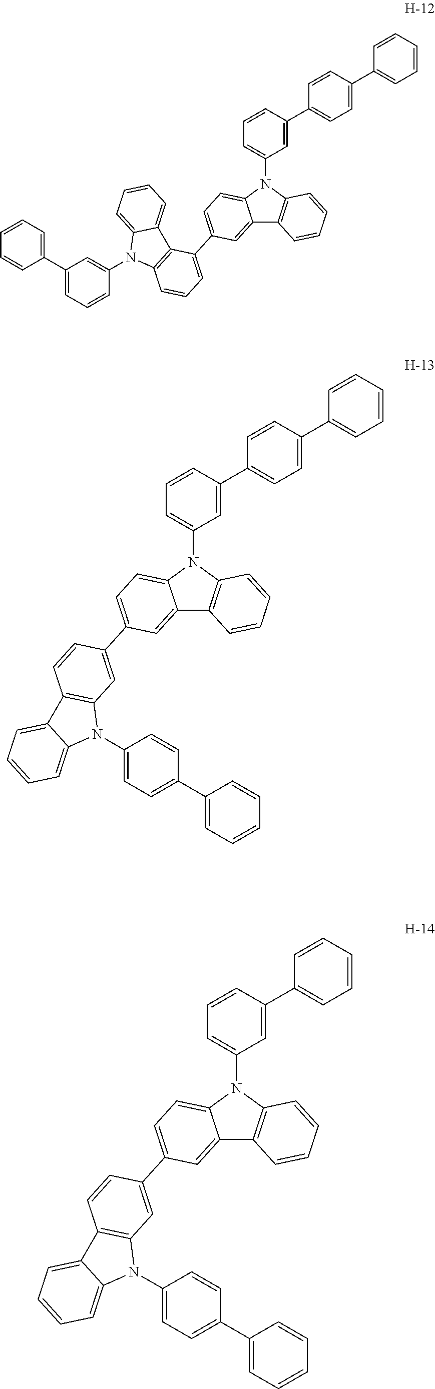 [SMILES: c1ccc(-c2ccc(-c3cccc(-n4c5ccccc5c5cc(-c6cccc7c6c6ccccc6n7-c6cccc(-c7ccccc7)c6)ccc54)c3)cc2)cc1 ; c1ccc(-c2ccc(-c3cccc(-n4c5ccccc5c5cc(-c6ccc7c8ccccc8n(-c8ccc(-c9ccccc9)cc8)c7c6)ccc54)c3)cc2)cc1 ; c1ccc(-c2ccc(-n3c4ccccc4c4ccc(-c5ccc6c(c5)c5ccccc5n6-c5cccc(-c6ccccc6)c5)cc43)cc2)cc1] 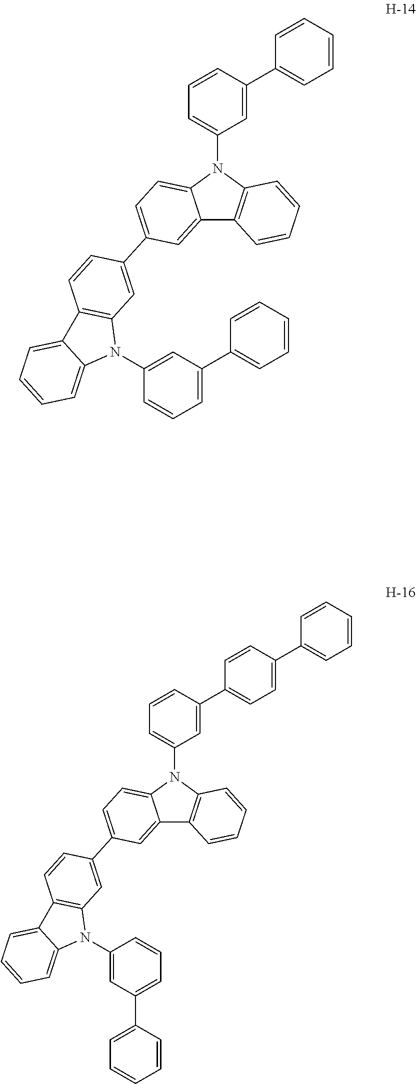 [SMILES: c1ccc(-c2cccc(-n3c4ccccc4c4cc(-c5ccc6c7ccccc7n(-c7cccc(-c8ccccc8)c7)c6c5)ccc43)c2)cc1 ; c1ccc(-c2ccc(-c3cccc(-n4c5ccccc5c5cc(-c6ccc7c8ccccc8n(-c8cccc(-c9ccccc9)c8)c7c6)ccc54)c3)cc2)cc1] 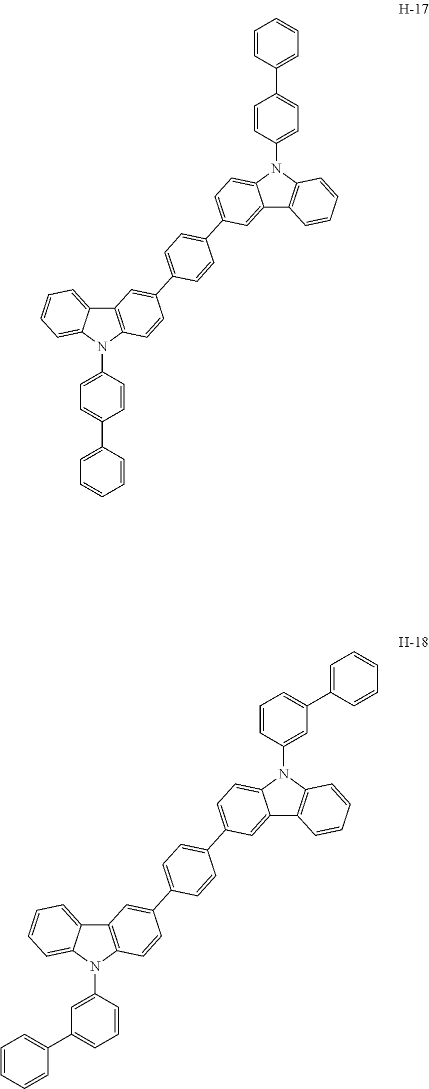 [SMILES: c1ccc(-c2ccc(-n3c4ccccc4c4cc(-c5ccc(-c6ccc7c(c6)c6ccccc6n7-c6ccc(-c7ccccc7)cc6)cc5)ccc43)cc2)cc1 ; c1ccc(-c2cccc(-n3c4ccccc4c4cc(-c5ccc(-c6ccc7c(c6)c6ccccc6n7-c6cccc(-c7ccccc7)c6)cc5)ccc43)c2)cc1] 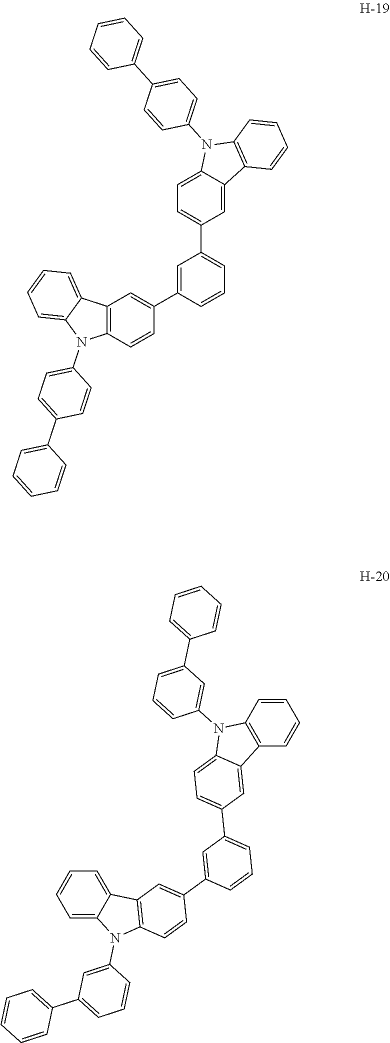 [SMILES: c1ccc(-c2ccc(-n3c4ccccc4c4cc(-c5cccc(-c6ccc7c(c6)c6ccccc6n7-c6ccc(-c7ccccc7)cc6)c5)ccc43)cc2)cc1 ; c1ccc(-c2cccc(-n3c4ccccc4c4cc(-c5cccc(-c6ccc7c(c6)c6ccccc6n7-c6cccc(-c7ccccc7)c6)c5)ccc43)c2)cc1] 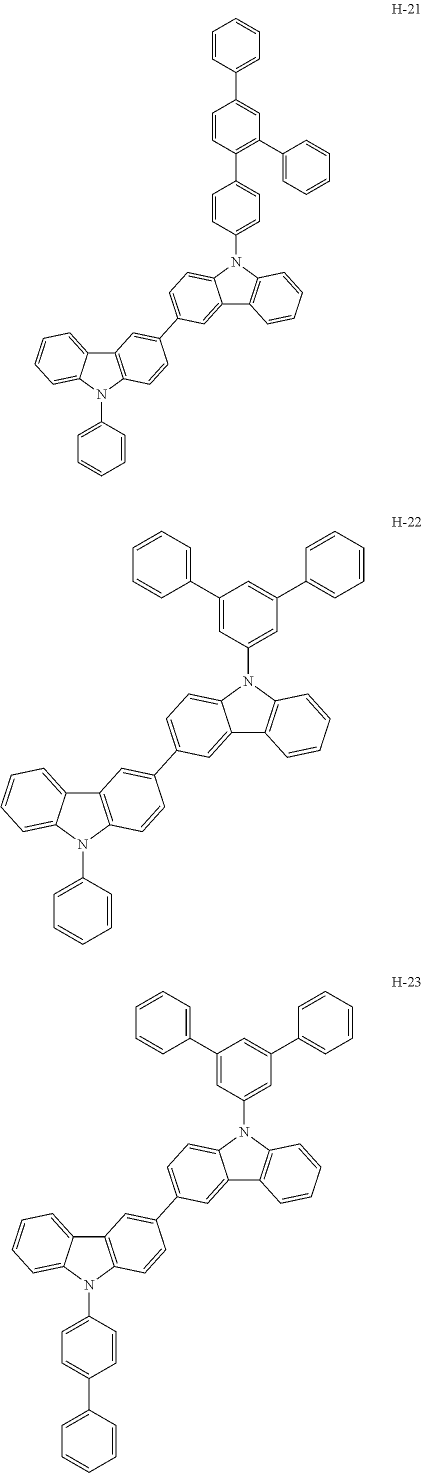 [SMILES: c1ccc(-c2ccc(-c3ccc(-n4c5ccccc5c5cc(-c6ccc7c(c6)c6ccccc6n7-c6ccccc6)ccc54)cc3)c(-c3ccccc3)c2)cc1 ; c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1] 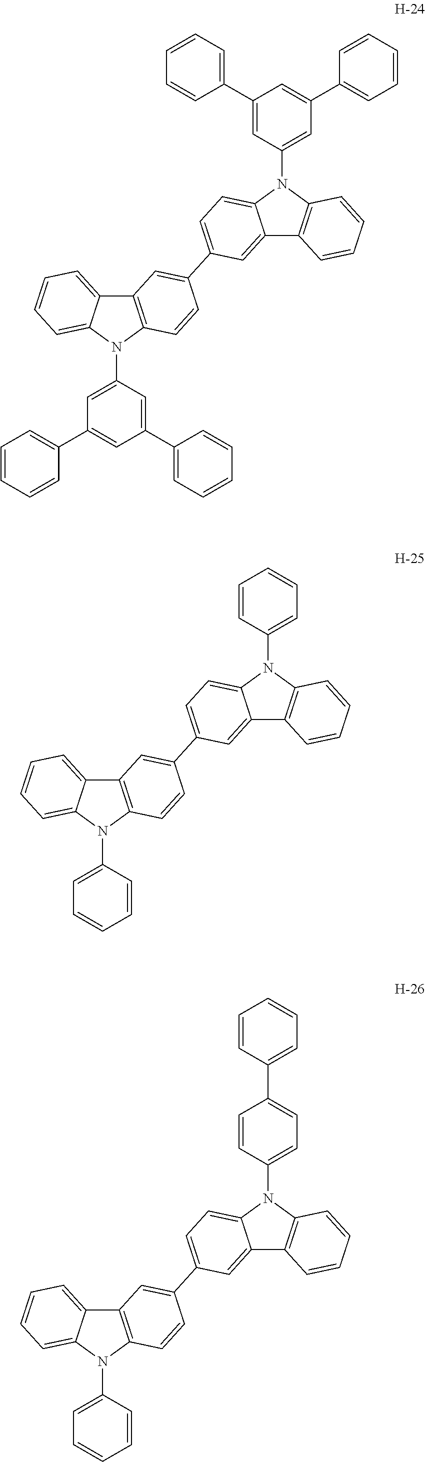 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)cc2)cc1] 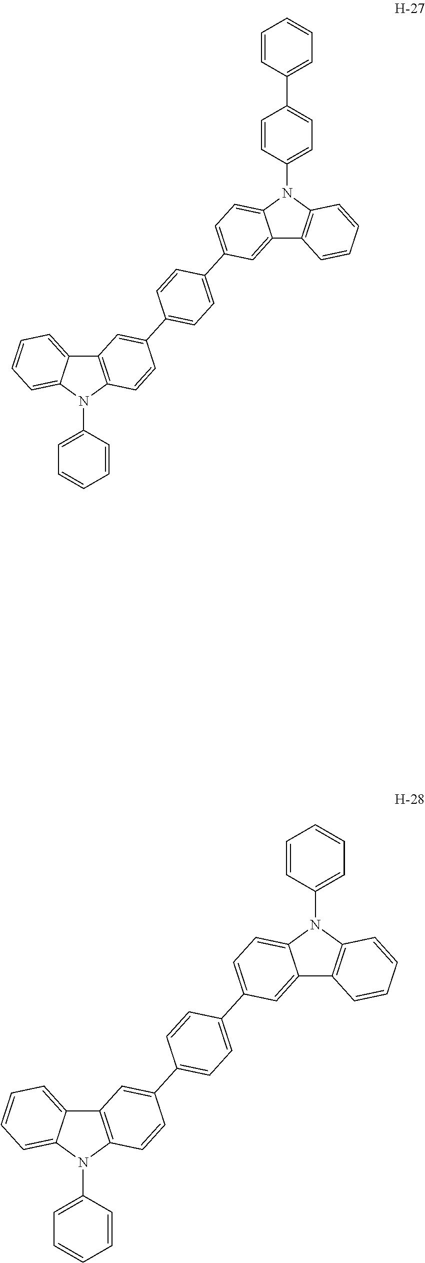 [SMILES: c1ccc(-c2ccc(-n3c4ccccc4c4cc(-c5ccc(-c6ccc7c(c6)c6ccccc6n7-c6ccccc6)cc5)ccc43)cc2)cc1 ; c1ccc(-n2c3ccccc3c3cc(-c4ccc(-c5ccc6c(c5)c5ccccc5n6-c5ccccc5)cc4)ccc32)cc1] 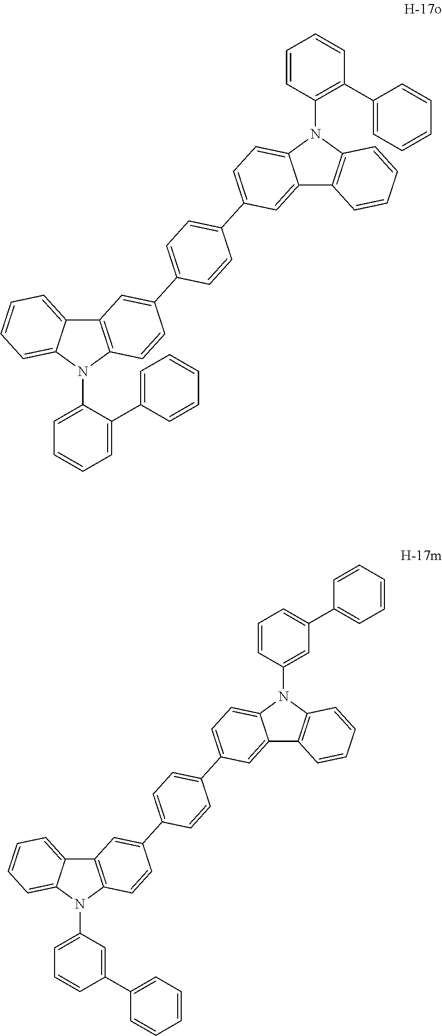 [SMILES: c1ccc(-c2ccccc2-n2c3ccccc3c3cc(-c4ccc(-c5ccc6c(c5)c5ccccc5n6-c5ccccc5-c5ccccc5)cc4)ccc32)cc1 ; c1ccc(-c2cccc(-n3c4ccccc4c4cc(-c5ccc(-c6ccc7c(c6)c6ccccc6n7-c6cccc(-c7ccccc7)c6)cc5)ccc43)c2)cc1] 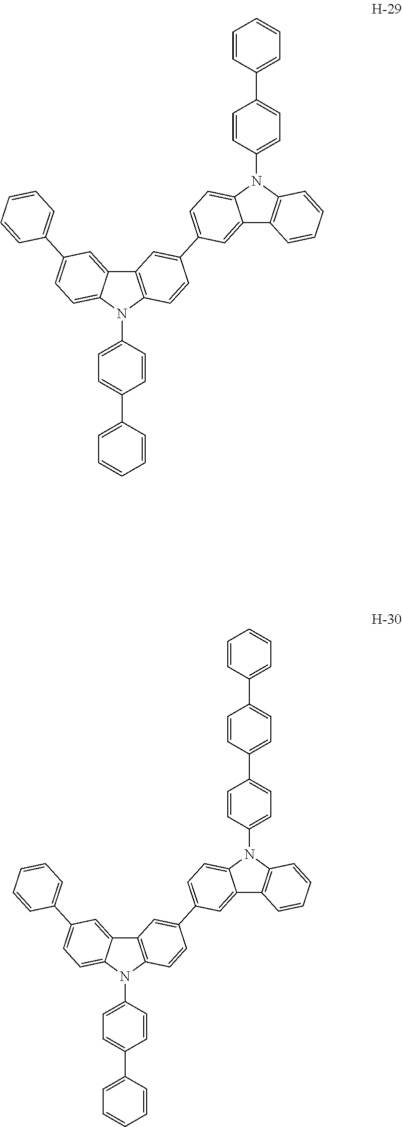 [SMILES: c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1 ; c1ccc(-c2ccc(-c3ccc(-n4c5ccccc5c5cc(-c6ccc7c(c6)c6cc(-c8ccccc8)ccc6n7-c6ccc(-c7ccccc7)cc6)ccc54)cc3)cc2)cc1] 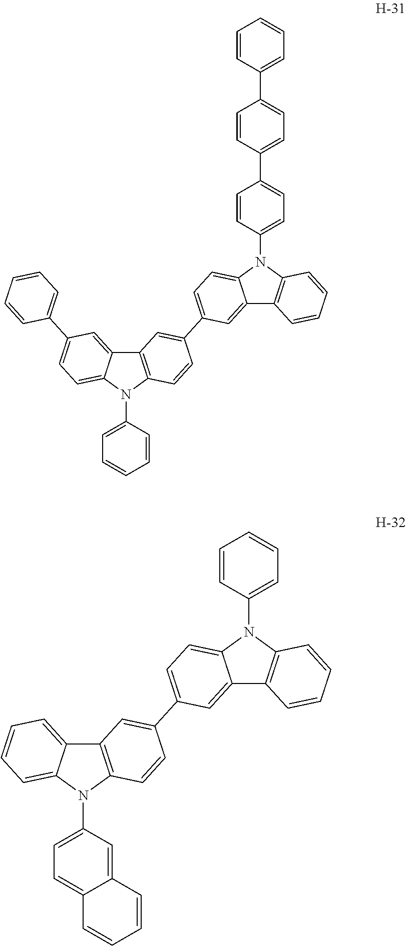 [SMILES: c1ccc(-c2ccc(-c3ccc(-n4c5ccccc5c5cc(-c6ccc7c(c6)c6cc(-c8ccccc8)ccc6n7-c6ccccc6)ccc54)cc3)cc2)cc1 ; c1ccc(-n2c3ccccc3c3cc(-c4ccc5c(c4)c4ccccc4n5-c4ccc5ccccc5c4)ccc32)cc1] 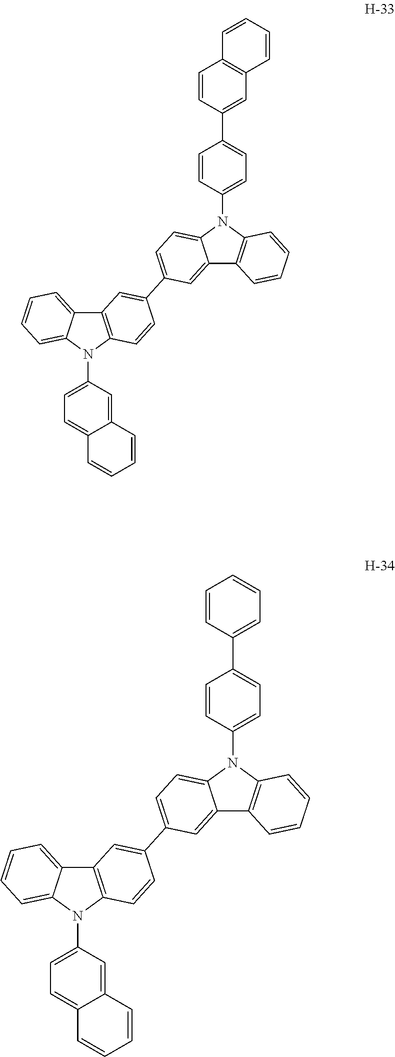 [SMILES: c1ccc2cc(-c3ccc(-n4c5ccccc5c5cc(-c6ccc7c(c6)c6ccccc6n7-c6ccc7ccccc7c6)ccc54)cc3)ccc2c1 ; c1ccc(-c2ccc(-n3c4ccccc4c4cc(-c5ccc6c(c5)c5ccccc5n6-c5ccc6ccccc6c5)ccc43)cc2)cc1] 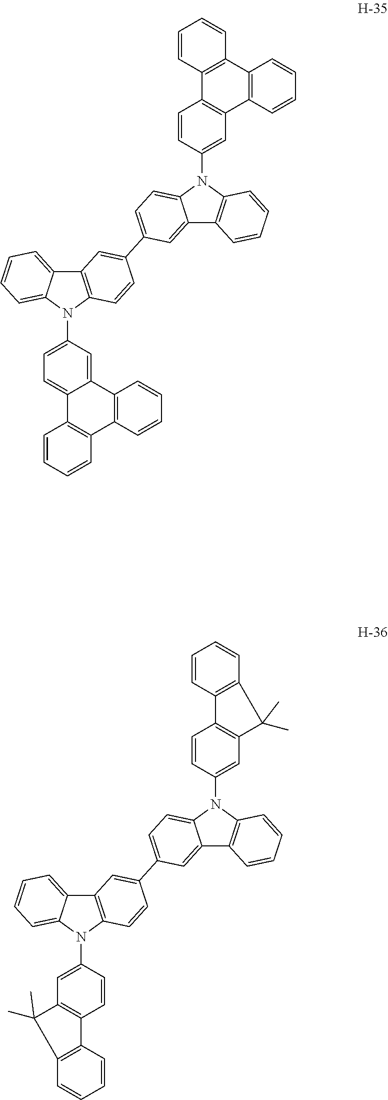 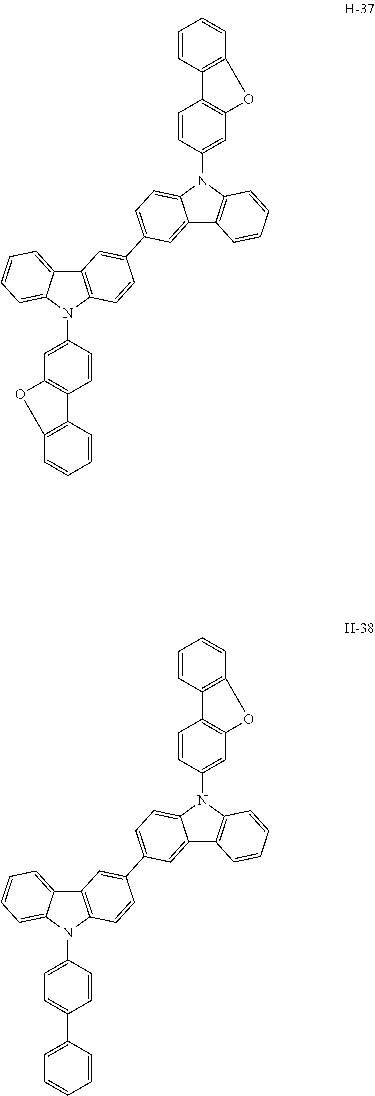 [SMILES: c1ccc2c(c1)oc1cc(-n3c4ccccc4c4cc(-c5ccc6c(c5)c5ccccc5n6-c5ccc6c(c5)oc5ccccc56)ccc43)ccc12 ; c1ccc(-c2ccc(-n3c4ccccc4c4cc(-c5ccc6c(c5)c5ccccc5n6-c5ccc6c(c5)oc5ccccc56)ccc43)cc2)cc1] 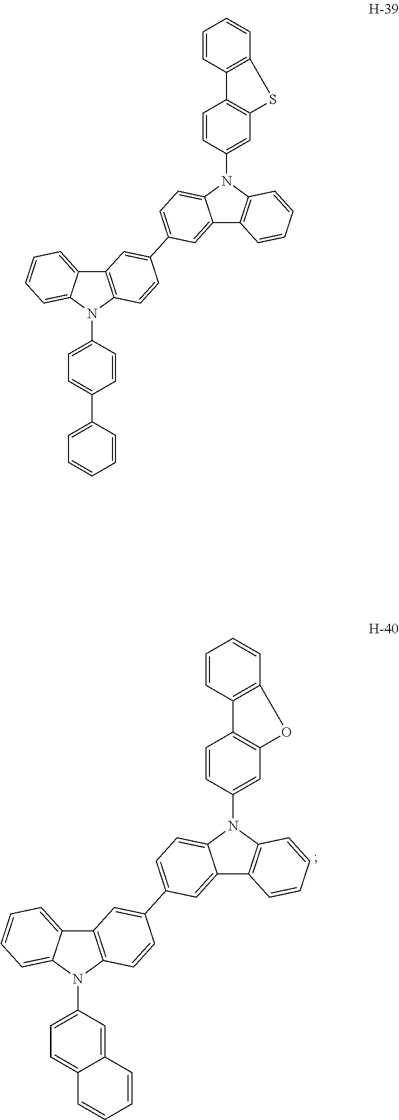 [SMILES: c1ccc(-c2ccc(-n3c4ccccc4c4cc(-c5ccc6c(c5)c5ccccc5n6-c5ccc6c(c5)sc5ccccc56)ccc43)cc2)cc1 ; c1ccc2cc(-n3c4ccccc4c4cc(-c5ccc6c(c5)c5ccccc5n6-c5ccc6c(c5)oc5ccccc56)ccc43)ccc2c1]
the “substituted” means that hydrogen atoms in the above compounds are each independently substituted with deuterium atoms.

6. The composition according to claim 1, wherein the compound represented by Formula II is selected from any one of the following substituted or unsubstituted compounds: 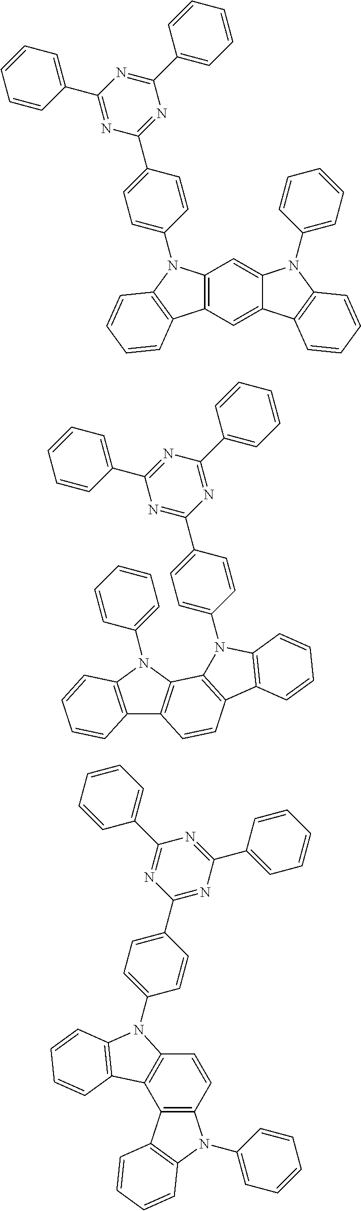 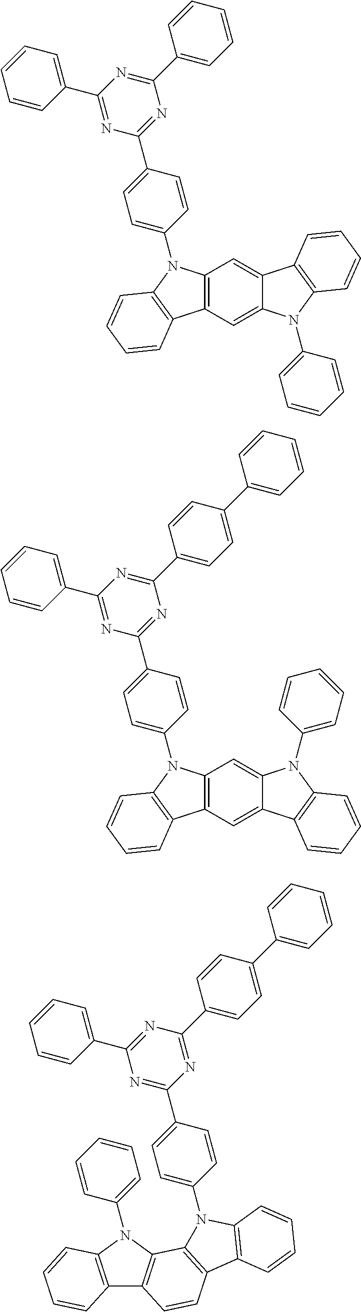 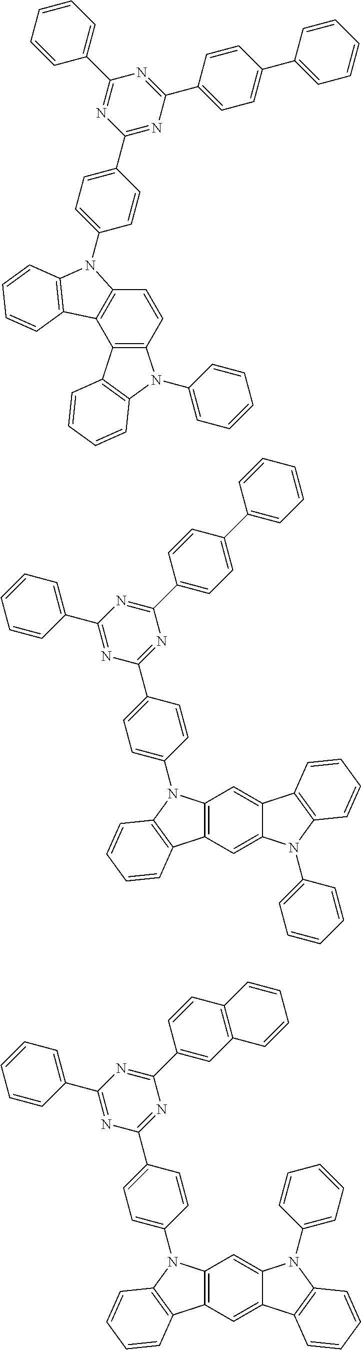 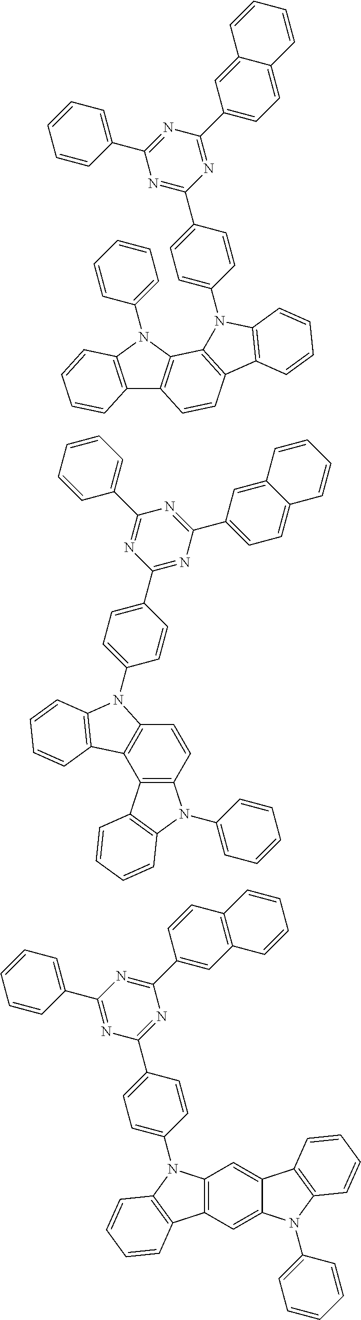 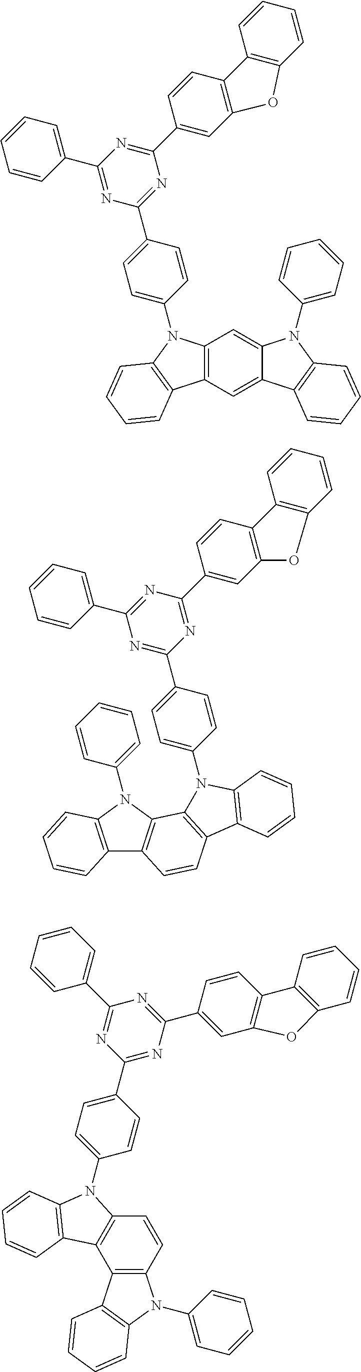 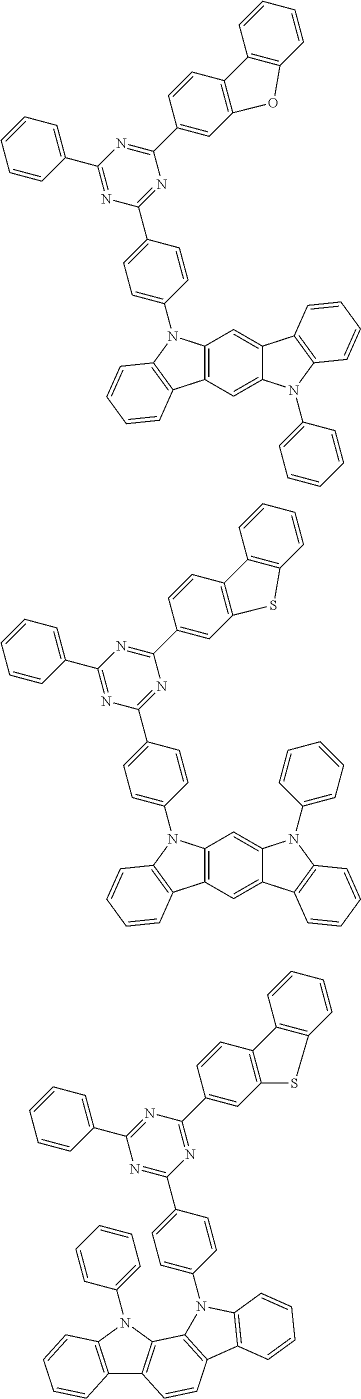 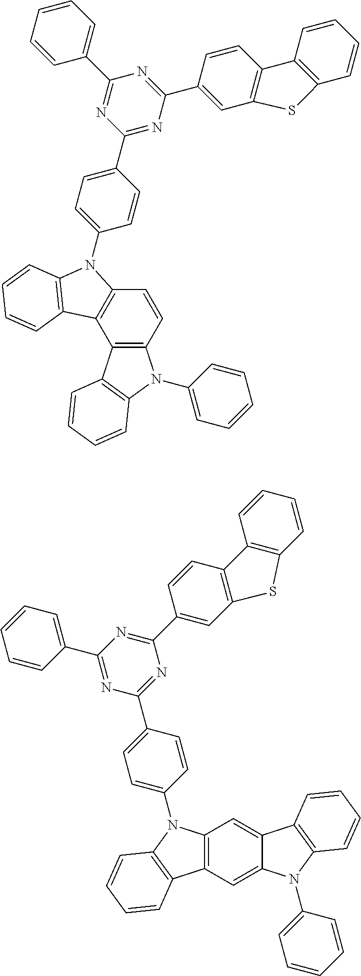 [SMILES: c1ccc(-c2nc(-c3ccc(-n4c5ccccc5c5c6c7ccccc7n(-c7ccccc7)c6ccc54)cc3)nc(-c3ccc4c(c3)sc3ccccc34)n2)cc1 ; c1ccc(-c2nc(-c3ccc(-n4c5ccccc5c5cc6c(cc54)c4ccccc4n6-c4ccccc4)cc3)nc(-c3ccc4c(c3)sc3ccccc34)n2)cc1] 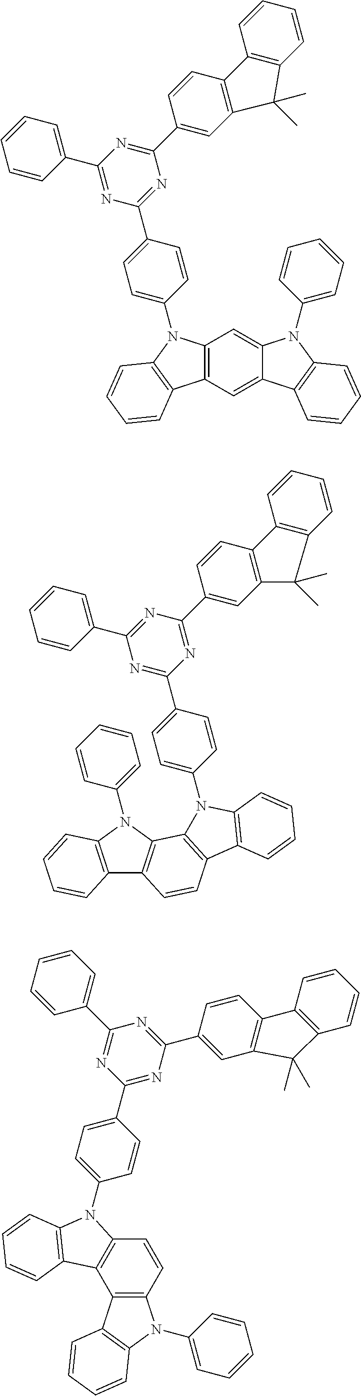 [SMILES: CC1(C)c2ccccc2-c2ccc(-c3nc(-c4ccccc4)nc(-c4ccc(-n5c6ccccc6c6cc7c8ccccc8n(-c8ccccc8)c7cc65)cc4)n3)cc21 ; CC1(C)c2ccccc2-c2ccc(-c3nc(-c4ccccc4)nc(-c4ccc(-n5c6ccccc6c6ccc7c8ccccc8n(-c8ccccc8)c7c65)cc4)n3)cc21 ; CC1(C)c2ccccc2-c2ccc(-c3nc(-c4ccccc4)nc(-c4ccc(-n5c6ccccc6c6c7c8ccccc8n(-c8ccccc8)c7ccc65)cc4)n3)cc21] 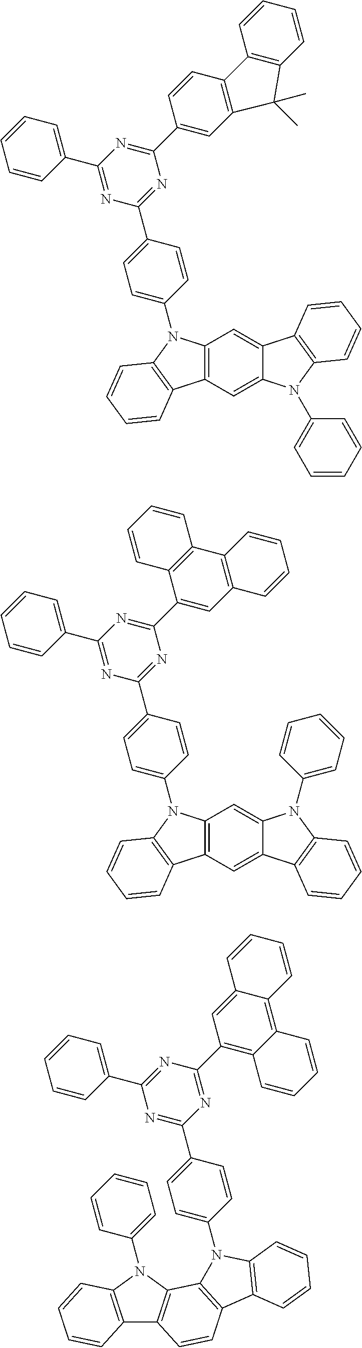 [SMILES: CC1(C)c2ccccc2-c2ccc(-c3nc(-c4ccccc4)nc(-c4ccc(-n5c6ccccc6c6cc7c(cc65)c5ccccc5n7-c5ccccc5)cc4)n3)cc21 ; c1ccc(-c2nc(-c3ccc(-n4c5ccccc5c5cc6c7ccccc7n(-c7ccccc7)c6cc54)cc3)nc(-c3cc4ccccc4c4ccccc34)n2)cc1 ; c1ccc(-c2nc(-c3ccc(-n4c5ccccc5c5ccc6c7ccccc7n(-c7ccccc7)c6c54)cc3)nc(-c3cc4ccccc4c4ccccc34)n2)cc1] 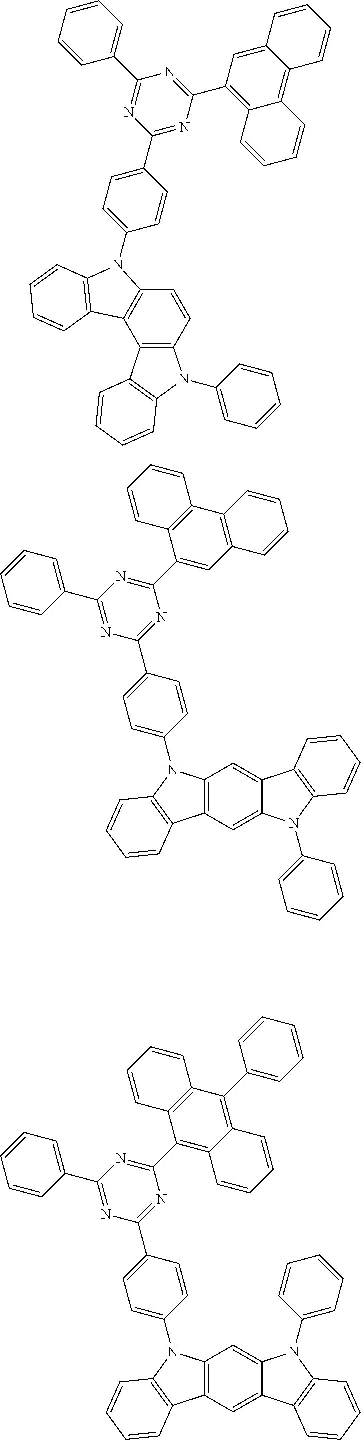 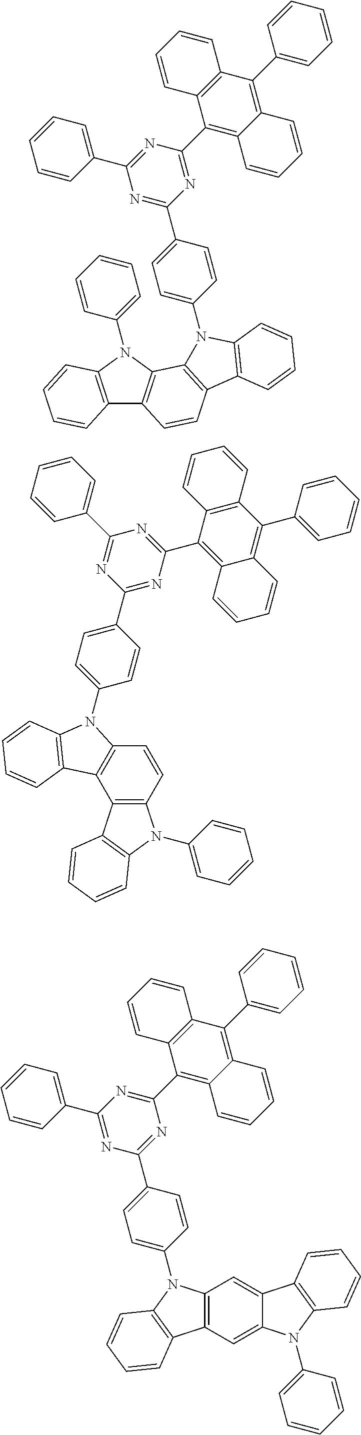 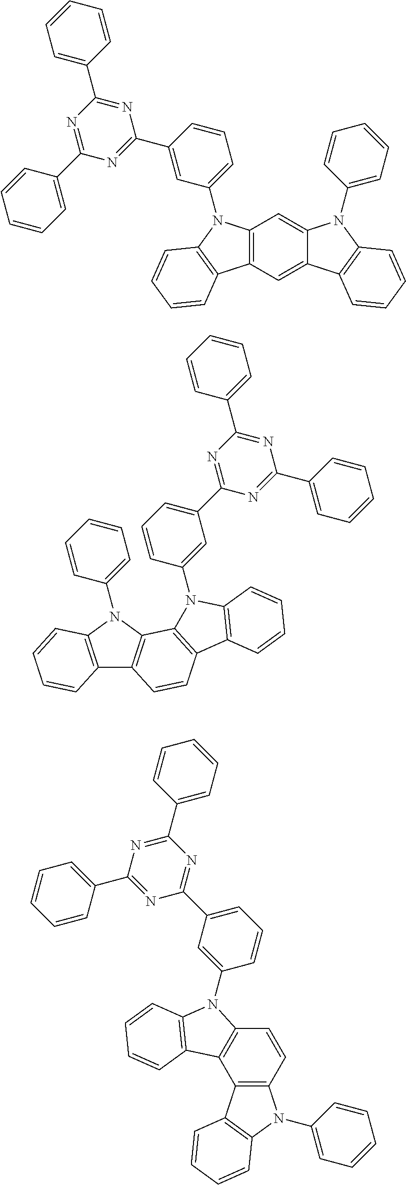 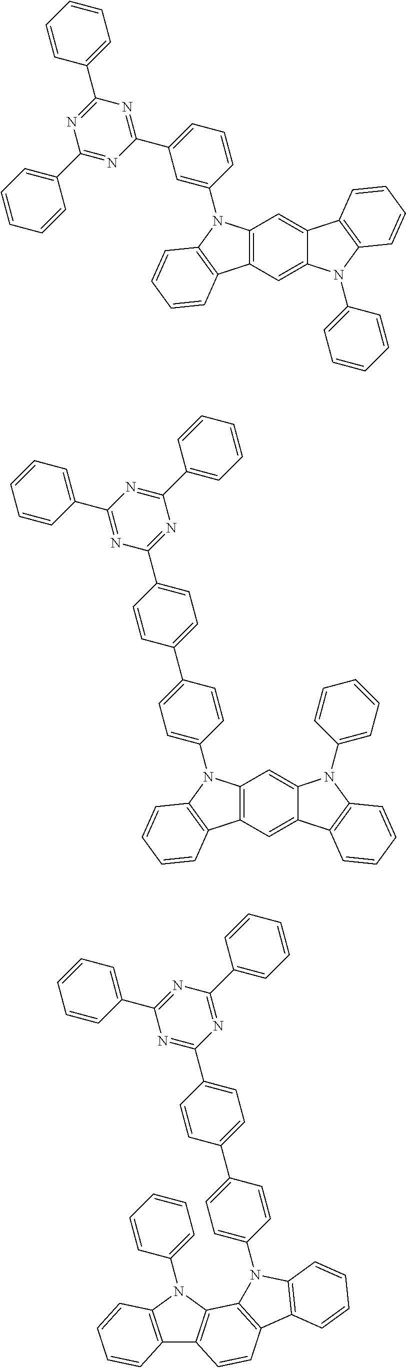 [SMILES: c1ccc(-c2nc(-c3ccccc3)nc(-c3cccc(-n4c5ccccc5c5cc6c(cc54)c4ccccc4n6-c4ccccc4)c3)n2)cc1 ; c1ccc(-c2nc(-c3ccccc3)nc(-c3ccc(-c4ccc(-n5c6ccccc6c6cc7c8ccccc8n(-c8ccccc8)c7cc65)cc4)cc3)n2)cc1 ; c1ccc(-c2nc(-c3ccccc3)nc(-c3ccc(-c4ccc(-n5c6ccccc6c6ccc7c8ccccc8n(-c8ccccc8)c7c65)cc4)cc3)n2)cc1] 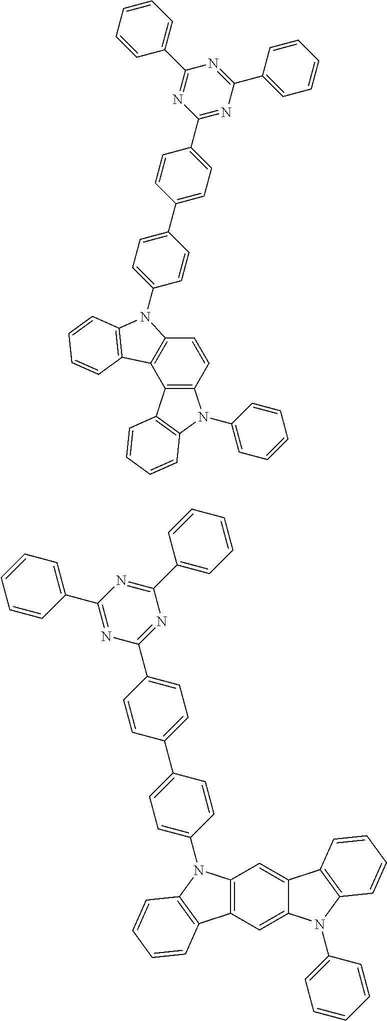 [SMILES: c1ccc(-c2nc(-c3ccccc3)nc(-c3ccc(-c4ccc(-n5c6ccccc6c6c7c8ccccc8n(-c8ccccc8)c7ccc65)cc4)cc3)n2)cc1 ; c1ccc(-c2nc(-c3ccccc3)nc(-c3ccc(-c4ccc(-n5c6ccccc6c6cc7c(cc65)c5ccccc5n7-c5ccccc5)cc4)cc3)n2)cc1] 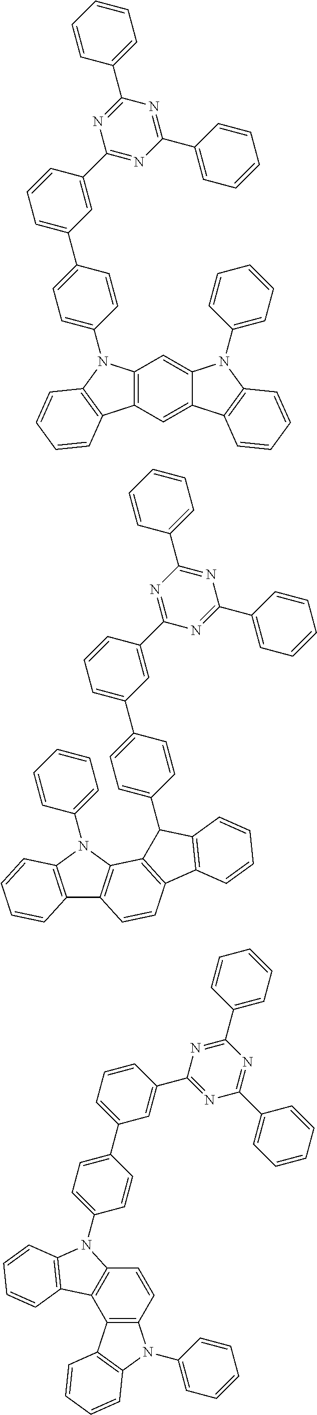 [SMILES: c1ccc(-c2nc(-c3ccccc3)nc(-c3cccc(-c4ccc(-n5c6ccccc6c6cc7c8ccccc8n(-c8ccccc8)c7cc65)cc4)c3)n2)cc1 ; c1ccc(-c2nc(-c3ccccc3)nc(-c3cccc(-c4ccc(C5c6ccccc6-c6ccc7c8ccccc8n(-c8ccccc8)c7c65)cc4)c3)n2)cc1 ; c1ccc(-c2nc(-c3ccccc3)nc(-c3cccc(-c4ccc(-n5c6ccccc6c6c7c8ccccc8n(-c8ccccc8)c7ccc65)cc4)c3)n2)cc1] 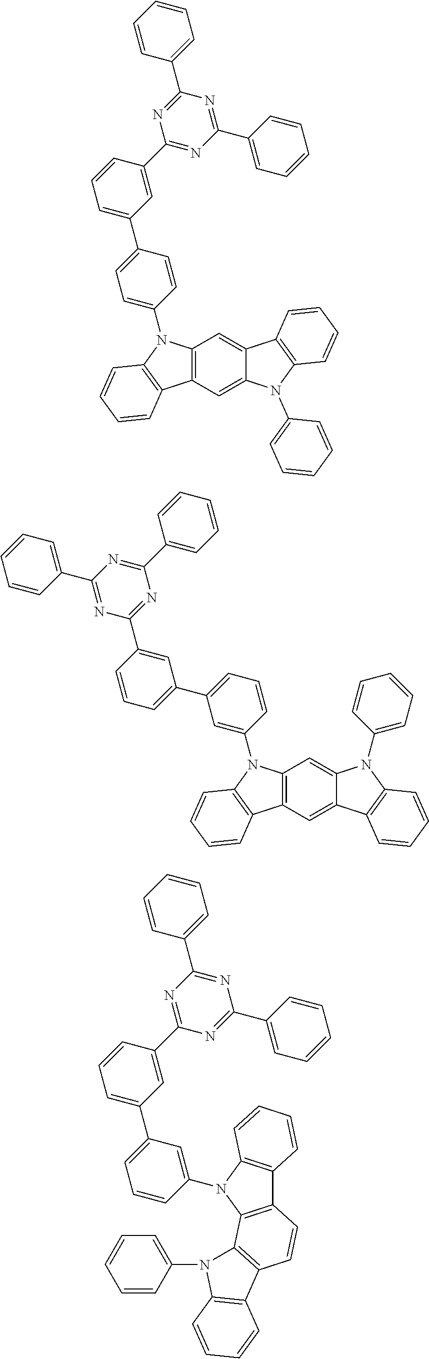 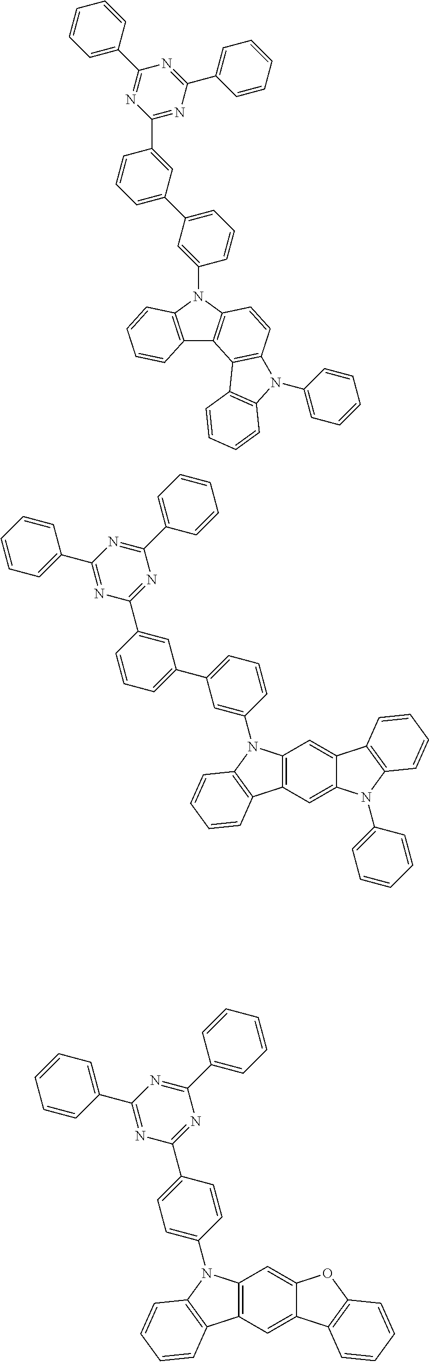 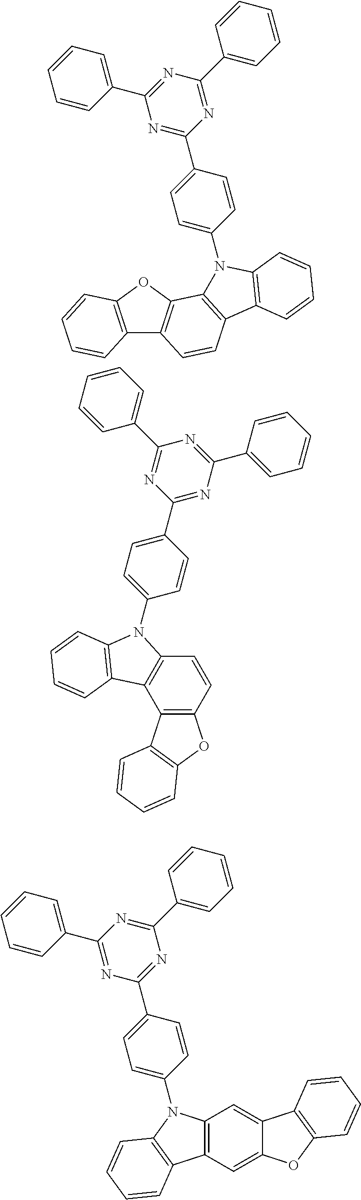 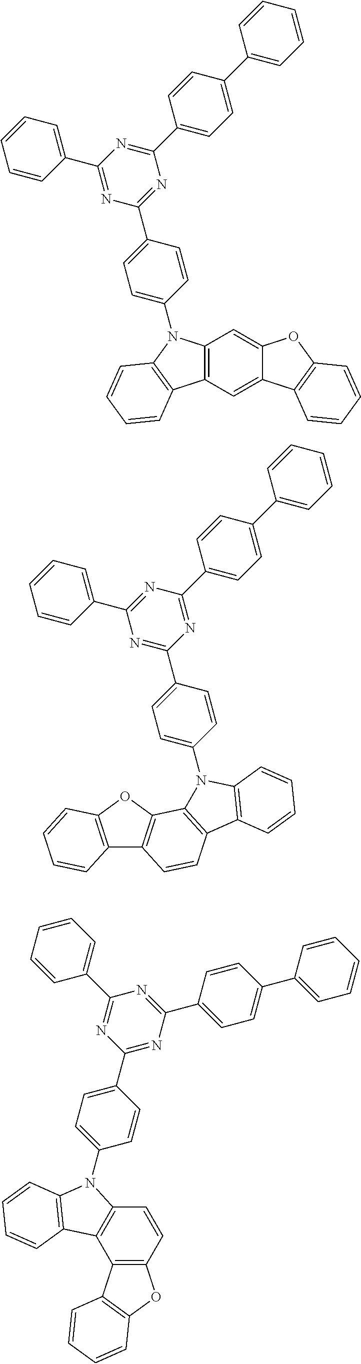 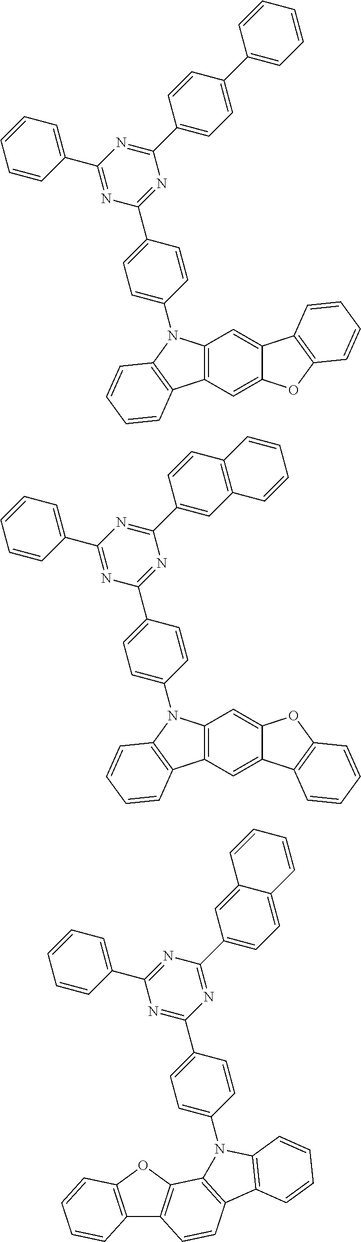 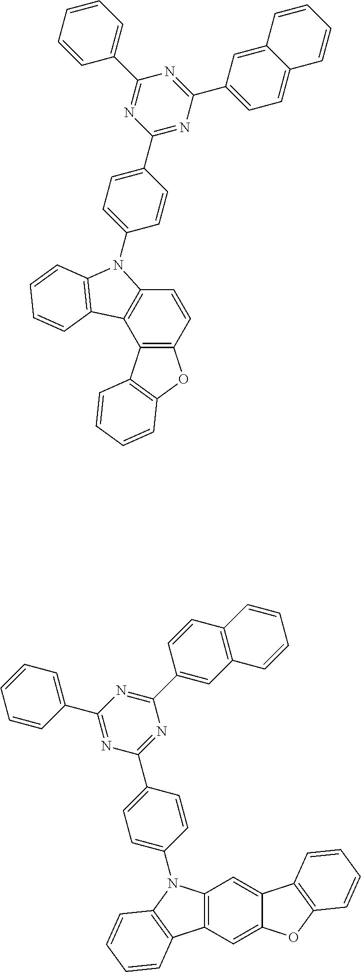 [SMILES: c1ccc(-c2nc(-c3ccc(-n4c5ccccc5c5c6c(ccc54)oc4ccccc46)cc3)nc(-c3ccc4ccccc4c3)n2)cc1 ; c1ccc(-c2nc(-c3ccc(-n4c5ccccc5c5cc6oc7ccccc7c6cc54)cc3)nc(-c3ccc4ccccc4c3)n2)cc1] 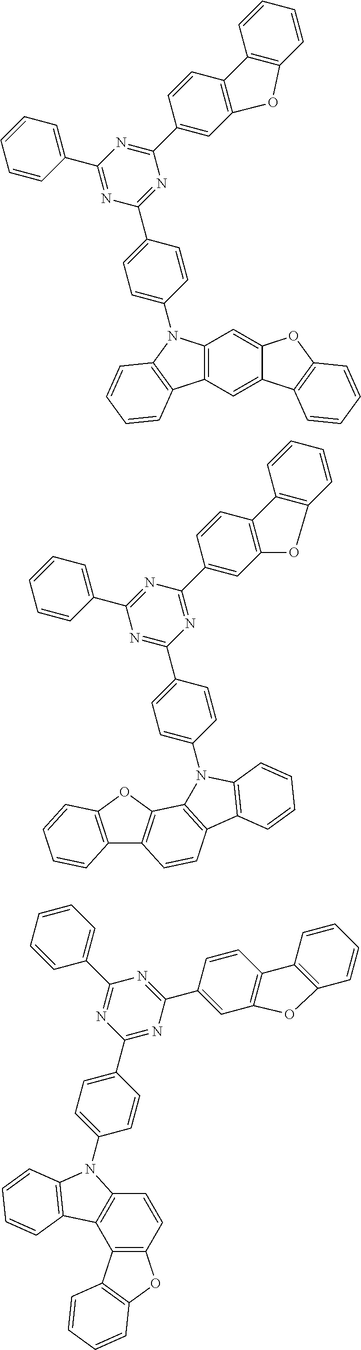 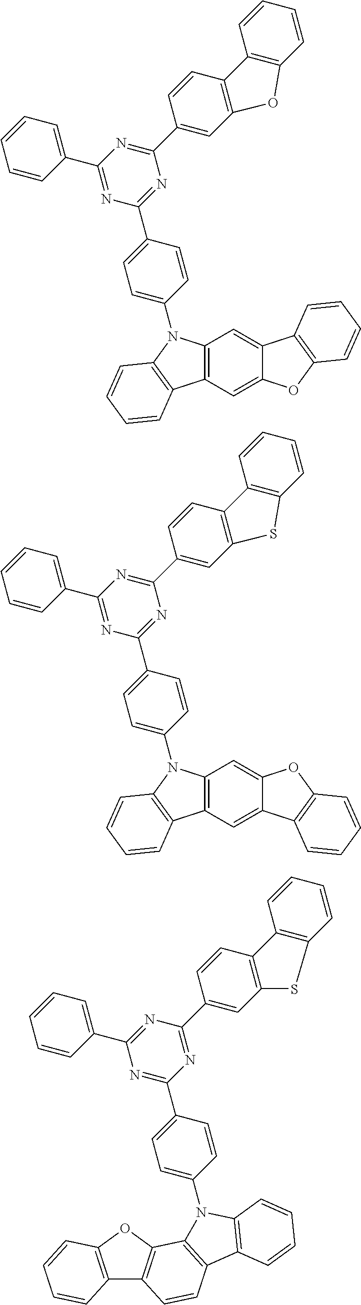 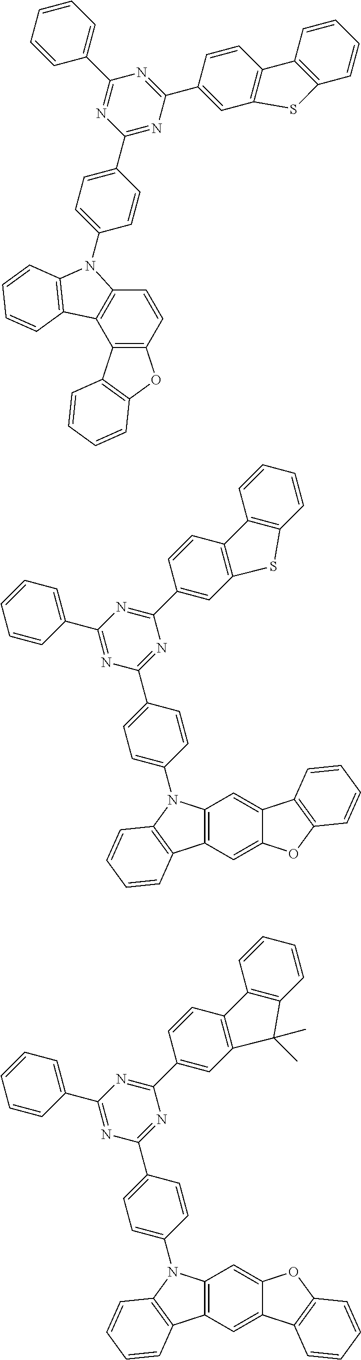 [SMILES: c1ccc(-c2nc(-c3ccc(-n4c5ccccc5c5c6c(ccc54)oc4ccccc46)cc3)nc(-c3ccc4c(c3)sc3ccccc34)n2)cc1 ; c1ccc(-c2nc(-c3ccc(-n4c5ccccc5c5cc6oc7ccccc7c6cc54)cc3)nc(-c3ccc4c(c3)sc3ccccc34)n2)cc1 ; CC1(C)c2ccccc2-c2ccc(-c3nc(-c4ccccc4)nc(-c4ccc(-n5c6ccccc6c6cc7c(cc65)oc5ccccc57)cc4)n3)cc21] 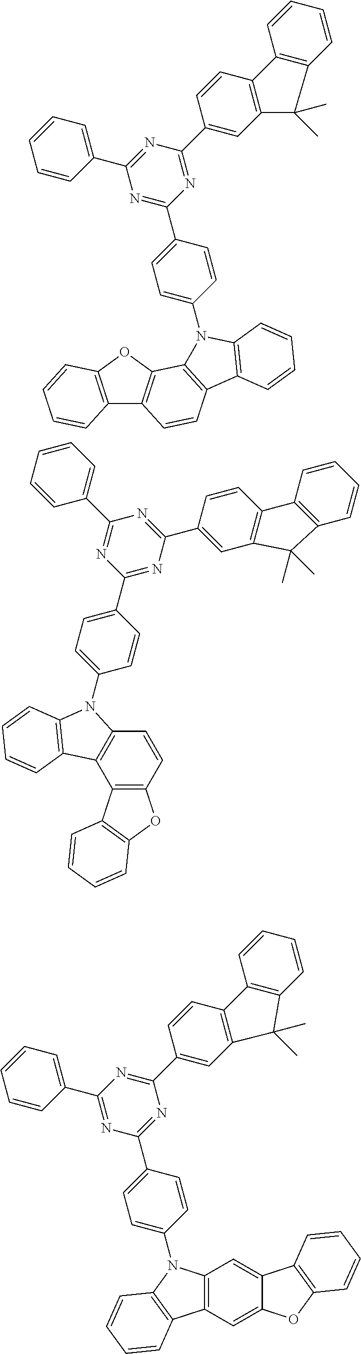 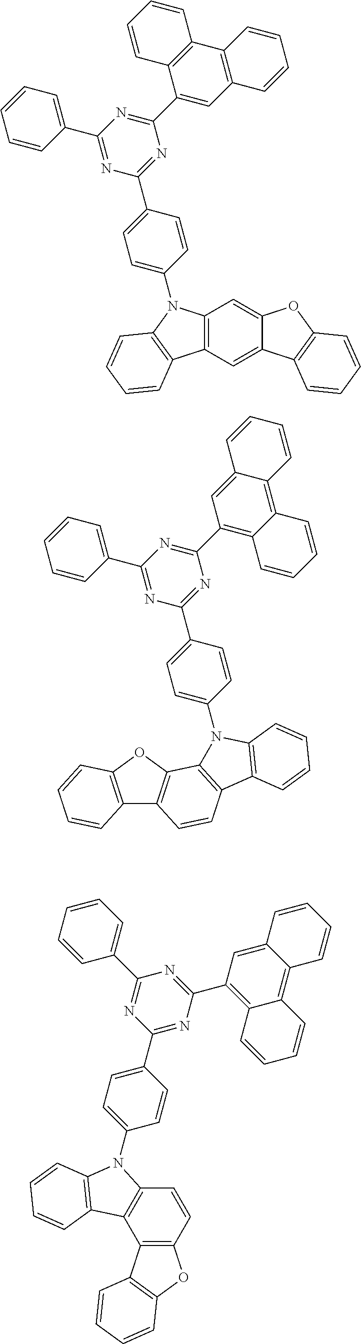 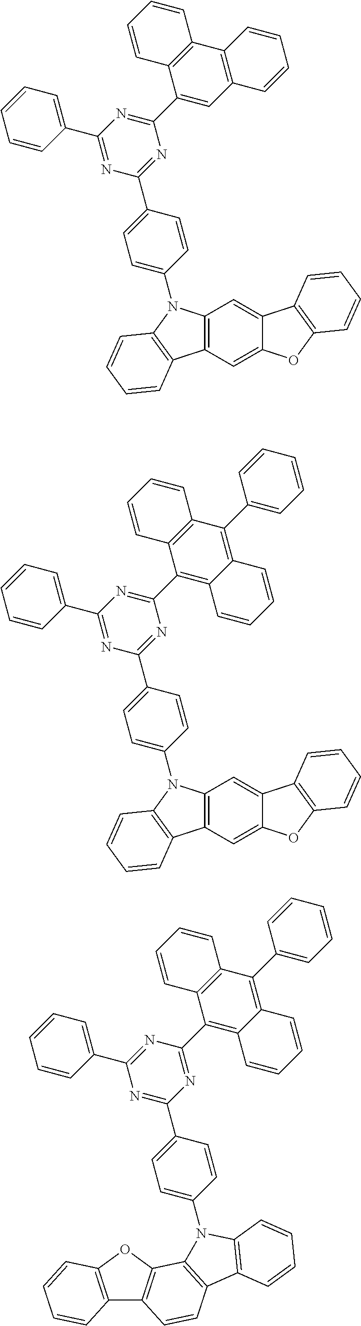 [SMILES: c1ccc(-c2nc(-c3ccc(-n4c5ccccc5c5cc6oc7ccccc7c6cc54)cc3)nc(-c3cc4ccccc4c4ccccc34)n2)cc1 ; c1ccc(-c2nc(-c3ccc(-n4c5ccccc5c5cc6oc7ccccc7c6cc54)cc3)nc(-c3c4ccccc4c(-c4ccccc4)c4ccccc34)n2)cc1 ; c1ccc(-c2nc(-c3ccc(-n4c5ccccc5c5ccc6c7ccccc7oc6c54)cc3)nc(-c3c4ccccc4c(-c4ccccc4)c4ccccc34)n2)cc1] 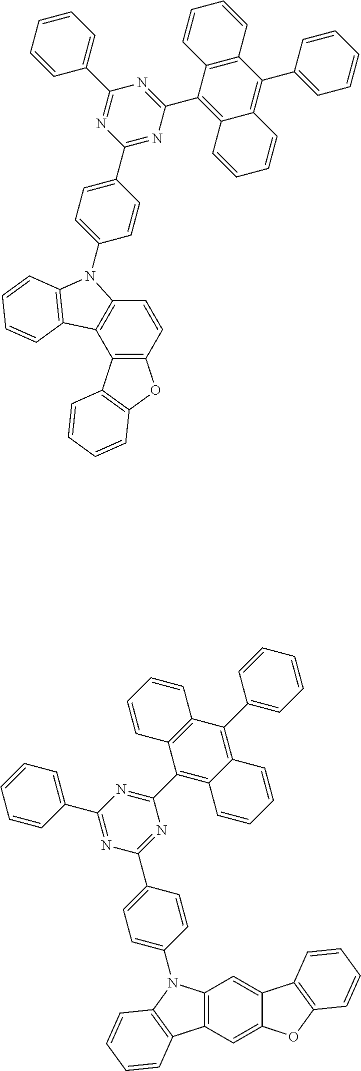 [SMILES: c1ccc(-c2nc(-c3ccc(-n4c5ccccc5c5c6c(ccc54)oc4ccccc46)cc3)nc(-c3c4ccccc4c(-c4ccccc4)c4ccccc34)n2)cc1 ; c1ccc(-c2nc(-c3ccc(-n4c5ccccc5c5cc6oc7ccccc7c6cc54)cc3)nc(-c3c4ccccc4c(-c4ccccc4)c4ccccc34)n2)cc1] 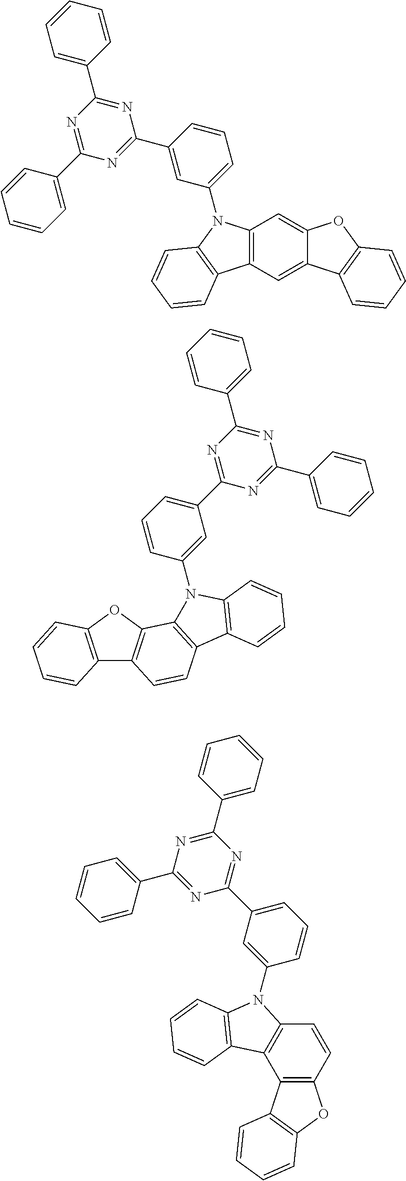 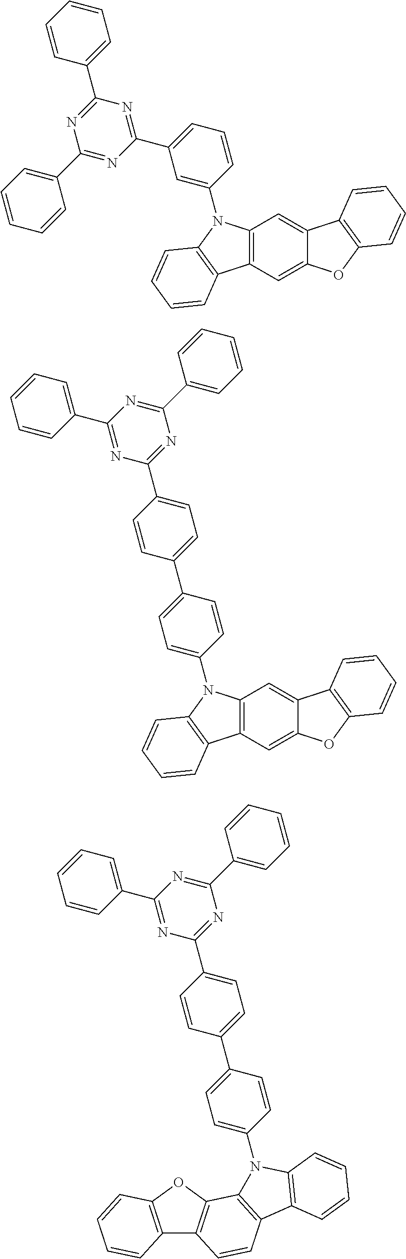 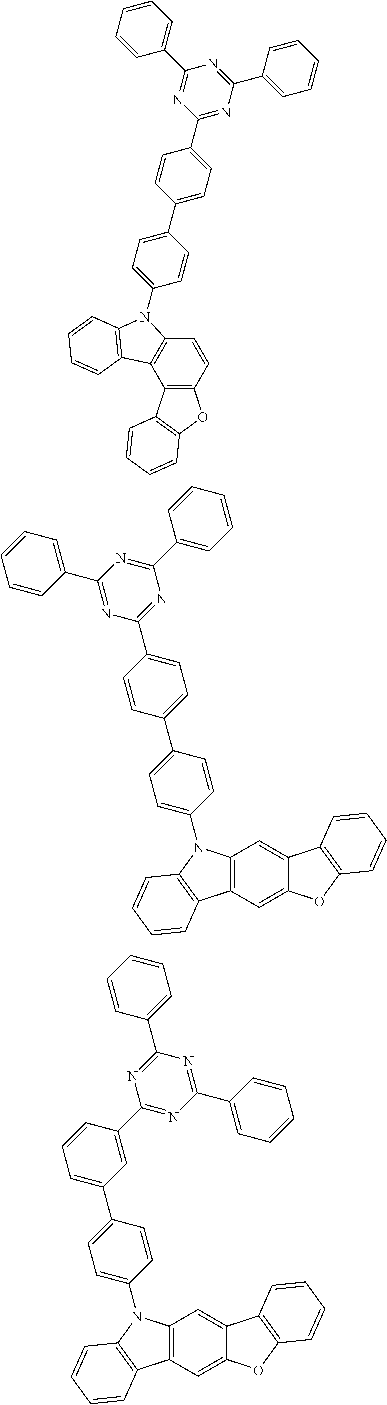 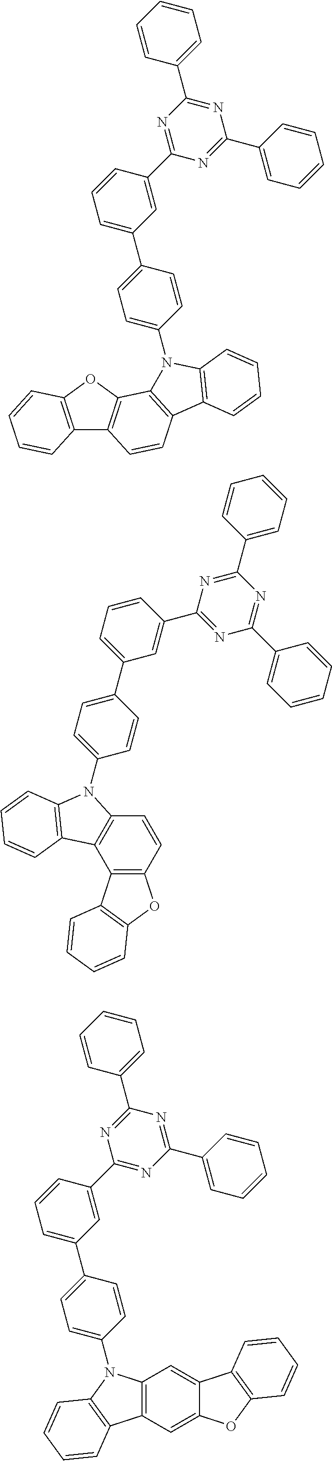 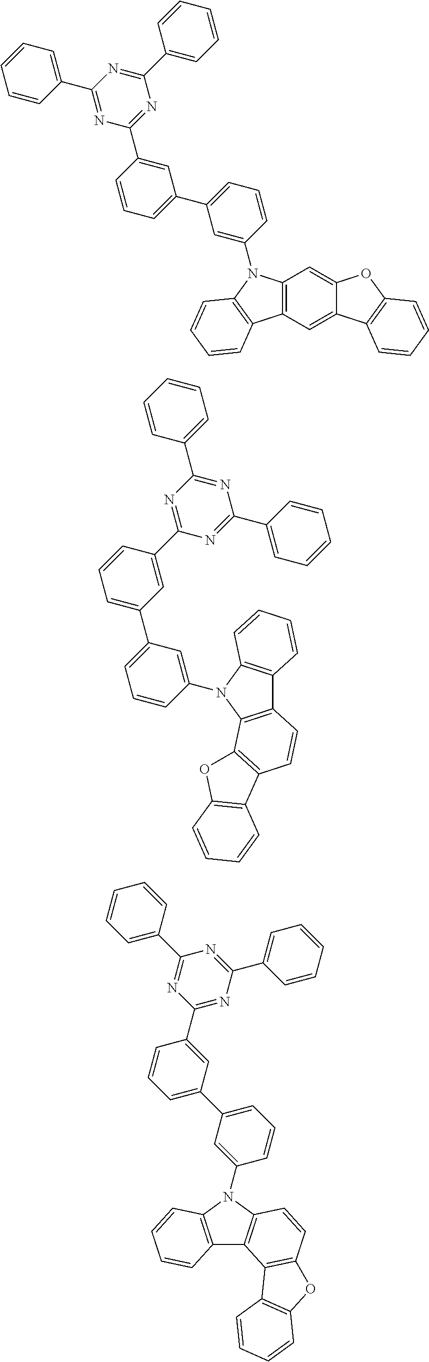 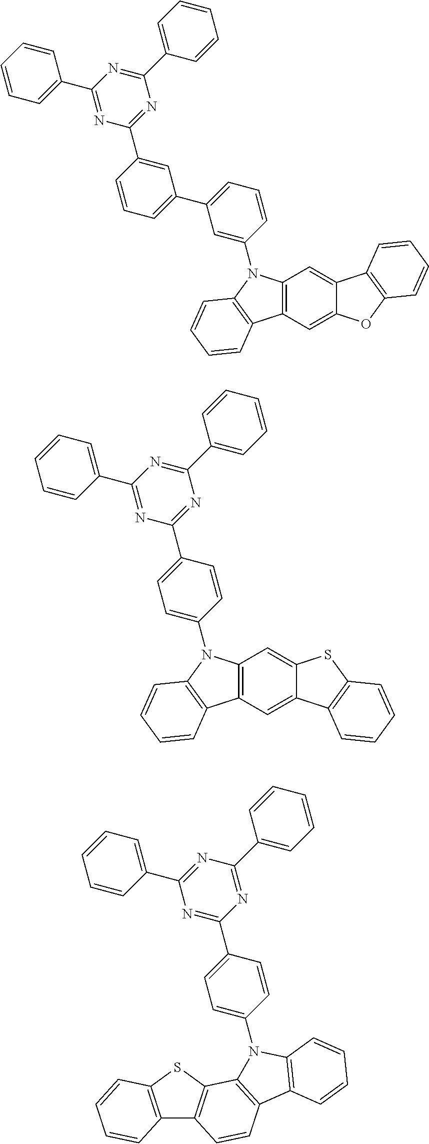 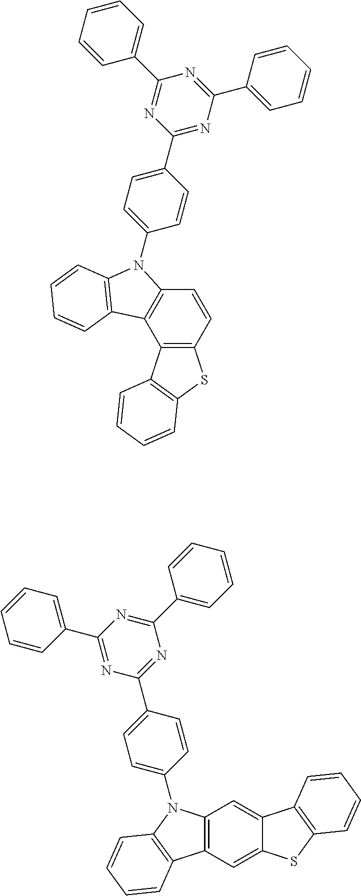 [SMILES: c1ccc(-c2nc(-c3ccccc3)nc(-c3ccc(-n4c5ccccc5c5c6c(ccc54)sc4ccccc46)cc3)n2)cc1 ; c1ccc(-c2nc(-c3ccccc3)nc(-c3ccc(-n4c5ccccc5c5cc6sc7ccccc7c6cc54)cc3)n2)cc1] 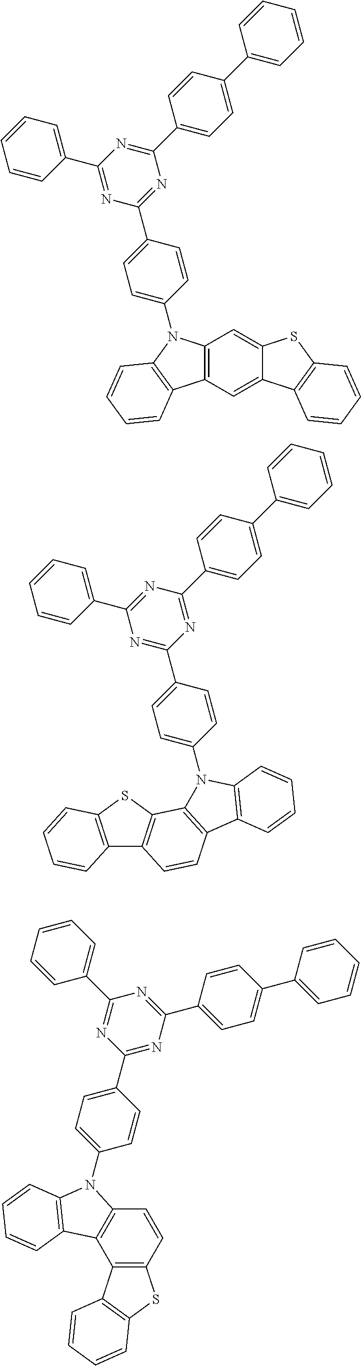 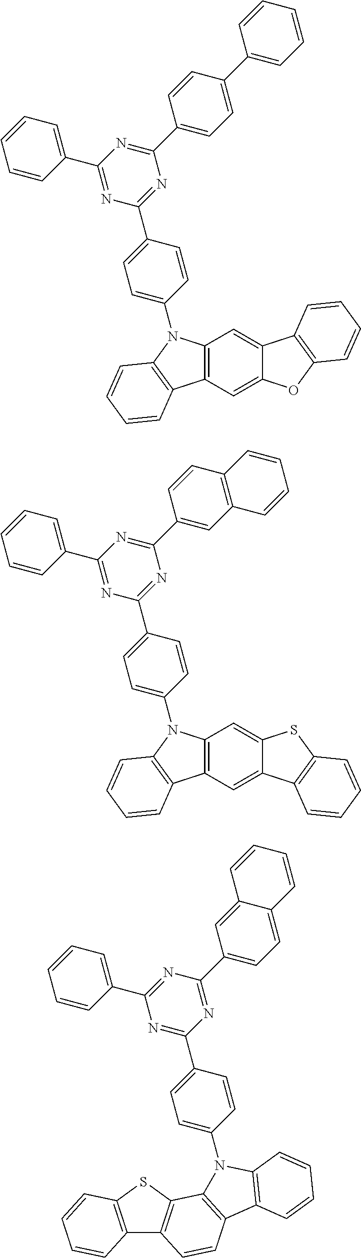 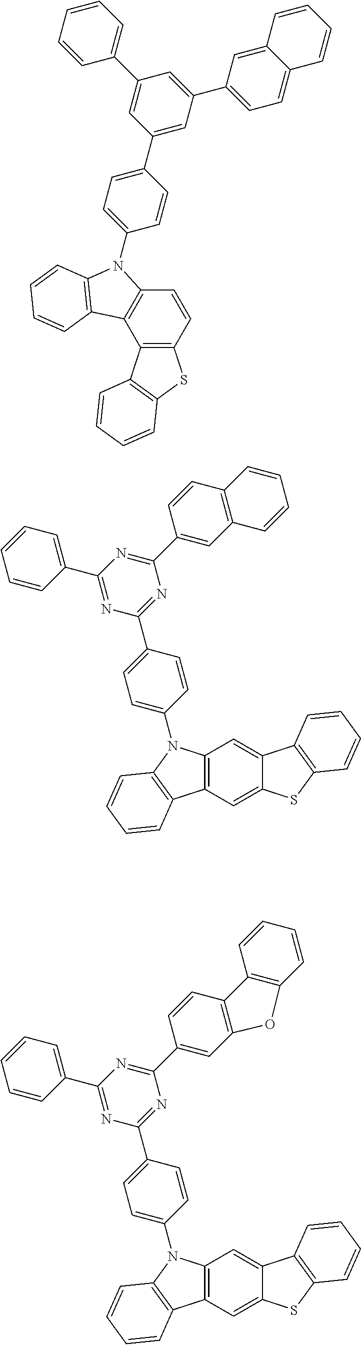 [SMILES: c1ccc(-c2cc(-c3ccc(-n4c5ccccc5c5c6c(ccc54)sc4ccccc46)cc3)cc(-c3ccc4ccccc4c3)c2)cc1 ; c1ccc(-c2nc(-c3ccc(-n4c5ccccc5c5cc6sc7ccccc7c6cc54)cc3)nc(-c3ccc4ccccc4c3)n2)cc1 ; c1ccc(-c2nc(-c3ccc(-n4c5ccccc5c5cc6sc7ccccc7c6cc54)cc3)nc(-c3ccc4c(c3)oc3ccccc34)n2)cc1] 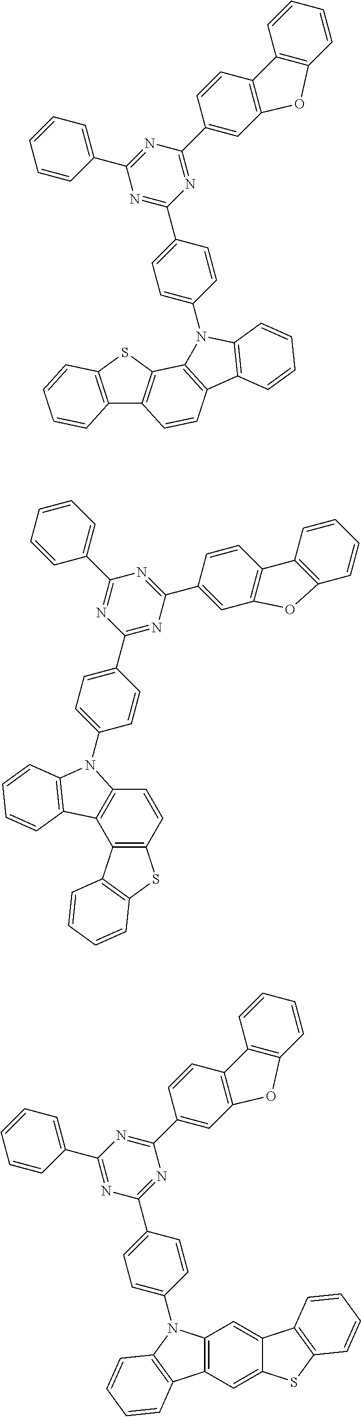 [SMILES: c1ccc(-c2nc(-c3ccc(-n4c5ccccc5c5ccc6c7ccccc7sc6c54)cc3)nc(-c3ccc4c(c3)oc3ccccc34)n2)cc1 ; c1ccc(-c2nc(-c3ccc(-n4c5ccccc5c5c6c(ccc54)sc4ccccc46)cc3)nc(-c3ccc4c(c3)oc3ccccc34)n2)cc1 ; c1ccc(-c2nc(-c3ccc(-n4c5ccccc5c5cc6sc7ccccc7c6cc54)cc3)nc(-c3ccc4c(c3)oc3ccccc34)n2)cc1] 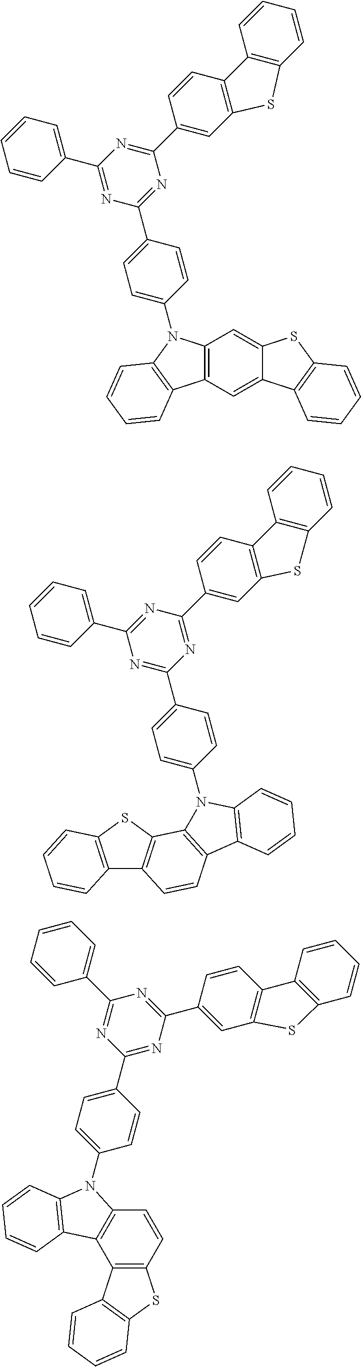 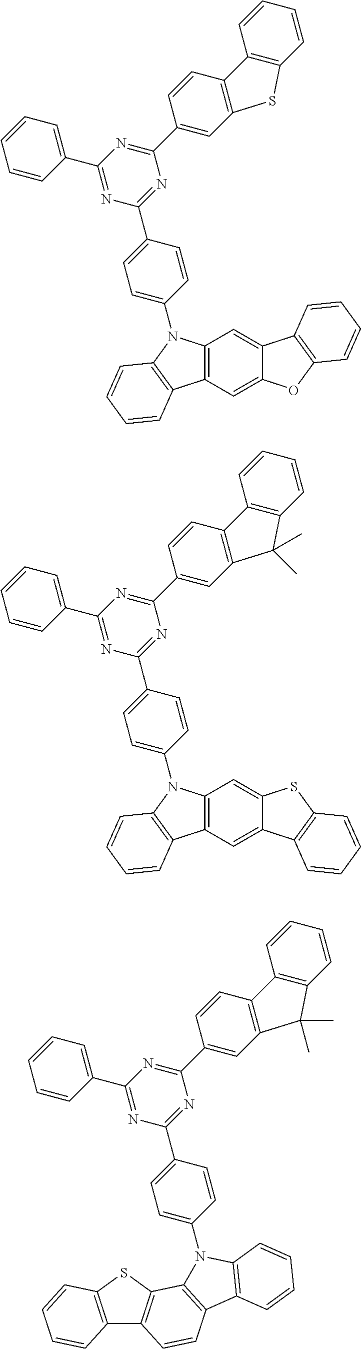 [SMILES: c1ccc(-c2nc(-c3ccc(-n4c5ccccc5c5cc6oc7ccccc7c6cc54)cc3)nc(-c3ccc4c(c3)sc3ccccc34)n2)cc1 ; CC1(C)c2ccccc2-c2ccc(-c3nc(-c4ccccc4)nc(-c4ccc(-n5c6ccccc6c6cc7c(cc65)sc5ccccc57)cc4)n3)cc21 ; CC1(C)c2ccccc2-c2ccc(-c3nc(-c4ccccc4)nc(-c4ccc(-n5c6ccccc6c6ccc7c8ccccc8sc7c65)cc4)n3)cc21] 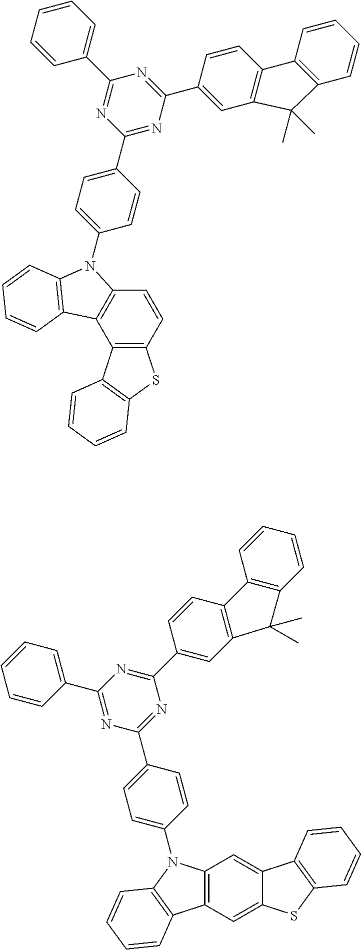 [SMILES: CC1(C)c2ccccc2-c2ccc(-c3nc(-c4ccccc4)nc(-c4ccc(-n5c6ccccc6c6c7c(ccc65)sc5ccccc57)cc4)n3)cc21 ; CC1(C)c2ccccc2-c2ccc(-c3nc(-c4ccccc4)nc(-c4ccc(-n5c6ccccc6c6cc7sc8ccccc8c7cc65)cc4)n3)cc21] 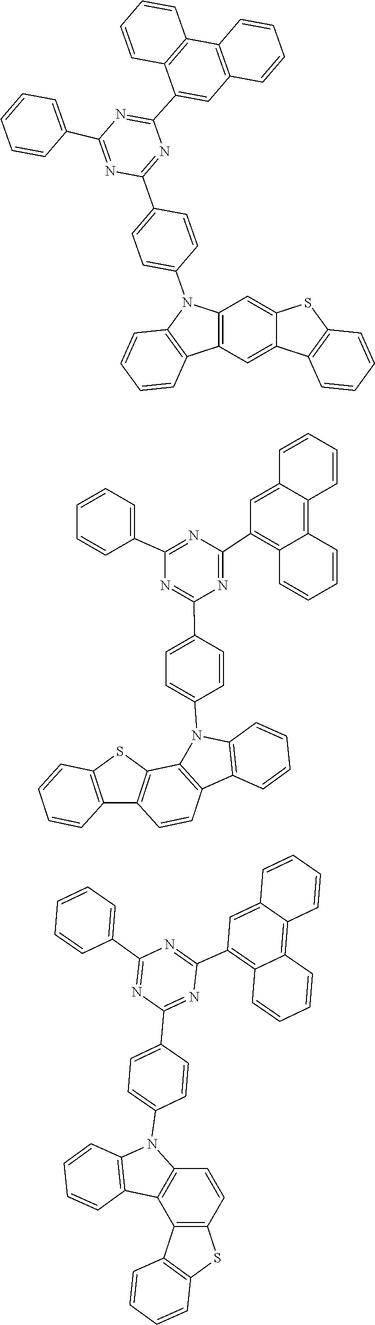 [SMILES: c1ccc(-c2nc(-c3ccc(-n4c5ccccc5c5cc6c(cc54)sc4ccccc46)cc3)nc(-c3cc4ccccc4c4ccccc34)n2)cc1 ; c1ccc(-c2nc(-c3ccc(-n4c5ccccc5c5ccc6c7ccccc7sc6c54)cc3)nc(-c3cc4ccccc4c4ccccc34)n2)cc1 ; c1ccc(-c2nc(-c3ccc(-n4c5ccccc5c5c6c(ccc54)sc4ccccc46)cc3)nc(-c3cc4ccccc4c4ccccc34)n2)cc1] 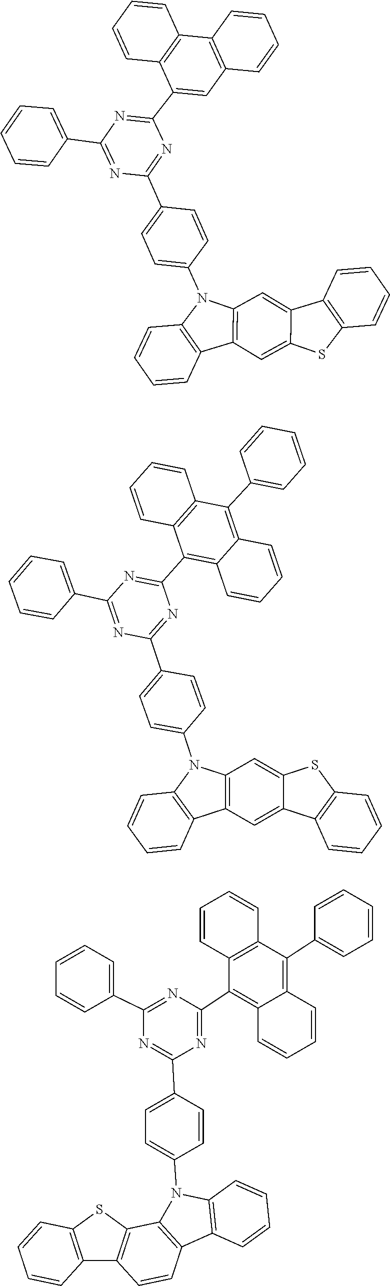 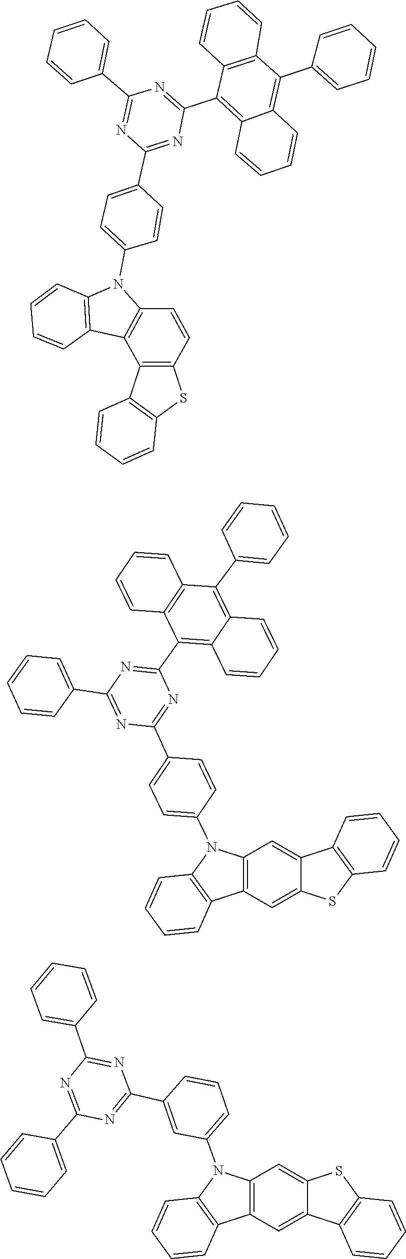 [SMILES: c1ccc(-c2nc(-c3ccc(-n4c5ccccc5c5c6c(ccc54)sc4ccccc46)cc3)nc(-c3c4ccccc4c(-c4ccccc4)c4ccccc34)n2)cc1 ; c1ccc(-c2nc(-c3ccc(-n4c5ccccc5c5cc6sc7ccccc7c6cc54)cc3)nc(-c3c4ccccc4c(-c4ccccc4)c4ccccc34)n2)cc1 ; c1ccc(-c2nc(-c3ccccc3)nc(-c3cccc(-n4c5ccccc5c5cc6c(cc54)sc4ccccc46)c3)n2)cc1] 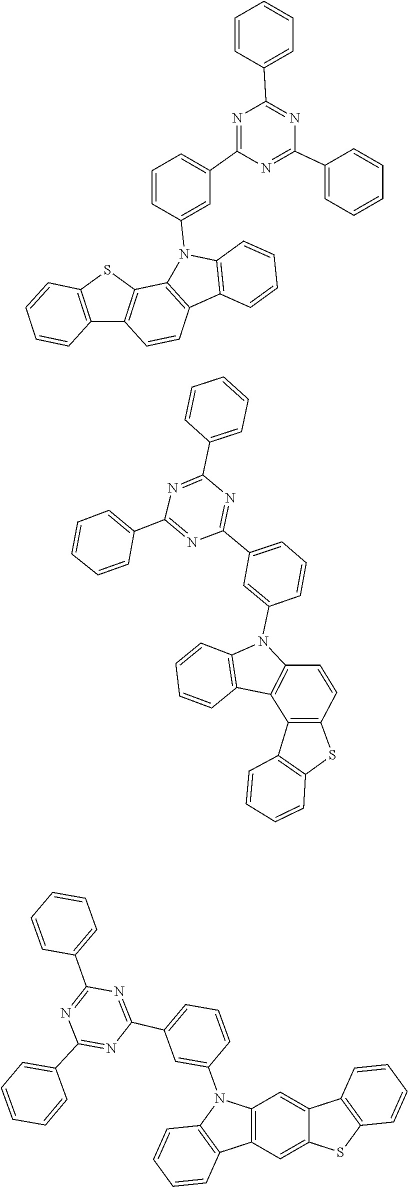 [SMILES: c1ccc(-c2nc(-c3ccccc3)nc(-c3cccc(-n4c5ccccc5c5ccc6c7ccccc7sc6c54)c3)n2)cc1 ; c1ccc(-c2nc(-c3ccccc3)nc(-c3cccc(-n4c5ccccc5c5c6c(ccc54)sc4ccccc46)c3)n2)cc1 ; c1ccc(-c2nc(-c3ccccc3)nc(-c3cccc(-n4c5ccccc5c5cc6sc7ccccc7c6cc54)c3)n2)cc1] 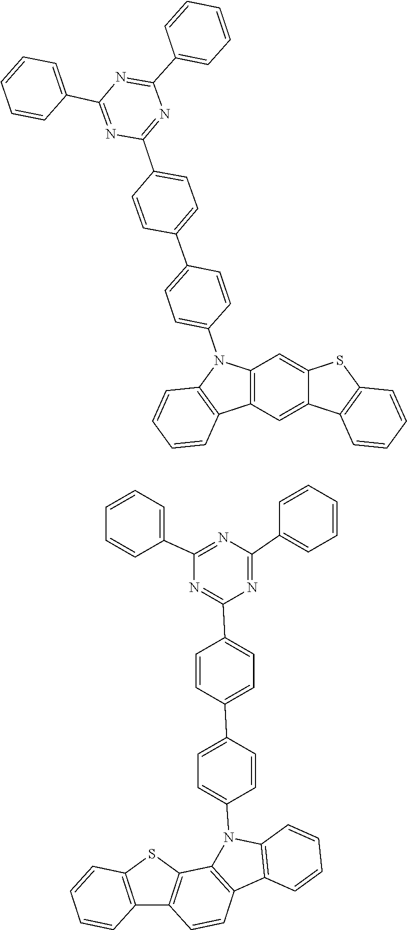 [SMILES: c1ccc(-c2nc(-c3ccccc3)nc(-c3ccc(-c4ccc(-n5c6ccccc6c6cc7c(cc65)sc5ccccc57)cc4)cc3)n2)cc1 ; c1ccc(-c2nc(-c3ccccc3)nc(-c3ccc(-c4ccc(-n5c6ccccc6c6ccc7c8ccccc8sc7c65)cc4)cc3)n2)cc1] 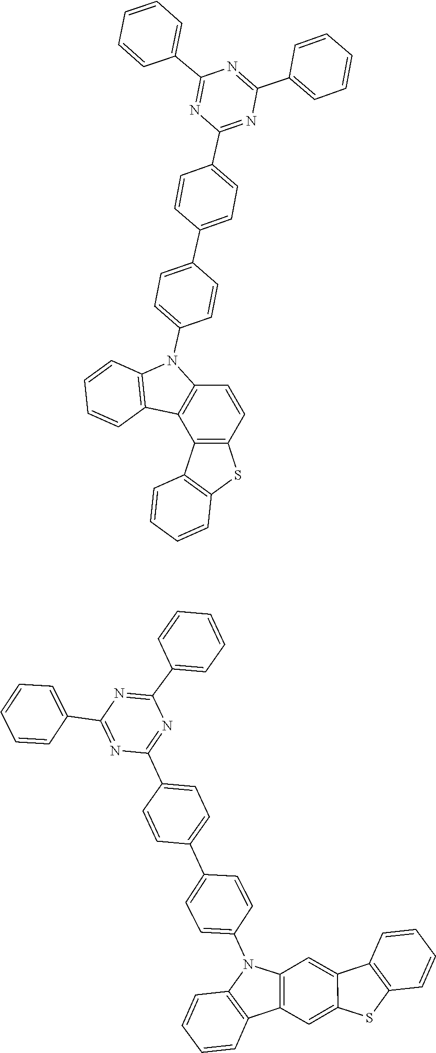 [SMILES: c1ccc(-c2nc(-c3ccccc3)nc(-c3ccc(-c4ccc(-n5c6ccccc6c6c7c(ccc65)sc5ccccc57)cc4)cc3)n2)cc1 ; c1ccc(-c2nc(-c3ccccc3)nc(-c3ccc(-c4ccc(-n5c6ccccc6c6cc7sc8ccccc8c7cc65)cc4)cc3)n2)cc1] 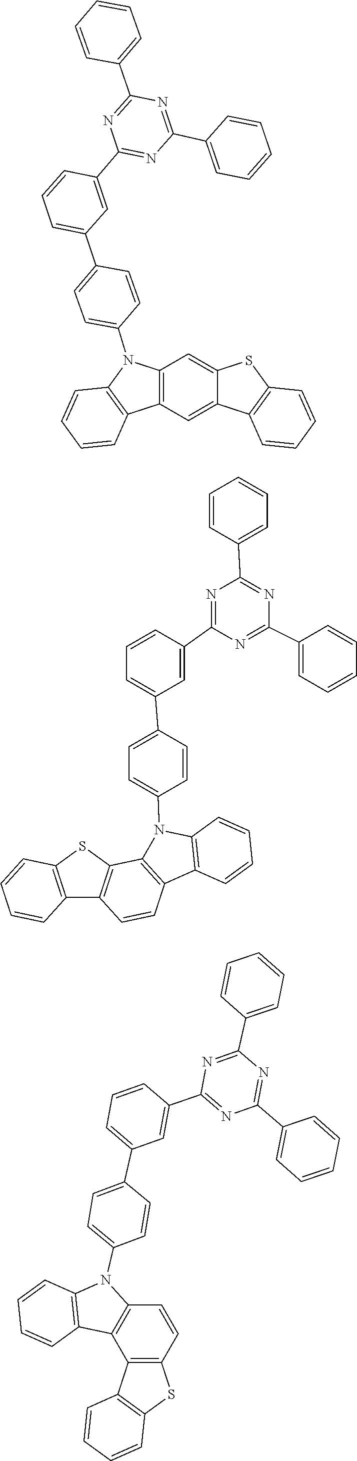 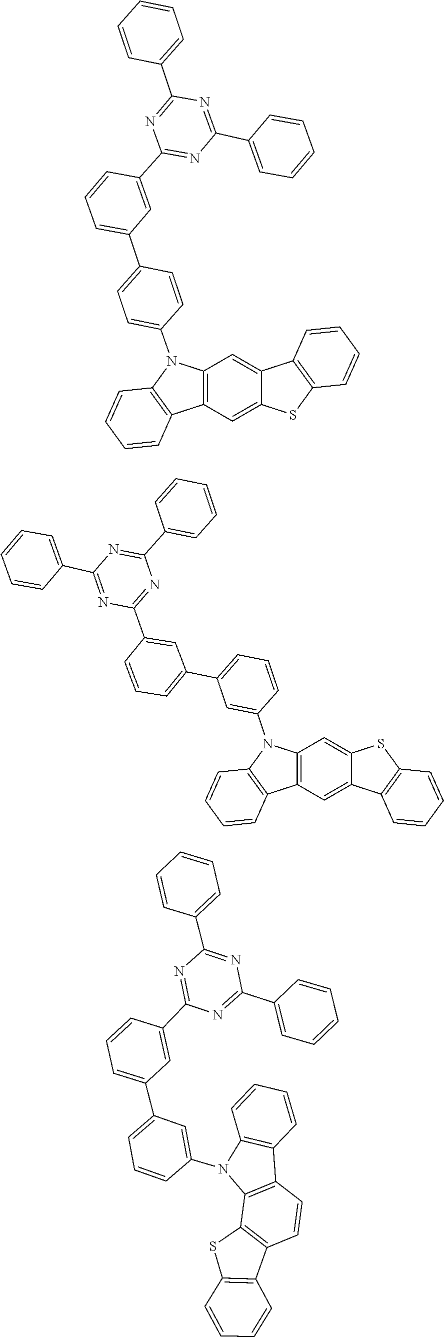 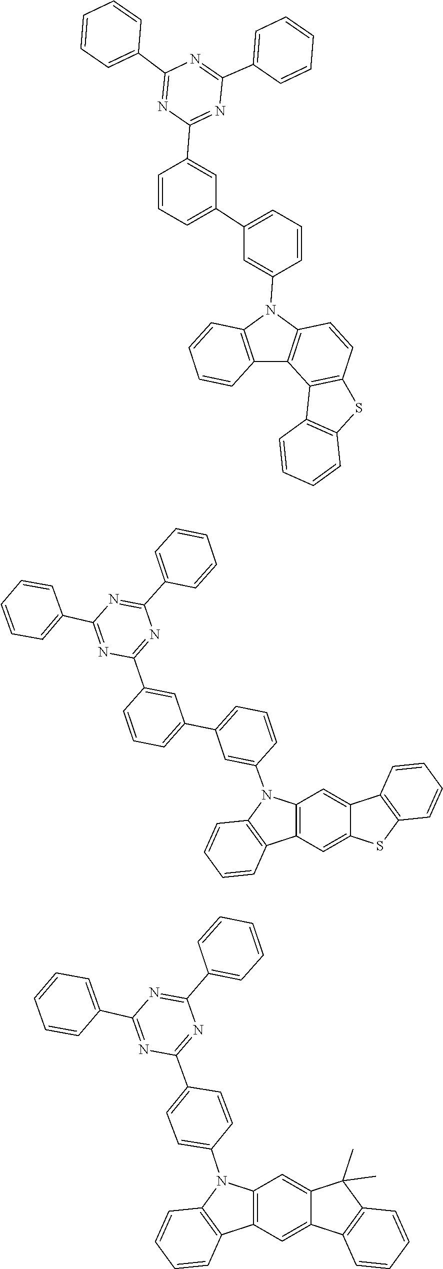 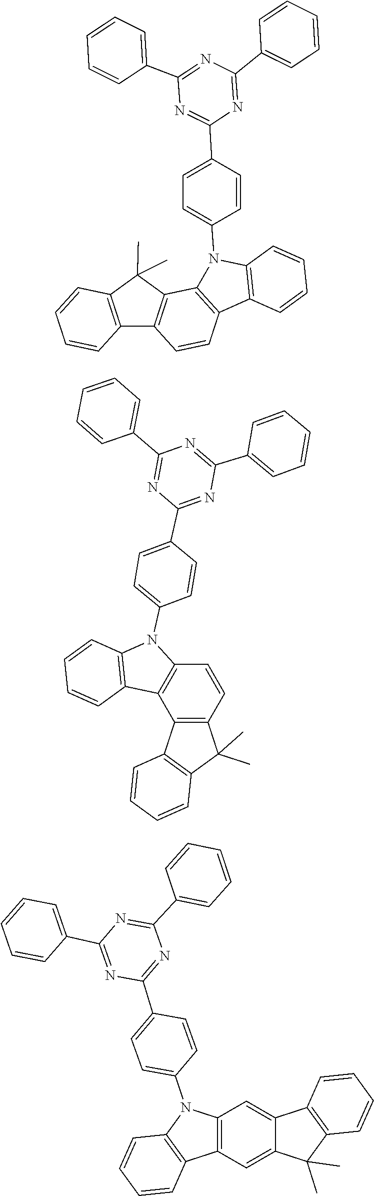 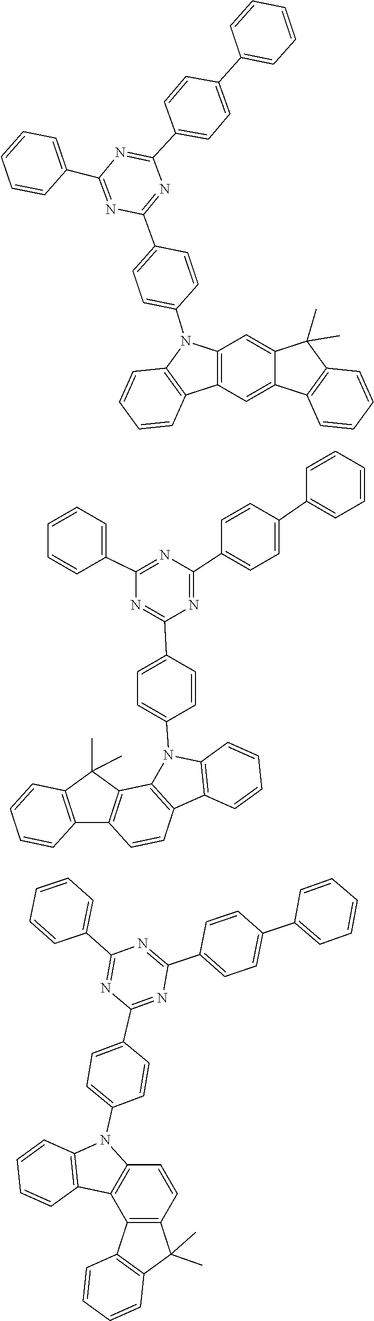 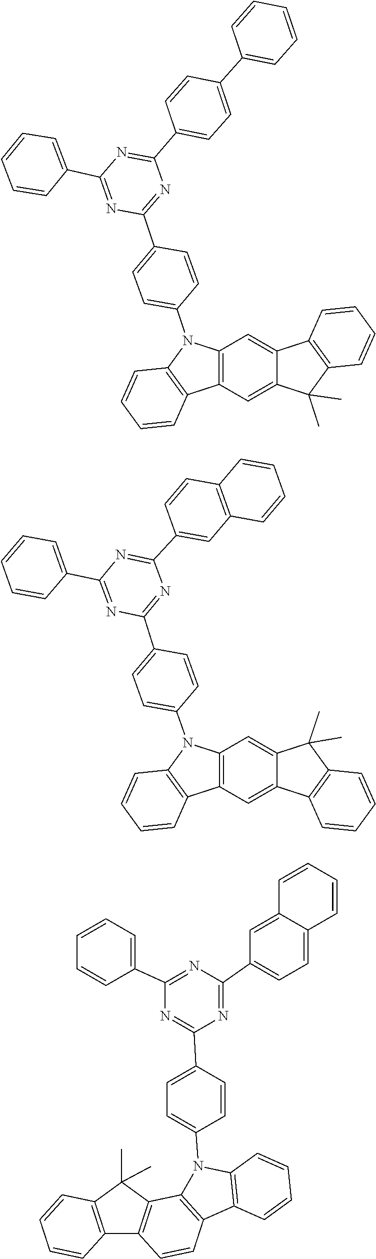 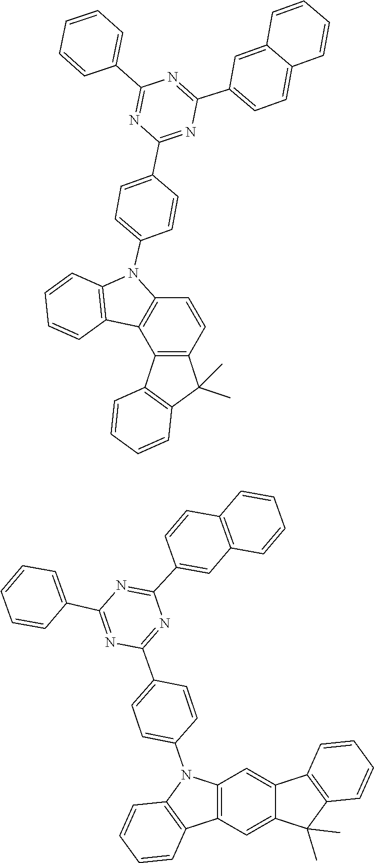 [SMILES: CC1(C)c2ccccc2-c2c1ccc1c2c2ccccc2n1-c1ccc(-c2nc(-c3ccccc3)nc(-c3ccc4ccccc4c3)n2)cc1 ; CC1(C)c2ccccc2-c2cc3c(cc21)c1ccccc1n3-c1ccc(-c2nc(-c3ccccc3)nc(-c3ccc4ccccc4c3)n2)cc1] 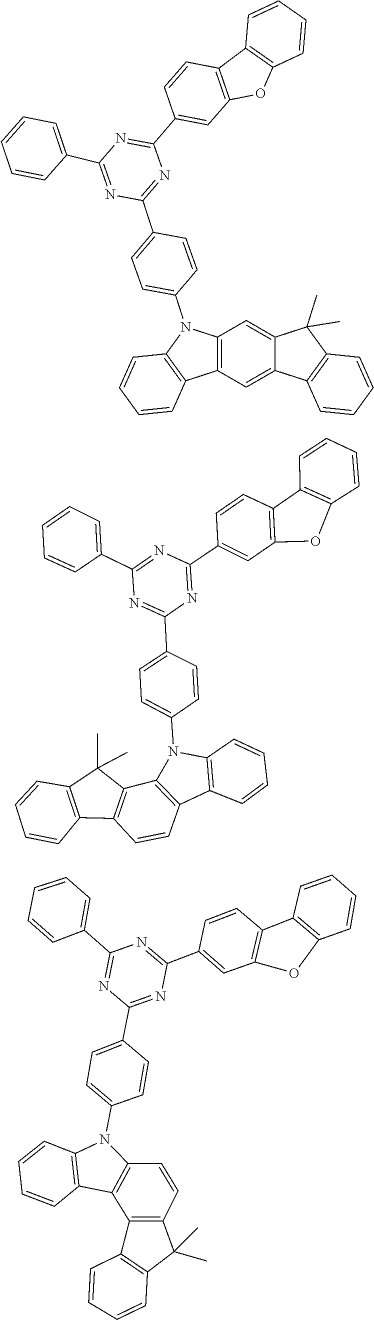 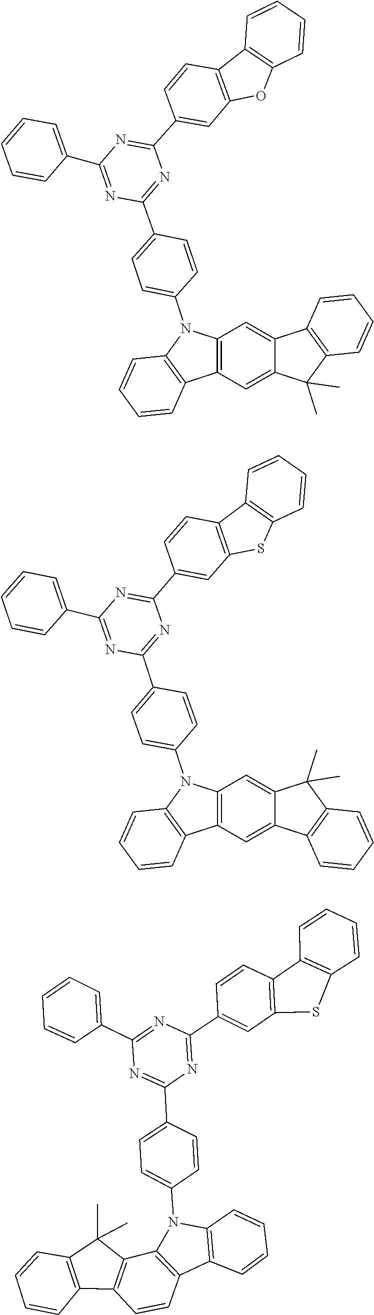 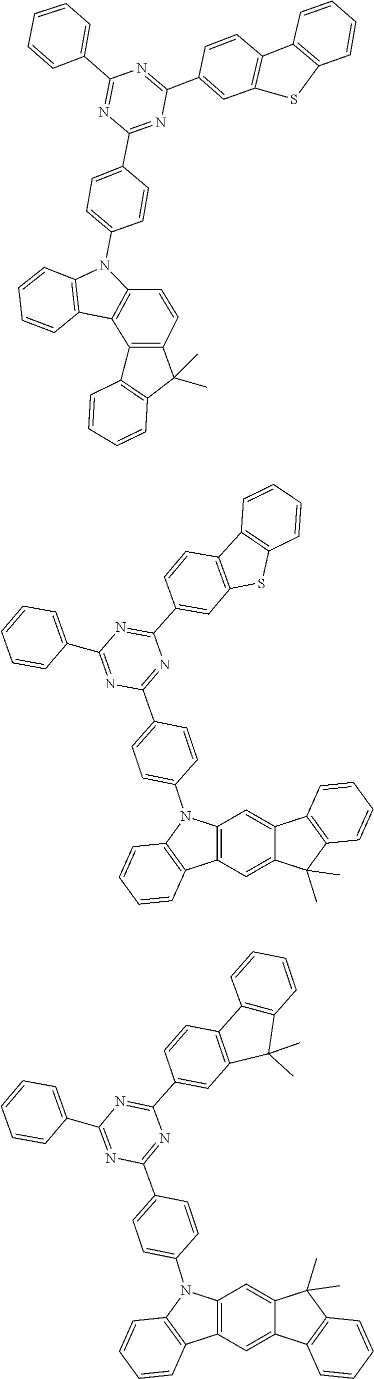 [SMILES: CC1(C)c2ccccc2-c2c1ccc1c2c2ccccc2n1-c1ccc(-c2nc(-c3ccccc3)nc(-c3ccc4c(c3)sc3ccccc34)n2)cc1 ; CC1(C)c2ccccc2-c2cc3c(cc21)c1ccccc1n3-c1ccc(-c2nc(-c3ccccc3)nc(-c3ccc4c(c3)sc3ccccc34)n2)cc1 ; CC1(C)c2ccccc2-c2ccc(-c3nc(-c4ccccc4)nc(-c4ccc(-n5c6ccccc6c6cc7c(cc65)C(C)(C)c5ccccc5-7)cc4)n3)cc21] 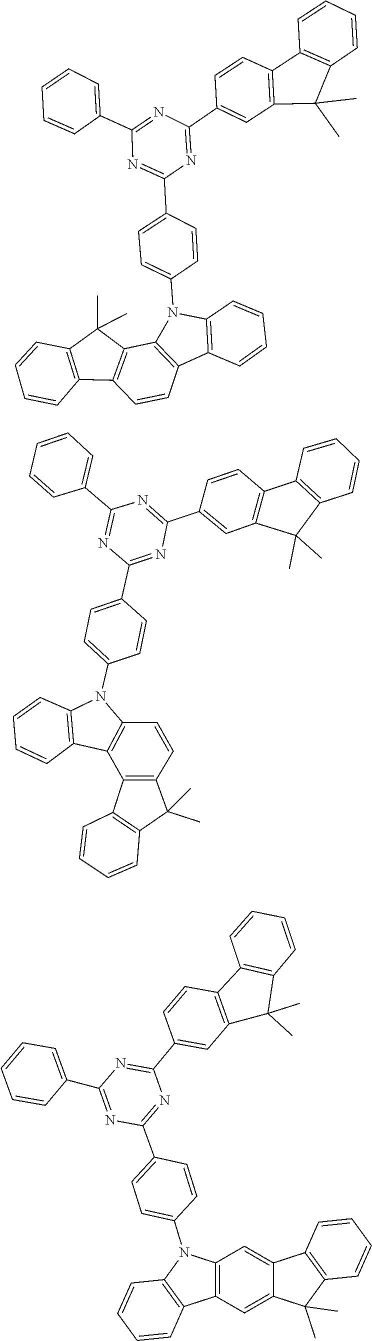 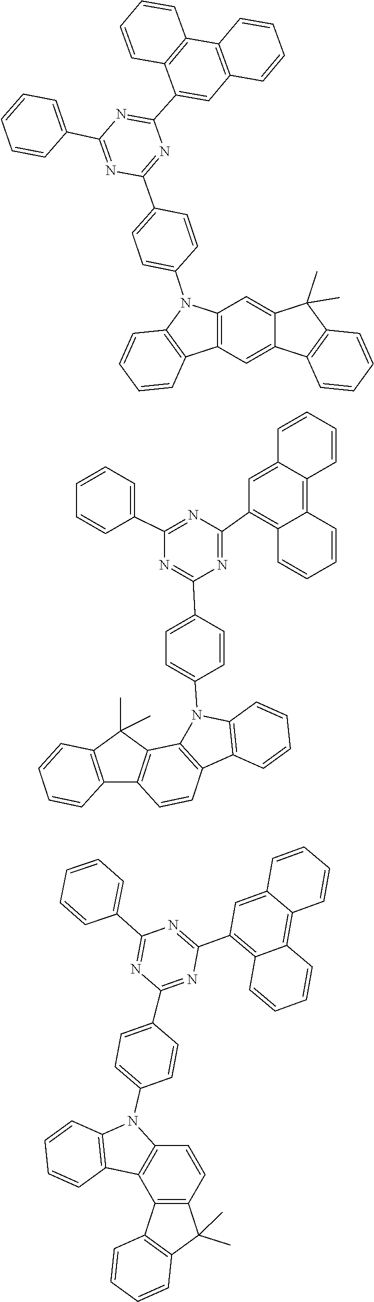 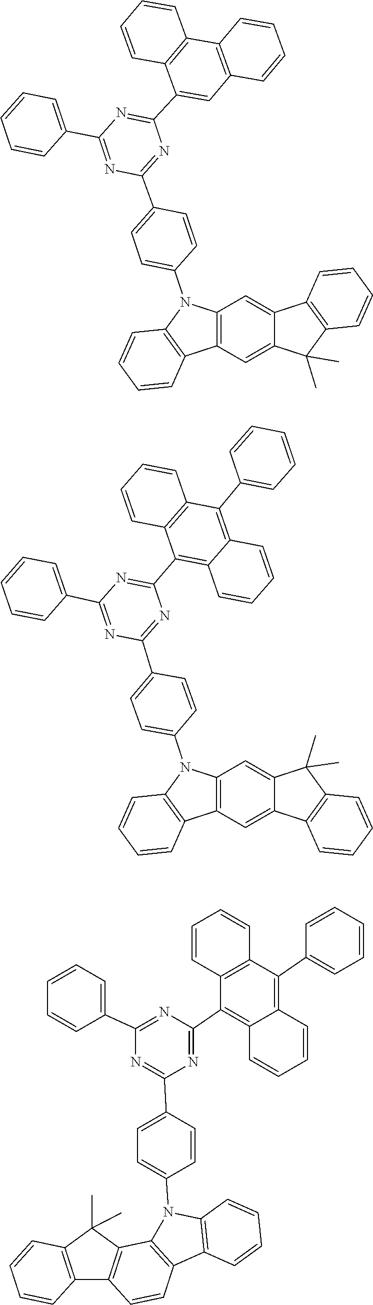 [SMILES: CC1(C)c2ccccc2-c2cc3c(cc21)c1ccccc1n3-c1ccc(-c2nc(-c3ccccc3)nc(-c3cc4ccccc4c4ccccc34)n2)cc1 ; CC1(C)c2ccccc2-c2cc3c4ccccc4n(-c4ccc(-c5nc(-c6ccccc6)nc(-c6c7ccccc7c(-c7ccccc7)c7ccccc67)n5)cc4)c3cc21 ; CC1(C)c2ccccc2-c2ccc3c4ccccc4n(-c4ccc(-c5nc(-c6ccccc6)nc(-c6c7ccccc7c(-c7ccccc7)c7ccccc67)n5)cc4)c3c21] 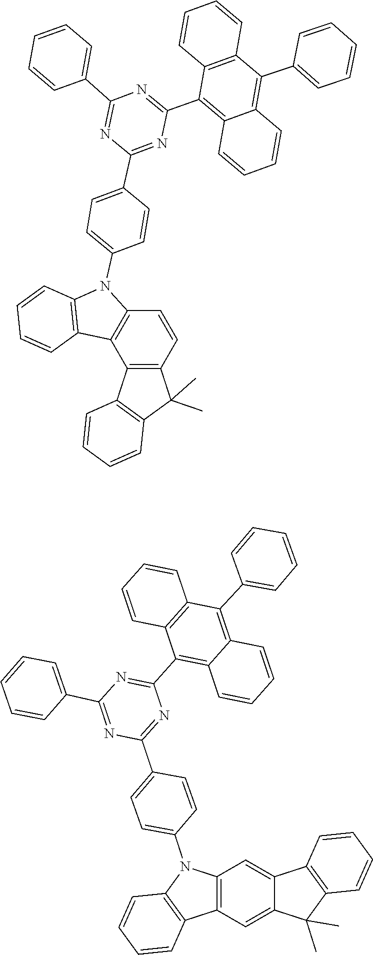 [SMILES: CC1(C)c2ccccc2-c2c1ccc1c2c2ccccc2n1-c1ccc(-c2nc(-c3ccccc3)nc(-c3c4ccccc4c(-c4ccccc4)c4ccccc34)n2)cc1 ; CC1(C)c2ccccc2-c2cc3c(cc21)c1ccccc1n3-c1ccc(-c2nc(-c3ccccc3)nc(-c3c4ccccc4c(-c4ccccc4)c4ccccc34)n2)cc1] 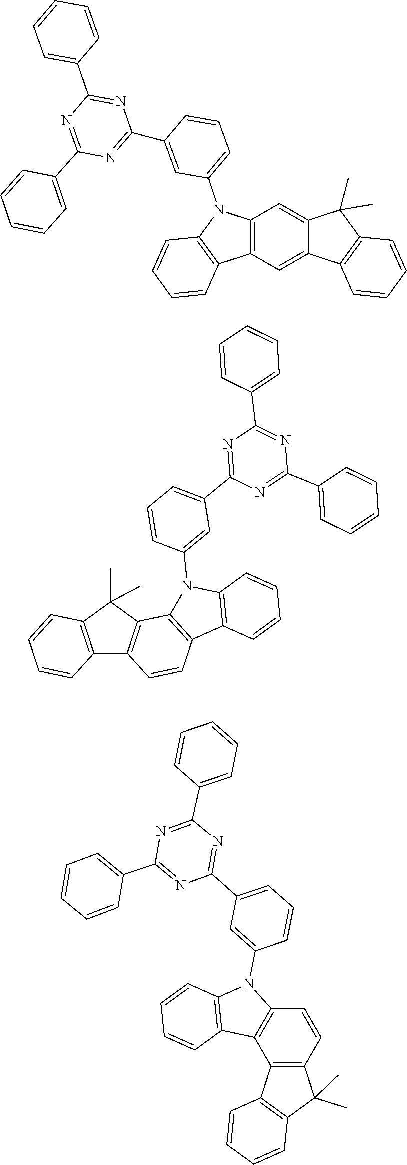 [SMILES: CC1(C)c2ccccc2-c2cc3c4ccccc4n(-c4cccc(-c5nc(-c6ccccc6)nc(-c6ccccc6)n5)c4)c3cc21 ; CC1(C)c2ccccc2-c2ccc3c4ccccc4n(-c4cccc(-c5nc(-c6ccccc6)nc(-c6ccccc6)n5)c4)c3c21 ; CC1(C)c2ccccc2-c2c1ccc1c2c2ccccc2n1-c1cccc(-c2nc(-c3ccccc3)nc(-c3ccccc3)n2)c1] 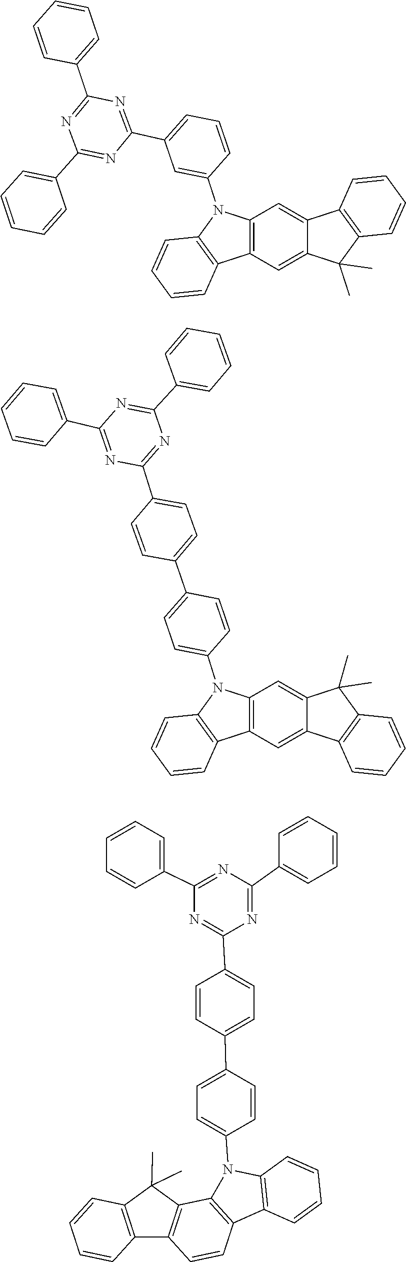 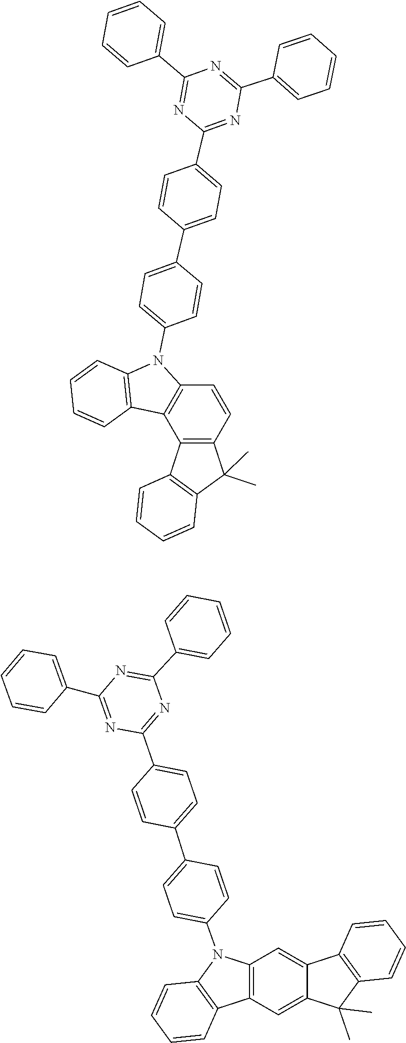 [SMILES: CC1(C)c2ccccc2-c2c1ccc1c2c2ccccc2n1-c1ccc(-c2ccc(-c3nc(-c4ccccc4)nc(-c4ccccc4)n3)cc2)cc1 ; CC1(C)c2ccccc2-c2cc3c(cc21)c1ccccc1n3-c1ccc(-c2ccc(-c3nc(-c4ccccc4)nc(-c4ccccc4)n3)cc2)cc1] 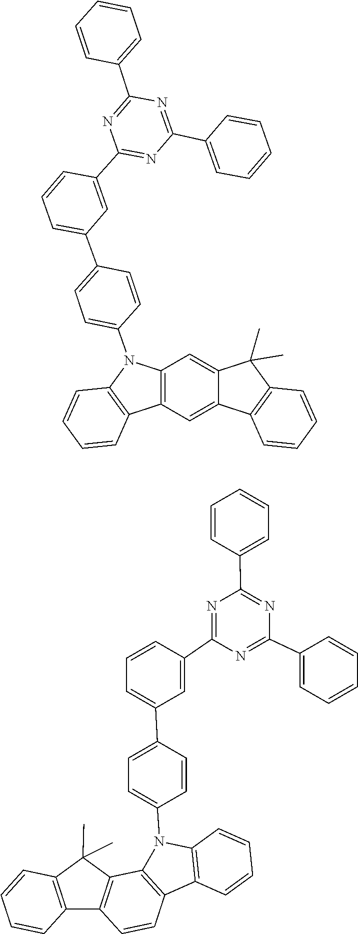 [SMILES: CC1(C)c2ccccc2-c2cc3c4ccccc4n(-c4ccc(-c5cccc(-c6nc(-c7ccccc7)nc(-c7ccccc7)n6)c5)cc4)c3cc21 ; CC1(C)c2ccccc2-c2ccc3c4ccccc4n(-c4ccc(-c5cccc(-c6nc(-c7ccccc7)nc(-c7ccccc7)n6)c5)cc4)c3c21] 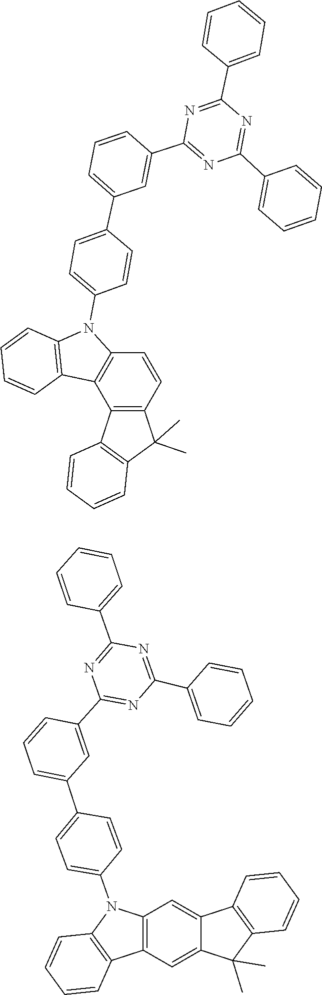 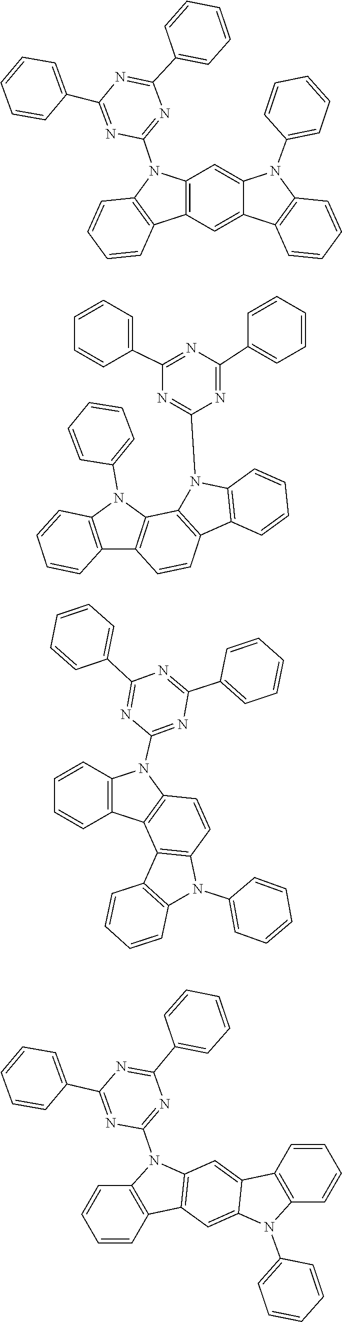 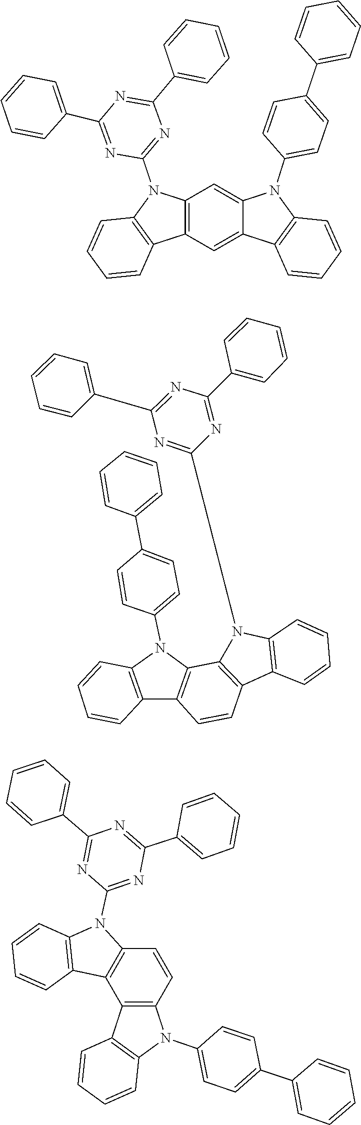 [SMILES: c1ccc(-c2ccc(-n3c4ccccc4c4cc5c6ccccc6n(-c6nc(-c7ccccc7)nc(-c7ccccc7)n6)c5cc43)cc2)cc1 ; c1ccc(-c2ccc(-n3c4ccccc4c4ccc5c6ccccc6n(-c6nc(-c7ccccc7)nc(-c7ccccc7)n6)c5c43)cc2)cc1 ; c1ccc(-c2ccc(-n3c4ccccc4c4c5c6ccccc6n(-c6nc(-c7ccccc7)nc(-c7ccccc7)n6)c5ccc43)cc2)cc1] 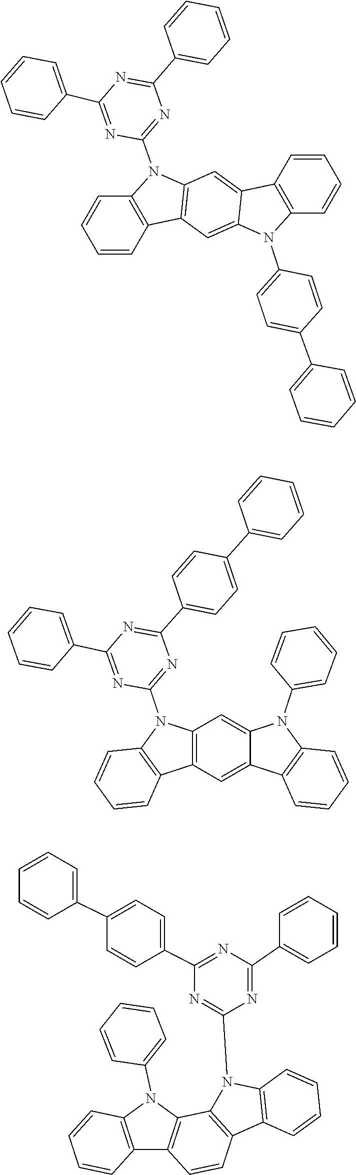 [SMILES: c1ccc(-c2ccc(-n3c4ccccc4c4cc5c(cc43)c3ccccc3n5-c3nc(-c4ccccc4)nc(-c4ccccc4)n3)cc2)cc1 ; c1ccc(-c2ccc(-c3nc(-c4ccccc4)nc(-n4c5ccccc5c5cc6c7ccccc7n(-c7ccccc7)c6cc54)n3)cc2)cc1 ; c1ccc(-c2ccc(-c3nc(-c4ccccc4)nc(-n4c5ccccc5c5ccc6c7ccccc7n(-c7ccccc7)c6c54)n3)cc2)cc1] 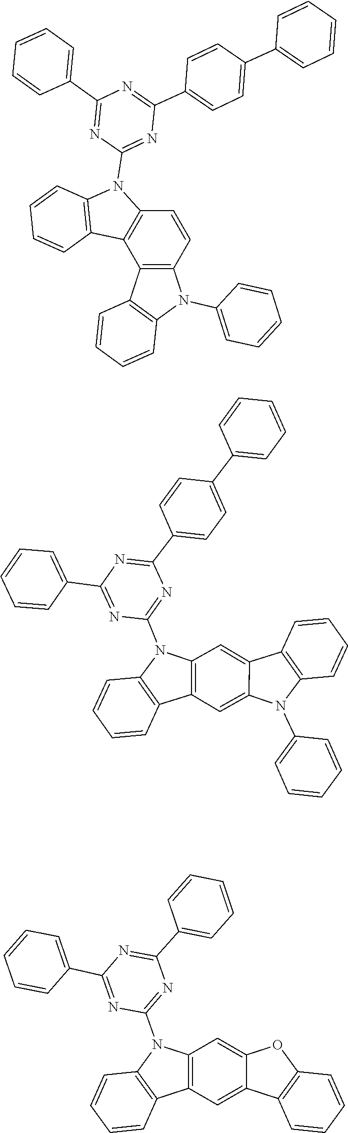 [SMILES: c1ccc(-c2ccc(-c3nc(-c4ccccc4)nc(-n4c5ccccc5c5c6c7ccccc7n(-c7ccccc7)c6ccc54)n3)cc2)cc1 ; c1ccc(-c2ccc(-c3nc(-c4ccccc4)nc(-n4c5ccccc5c5cc6c(cc54)c4ccccc4n6-c4ccccc4)n3)cc2)cc1 ; c1ccc(-c2nc(-c3ccccc3)nc(-n3c4ccccc4c4cc5c(cc43)oc3ccccc35)n2)cc1] 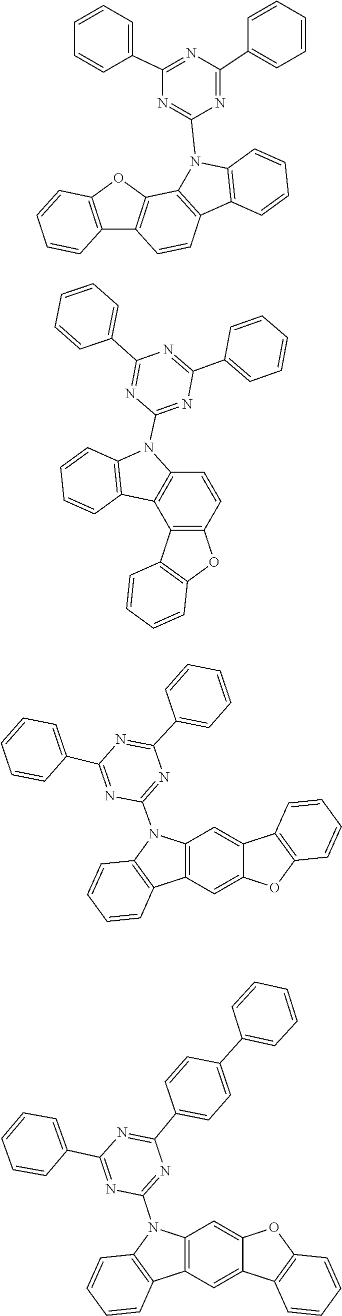 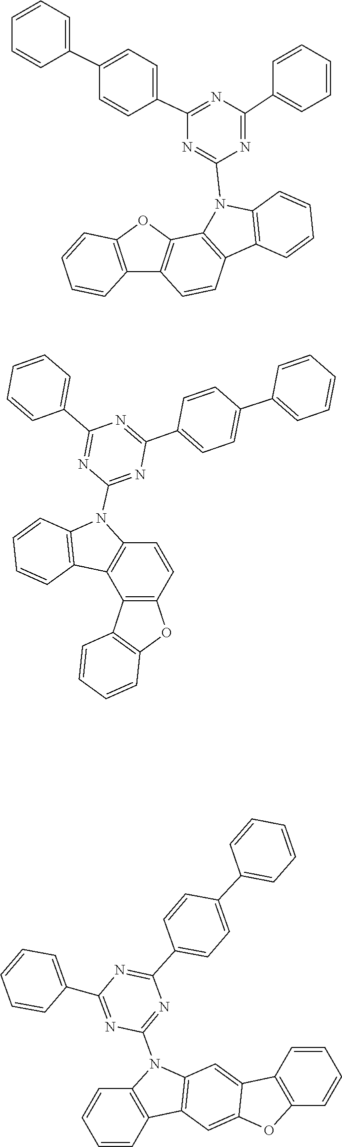 [SMILES: c1ccc(-c2ccc(-c3nc(-c4ccccc4)nc(-n4c5ccccc5c5ccc6c7ccccc7oc6c54)n3)cc2)cc1 ; c1ccc(-c2ccc(-c3nc(-c4ccccc4)nc(-n4c5ccccc5c5c6c(ccc54)oc4ccccc46)n3)cc2)cc1 ; c1ccc(-c2ccc(-c3nc(-c4ccccc4)nc(-n4c5ccccc5c5cc6oc7ccccc7c6cc54)n3)cc2)cc1] 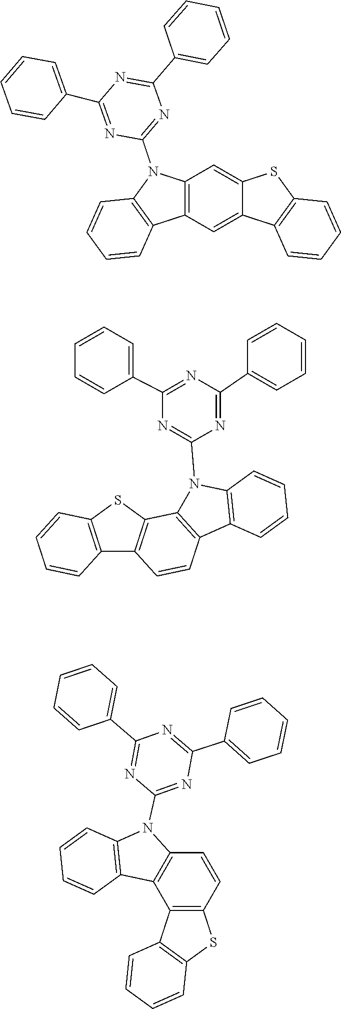 [SMILES: c1ccc(-c2nc(-c3ccccc3)nc(-n3c4ccccc4c4cc5c(cc43)sc3ccccc35)n2)cc1 ; c1ccc(-c2nc(-c3ccccc3)nc(-n3c4ccccc4c4ccc5c6ccccc6sc5c43)n2)cc1 ; c1ccc(-c2nc(-c3ccccc3)nc(-n3c4ccccc4c4c5c(ccc43)sc3ccccc35)n2)cc1] 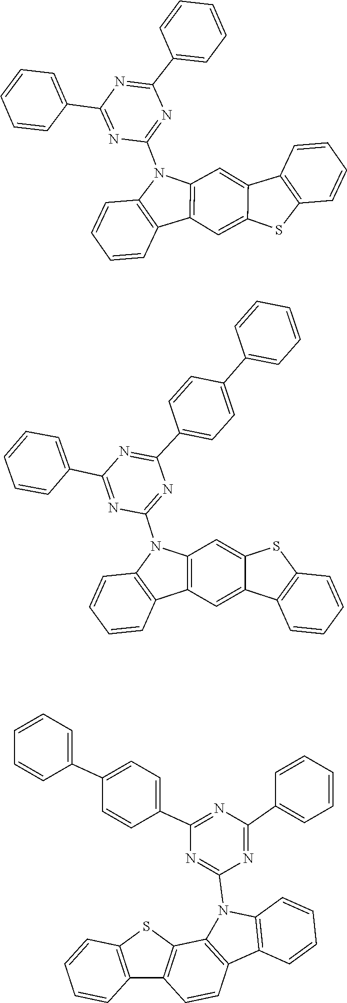 [SMILES: c1ccc(-c2nc(-c3ccccc3)nc(-n3c4ccccc4c4cc5sc6ccccc6c5cc43)n2)cc1 ; c1ccc(-c2ccc(-c3nc(-c4ccccc4)nc(-n4c5ccccc5c5cc6c(cc54)sc4ccccc46)n3)cc2)cc1 ; c1ccc(-c2ccc(-c3nc(-c4ccccc4)nc(-n4c5ccccc5c5ccc6c7ccccc7sc6c54)n3)cc2)cc1] 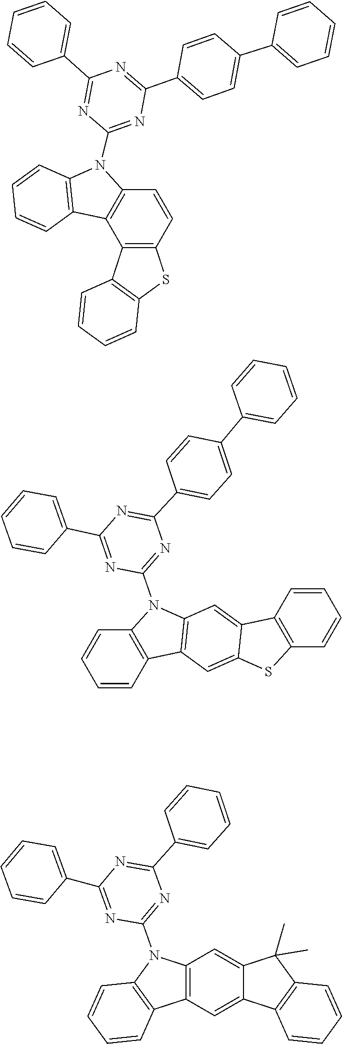 [SMILES: c1ccc(-c2ccc(-c3nc(-c4ccccc4)nc(-n4c5ccccc5c5c6c(ccc54)sc4ccccc46)n3)cc2)cc1 ; c1ccc(-c2ccc(-c3nc(-c4ccccc4)nc(-n4c5ccccc5c5cc6sc7ccccc7c6cc54)n3)cc2)cc1 ; CC1(C)c2ccccc2-c2cc3c4ccccc4n(-c4nc(-c5ccccc5)nc(-c5ccccc5)n4)c3cc21] 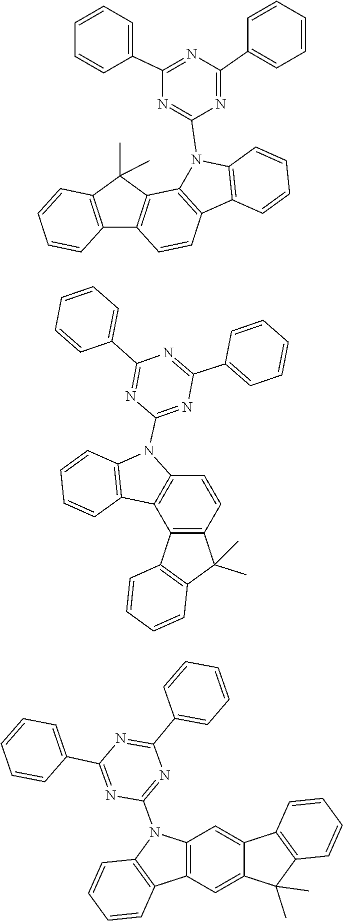 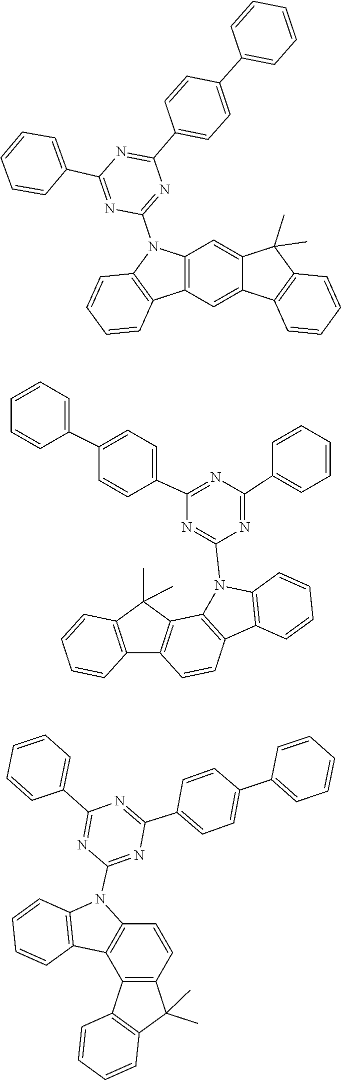 [SMILES: CC1(C)c2ccccc2-c2cc3c4ccccc4n(-c4nc(-c5ccccc5)nc(-c5ccc(-c6ccccc6)cc5)n4)c3cc21 ; CC1(C)c2ccccc2-c2ccc3c4ccccc4n(-c4nc(-c5ccccc5)nc(-c5ccc(-c6ccccc6)cc5)n4)c3c21 ; CC1(C)c2ccccc2-c2c1ccc1c2c2ccccc2n1-c1nc(-c2ccccc2)nc(-c2ccc(-c3ccccc3)cc2)n1] 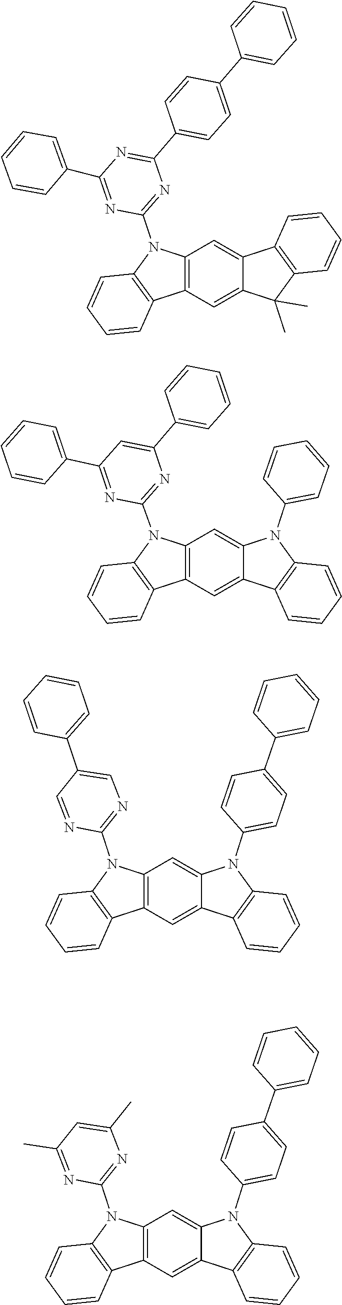 [SMILES: CC1(C)c2ccccc2-c2cc3c(cc21)c1ccccc1n3-c1nc(-c2ccccc2)nc(-c2ccc(-c3ccccc3)cc2)n1 ; c1ccc(-c2cc(-c3ccccc3)nc(-n3c4ccccc4c4cc5c6ccccc6n(-c6ccccc6)c5cc43)n2)cc1 ; c1ccc(-c2ccc(-n3c4ccccc4c4cc5c6ccccc6n(-c6ncc(-c7ccccc7)cn6)c5cc43)cc2)cc1 ; Cc1cc(C)nc(-n2c3ccccc3c3cc4c5ccccc5n(-c5ccc(-c6ccccc6)cc5)c4cc32)n1] 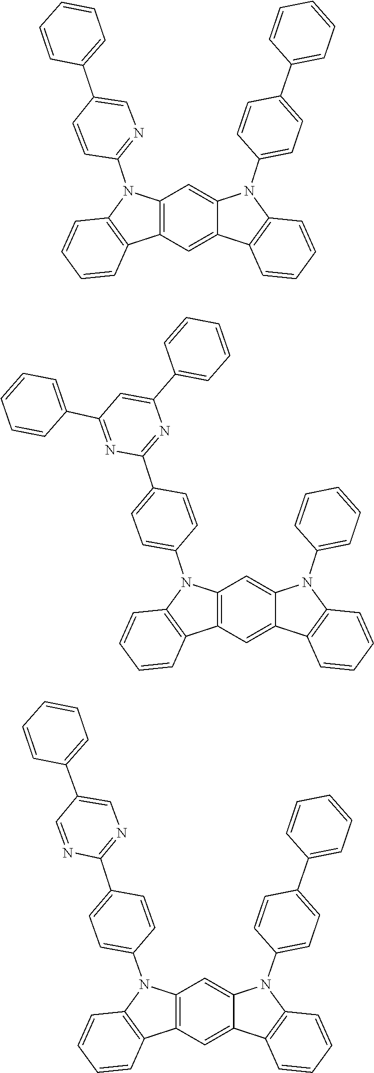 [SMILES: c1ccc(-c2ccc(-n3c4ccccc4c4cc5c6ccccc6n(-c6ccc(-c7ccccc7)cn6)c5cc43)cc2)cc1 ; c1ccc(-c2cc(-c3ccccc3)nc(-c3ccc(-n4c5ccccc5c5cc6c7ccccc7n(-c7ccccc7)c6cc54)cc3)n2)cc1 ; c1ccc(-c2ccc(-n3c4ccccc4c4cc5c6ccccc6n(-c6ccc(-c7ncc(-c8ccccc8)cn7)cc6)c5cc43)cc2)cc1] 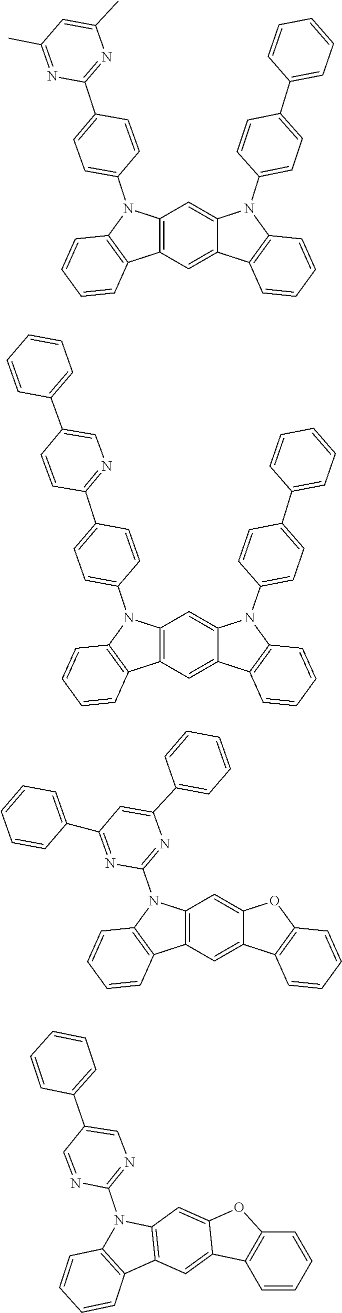 [SMILES: Cc1cc(C)nc(-c2ccc(-n3c4ccccc4c4cc5c6ccccc6n(-c6ccc(-c7ccccc7)cc6)c5cc43)cc2)n1 ; c1ccc(-c2ccc(-n3c4ccccc4c4cc5c6ccccc6n(-c6ccc(-c7ccc(-c8ccccc8)cn7)cc6)c5cc43)cc2)cc1 ; c1ccc(-c2cc(-c3ccccc3)nc(-n3c4ccccc4c4cc5c(cc43)oc3ccccc35)n2)cc1 ; c1ccc(-c2cnc(-n3c4ccccc4c4cc5c(cc43)oc3ccccc35)nc2)cc1] 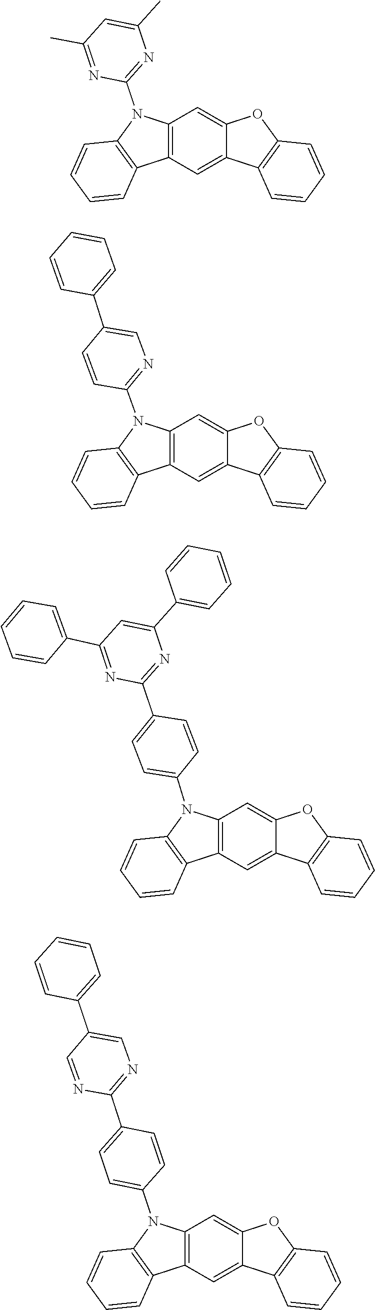 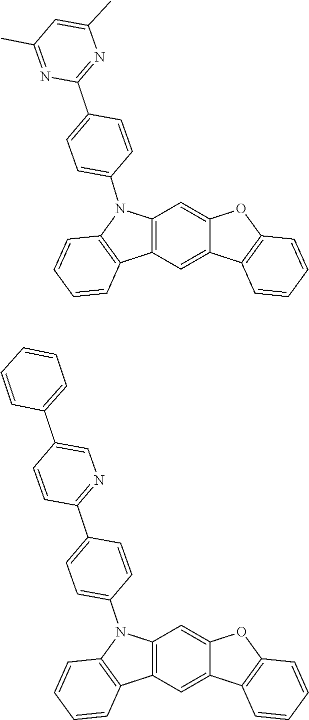 [SMILES: Cc1cc(C)nc(-c2ccc(-n3c4ccccc4c4cc5c(cc43)oc3ccccc35)cc2)n1 ; c1ccc(-c2ccc(-c3ccc(-n4c5ccccc5c5cc6c(cc54)oc4ccccc46)cc3)nc2)cc1] 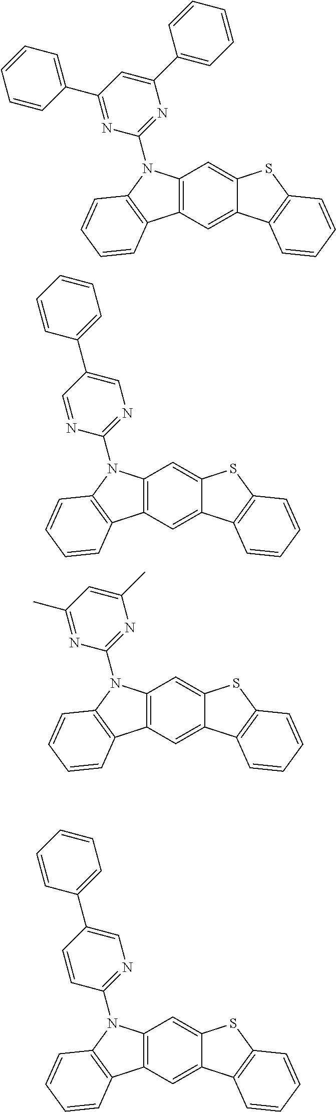 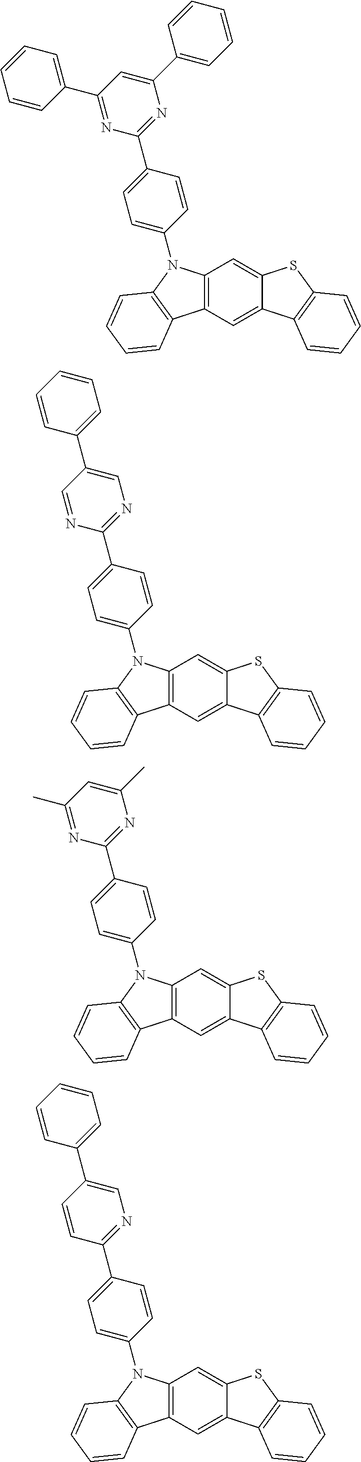 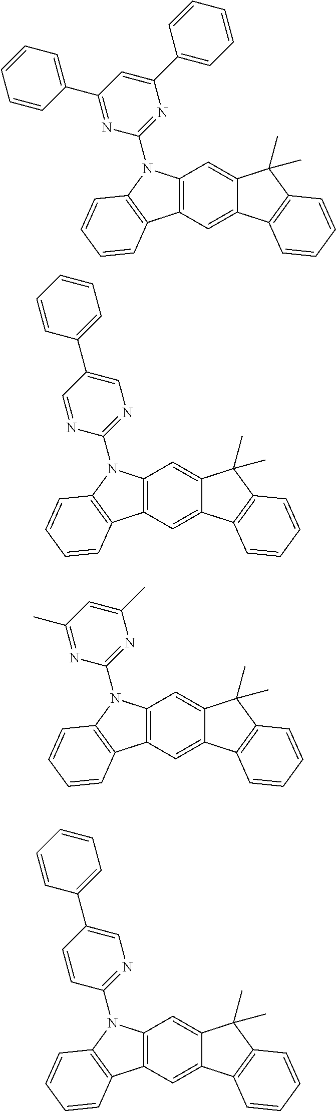 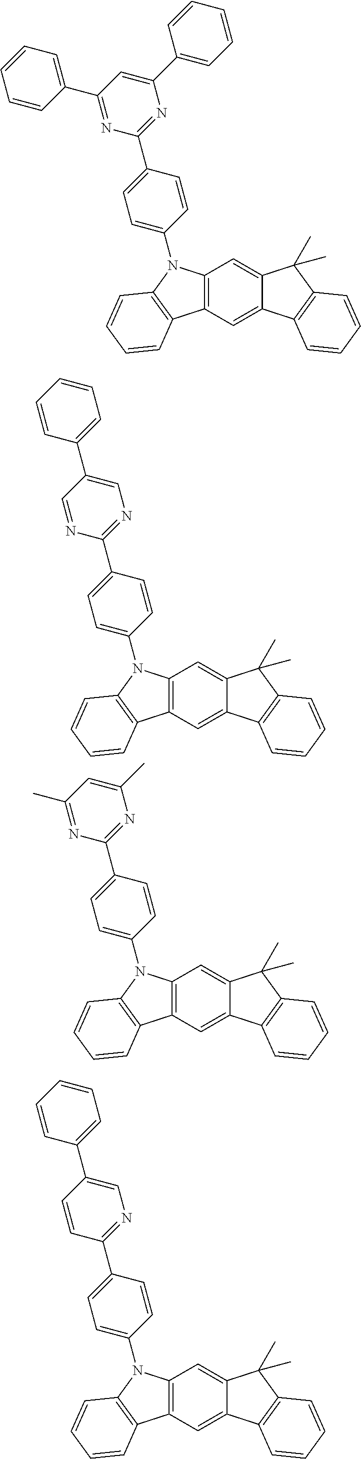 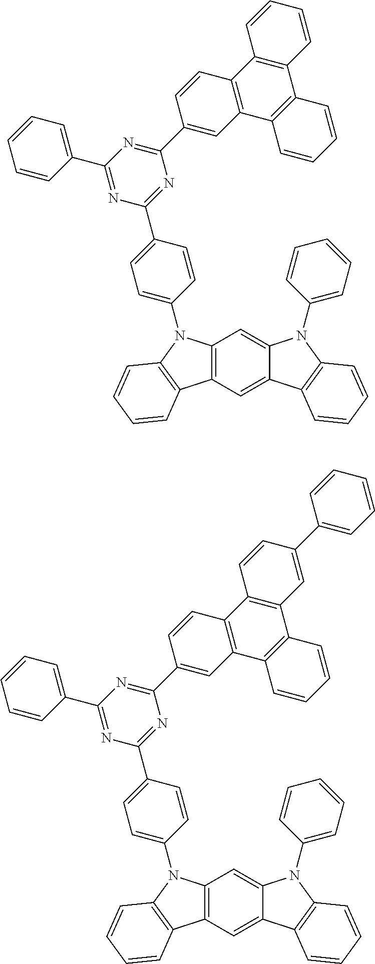 [SMILES: c1ccc(-c2nc(-c3ccc(-n4c5ccccc5c5cc6c7ccccc7n(-c7ccccc7)c6cc54)cc3)nc(-c3ccc4c5ccccc5c5ccccc5c4c3)n2)cc1 ; c1ccc(-c2ccc3c4ccc(-c5nc(-c6ccccc6)nc(-c6ccc(-n7c8ccccc8c8cc9c%10ccccc%10n(-c%10ccccc%10)c9cc87)cc6)n5)cc4c4ccccc4c3c2)cc1] 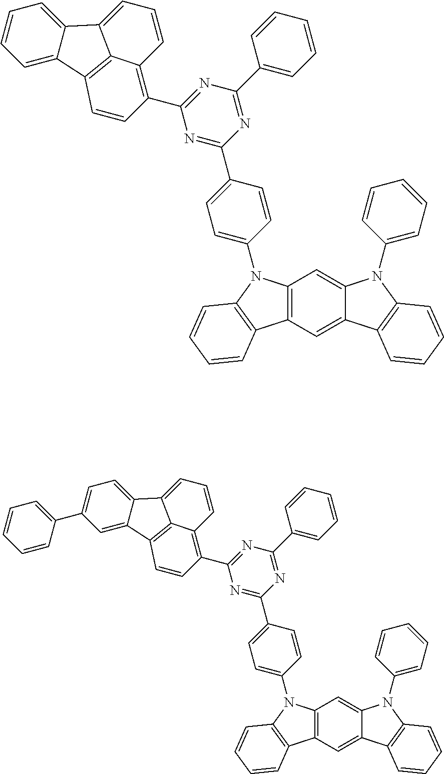 [SMILES: c1ccc(-c2nc(-c3ccc(-n4c5ccccc5c5cc6c7ccccc7n(-c7ccccc7)c6cc54)cc3)nc(-c3ccc4c5c(cccc35)-c3ccccc3-4)n2)cc1 ; c1ccc(-c2ccc3c(c2)-c2ccc(-c4nc(-c5ccccc5)nc(-c5ccc(-n6c7ccccc7c7cc8c9ccccc9n(-c9ccccc9)c8cc76)cc5)n4)c4cccc-3c24)cc1] 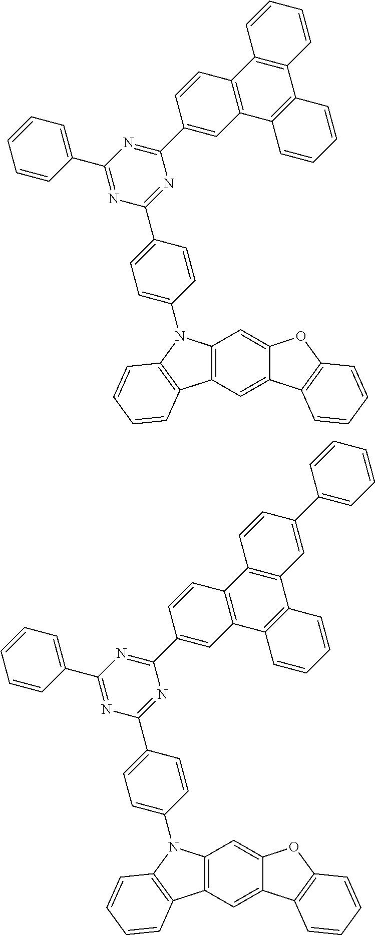 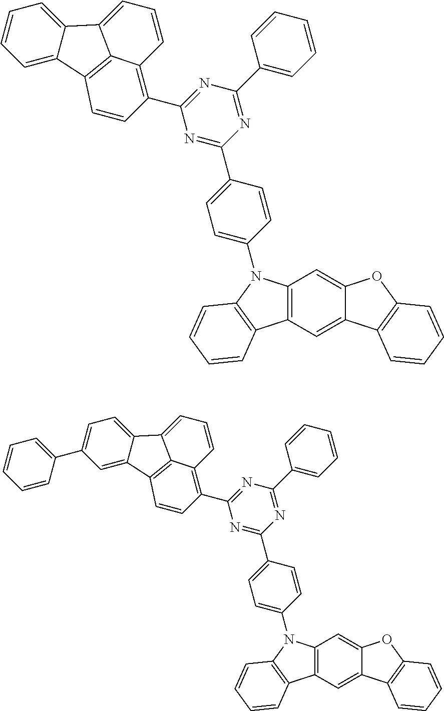 [SMILES: c1ccc(-c2nc(-c3ccc(-n4c5ccccc5c5cc6c(cc54)oc4ccccc46)cc3)nc(-c3ccc4c5c(cccc35)-c3ccccc3-4)n2)cc1 ; c1ccc(-c2ccc3c(c2)-c2ccc(-c4nc(-c5ccccc5)nc(-c5ccc(-n6c7ccccc7c7cc8c(cc76)oc6ccccc68)cc5)n4)c4cccc-3c24)cc1] 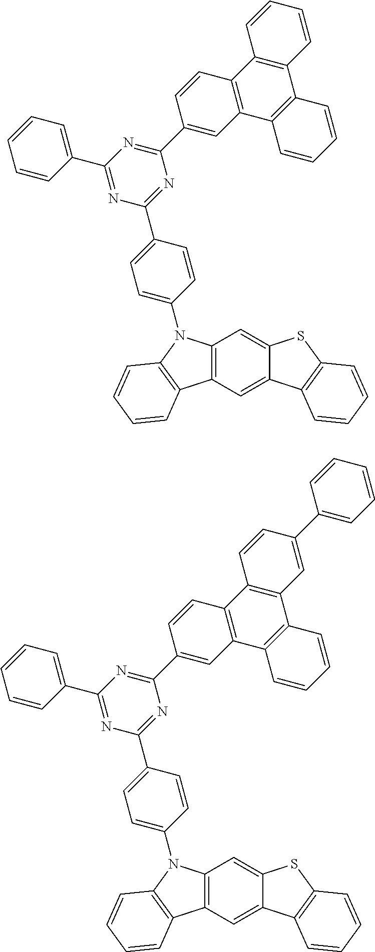 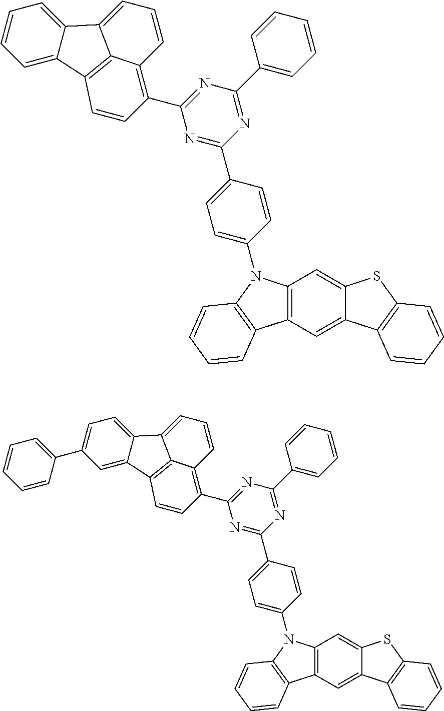 [SMILES: c1ccc(-c2nc(-c3ccc(-n4c5ccccc5c5cc6c(cc54)sc4ccccc46)cc3)nc(-c3ccc4c5c(cccc35)-c3ccccc3-4)n2)cc1 ; c1ccc(-c2ccc3c(c2)-c2ccc(-c4nc(-c5ccccc5)nc(-c5ccc(-n6c7ccccc7c7cc8c(cc76)sc6ccccc68)cc5)n4)c4cccc-3c24)cc1] 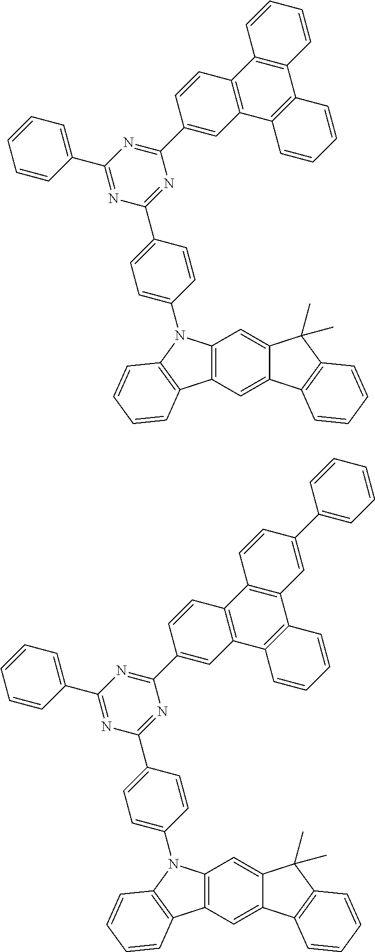 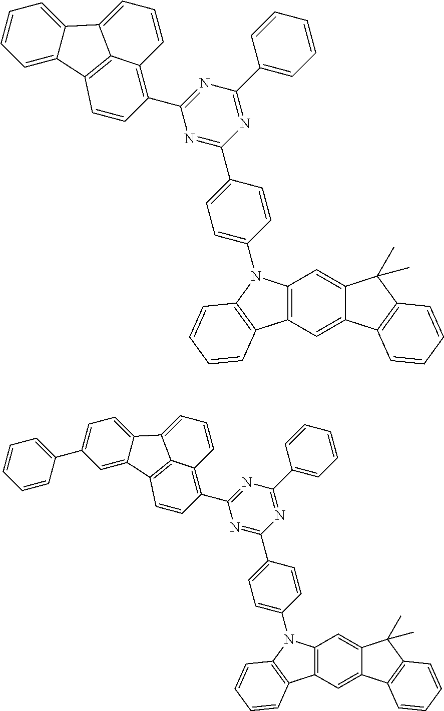 [SMILES: CC1(C)c2ccccc2-c2cc3c4ccccc4n(-c4ccc(-c5nc(-c6ccccc6)nc(-c6ccc7c8c(cccc68)-c6ccccc6-7)n5)cc4)c3cc21 ; CC1(C)c2ccccc2-c2cc3c4ccccc4n(-c4ccc(-c5nc(-c6ccccc6)nc(-c6ccc7c8c(cccc68)-c6ccc(-c8ccccc8)cc6-7)n5)cc4)c3cc21] 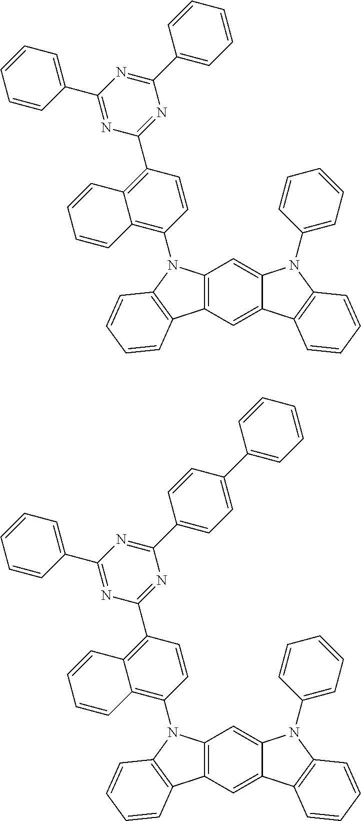 [SMILES: c1ccc(-c2nc(-c3ccccc3)nc(-c3ccc(-n4c5ccccc5c5cc6c7ccccc7n(-c7ccccc7)c6cc54)c4ccccc34)n2)cc1 ; c1ccc(-c2ccc(-c3nc(-c4ccccc4)nc(-c4ccc(-n5c6ccccc6c6cc7c8ccccc8n(-c8ccccc8)c7cc65)c5ccccc45)n3)cc2)cc1] 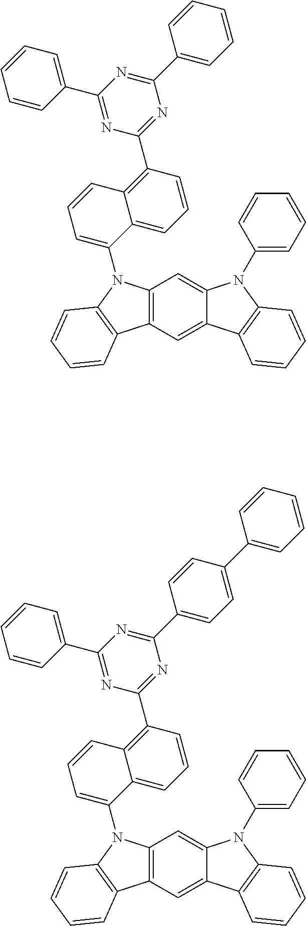 [SMILES: c1ccc(-c2nc(-c3ccccc3)nc(-c3cccc4c(-n5c6ccccc6c6cc7c8ccccc8n(-c8ccccc8)c7cc65)cccc34)n2)cc1 ; c1ccc(-c2ccc(-c3nc(-c4ccccc4)nc(-c4cccc5c(-n6c7ccccc7c7cc8c9ccccc9n(-c9ccccc9)c8cc76)cccc45)n3)cc2)cc1] 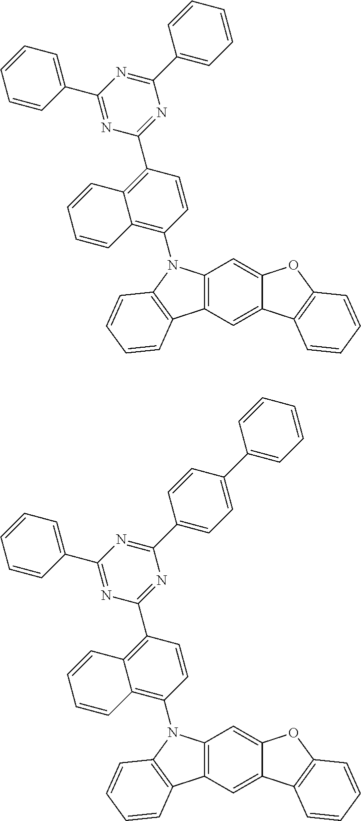 [SMILES: c1ccc(-c2nc(-c3ccccc3)nc(-c3ccc(-n4c5ccccc5c5cc6c(cc54)oc4ccccc46)c4ccccc34)n2)cc1 ; c1ccc(-c2ccc(-c3nc(-c4ccccc4)nc(-c4ccc(-n5c6ccccc6c6cc7c(cc65)oc5ccccc57)c5ccccc45)n3)cc2)cc1] 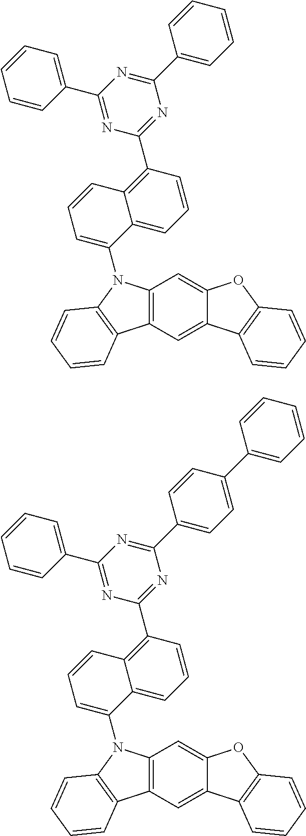 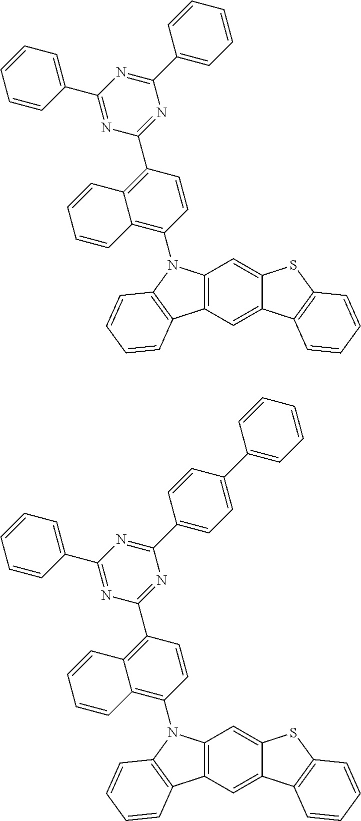 [SMILES: c1ccc(-c2nc(-c3ccccc3)nc(-c3ccc(-n4c5ccccc5c5cc6c(cc54)sc4ccccc46)c4ccccc34)n2)cc1 ; c1ccc(-c2ccc(-c3nc(-c4ccccc4)nc(-c4ccc(-n5c6ccccc6c6cc7c(cc65)sc5ccccc57)c5ccccc45)n3)cc2)cc1] 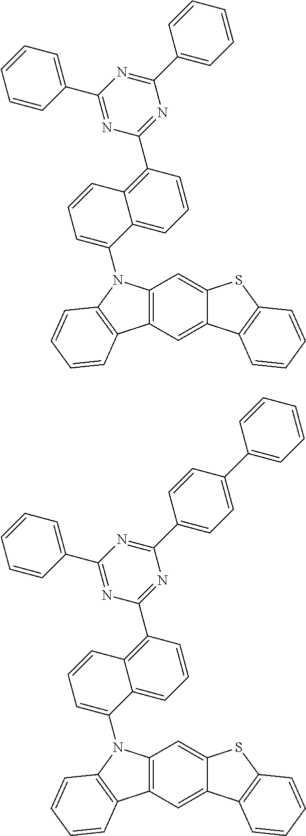 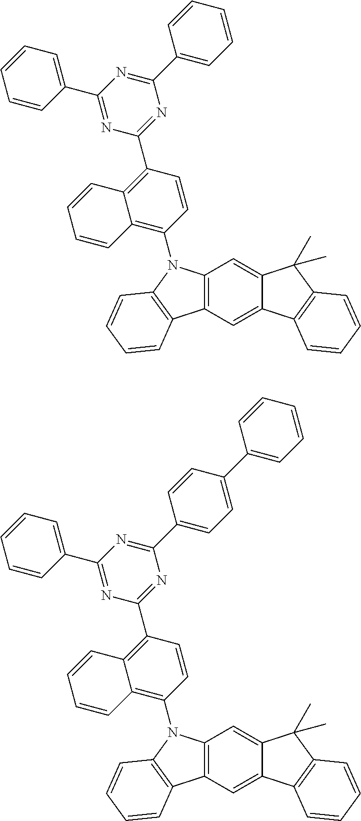 [SMILES: CC1(C)c2ccccc2-c2cc3c4ccccc4n(-c4ccc(-c5nc(-c6ccccc6)nc(-c6ccccc6)n5)c5ccccc45)c3cc21 ; CC1(C)c2ccccc2-c2cc3c4ccccc4n(-c4ccc(-c5nc(-c6ccccc6)nc(-c6ccc(-c7ccccc7)cc6)n5)c5ccccc45)c3cc21] 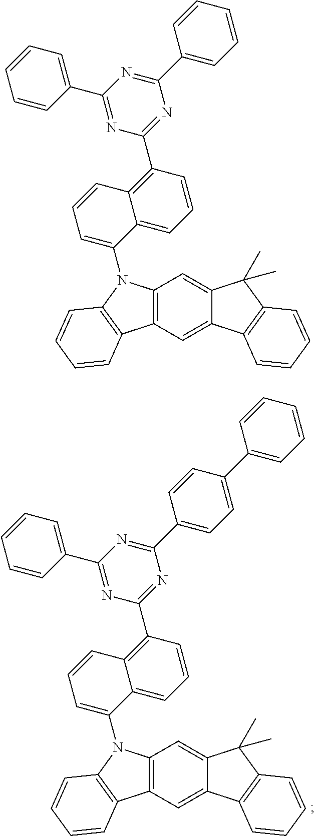
the “substituted” means that hydrogen atoms in the above compounds are each independently substituted with deuterium atoms.

7. A compound, comprising the following compounds: 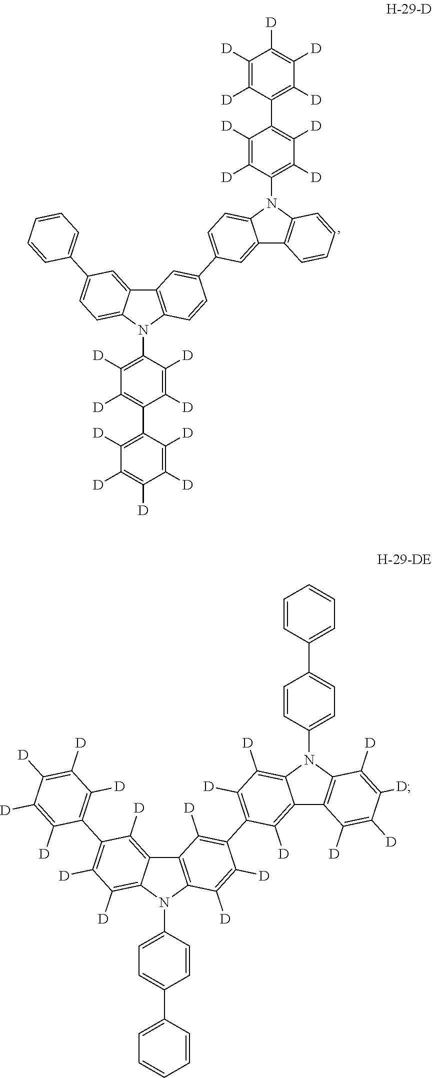
wherein the compound is used for preparing the composition according to claim 1.

8. An intermediate, comprising the following compounds: 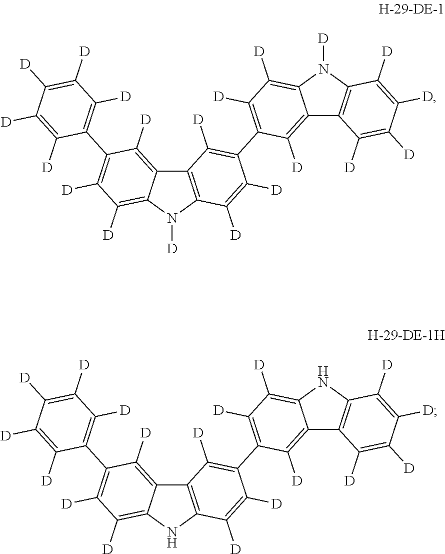
wherein the intermediate is used for preparing the compound represented by Formula I in a composition according to claim 1.

9. An organic electroluminescent device, comprising an anode, a cathode, and an organic thin film layer arranged between the anode and the cathode;
wherein a material of the organic thin film layer comprises the composition according to claim 1;
optionally, the organic thin film layer comprises an emissive layer, and a material of the emissive layer comprises the composition according to claim 1;
optionally, the organic thin film layer comprises a hole layer;
optionally, the hole layer comprises an electron blocking layer; a material of the electron blocking layer comprises a spirofluorene compound;
the spirofluorene compound has a specific structure as represented by Formula III below: 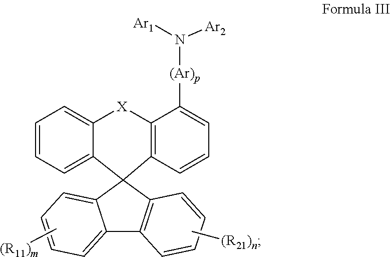
wherein X is selected from O or S;
R₁₁ and R₂₁ are each independently selected from hydrogen, deuterium, fluorine, CN, substituted or unsubstituted C1 to C20 linear or branched alkyl, substituted or unsubstituted C1 to C20 alkoxy, or substituted or unsubstituted C6 to C40 aryl;
Ar is selected from substituted or unsubstituted C6 to C40 arylene;
Ar₁ and Ar₂ are each independently selected from substituted or unsubstituted C6 to C40 aryl, substituted or unsubstituted C12 to C40 oxa-aryl, or substituted or unsubstituted C12 to C40 thioaryl, and at least one of Ar₁ or Ar₂ is selected from any one of phenyl, naphthyl, triphenylenyl, or fluoranthenyl;
p is selected from 0 or 1; and
m and n are each independently selected from an integer from 0 to 4.

10. A display apparatus, comprising the organic electroluminescent device according to claim 9.
